(19) United States
(12) Patent Application Publication
(10) Pub. No.: US 2026/0229358 A1
(43) Pub. Date: Aug. 6, 2026

(54) METHODS AND SYSTEMS FOR DETERMINING GENE SETS FOR DIAGNOSIS AND TREATMENT OF DISEASE STATES

(71) Applicant: AMPEL BioSolutions, LLC, Charlottesville, VA (US)

(72) Inventors: Andrea DAAMEN, Charlottesville, VA (US); Prathyusha BACHALI, Charlottesville, VA (US); Amrie C. GRAMMER, Charlottesville, VA (US); Peter E. LIPSKY, Charlottesville, VA (US)

(21) Appl. No.: 19/317,559

(22) Filed: Sep. 3, 2025

Related U.S. Application Data
(63) Continuation of application No. PCT/US2024/017824, filed on Feb. 29, 2024.
(60) Provisional application No. 63/449,882, filed on Mar. 3, 2023.

(51) Int. Cl. G16H 50/20

(52) U.S. Cl. CPC G16H 50/20

(57) ABSTRACT

The present disclosure provides systems and methods for obtaining a gene set capable of classifying a patient between a first state and a second state. The method comprises training a first machine learning model to select N gene modules from a plurality of gene modules, optionally removing redundant genes from genes within the N gene modules to obtain a first gene set, training a second machine learning model to select M genes from the genes within the first gene set, wherein the selected M genes form the gene set capable of classifying the patient between the first state and the second state. The gene set can be used to diagnose and/or treat a patient classified between the first state and the second state.

### CROSS REFERENCE

This application is a continuation application of International Patent Application No. PCT/US2024/017824, filed Feb. 29, 2024, which claims priority from U.S. Provisional Application No. 63/449,882 filed on Mar. 3, 2023, which are incorporated herein by reference in their entirety.

### BACKGROUND

Recent advances in computer science and the use of artificial intelligence and machine learning for clinical applications offer a promising approach to identify biomarkers capable of diagnose a disease state in a patient. However, small sets of biomarkers, such as gene sets, that can be used to diagnose, and predict course and/or responsiveness to therapy for various disease states are still lacking. There is a need for small sets of biomarkers that can allow for rapid diagnosis and therapeutic intervention of a various disease states.

### SUMMARY

One aspect of the present invention is directed to a method for obtaining a gene set capable of classifying a patient between a first state and a second state. The method can include any one of, any combination of, or all of steps (a) to (e). Step (a) can include training a first machine learning model based on a first data set comprising or derived from gene expression measurements of genes within a plurality of gene modules, to classify a plurality of reference patients between the first state and the second state. The gene expression measurements can be obtained from a plurality of reference biological samples derived or obtained from the plurality of reference patients. A reference biological sample can be obtained from each reference patient. Gene expression measurements of the genes within the plurality of gene modules can be obtained from each reference biological sample. Step (b) can include selecting N gene modules from the plurality of gene modules. Optional step (c) can include removing redundant genes from genes within the N gene modules to obtain a first gene set. Step (d) can include training a second machine learning model based on a second data set comprising gene expression measurements of genes within the first gene set to classify the plurality of reference patients between the first state and the second state. In certain embodiments, step (c) is not performed, wherein the first gene set comprises the genes within the N gene modules. Step (e) can include selecting M genes from the genes within the first gene set. The M genes can be selected from the genes within the first gene set based on feature importance values and/or SHAP values of input features of the second machine learning model for classifying the plurality of reference patients between the first state and the second state. M can be an integer. The selected M genes can form the gene set capable of classifying the patient between the first state and the second state. A first portion of the plurality of the reference patients can belong to the first state and a second portion of the plurality of the reference patients can belong to the second state. The plurality of gene modules can form the input features of the first machine learning model. For each gene module of the plurality of gene modules, the gene expression and/or enrichment value of the gene module in a reference biological sample of the plurality of reference biological samples, forms input feature value of the gene module for the reference biological sample, for the training of the first machine learning model. In certain embodiments, for each gene module of the plurality of gene modules, enrichment value of the gene module in a reference biological sample of the plurality of reference biological samples, forms input feature value of the respective gene module for the reference biological sample, for the training of the first machine learning model. The enrichment value of a gene module in a reference biological sample can be determined from the gene expression measurements of the genes within the plurality of the gene module in the reference biological sample, using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log2 expression analysis, or any combination thereof. In certain embodiments, the enrichment value of a gene module in a reference biological sample can be determined from the gene expression measurements of the genes within the plurality of the gene modules in the reference biological sample, using GSVA wherein the enrichment value can be GSVA score. Enrichment value of a gene module in a reference biological sample can be with respect to the cohort (e.g., plurality of the reference biological sample). In certain embodiments, for each gene module of the plurality of gene modules, gene expression value of the gene module in a reference biological sample of the plurality of reference biological samples, forms input feature value of the gene module for the reference biological sample, for the training of the first machine learning model. In certain embodiments, the gene expression value of a gene module in a reference biological sample can be eigengene (ME) of the gene module in the reference biological sample. The method can be performed and/or implemented in a computer.

In certain embodiments, the N gene modules can be selected from the plurality of gene modules based on feature importance values and/or SHAP values (e.g., global SHAP values) of input features (e.g., the plurality of gene modules) of the first machine learning model for classifying the plurality of reference patients between the first state and the second state. N can be an integer. In certain embodiments, the N gene modules are selected from the plurality of gene modules based on the feature importance values of the input features of the first machine learning model for classifying the plurality of reference patients between the first state and the second state. In certain embodiments, the N gene modules are selected from the plurality of gene modules based on the SHAP values of the input features of the first machine learning model for classifying the plurality of reference patients between the first state and the second state. In certain embodiments, the N gene modules have top N feature importance values and/or SHAP values among the input features of the first machine learning model for classifying the plurality of reference patients between the first state and the second state. In certain embodiments, the N gene modules have (e.g., are the gene modules having) top N feature importance values among the feature importance values of the input features of the first machine learning model for classifying the plurality of reference patients between the first state and the second state. In certain embodiments, the N gene modules have top N SHAP values among the SHAP values of the input features of the first machine learning model for classifying the plurality of reference patients between the first state and the second state. As a non-limiting example, N can be 10, wherein gene modules having top 10 feature importance values and/or SHAP values among the input features (e.g., the plurality of gene modules of step (a)) of the first machine learning model for classifying the plurality of reference patients between the first state and the second state are selected in step (b). In certain embodiments, the first machine learning model is trained using multiple machine learning algorithms, and N features can be selected based on the features importance values obtained using multiple machine learning algorithms. Gene modules selected in step (b) may or may not contain any additional gene modules (e.g., from the plurality of gene modules) over the N gene modules. In certain embodiments, the plurality of gene modules contains X gene modules, wherein X is an integer, and N/X can be 0.05 to 0.4.

The genes removed in step (c), can be determined to be redundant with respect to one or more genes (e.g., within the genes within the N gene modules), based on expression of the genes in the plurality of reference biological samples. In certain embodiments, genes removed in step (c) can have a correlation coefficient greater than a threshold value. The correlation coefficient can be determined based on the expression of the genes (e.g., within the genes within the N gene modules), in the plurality of reference biological samples. The threshold value can be 0.6, 0.65, 0.7, 0.75, 0.8, 0.85, 0.9 or 0.95. In certain embodiments, the threshold value is 0.8.

The genes in the first gene set can form the input features of the second machine learning model. For each gene in the first gene set, expression (e.g., gene expression) value of the gene in a reference biological sample of the plurality of reference biological samples, can form input feature value of the respective gene for the reference biological sample, for the training of the second machine learning model. In certain embodiments, the expression value can be a log2 expression value. M genes can be selected based on the feature importance values and/or SHAP values of the input features of the second machine learning model for classifying the plurality of reference patients between the first state and the second state. In certain embodiments, the M genes have (e.g., are the genes having) top M feature importance values and/or SHAP values (e.g., global SHAP values) among the input features of the second machine learning model for classifying the plurality of reference patients between the first state and the second state. In certain embodiments, the M genes have top M feature importance values among the feature importance values of the input features of the second machine learning model for classifying the plurality of reference patients between the first state and the second state. In certain embodiments, the M genes have top M SHAP values among the SHAP values of the input features of the second machine learning model for classifying the plurality of reference patients between the first state and the second state. In certain embodiments, the second machine learning model is trained using multiple machine learning algorithms, and M genes can be selected based on the features importance values obtained using multiple machine learning algorithms. Genes selected in step (e) may or may not contain any additional genes (e.g., from the first gene set) over the M genes.

M can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or any range therebetween. In certain embodiments, M is 10. In certain embodiments, M is 15. In certain embodiments, M is 20. In certain embodiments, M is 25. In certain embodiments, M is 30.

The plurality of biological samples can comprise blood samples, isolated peripheral blood mononuclear cells (PBMCs), tissue biopsy samples, nasal fluids, saliva, urine, stool, or any derivative thereof. In certain embodiments, the plurality of biological samples comprises blood samples or any derivative thereof. In certain embodiments, the plurality of biological samples comprises PBMCs or any derivative thereof. In certain embodiments, the plurality of biological samples comprises tissue biopsy samples or any derivative thereof. In certain embodiments, the tissue biopsy samples comprise skin biopsy samples. In certain embodiments, the plurality of biological samples comprises nasal fluid samples or any derivative thereof. In certain embodiments, the plurality of biological samples comprises saliva samples or any derivative thereof. In certain embodiments, the plurality of biological samples comprises urine samples or any derivative thereof.

In some embodiments, the first state and second state are disease states. In some embodiments, the first state is having a disease state, and the second state is not having the disease state. In some embodiments, the first state is responsiveness to a drug for a disease state, and the second state nonresponsiveness to the drug for the disease state. In some embodiments, the disease states are inflammatory disease states. In some embodiments, the disease states are infectious disease states. In certain embodiments, the first state is having COVID-19 disease, the second state is not having COVID-19 disease, and the plurality of gene modules are the gene modules listed in Table 1. In certain embodiments, the first state is having COVID-19 disease, the second state is not having COVID-19 disease, and the plurality of gene modules are the gene modules listed in Table 1, wherein the plurality of reference patients are ICU patients. In certain embodiments, the first state is having critical COVID-19 disease, second state is having non-critical COVID-19 disease, and the plurality of gene modules are the gene modules listed in Table 1. In certain embodiments, the first state is having non-critical COVID-19 disease, second state is not having COVID-19 disease, and the plurality of gene modules are the gene modules listed in Table 1.

In certain embodiments, the first state is having lupus, the second state is not having lupus, and the plurality of gene modules are the gene modules listed in Table 9. In certain embodiments, the first state is having psoriasis, the second state is not having psoriasis, and the plurality of gene modules are the gene modules listed in Table 9. In certain embodiments, the first state is having atopic dermatitis, the second state is not having atopic dermatitis, and the plurality of gene modules are the gene modules listed in Table 9. In certain embodiments, the first state is having systemic sclerosis, the second state is not having systemic sclerosis, and the plurality of gene modules are the gene modules listed in Table 9. In certain embodiments, the first state is having lupus, the second state is having psoriasis, and the plurality of gene modules are the gene modules listed in Table 9. In certain embodiments, the first state is having lupus, the second state is having atopic dermatitis, and the plurality of gene modules are the gene modules listed in Table 9. In certain embodiments, the first state is having lupus, the second state is having systemic sclerosis, and the plurality of gene modules are the gene modules listed in Table 9.

In certain embodiments, the first state is having lupus, the second state is not having lupus, and the plurality of gene modules are the gene modules listed in Table 10. In certain embodiments, the first state is responsiveness to a lupus drug selected from a neutrophil function inhibitor, a TNF inhibitor, an IL1 inhibitor, a Plasma cell inhibitor or deleter, a NK cell inhibitor or deleter, or a B Cell inhibitor or deleter, the second state is non-responsiveness to a lupus drug selected from neutrophil function inhibitor, a TNF inhibitor, an IL1 inhibitor, a Plasma cell inhibitor or deleter, a NK cell inhibitor or deleter, or a B Cell inhibitor or deleter, and the plurality of gene modules are the gene modules listed in Table 10. In certain embodiments, the first state is responsiveness to a lupus drug selected from a neutrophil function inhibitor, a TNF inhibitor, an IL1 inhibitor, a Plasma cell inhibitor, a NK cell inhibitor, or a B Cell Inhibitor, the second state is nonresponsiveness to a lupus drug selected from neutrophil function inhibitor, a TNF inhibitor, an IL1 inhibitor, a Plasma cell inhibitor, a NK cell inhibitor, or a B Cell Inhibitor, and the plurality of gene modules are the gene modules listed in Table 10, wherein the plurality of reference patients have lupus.

A lupus patient can be classified as responsive or nonresponsive to a lupus drug. In some embodiments, a lupus disease condition of the lupus patient responsive to the lupus drug improves with administration of the drug to the patient. In some embodiments, a lupus disease condition of the lupus patient nonresponsive to the lupus drug does not improve with administration of the drug to the patient. In some embodiments, a disease condition of a patient responsive to a drug for the disease condition improves with administration of the drug to the patient. In some embodiments, a disease condition of a patient nonresponsive to the drug for the disease condition does not improve with administration of the drug to the patient.

Non-limiting examples of a plasma cell inhibitor or deleter comprises Mycophenolate, Bortezomib, Carfilzomib, Ixazomib, Daratumumab, Isatuximab, Elotuzumab, or any combinations thereof. Non-limiting examples of an IL1 inhibitor comprises Anakinra, Canakinumab, or any combinations thereof. Non-limiting examples of a TNF inhibitor comprises Adalimumab, Certolizumab pegol, Etanercept, Golimumab, Infliximab, or any combinations thereof. Non-limiting examples of a Neutrophil function inhibitor comprise Dasatinib, Apremilast, Roflumilast, or any combinations thereof. Non-limiting examples of a NK cell inhibitor or deleter comprises Azathioprine. Non-limiting examples of a B cell inhibitor or deleter comprises Belimumab, Rituximab, Obinutuzumab, Ocrelizumab, Ofatumumab, Inebilizumab, or any combinations thereof.

Certain aspects are directed to a method for classifying a patient between the first state and the second state. The method can include analyzing a patient data set comprising gene expression measurements of the 2 or more genes of a gene set obtained using the method containing the steps (a), (b), (c), (d) and/or (e), as described herein, from a biological sample obtained or derived from the patient. In certain embodiments, analyzing the patient data set can comprise providing the patient data set as an input to a machine learning model trained to generate an inference of whether the patient data set is indicative of the patient belonging to the first state or the patient belonging to the second state. The inference can be whether the patient data set is indicative of patient classification to the first state, or patient classification to the second state. In certain embodiments, the method further comprise receiving, as an output of the machine-learning model, the inference; and/or electronically outputting a report classifying the patient between the first state and the second state. The biological samples can comprise a blood sample, isolated peripheral blood mononuclear cells (PBMCs), a tissue biopsy sample, a nasal fluid sample, a saliva sample, a urine sample, a stool sample, or any derivative thereof. In certain embodiments, the biological samples comprise a blood sample or any derivative thereof. In certain embodiments, the biological sample comprise PBMCs or any derivative thereof. In certain embodiments, the biological sample comprise a tissue biopsy sample or any derivative thereof. In certain embodiments, the tissue biopsy sample comprise a skin biopsy sample. In certain embodiments, the method further comprises recommending, selecting and/or administering a treatment to the patient based on the classification of the patient. In certain embodiments, the method further comprises administering a treatment to the patient based on the classification of the patient.

Another aspect of the present disclosure provides a non-transitory computer-readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### BRIEF DESCRIPTION OF THE DRAWINGS

The patent application file contains at least one drawing executed in color. Copies of this patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: A flow diagram according to one example of the disclosure showing of the iterative ML pipeline according to one example of the disclosure. Graphic depicting steps in the iterative ML pipeline used for classification of COVID-19 patients. GSVA scores of gene modules representing immune cells and pathways were used as input for 9 different ML algorithms in 2 iterations after which a final ML iteration used z-score normalized gene expression values.

FIGS. 2A-B: Normalized gene expression values from classification of all COVID patients vs healthy individuals. FIG. 2A show pre- and post-normalization expression values for the dataset combinations are summarized as boxplots. FIG. 2B shows z-score normalization expression values of the final 20 genes with greatest performance in classifying COVID-19 patients from healthy individuals.

FIGS. 3A-B: Normalized gene expression values from classification of non-critical COVID patients vs healthy individuals. FIG. 3A show pre- and post-normalization expression values for the dataset combinations are summarized as boxplots. FIG. 3B shows z-score normalization expression values of the final 20 genes with greatest performance in classifying non-critical COVID patients vs healthy individuals.

FIGS. 4A-B: Normalized gene expression values from classification of critical vs non-critical COVID patients. FIG. 4A show pre- and post-normalization expression values for the dataset combinations are summarized as boxplots. FIG. 4B shows z-score normalization expression values of the final 20 genes with greatest performance in classifying critical vs non-critical COVID patients.

FIGS. 5A-B: Normalized gene expression values from classification of COVID vs non-COVID ICU patients. FIG. 5A show pre- and post-normalization expression values for the dataset combinations are summarized as boxplots. FIG. 5B shows z-score normalization expression values of the final 20 genes with greatest performance in classifying COVID vs non-COVID ICU patients.

FIGS. 6A-E: Iterative ML COVID-19 patient vs healthy individual classifier results. FIG. 6A. Representative ROC and PR curves from the first ML pipeline iteration for the all COVID-19 patients vs healthy control classifier. FIG. 6B. Bar graph of feature importance values for the top 10 gene modules resulting from the second ML pipeline iteration. FIG. 6C. Representative ROC and PR curves from the third ML pipeline iteration for the all COVID-19 patients vs healthy control classifier. FIG. 6D. Top 20 gene features resulting from the third ML pipeline iteration. FIG. 6E. SHAP summary plot depicting the magnitude and directionality of relationships between the top 20 gene features and COVID-19 classification.

FIGS. 7A-E: Iterative ML non-critical COVID-19 patient vs healthy individual classifier results. FIG. 7A. Representative ROC and PR curves from the first ML pipeline iteration for the non-critical COVID-19 patient vs healthy control classifier. FIG. 7B. Bar graph of feature importance values for the top 10 gene modules resulting from the second ML pipeline iteration. FIG. 7C. Representative ROC and PR curves from the third ML pipeline iteration for the non-critical COVID-19 patient vs healthy control classifier. FIG. 7D. Top 20 gene features resulting from the third ML pipeline iteration. FIG. 7E. SHAP summary plot depicting the magnitude and directionality of relationships between the top 20 gene features and non-critical COVID-19 classification.

FIGS. 8A-E: Iterative ML critical vs non-critical COVID-19 patient classifier results. FIG. 8A. Representative ROC and PR curves from the first ML pipeline iteration for the critical vs non-critical COVID-19 patient classifier. FIG. 8B. Bar graph of feature importance values for the top 10 gene modules resulting from the second ML pipeline iteration. FIG. 8C. Representative ROC and PR curves from the third ML pipeline iteration for critical vs non-critical COVID-19 patient classifier. FIG. 8D. Top 20 gene features resulting from the third ML pipeline iteration. FIG. 8E. SHAP summary plot depicting the magnitude and directionality of relationships between the top 20 gene features and critical COVID-19 classification.

FIG. 9A-E: Iterative ML COVID-19 vs non-COVID-19 ICU patient classifier results. FIG. 9A. Representative ROC and PR curves from the first ML pipeline iteration for the COVID-19 vs non-COVID-19 ICU patient classifier. FIG. 9B. Bar graph of feature importance values for the top 10 gene modules resulting from the second ML pipeline iteration. FIG. 9C. Representative ROC and PR curves from the third ML pipeline iteration for the COVID-19 vs non-COVID-19 ICU patient classifier. FIG. 9D. Top 20 gene features resulting from the third ML pipeline iteration. FIG. 9E. SHAP summary plot depicting the magnitude and directionality of relationships between the top 20 gene features and COVID-19 ICU classification.

FIG. 10A-B: Critical vs non-Critical COVID-19 classifier gene signature validation results. FIG. 10A, ROC and PR curves and FIG. 10B, table of performance metrics for ML results using the 20 gene signature from FIG. 8D to compare ICU and non-ICU COVID-19 patients in an independent validation cohort.

FIG. 11: A computer system 1601 that is programmed or otherwise configured to, for example, perform methods of the disclosure.

### DETAILED DESCRIPTION

Unless otherwise defined, all technical terms used herein have the same meaning as commonly understood by one of ordinary skill in the art to which this disclosure belongs.

### Certain Terms

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a,” “an,” and “the” include plural references unless the context clearly dictates otherwise. Any reference to “or” herein is intended to encompass “and/or” unless otherwise stated.

A “plurality” may refer to two or more. For example, a plurality of genes, e.g., COVID-19 disease-associated genes, may refer to 2 to at least 500 genes. In some embodiments, a data set used in the methods and systems described herein comprises gene expression measurements of a biological sample of each of about 2 genes to about 500 genes. In some embodiments, a data set used in the methods and systems described herein comprises gene expression measurements of a biological sample of each of about 2 genes to about 5 genes, about 2 genes to about 10 genes, about 2 genes to about 15 genes, about 2 genes to about 20 genes, about 2 genes to about 25 genes, about 2 genes to about 50 genes, about 2 genes to about 75 genes, about 2 genes to about 100 genes, about 2 genes to about 200 genes, about 2 genes to about 300 genes, about 2 genes to about 500 genes, about 5 genes to about 10 genes, about 5 genes to about 15 genes, about 5 genes to about 20 genes, about 5 genes to about 25 genes, about 5 genes to about 50 genes, about 5 genes to about 75 genes, about 5 genes to about 100 genes, about 5 genes to about 200 genes, about 5 genes to about 300 genes, about 5 genes to about 500 genes, about 10 genes to about 15 genes, about 10 genes to about 20 genes, about 10 genes to about 25 genes, about 10 genes to about 50 genes, about 10 genes to about 75 genes, about 10 genes to about 100 genes, about 10 genes to about 200 genes, about 10 genes to about 300 genes, about 10 genes to about 500 genes, about 15 genes to about 20 genes, about 15 genes to about 25 genes, about 15 genes to about 50 genes, about 15 genes to about 75 genes, about 15 genes to about 100 genes, about 15 genes to about 200 genes, about 15 genes to about 300 genes, about 15 genes to about 500 genes, about 20 genes to about 25 genes, about 20 genes to about 50 genes, about 20 genes to about 75 genes, about 20 genes to about 100 genes, about 20 genes to about 200 genes, about 20 genes to about 300 genes, about 20 genes to about 500 genes, about 25 genes to about 50 genes, about 25 genes to about 75 genes, about 25 genes to about 100 genes, about 25 genes to about 200 genes, about 25 genes to about 300 genes, about 25 genes to about 500 genes, about 50 genes to about 75 genes, about 50 genes to about 100 genes, about 50 genes to about 200 genes, about 50 genes to about 300 genes, about 50 genes to about 500 genes, about 75 genes to about 100 genes, about 75 genes to about 200 genes, about 75 genes to about 300 genes, about 75 genes to about 500 genes, about 100 genes to about 200 genes, about 100 genes to about 300 genes, about 100 genes to about 500 genes, about 200 genes to about 300 genes, about 200 genes to about 500 genes, or about 300 genes to about 500 genes. In some embodiments, a data set used in the methods and systems described herein comprises gene expression measurements of a biological sample of each of about 2 genes, about 5 genes, about 10 genes, about 15 genes, about 20 genes, about 25 genes, about 50 genes, about 75 genes, about 100 genes, about 200 genes, about 300 genes, or about 500 genes. In some embodiments, a data set used in the methods and systems described herein comprises gene expression measurements of a biological sample of each of at least about 2 genes, about 5 genes, about 10 genes, about 15 genes, about 20 genes, about 25 genes, about 50 genes, about 75 genes, about 100 genes, about 200 genes, or about 300 genes. In some embodiments, a data set used in the methods and systems described herein comprises gene expression measurements of a biological sample of each of at most about 5 genes, about 10 genes, about 15 genes, about 20 genes, about 25 genes, about 50 genes, about 75 genes, about 100 genes, about 200 genes, about 300 genes, or about 500 genes.

As used herein, the term “subject” refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject may be displaying a symptom(s) indicative of a health or physiological state or condition of the subject, such as a disease or disorder of the subject. As an alternative, the subject can be asymptomatic with respect to such health or physiological state or condition.

As used herein, the term “sample,” generally refers to a biological sample obtained from or derived from one or more subjects. Biological samples may be processed or fractionated before further analysis. Biological samples may include a whole blood (WB) sample, a PBMC sample, a tissue sample, a purified cell sample, Bronchoalveolar lavage, nasal fluid, or derivatives thereof. In some embodiments, a whole blood sample may be purified to obtain the purified cell sample. The term “derived from” used herein refers to an origin or source, and may include naturally occurring, recombinant, unpurified or purified molecules.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

As used herein the term “diagnose,” “diagnosis,” “determine,” or “determining” of a status or outcome includes predicting or diagnosing the status or outcome, determining predisposition to a status or outcome, monitoring treatment of patient, diagnosing a therapeutic response of a patient, and prognosis of status or outcome, progression, and response to particular treatment.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and assayed, and then another sample can be taken at a subsequent time point and assayed. Such methods can be used, for example, for longitudinal monitoring purposes to track the development or progression of a disease. In some embodiments, the progression of a disease can be tracked before treatment, after treatment, or during the course of treatment, to determine the treatment's effectiveness. For example, a method as described herein can be performed on a subject prior to, and after, treatment with a disease state or condition therapy to measure the disease's progression or regression in response to the disease state or condition therapy.

After obtaining a sample from the subject, the sample may be processed to generate datasets indicative of a disease or disorder of the subject. For example, a presence, absence, or quantitative assessment of nucleic acid molecules of the sample at a panel of disease state or condition-associated or interferon-associated genes or may be indicative of a disease state or condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

COVID-19 patients can be categorized as “non-critical” if they exhibit symptoms, but are not hospitalized, does not require hospitalization, are admitted to non-critical care ward, or does not require admittance to ICU. COVID-19 patients can be categorized as “critical” if they exhibit more severe symptoms requiring hospitalization and admittance to the ICU.

The sample may be processed without any nucleic acid extraction. For example, the disease or disorder may be identified or monitored in the subject by using probes configured to selectively enrich nucleic acid (e.g., RNA or DNA) molecules corresponding to a panel of disease state or condition-associated or interferon-associated genes. The probes may be nucleic acid primers. The probes may have sequence complementarity with nucleic acid sequences from one or more of the panel of disease state or condition-associated or interferon-associated genes. The panel of disease state or condition-associated or interferon-associated genes may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more disease state or condition-associated or interferon-associated genes.

The probes may be nucleic acid molecules (e.g., RNA or DNA) having sequence complementarity with nucleic acid sequences (e.g., RNA or DNA) of one or more genes (e.g., disease state or condition-associated or interferon-associated genes). These nucleic acid molecules may be primers or enrichment sequences. The assaying of the sample using probes that are selective for the one or more genes (e.g., disease state or condition-associated or interferon-associated genes) or RNA transcripts therefrom may comprise use of array hybridization, polymerase chain reaction (PCR), or nucleic acid sequencing (e.g., RNA sequencing or DNA sequencing, such as RNA-Seq).

The assay readouts may be quantified gene expression measurements from one or more gene (e.g., disease state or condition-associated or interferon-associated genes) to generate the data indicative of the disease or disorder. For example, quantification of array hybridization or polymerase chain reaction (PCR) corresponding to expression from a plurality of genes (e.g., disease state or condition-associated or interferon-associated genes) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

### Methods for Obtaining a Gene Set Capable of Classifying a Patient Between a First State and a Second State.

One aspect of the present invention is directed to a method for obtaining a gene set capable of classifying a patient between a first state and a second state. The method can include any one of, any combination of, or all of steps (a) to (e). Step (a) can include training a first machine learning model based on a first data set comprising or derived from gene expression measurements of genes within a plurality of gene modules, to classify a plurality of reference patients between the first state and the second state. The gene expression measurements can be obtained from a plurality of reference biological samples derived or obtained from the plurality of reference patients. A reference biological sample can be obtained from each reference patients. Gene expression measurements of the genes within the plurality of gene modules can be obtained from each reference biological samples. Step (b) can include selecting N gene modules from the plurality of gene modules. Optional step (c) can include removing redundant genes from genes within the N gene modules to obtain a first gene set. Step (d) can include training a second machine learning model based on a second data set comprising gene expression measurements of genes within the first gene set to classify the plurality of reference patients between the first state and the second state. In certain embodiments, step (c) is not performed, wherein the first gene set comprises the genes within the N gene modules. Step (e) can include selecting M genes from the genes within the first gene set. The M genes can be selected from the genes within the first gene set based on feature importance values and/or SHAP values of input features of the second machine learning model for classifying the plurality of reference patients between the first state and the second state. M can be an integer. The M genes can form the gene set capable of classifying the patient between the first state and the second state. A first portion of the plurality of reference patients can belong to the first state and a second portion of the plurality of reference patients can belong to the second state. The plurality of gene modules forms the input features of the first machine learning model. For each gene module of the plurality of gene modules, gene expression and/or enrichment value of the gene module in a reference biological sample of the plurality of reference biological samples, forms input feature value of the gene module for the reference biological sample, for the training of the first machine learning model. In certain embodiments, for each gene module of the plurality of gene modules, enrichment value of the gene module in a reference biological sample of the plurality of reference biological samples, forms input feature value of the respective gene module for the reference biological sample, for the training of the first machine learning model. The enrichment value of a gene module in a reference biological sample can be determined from the gene expression measurements of genes within the gene module in the reference biological sample, using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log2 expression analysis, or any combination thereof. In certain embodiments, the enrichment value of a gene module in a reference biological sample can be determined from the gene expression measurements of genes within the gene module in the reference biological sample, using GSVA wherein the enrichment value can be GSVA score. Enrichment value of a gene module in a reference biological sample can be with respect to the cohort (e.g., plurality of the reference biological sample). In certain embodiments, for each gene module of the plurality of gene modules, gene expression value of the gene module in a reference biological sample of the plurality of reference biological samples, forms input feature value of the respective gene module for the reference biological sample, for the training of the first machine learning model. In certain embodiments, the gene expression value of a gene module in a reference biological sample can be eigengene (ME) of the gene module in the reference biological sample. The method can be performed and/or implemented in a computer.

In certain embodiments, the N gene modules can be selected from the plurality of gene modules based on feature importance values and/or SHAP values of input features (e.g., the plurality of gene modules) of the first machine learning model for classifying the plurality of reference patients between the first state and the second state. N can be an integer. In certain embodiments, the N gene modules have top N feature importance values and/or SHAP values among the input features of the first machine learning model for classifying the plurality of reference patients between the first state and the second state. In certain embodiments, the N gene modules have top N feature importance values among the feature importance values of the input features of the first machine learning model for classifying the plurality of reference patients between the first state and the second state. In certain embodiments, the N gene modules have top N SHAP values among the SHAP values of the input features of the first machine learning model for classifying the plurality of reference patients between the first state and the second state. As a non-limiting example, N can be 10, wherein gene modules having top 10 feature importance values and/or SHAP values among the input features (e.g., the plurality of gene modules of step (a)) of the first machine learning model for classifying the plurality of reference patients between the first state and the second state are selected in step (b). Gene modules selected in step (b) may or may not contain any additional gene modules (e.g., from the plurality of gene modules) over the N genes modules. N can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or any range therebetween. In certain embodiments, N is about 2 to about 40. In certain embodiments, N is about 2 to about 3, about 2 to about 5, about 2 to about 8, about 2 to about 10, about 2 to about 12, about 2 to about 15, about 2 to about 18, about 2 to about 20, about 2 to about 25, about 2 to about 30, about 2 to about 40, about 3 to about 5, about 3 to about 8, about 3 to about 10, about 3 to about 12, about 3 to about 15, about 3 to about 18, about 3 to about 20, about 3 to about 25, about 3 to about 30, about 3 to about 40, about 5 to about 8, about 5 to about 10, about 5 to about 12, about 5 to about 15, about 5 to about 18, about 5 to about 20, about 5 to about 25, about 5 to about 30, about 5 to about 40, about 8 to about 10, about 8 to about 12, about 8 to about 15, about 8 to about 18, about 8 to about 20, about 8 to about 25, about 8 to about 30, about 8 to about 40, about 10 to about 12, about 10 to about 15, about 10 to about 18, about 10 to about 20, about 10 to about 25, about 10 to about 30, about 10 to about 40, about 12 to about 15, about 12 to about 18, about 12 to about 20, about 12 to about 25, about 12 to about 30, about 12 to about 40, about 15 to about 18, about 15 to about 20, about 15 to about 25, about 15 to about 30, about 15 to about 40, about 18 to about 20, about 18 to about 25, about 18 to about 30, about 18 to about 40, about 20 to about 25, about 20 to about 30, about 20 to about 40, about 25 to about 30, about 25 to about 40, or about 30 to about 40. In certain embodiments, N is about 2, about 3, about 5, about 8, about 10, about 12, about 15, about 18, about 20, about 25, about 30, or about 40. In certain embodiments, N is at least about 2, about 3, about 5, about 8, about 10, about 12, about 15, about 18, about 20, about 25, or about 30. In certain embodiments, N is at most about 3, about 5, about 8, about 10, about 12, about 15, about 18, about 20, about 25, about 30, or about 40. In certain embodiments, N is about 3 to about 15. In certain embodiments, N is about 3 to about 5, about 3 to about 6, about 3 to about 7, about 3 to about 8, about 3 to about 9, about 3 to about 10, about 3 to about 11, about 3 to about 12, about 3 to about 13, about 3 to about 14, about 3 to about 15, about 5 to about 6, about 5 to about 7, about 5 to about 8, about 5 to about 9, about 5 to about 10, about 5 to about 11, about 5 to about 12, about 5 to about 13, about 5 to about 14, about 5 to about 15, about 6 to about 7, about 6 to about 8, about 6 to about 9, about 6 to about 10, about 6 to about 11, about 6 to about 12, about 6 to about 13, about 6 to about 14, about 6 to about 15, about 7 to about 8, about 7 to about 9, about 7 to about 10, about 7 to about 11, about 7 to about 12, about 7 to about 13, about 7 to about 14, about 7 to about 15, about 8 to about 9, about 8 to about 10, about 8 to about 11, about 8 to about 12, about 8 to about 13, about 8 to about 14, about 8 to about 15, about 9 to about 10, about 9 to about 11, about 9 to about 12, about 9 to about 13, about 9 to about 14, about 9 to about 15, about 10 to about 11, about 10 to about 12, about 10 to about 13, about 10 to about 14, about 10 to about 15, about 11 to about 12, about 11 to about 13, about 11 to about 14, about 11 to about 15, about 12 to about 13, about 12 to about 14, about 12 to about 15, about 13 to about 14, about 13 to about 15, or about 14 to about 15. In certain embodiments, N is about 3, about 5, about 6, about 7, about 8, about 9, about 10, about 11, about 12, about 13, about 14, or about 15. In certain embodiments, N is at least about 3, about 5, about 6, about 7, about 8, about 9, about 10, about 11, about 12, about 13, or about 14. In certain embodiments, N is at most about 5, about 6, about 7, about 8, about 9, about 10, about 11, about 12, about 13, about 14, or about 15. In certain embodiments, Nis 10. In certain embodiments, the plurality of gene modules contains X gene modules, wherein X is an integer, and N/X can be 0.05 to 0.4. N/X can be 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39 or 0.4, or any value or range therebetween. In certain embodiments, N/X is about 0.05 to about 0.4. In certain embodiments, N/X is about 0.05 to about 0.1, about 0.05 to about 0.15, about 0.05 to about 0.17, about 0.05 to about 0.19, about 0.05 to about 0.2, about 0.05 to about 0.21, about 0.05 to about 0.23, about 0.05 to about 0.25, about 0.05 to about 0.3, about 0.05 to about 0.35, about 0.05 to about 0.4, about 0.1 to about 0.15, about 0.1 to about 0.17, about 0.1 to about 0.19, about 0.1 to about 0.2, about 0.1 to about 0.21, about 0.1 to about 0.23, about 0.1 to about 0.25, about 0.1 to about 0.3, about 0.1 to about 0.35, about 0.1 to about 0.4, about 0.15 to about 0.17, about 0.15 to about 0.19, about 0.15 to about 0.2, about 0.15 to about 0.21, about 0.15 to about 0.23, about 0.15 to about 0.25, about 0.15 to about 0.3, about 0.15 to about 0.35, about 0.15 to about 0.4, about 0.17 to about 0.19, about 0.17 to about 0.2, about 0.17 to about 0.21, about 0.17 to about 0.23, about 0.17 to about 0.25, about 0.17 to about 0.3, about 0.17 to about 0.35, about 0.17 to about 0.4, about 0.19 to about 0.2, about 0.19 to about 0.21, about 0.19 to about 0.23, about 0.19 to about 0.25, about 0.19 to about 0.3, about 0.19 to about 0.35, about 0.19 to about 0.4, about 0.2 to about 0.21, about 0.2 to about 0.23, about 0.2 to about 0.25, about 0.2 to about 0.3, about 0.2 to about 0.35, about 0.2 to about 0.4, about 0.21 to about 0.23, about 0.21 to about 0.25, about 0.21 to about 0.3, about 0.21 to about 0.35, about 0.21 to about 0.4, about 0.23 to about 0.25, about 0.23 to about 0.3, about 0.23 to about 0.35, about 0.23 to about 0.4, about 0.25 to about 0.3, about 0.25 to about 0.35, about 0.25 to about 0.4, about 0.3 to about 0.35, about 0.3 to about 0.4, or about 0.35 to about 0.4. In certain embodiments, N/X is about 0.05, about 0.1, about 0.15, about 0.17, about 0.19, about 0.2, about 0.21, about 0.23, about 0.25, about 0.3, about 0.35, or about 0.4. In certain embodiments, N/X is at least about 0.05, about 0.1, about 0.15, about 0.17, about 0.19, about 0.2, about 0.21, about 0.23, about 0.25, about 0.3, or about 0.35. In certain embodiments, N/X is at most about 0.1, about 0.15, about 0.17, about 0.19, about 0.2, about 0.21, about 0.23, about 0.25, about 0.3, about 0.35, or about 0.4. In certain embodiments, the first machine learning model is trained using multiple machine learning algorithms, and N features can be selected based on the feature importance values and/or SHAP values obtained using multiple machine learning algorithms. In certain embodiments, the first machine learning model is trained using multiple machine learning algorithms, and N features (e.g., gene modules) can be selected based on the overall feature importance values, wherein for each feature the overall features importance value is obtained by summing the feature importance values of the feature of the top performing machine learning algorithms. In certain embodiments, the first machine learning model is trained using multiple machine learning algorithms, and N features (e.g., gene modules) can be selected based on the overall SHAP values, wherein for each feature the overall SHAP value is obtained by summing the SHAP values of the feature of the top performing machine learning algorithms. In certain embodiments, the N gene modules have top N overall feature importance values and/or overall SHAP values among the input features of the first machine learning model. Top performing machine learning algorithms can be machine learning algorithms having top, such as top 2, 3, 4, 5, 6, 7, or 8 ROC-AUC values among the multiple machine learning algorithms. In certain embodiments, N modules are obtained from the plurality of gene modules in multiple steps, wherein based on the training of first machine learning model with the first data set N1 modules can be selected from the plurality of gene modules, and N modules can be selected from the N1 modules. N1 can be an integer and X>N1>N. The N1 gene modules can have top N1 feature importance values and/or SHAP values among the input features of the first machine learning model for classifying the plurality of reference patients between the first state and the second state. In certain embodiments, a third machine learning model can be trained with a third data set comprising or derived from gene expression measurements of genes within the N1 gene modules, to classify a plurality of reference patients between the first state and the second state. In certain embodiments, the N gene modules have top N feature importance values and/or SHAP values among the input features of the third machine learning model for classifying the plurality of reference patients between the first state and the second state. It evident to a skilled artisan, additional steps can used to get N gene modules from the plurality of gene modules, for example, N2 modules can be selected from the N1 modules based on the input features of the third machine learning model for classifying the plurality of reference patients between the first state and the second state; a fourth machine learning model can be trained with a fourth data set comprising or derived from gene expression measurements of genes within the N2 gene modules, to classify a plurality of reference patients between the first state and the second state; and N modules can be selected from the N2 modules, wherein N gene modules have top N feature importance values and/or SHAP values among the input features of the fourth machine learning model for classifying the plurality of reference patients between the first state and the second state, and X>N1> N2>N.

The genes removed in step (c) can be determined to be redundant with respect to one or more genes (e.g., the genes within the N gene modules) based on the expression values of the genes, in the plurality of reference biological samples. In certain embodiments, genes removed in step (c) can have a correlation coefficient greater than a threshold value. The correlation coefficient can be determined based on the expression values of the genes, in the plurality of reference biological samples. The threshold value can be 0.6, 0.65, 0.7, 0.75, 0.8, 0.85, 0.9 or 0.95. In certain embodiments, the threshold value is 0.8. In certain embodiments, the threshold value is 0.75. In certain embodiments, the threshold value is 0.85.

The genes in the first gene set can form input features of the second machine learning model. For each gene in the first gene set, expression (e.g., gene expression) value of the gene in a reference biological sample of the plurality of reference biological samples, can form input feature value of the respective gene for the reference biological sample, for the training of the second machine learning model. In certain embodiments, the expression value can be a log2 expression value. M genes can be selected based on the feature importance values and/or SHAP values of the input features of the second machine learning model for classifying the plurality of reference patients between the first state and the second state. In certain embodiments, the M genes have top M feature importance values and/or SHAP values among the input features of the second machine learning model for classifying the plurality of reference patients between the first state and the second state. In certain embodiments, the M genes have top M feature importance values among the feature importance values of the input features of the second machine learning model for classifying the plurality of reference patients between the first state and the second state. In certain embodiments, the M genes have top M SHAP values among the SHAP values of the input features of the second machine learning model for classifying the plurality of reference patients between the first state and the second state. Genes selected in step (e) may or may not contain any additional gene (e.g., from the first gene set) over the M genes. M can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or any range therebetween. In certain embodiments, M is about 2 to about 40. In certain embodiments, M is about 2 to about 3, about 2 to about 5, about 2 to about 8, about 2 to about 10, about 2 to about 12, about 2 to about 15, about 2 to about 18, about 2 to about 20, about 2 to about 25, about 2 to about 30, about 2 to about 40, about 3 to about 5, about 3 to about 8, about 3 to about 10, about 3 to about 12, about 3 to about 15, about 3 to about 18, about 3 to about 20, about 3 to about 25, about 3 to about 30, about 3 to about 40, about 5 to about 8, about 5 to about 10, about 5 to about 12, about 5 to about 15, about 5 to about 18, about 5 to about 20, about 5 to about 25, about 5 to about 30, about 5 to about 40, about 8 to about 10, about 8 to about 12, about 8 to about 15, about 8 to about 18, about 8 to about 20, about 8 to about 25, about 8 to about 30, about 8 to about 40, about 10 to about 12, about 10 to about 15, about 10 to about 18, about 10 to about 20, about 10 to about 25, about 10 to about 30, about 10 to about 40, about 12 to about 15, about 12 to about 18, about 12 to about 20, about 12 to about 25, about 12 to about 30, about 12 to about 40, about 15 to about 18, about 15 to about 20, about 15 to about 25, about 15 to about 30, about 15 to about 40, about 18 to about 20, about 18 to about 25, about 18 to about 30, about 18 to about 40, about 20 to about 25, about 20 to about 30, about 20 to about 40, about 25 to about 30, about 25 to about 40, or about 30 to about 40. In certain embodiments, M is about 2, about 3, about 5, about 8, about 10, about 12, about 15, about 18, about 20, about 25, about 30, or about 40. In certain embodiments, M is at least about 2, about 3, about 5, about 8, about 10, about 12, about 15, about 18, about 20, about 25, or about 30. In certain embodiments, M is at most about 3, about 5, about 8, about 10, about 12, about 15, about 18, about 20, about 25, about 30, or about 40. In certain embodiments, M is about 15 to about 25. In certain embodiments, M is about 15 to about 16, about 15 to about 17, about 15 to about 18, about 15 to about 19, about 15 to about 20, about 15 to about 21, about 15 to about 22, about 15 to about 23, about 15 to about 24, about 15 to about 25, about 16 to about 17, about 16 to about 18, about 16 to about 19, about 16 to about 20, about 16 to about 21, about 16 to about 22, about 16 to about 23, about 16 to about 24, about 16 to about 25, about 17 to about 18, about 17 to about 19, about 17 to about 20, about 17 to about 21, about 17 to about 22, about 17 to about 23, about 17 to about 24, about 17 to about 25, about 18 to about 19, about 18 to about 20, about 18 to about 21, about 18 to about 22, about 18 to about 23, about 18 to about 24, about 18 to about 25, about 19 to about 20, about 19 to about 21, about 19 to about 22, about 19 to about 23, about 19 to about 24, about 19 to about 25, about 20 to about 21, about 20 to about 22, about 20 to about 23, about 20 to about 24, about 20 to about 25, about 21 to about 22, about 21 to about 23, about 21 to about 24, about 21 to about 25, about 22 to about 23, about 22 to about 24, about 22 to about 25, about 23 to about 24, about 23 to about 25, or about 24 to about 25. In certain embodiments, M is about 15, about 16, about 17, about 18, about 19, about 20, about 21, about 22, about 23, about 24, or about 25. In certain embodiments, M is at least about 15, about 16, about 17, about 18, about 19, about 20, about 21, about 22, about 23, or about 24. In certain embodiments, M is at most about 16, about 17, about 18, about 19, about 20, about 21, about 22, about 23, about 24, or about 25. In certain embodiments, M is 20. In certain embodiments, M is 10 to 30. In certain embodiments, the second machine learning model is trained using multiple machine learning algorithms, and M features (e.g., genes) can be selected based on the overall feature importance values, wherein for each feature the overall feature importance value is obtained by summing the feature importance values of the feature of the top performing machine learning algorithms. In certain embodiments, the second machine learning model is trained using multiple machine learning algorithms, and M features (e.g., genes) can be selected based on the overall SHAP values, wherein for each feature the overall SHAP value is obtained by summing the SHAP values of the feature of the top performing machine learning algorithms. In certain embodiments, the M genes have top M overall feature importance values and/or overall SHAP values among the input features of the second machine learning model. Top performing machine learning algorithms can be machine learning algorithms having top, such as top 2, 3, 4, 5, 6, 7, or 8 ROC-AUC values among the multiple machine learning algorithms.

The plurality of biological samples can comprise blood samples, isolated peripheral blood mononuclear cells (PBMCs), tissue biopsy samples, nasal fluids, saliva, urine, stool, or any derivative thereof. In certain embodiments, the plurality of biological samples comprises blood samples or any derivative thereof. In certain embodiments, the plurality of biological samples comprises PBMCs or any derivative thereof. In certain embodiments, the plurality of biological samples comprises tissue biopsy samples or any derivative thereof. In certain embodiments, the tissue biopsy samples comprise skin biopsy samples. In certain embodiments, the plurality of biological samples comprises nasal fluid samples or any derivative thereof. In certain embodiments, the plurality of biological samples comprises saliva samples or any derivative thereof. In certain embodiments, the plurality of biological samples comprises urine samples or any derivative thereof.

The first, second, third and/or fourth machine learning model can be trained with a machine learning classifier independently selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), Classification and Regression Tree (CART), and a combination thereof.

The trained first, second, third and/or fourth machine learning model can classify the plurality of reference patients between the first state and the second state with an accuracy of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The trained first, second, third and/or fourth machine learning model can classify the plurality of reference patients between the first state and the second state with a sensitivity of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The trained first, second, third and/or fourth machine learning model can classify the plurality of reference patients between the first state and the second state with a specificity of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The trained first, second, third and/or fourth machine learning model can classify the plurality of reference patients between the first state and the second state with a positive predictive value of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The trained first, second, third and/or fourth machine learning model can classify the plurality of reference patients between the first state and the second state with a negative predictive value of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the first state is having COVID-19 disease, and the second state is not having COVID-19 disease, and the plurality of gene modules are the gene modules listed in Table 1. In certain embodiments, the first state is having COVID-19 disease, and the second state is not having COVID-19 disease, and the plurality of gene modules are the gene modules listed in Table 1, wherein the plurality of patients of reference patients are ICU patients. In certain embodiments, the first state is having critical COVID-19 disease, and second state is having non-critical COVID-19 disease, and the plurality of gene modules are the gene modules listed in Table 1. In certain embodiments, the first state is not having COVID-19 disease, and second state is having non-critical COVID-19 disease, and the plurality of gene modules are the gene modules listed in Table 1.

In certain embodiments, the first state is having lupus and the second state is not having lupus, and the plurality of gene modules are the gene modules listed in Table 9. In certain embodiments, the first state is having psoriasis and the second state is not having psoriasis, and the plurality of gene modules are the gene modules listed in Table 9. In certain embodiments, the first state is having atopic dermatitis and the second state is not having atopic dermatitis, and the plurality of gene modules are the gene modules listed in Table 9. In certain embodiments, the first state is having systemic sclerosis and the second state is not having systemic sclerosis, and the plurality of gene modules are the gene modules listed in Table 9. In certain embodiments, the first state is having lupus and the second state is having psoriasis, and the plurality of gene modules are the gene modules listed in Table 9. In certain embodiments, the first state is having lupus and the second state is having atopic dermatitis, and the plurality of gene modules are the gene modules listed in Table 9. In certain embodiments, the first state is having lupus and the second state is having systemic sclerosis, and the plurality of gene modules are the gene modules listed in Table 9.

In certain embodiments, the first state is having lupus and the second state is not having lupus, and the plurality of gene modules are the gene modules listed in Table 10. In certain embodiments, the first state is responsiveness to a lupus drug selected from a neutrophil function inhibitor, a TNF inhibitor, an IL1 inhibitor, a Plasma cell inhibitor or deleter, a NK cell inhibitor or deleter, or a B Cell inhibitor or deleter, and the second state is non responsiveness to a lupus drug selected from neutrophil function inhibitor, a TNF inhibitor, an IL1 inhibitor, a Plasma cell inhibitor or deleter, a NK cell inhibitor or deleter, or a B Cell inhibitor or deleter, and the plurality of gene modules are the gene modules listed in Table 10. In certain embodiments, the first state is responsiveness to a lupus drug selected from a neutrophil function inhibitor, a TNF inhibitor, an IL1 inhibitor, a Plasma cell inhibitor, a NK cell inhibitor, or a B Cell Inhibitor and the second state is non responsiveness to a lupus drug selected from neutrophil function inhibitor, a TNF inhibitor, an IL1 inhibitor, a Plasma cell inhibitor, a NK cell inhibitor, or a B Cell Inhibitor, and the plurality of gene modules are the gene modules listed in Table 10, wherein the plurality of reference patients have lupus. In certain embodiments, the first state is responsiveness to a lupus drug comprising a neutrophil function inhibitor and the second state is non responsivenessto the lupus drug, and the plurality of gene modules are the gene modules listed in Table 10. In certain embodiments, the first state is responsiveness to a lupus drug comprising a neutrophil function inhibitor, and the second state is non responsivenessto the lupus drug, and the plurality of gene modules are the gene modules listed in Table 10, wherein the plurality of reference patients have lupus. In certain embodiments, the first state is responsiveness to a lupus drug comprising a TNF inhibitor and the second state is non responsivenessto the lupus drug, and the plurality of gene modules are the gene modules listed in Table 10. In certain embodiments, the first state is responsiveness to a lupus drug comprising a TNF inhibitor, and the second state is non responsivenessto the lupus drug, and the plurality of gene modules are the gene modules listed in Table 10, wherein the plurality of reference patients have lupus. In certain embodiments, the first state is responsiveness to a lupus drug comprising an IL1 inhibitor and the second state is non responsivenessto the lupus drug, and the plurality of gene modules are the gene modules listed in Table 10. In certain embodiments, the first state is responsiveness to a lupus drug comprising an IL1 inhibitor, and the second state is non responsivenessto the lupus drug, and the plurality of gene modules are the gene modules listed in Table 10, wherein the plurality of reference patients have lupus. In certain embodiments, the first state is responsiveness to a lupus drug comprising a Plasma cell inhibitor or deleter, and the second state is non responsivenessto the lupus drug, and the plurality of gene modules are the gene modules listed in Table 10. In certain embodiments, the first state is responsiveness to a lupus drug comprising a Plasma cell inhibitor or deleter, and the second state is non responsivenessto the lupus drug, and the plurality of gene modules are the gene modules listed in Table 10, wherein the plurality of reference patients have lupus. In certain embodiments, the first state is responsiveness to a lupus drug comprising a NK cell inhibitor or deleter and the second state is non responsivenessto the lupus drug, and the plurality of gene modules are the gene modules listed in Table 10. In certain embodiments, the first state is responsiveness to a lupus drug comprising a NK cell inhibitor or deleter, and the second state is non responsivenessto the lupus drug, and the plurality of gene modules are the gene modules listed in Table 10, wherein the plurality of reference patients have lupus. In certain embodiments, the first state is responsiveness to a lupus drug comprising a B Cell inhibitor or deleter, and the second state is non responsivenessto the lupus drug, and the plurality of gene modules are the gene modules listed in Table 10. In certain embodiments, the first state is responsiveness to a lupus drug comprising a B cell inhibitor or deleter, and the second state is non responsivenessto the lupus drug, and the plurality of gene modules are the gene modules listed in Table 10, wherein the plurality of reference patients have lupus. Non-limiting examples of a plasma cell inhibitor or deleter comprises Mycophenolate, Bortezomib, Carfilzomib, Ixazomib, Daratumumab, Isatuximab, Elotuzumab, or any combinations thereof. Non-limiting examples of an IL1 inhibitor comprises Anakinra, Canakinumab, or any combinations thereof. Non-limiting examples of a TNF inhibitor comprises Adalimumab, Certolizumab pegol, Etanercept, Golimumab, Infliximab, or any combinations thereof. Non-limiting examples of a Neutrophil function inhibitor comprise Dasatinib, Apremilast, Roflumilast, or any combinations thereof. Non-limiting examples of a NK cell inhibitor or deleter comprises Azathioprine. Non-limiting examples of a B cell inhibitor or deleter comprises Belimumab, Rituximab, Obinutuzumab, Ocrelizumab, Ofatumumab, Inebilizumab, or any combinations thereof.

### Methods for Diagnosis and Treatment of a Disease State

Certain aspects are directed to a method for classifying a patient between the first state and the second state. The method can include analyzing a patient data set comprising gene expression measurements of the 2 or more genes of a gene set obtained using the method containing the steps (a), (b), (c), (d) and/or (e), as described herein, from a biological sample obtained or derived from the patient. In certain embodiments, the patient data set comprises gene expression measurements of 2, 3, 4, 5, 6, 7, 8, 9, 10, 11, 12, 13, 14, 15, 16, 17, 18, 19, 20, 21, 22, 23, 24, 25, 26, 27, 28, 29, 30, or all or any value or range therebetween genes of a gene set obtained using the method containing the steps (a), (b), (c), (d) and/or (e), as described herein, from a biological sample obtained or derived from the patient. In certain embodiments, the patient data set comprises gene expression measurements of the genes of a gene set obtained using the method containing the steps (a), (b), (c), (d) and/or (e), as described herein, from a biological sample obtained or derived from the patient. Analyzing the patient data set can comprise providing the patient data set as an input to a machine learning model trained to generate an inference of whether the patient data set is indicative of the patient belonging to the first state or the patient belonging to the second state. In certain embodiments, the method further comprise receiving, as an output of the machine-learning model, the inference; and/or electronically outputting a report classifying the patient between the first state and the second state. The method can classify the patient between the first state and the second state based on the inference of the machine learning model.

The biological sample can comprise a blood sample, isolated peripheral blood mononuclear cells (PBMCs), tissue biopsy sample, nasal fluid, saliva, urine, stool, or any derivative thereof. In certain embodiments, the biological sample comprise a blood sample or any derivative thereof. In certain embodiments, the biological sample comprise a blood sample or any derivative thereof. In certain embodiments, the biological sample comprise isolated peripheral blood mononuclear cells (PBMCs) or any derivative thereof. In certain embodiments, the biological sample comprise a tissue biopsy sample or any derivative thereof. The tissue can be skin tissue. In certain embodiments, the biological sample comprise a nasal fluid sample or any derivative thereof. In certain embodiments, the biological sample comprise a saliva sample or any derivative thereof.

In certain embodiment, the method comprises recommending, selecting and/or administering a treatment to the patient based on the classification of the patient. In certain embodiment, the method comprises administering a treatment to the patient based on the classification of the patient. A treatment used in the context of the present methods may be any known to those of skill in the art for treating, e.g., reducing the severity of or reducing the risk of, the disease state in the patient.

The inference of the machine learning model can include a confidence value between 0 and 1. In certain embodiments, the confidence value of the inference of the machine learning model is between 0 and 1, such as 0.1, 0.2, 0.3, 0.4, 0.5, 0.6, 0.7, 0.8, 0.9 or 1, or any value or range therebetween

The machine learning model can be trained with a machine learning classifier independently selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), Classification and Regression Tree (CART), and a combination thereof.

The trained machine learning model can have a receiver operating characteristic curve (ROC) curve with an area under the curve (AUC) at least about 0.6, at least about 0.65, at least about 0.7, at least about 0.75, at least about 0.8, at least about 0.85, at least about 0.9, at least about 0.91, at least about 0.92, at least about 0.93, at least about 0.94, at least about 0.95, at least about 0.96, at least about 0.97, at least about 0.98, at least about 0.99, or more than about 0.99.

The method can classify the patient between the first state and the second state with an accuracy of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the patient between the first state and the second state with a sensitivity of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the patient between the first state and the second state with a specificity of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the patient between the first state and the second state with a positive predictive value of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the patient between the first state and the second state with a negative predictive value of at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the first state is having COVID-19 disease, and the second state is not having COVID-19 disease, and the patient dataset comprises gene expression measurements of the 2 or more genes selected from the genes listed in Table 11A, from a biological sample obtained or derived from the patient. In certain embodiments, the first state is having COVID-19 disease, and the second state is not having COVID-19 disease, and the patient dataset comprises gene expression measurements of the 2, 3, 4, 5, 6, 7, 8, 9, 10, 11, 12, 13, 14, 15, 16, 17, 18, 19 or 20 genes selected from the genes listed in Table 11A, from a biological sample obtained or derived from the patient.

In certain embodiments, the first state is having non-critical COVID-19 disease, and second state is not having COVID-19 disease, and the patient dataset comprises gene expression measurements of the 2 or more genes selected from the genes listed in Table 11B, from a biological sample obtained or derived from the patient. In certain embodiments, the first state is having non-critical COVID-19 disease, and second state is not having COVID-19 disease, and the patient dataset comprises gene expression measurements of the 2, 3, 4, 5, 6, 7, 8, 9, 10, 11, 12, 13, 14, 15, 16, 17, 18, 19 or 20 genes selected from the genes listed in Table 11B, from a biological sample obtained or derived from the patient.

In certain embodiments, the first state is having critical COVID-19 disease, and second state is having non-critical COVID-19 disease, and the patient dataset comprises gene expression measurements of the 2 or more genes selected from the genes listed in Table 11C, from a biological sample obtained or derived from the patient. In certain embodiments, the first state is having critical COVID-19 disease, and second state is having non-critical COVID-19 disease, and the patient dataset comprises gene expression measurements of the 2, 3, 4, 5, 6, 7, 8, 9, 10, 11, 12, 13, 14, 15, 16, 17, 18, 19 or 20 genes selected from the genes listed in Table 11C, from a biological sample obtained or derived from the patient.

In certain embodiments, the first state is having COVID-19 disease, and the second state is not having COVID-19 disease, and the patient dataset comprises gene expression measurements of the 2 or more genes selected from the genes listed in Table 11D, from a biological sample obtained or derived from the patient, wherein the patient is a ICU patient. In certain embodiments, the first state is having COVID-19 disease, and the second state is not having COVID-19 disease, and the patient dataset comprises gene expression measurements of the 2, 3, 4, 5, 6, 7, 8, 9, 10, 11, 12, 13, 14, 15, 16, 17, 18, 19 or 20 genes selected from the genes listed in Table 11D, from a biological sample obtained or derived from the patient, wherein the patient is a ICU patient.

In some embodiments, the subject has received a diagnosis of the COVID-19 disease. In some embodiments, the subject is suspected of having the COVID-19 disease. In some embodiments, the subject is at elevated risk of experiencing severe complications from the COVID-19 disease. In some embodiments, the subject is at elevated risk of having severe COVID-19 disease. Severe disease can comprise more severe disease or less severe disease. In some embodiments, the clinical feature is selected from: days of symptoms prior to admission to ICU; length of hospital stay; length of intubation; number of vent-free days; mortality; 30-day hospital mortality; admission APACHE score; admission SOFA score; admission BUN; admission CR; admission ferritin; admission CRP; admission ALT; admission AST; admission PF ratio; Max CR; Max Ferritin; Max CRP; Max ALT; and Max AST. In some embodiments, more severe disease is associated with at least one of: fewer days of symptoms prior to admission to ICU; greater length of hospital stay; greater length of intubation; lower number of vent-free days; higher mortality; higher 30-day hospital mortality; higher admission APACHE score; higher admission SOFA score; higher admission BUN; higher admission CRP; higher admission ferritin; higher admission CRP; higher admission ALT; and higher admission AST. In embodiments the comparison is to reference range. In some embodiments, the subject is asymptomatic for the COVID-19 disease. In some embodiments, the subject has been diagnosed with long COVID, is suspected of having long COVID, or is at high risk for developing long COVID.

In some embodiments, the patient has received a diagnosis of COVID-19 disease state. In some embodiments, the COVID-19 disease state of the patient is selected from: a predicted severity of disease, severity of disease, presence of disease, presence of long COVID, and predicted development of long COVID. Long COVID is understood to include any manifestations known to those of skill in the art, e.g., symptoms including fatigue, post-exertional malaise, fever, difficulty breathing or shortness of breath, cough, chest pain, heart palpitations, difficulty thinking or concentrating, headache, sleep problems, orthostatic hypotension (lightheadedness), neuropathic pain, e.g., pins-and-needles, change in smell or taste, depression or anxiety, diarrhea, stomach pain, Joint or muscle pain, rash, and changes in menstrual cycles, lasting more than four weeks after infection. Long COVID is described by, e.g., the Centers for Disease Control on their website, available at cdc.gov and incorporated herein by reference in its entirety. In some embodiments, gene enrichment is determined 1-21 days since COVID symptom onset. In some embodiments, gene enrichment is determined a period after symptom onset of about 1 day to about 21 days. In some embodiments, gene enrichment is determined a period after symptom onset of about 1 day to about 2 days, about 1 day to about 3 days, about 1 day to about 4 days, about 1 day to about 5 days, about 1 day to about 7 days, about 1 day to about 10 days, about 1 day to about 12 days, about 1 day to about 15 days, about 1 day to about 17 days, about 1 day to about 20 days, about 1 day to about 21 days, about 2 days to about 3 days, about 2 days to about 4 days, about 2 days to about 5 days, about 2 days to about 7 days, about 2 days to about 10 days, about 2 days to about 12 days, about 2 days to about 15 days, about 2 days to about 17 days, about 2 days to about 20 days, about 2 days to about 21 days, about 3 days to about 4 days, about 3 days to about 5 days, about 3 days to about 7 days, about 3 days to about 10 days, about 3 days to about 12 days, about 3 days to about 15 days, about 3 days to about 17 days, about 3 days to about 20 days, about 3 days to about 21 days, about 4 days to about 5 days, about 4 days to about 7 days, about 4 days to about 10 days, about 4 days to about 12 days, about 4 days to about 15 days, about 4 days to about 17 days, about 4 days to about 20 days, about 4 days to about 21 days, about 5 days to about 7 days, about 5 days to about 10 days, about 5 days to about 12 days, about 5 days to about 15 days, about 5 days to about 17 days, about 5 days to about 20 days, about 5 days to about 21 days, about 7 days to about 10 days, about 7 days to about 12 days, about 7 days to about 15 days, about 7 days to about 17 days, about 7 days to about 20 days, about 7 days to about 21 days, about 10 days to about 12 days, about 10 days to about 15 days, about 10 days to about 17 days, about 10 days to about 20 days, about 10 days to about 21 days, about 12 days to about 15 days, about 12 days to about 17 days, about 12 days to about 20 days, about 12 days to about 21 days, about 15 days to about 17 days, about 15 days to about 20 days, about 15 days to about 21 days, about 17 days to about 20 days, about 17 days to about 21 days, or about 20 days to about 21 days. In some embodiments, gene enrichment is determined a period after symptom onset of about 1 day, about 2 days, about 3 days, about 4 days, about 5 days, about 7 days, about 10 days, about 12 days, about 15 days, about 17 days, about 20 days, or about 21 days. In some embodiments, gene enrichment is determined a period after symptom onset of at least about 1 day, about 2 days, about 3 days, about 4 days, about 5 days, about 7 days, about 10 days, about 12 days, about 15 days, about 17 days, or about 20 days. In some embodiments, gene enrichment is determined a period after symptom onset of at most about 2 days, about 3 days, about 4 days, about 5 days, about 7 days, about 10 days, about 12 days, about 15 days, about 17 days, about 20 days, or about 21 days. In some embodiments, gene enrichment is determined a period after symptom onset of about 1 day to about 12 days. In some embodiments, gene enrichment is determined a period after symptom onset of about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 5 days to about 6 days, about 5 days to about 7 days, about 5 days to about 8 days, about 5 days to about 9 days, about 5 days to about 10 days, about 5 days to about 11 days, about 5 days to about 12 days, about 6 days to about 7 days, about 6 days to about 8 days, about 6 days to about 9 days, about 6 days to about 10 days, about 6 days to about 11 days, about 6 days to about 12 days, about 7 days to about 8 days, about 7 days to about 9 days, about 7 days to about 10 days, about 7 days to about 11 days, about 7 days to about 12 days, about 8 days to about 9 days, about 8 days to about 10 days, about 8 days to about 11 days, about 8 days to about 12 days, about 9 days to about 10 days, about 9 days to about 11 days, about 9 days to about 12 days, about 10 days to about 11 days, about 10 days to about 12 days, or about 11 days to about 12 days. In some embodiments, gene enrichment is determined a period after symptom onset of about 1 day, about 2 days, about 3 days, about 4 days, about 5 days, about 6 days, about 7 days, about 8 days, about 9 days, about 10 days, about 11 days, or about 12 days. In some embodiments, gene enrichment is determined a period after symptom onset of at least about 1 day, about 2 days, about 3 days, about 4 days, about 5 days, about 6 days, about 7 days, about 8 days, about 9 days, about 10 days, or about 11 days. In some embodiments, gene enrichment is determined a period after symptom onset of at most about 2 days, about 3 days, about 4 days, about 5 days, about 6 days, about 7 days, about 8 days, about 9 days, about 10 days, about 11 days, or about 12 days. In some embodiments, a subject predicted to have a critical COVID disease or outcome is administered a treatment. In some embodiments, the treatment for COVID comprises at least one drug selected from the drugs listed in Tables 8A and 8B. In some embodiments, the treatment for critical COVID comprises at least one drug selected from the drugs listed in Tables 8A and 8B. In some embodiments, the treatment for COVID comprises paxlovid, molnupiravir, and/or remdesivir.

In certain embodiments, the first state is having lupus and the second state is not having lupus. In certain embodiments, the first state is having psoriasis and the second state is not having psoriasis. In certain embodiments, the first state is having atopic dermatitis and the second state is not having atopic dermatitis. In certain embodiments, the first state is having systemic sclerosis and the second state is not having systemic sclerosis. In certain embodiments, the first state is having lupus and the second state is having psoriasis. In certain embodiments, the first state is having lupus and the second state is having atopic dermatitis 9. In certain embodiments, the first state is having lupus and the second state is having systemic sclerosis. In certain embodiments, the first state is responsiveness to a lupus drug selected from a neutrophil function inhibitor, a TNF inhibitor, an IL1 inhibitor, a Plasma cell inhibitor or deleter, a NK cell inhibitor or deleter, or a B Cell inhibitor or deleter, and the second state is non responsivenessto a lupus drug selected from neutrophil function inhibitor, a TNF inhibitor, an IL1 inhibitor, a Plasma cell inhibitor or deleter, a NK cell inhibitor or deleter, or a B Cell inhibitor or deleter. In certain embodiments, the first state is responsiveness to a lupus drug selected from a neutrophil function inhibitor, a TNF inhibitor, an IL1 inhibitor, a Plasma cell inhibitor or deleter, a NK cell inhibitor or deleter, or a B Cell Inhibitor or deleter and the second state is non responsivenessto a lupus drug selected from neutrophil function inhibitor, a TNF inhibitor, an IL1 inhibitor, a Plasma cell inhibitor or deleter, a NK cell inhibitor or deleter, or a B Cell Inhibitor or deleter, wherein the patient has lupus. In certain embodiments, the first state is responsiveness to a lupus drug comprising a neutrophil function inhibitor and the second state is non responsivenessto the lupus drug. In certain embodiments, the first state is responsiveness to a lupus drug comprising a neutrophil function inhibitor, and the second state is non responsivenessto the lupus drug, wherein the patients has lupus. In certain embodiments, the first state is responsiveness to a lupus drug comprising a TNF inhibitor and the second state is non responsivenessto the lupus drug. In certain embodiments, the first state is responsiveness to a lupus drug comprising a TNF inhibitor, and the second state is non responsivenessto the lupus drug, wherein the patient has lupus. In certain embodiments, the first state is responsiveness to a lupus drug comprising an IL1 inhibitor and the second state is non responsivenessto the lupus drug. In certain embodiments, the first state is responsiveness to a lupus drug comprising an IL1 inhibitor, and the second state is non responsivenessto the lupus drug, wherein the patient has lupus. In certain embodiments, the first state is responsiveness to a lupus drug comprising a Plasma cell inhibitor or deleter, and the second state is non responsivenessto the lupus drug. In certain embodiments, the first state is responsiveness to a lupus drug comprising a Plasma cell inhibitor or deleter, and the second state is non responsivenessto the lupus drug, wherein the patient has lupus. In certain embodiments, the first state is responsiveness to a lupus drug comprising a NK cell inhibitor or deleter and the second state is non responsivenessto the lupus drug. In certain embodiments, the first state is responsiveness to a lupus drug comprising a NK cell inhibitor or deleter, and the second state is non responsivenessto the lupus drug, wherein the patient has lupus. In certain embodiments, the first state is responsiveness to a lupus drug comprising a B Cell inhibitor or deleter, and the second state is non responsivenessto the lupus drug. In certain embodiments, the first state is responsiveness to a lupus drug comprising a B cell inhibitor or deleter, and the second state is non responsivenessto the lupus drug, wherein the patients has lupus.

In certain embodiments, the treatment for lupus comprises a neutrophil function inhibitor, a TNF inhibitor, an IL1 inhibitor, a Plasma cell inhibitor or deleter, a NK cell inhibitor or deleter, a B Cell Inhibitor or deleter, or any combination thereof. In some embodiments, a treatment for psoriasis comprises an agent that targets neutrophils. In some embodiments, a treatment for systemic sclerosis comprises an agent that targets TGFB fibroblasts and/or dendritic cells. In some embodiments, a treatment for atopic dermatitis comprises an agent that targets IL23. In certain embodiments, the treatment for a patient classified as responsive to a lupus drug, comprises the lupus drug.

Lupus can be systemic lupus erythematosus (SLE), cutaneous lupus erythematosus (CLE), discoid lupus erythematosus (DLE), acute cutaneous lupus erythematosus (ACLE), and/or subacute cutaneous lupus erythematosus (SCLE). In certain embodiments, lupus is SLE. In certain embodiments, lupus is CLE. In certain embodiments, lupus is DLE. In certain embodiments, lupus is SCLE. In certain embodiments, lupus is CCLE. In certain embodiments, lupus is lupus nephritis.

Non-limiting examples of a plasma cell inhibitor or deleter comprises Mycophenolate, Bortezomib, Carfilzomib, Ixazomib, Daratumumab, Isatuximab, Elotuzumab, or any combinations thereof. Non-limiting examples of an IL1 inhibitor comprises Anakinra, Canakinumab, or any combinations thereof. Non-limiting examples of a TNF inhibitor comprises Adalimumab, Certolizumab pegol, Etanercept, Golimumab, Infliximab, or any combinations thereof. Non-limiting examples of a Neutrophil function inhibitor comprise Dasatinib, Apremilast, Roflumilast, or any combinations thereof. Non-limiting examples of a NK cell inhibitor or deleter comprises Azathioprine. Non-limiting examples of a B cell inhibitor or deleter comprises Belimumab, Rituximab, Obinutuzumab, Ocrelizumab, Ofatumumab, Inebilizumab, or any combinations thereof.

TABLE 8A
(Drugs and compounds targeting IPA upstream regulators via blood, lung, or airway)

PBMC-CoV2 vs. PBMC-CTL (Blood)
IFNG: Amg 811‡
CSF1: Sunitinib†
Anti-IL6: Imiquimod†, PF-04236921‡, Siltuximab†, Sirukumab‡, Sarilumab†, Tocilizumab†,
Vobarilizumab‡
Anti-IFNA: AGS-009‡, Rontalizumab‡, Sifalimumab‡, Anifrolumab‡
CD38: Daratumumab†, TAK-079‡
TNKS: XAV-939^{P}
TGFB1: SB-431542^{P}, SB-525334^{P},
EGFR inhibitors: Brigatinib†, Dacomitinib†, Erlotinib†, Gefitinib†, Lapatinib†, Osimertinib†,
Vandetanib†, CGP-52411^{P}, Afatinib†, AG-490^{P}, AG-494^{P}, BIBU-1361^{P}, BIBX-1382‡,
Butein^{P}, CP-724714‡, Erbstatin-analog^{P}, Lavendustin-a, Lavendustin-c, RG-13022,
Tyrphostin, TYRPHOSTIN-1, TYRPHOSTIN-AG-1478^{P}, TYRPHOSTIN-AG-18^{P},
TYRPHOSTIN-AG-494^{P}, TYRPHOSTIN-AG-555, TYRPHOSTIN-AG-82, TYRPHOSTIN-
AG-835^{P}, WZ-3146^{P}, WZ-4002^{P}, Neratinib†, AEE788‡, ARRY-334543‡, AST-1306‡, AV-
412‡, AZD8931‡, BMS-599626‡, BMS-690514‡, Canertinib‡, CUDC-101‡, FERb-033^{P},
GW-583340^{P}, Mubritinib‡, PD-168393^{P}, Poziotinib‡, TAK-285‡, Tucatinib‡, Tyrphostin-AG-
825^{P}, Tyrphostin-AG-879^{P}, XL647‡
ERBB2: EGFR inhibitors
INSR inhibitors: Ceritinib†, Dovitinib‡, GSK1838705A^{P}, GSK1904529A^{P}, Linsitinib‡, NVP-
AEW541^{P}, NVP-TAE684^{P}
BRD4: LY-303511^{P}, AT-7519‡, Alvocidib‡, Roscovitine‡
VEGFR inhibitors: Lenvatinib†, Pazopanib†, Sorafenib†, Sunitinib†, ZM-306416^{P}
AKT inhibitors: 10-DEBC^{P}, A-443644, BML-257, Deguelin, Lonidamine†, Miltefosine†,
MK-2206‡
PPARG: Acetyl-farnesyl-cysteine†, T-0070907^{P}
PPARA: 2-fluoropalmitic-acid, GW-6471^{P}
HIFIA: CAY-10585, YC-1^{P}, 1-Benzylimidazole
STAT1: Sinensetin
IGF1R inhibitors: BMS-536924^{P}, BMS-754807‡, Ceritinib†, GSK-1904529A^{P}, Linsitinib‡,
PQ-401^{P}
BAX: BAX-Channel-Blocker
Drugs Predicted As Upstream Regulators (Inhibited): GnRH analog, Levodopa†, MEL S3,
Calcitriol†, CD 437, Ciprofloxacin†, ST1926, 5-fluorouracil†, Sirolimus†,
Medroxyprogesterone acetate†, Valproic acid†, BMS-690514‡, Panobinostat†, BMS-754807‡
Lung-CoV2 vs. Lung-CTL (Lung)
NFxB pathway inhibitors: CAY-10470^{P}, Quinacrine†, Quinoclamine, Auranofin†, BAY-11-
7082^{P}, BAY-11-7085^{P}, Betulinic-acid‡, Bicyclol†, Bindarit‡, Bortezomib†, Cepharanthine‡,
CKD-712‡, Closantel†, Curcumin†, Edasalonexent‡, Erythromycin†, EVP4593^{P}, E3330^{P},
Ginsenoside-C-K‡, Hexamethylenebisacetamide‡, Iguratimod†, IKK-2-inhibitor-V‡, MD-
920‡, NFKB-activation-inhibitor-II^{P}, Parthenolid‡, Parthenolide-(−)^{P}, Parthenolide-
(alternate-stereo)^{P}, Pyrrolidine-dithiocarbamate^{P}, Ro-106-9920^{P}, Sasapyrine†, Sesamin^{P}, SP-
100030^{P}, Ursolic-acid‡, Verbascoside‡
IL12: Ustekinumab†
Lymphotoxin: Etanercept†
IFNG: Amg 811‡
Anti-IL17: Brodalumab†, Ixekizumab†, Secukinumab†
Anti-TNF: Etanercept†, Adalimumab†, Certolizumab pegol†, Golimumab†, Infliximab†
Anti-IFNA: AGS-009‡, Rontalizumab‡, Sifalimumab‡, Anifrolumab‡
Anti-IL1: Canakinumab†, Anakinra†
IFNB1: PF-06823859‡
TNFSF12: BIIB023‡
TGM2: GK921^{P}, LDN-27219^{P}
RNASE1: Citric acid^{P}, Guanidine†, L-aspartic-acid†
APC: JW-67^{P}
NOS2: AR-C133057XX, Diphenyleneiodonium^{P}, Pyrrolidine-dithiocarbamate^{P}
ETV6-NTRK3: Entrectinib†, GNF-5837^{P}, Larotrectinib†
Interferon: Anti-IFNA, Amg 811‡, Anti-IFN OMEGA‡, PF-06823859‡
JAK inhibitors: AT-9283‡, Baricitinib†, Cucurbitacin-I, Filgotinib‡, Ruxolitinib†, Solcitinib‡,
Tofacitinib†, Upadacitinib†, AG-490^{P}, Atiprimod‡, Atractylenolide-i^{P}, AZD1480‡, AZ960^{P},
BMS-911543‡, CEP-33779^{P}, Curcumol‡, cyt387‡, Decernotinib‡, Delgocitini‡, Fedratinib†,
Ganoderic-acid-a^{P}, Itacitinib‡, JAK3-inhibitor-V^{P}, LY2784544‡, NS-018‡, NVP-BSK805^{P},
Pacritinib‡, Peficitinib†, PF-06651600‡, Ruxolitinib-(S)^{P}, TCS-21311^{P}, TG-02‡, TG-
101209^{P}, Thiram†, WHI-P154^{P}, XL019‡, ZM-39923^{P}, 1,2,3,4,5,6-hexabromocyclohexane^{P}
Tlr: Hydroxychloroquine†
Tnf (family) inhibitors: Anti-TNF, Dapirolizumab pegol‡, BIIB023‡, Atacicept‡, RC18‡,
Amg 570‡, Belimumab†, Blisibimod‡, Tabalumab‡
Drugs Predicted As Upstream Regulators (Inhibited): Alefacept, Etanercept†, Fontolizumab,
Leuprolide†, Hydrogen peroxide, AT^{P}, Hydroquinone†, Nitric oxide, Estriol†,
Norepinephrine†, D-glucose, Sphingosine-1-phosphate, Dexamethasone†,
Methylprednisolone†, Raloxifene†, 3-deazaneplanocin^{P}, Dalfampridine†,
Medroxyprogesterone acetate†, Lithium chloride, Chloropromazine, Tofacitinib†,
Nelfinavir†, Chloroquine†, Quercetin†
BAL-CoV2 vs. PBMC-CoV2 (Airway)
Anti-IL17: Brodalumab†, Ixekizumab†, Secukinumab†
CD3: Blinatumomab†
FCER1: Omalizumab
PXR-ligand-PXR-Retinoic acid-RXRα: Retinol†, Fenretinide‡
IL13: Lebrikizumab†
IL2: Daclizumab†
FLT3LG: Midostaurin†, Quizartinib‡, Sorafenib†, TCS-359^{P}
IL3: Talacotuzumab†
LIPE: Oxybenzone†, Benzyl-benzoate†
B4GALNT1: Dinutuximab†
SIRT3: Tenovin-6^{P}
RNASE1: Citric acid^{P}, Guanidine†, L-aspartic-acid†
EWSR1-FLI1: YK-4-279^{P}
Nr1h: GW-4064^{P}
Anti-Hsp90: Alvespimycin‡, BIIB021‡, Gedunin^{P}, Geldanamycin^{P}, NVP-AUY922‡, PU-
H71‡, Radicicol, Tanespimycin‡
IL12: Ustekinumab†
JAK inhibitors: AT-9283‡, Baricitinib†, Cucurbitacin-I, Filgotinib‡, Ruxolitinib†, Solcitinib‡,
Tofacitinib†, Upadacitinib†, AG-490^{P}, Atiprimod‡, Atractylenolide-i^{P}, AZD1480‡, AZ960^{P},
BMS-911543‡, CEP-33779^{P}, Curcumol‡, cyt387‡, Decernotinib‡, Delgocitinib‡, Fedratinib†,
Ganoderic-acid-a^{P}, Itacitinib‡, JAK3-inhibitor-V^{P}, LY2784544‡, NS-018‡, NVP-BSK805^{P},
Pacritinib‡, Peficitinib†, PF-06651600‡, Ruxolitinib-(S)^{P}, TCS-21311^{P}, TG-02‡, TG-
101209^{P}, Thiram†, WHI-P154^{P}, XL019^{‡}, ZM-39923^{P}, 1,2,3,4,5,6-hexabromocyclohexane^{P}
PRKCQ: Afimoxifene‡, Dexfosfoserine^{P}, GSK690693‡, Sotrastaurin‡, Tamoxifen†
LCK: Aminogenistein, JW-7-24-1, PP-2^{P}, WH-4023
ROCK inhibitors: Fasudil†, Ripasudil†, GSK-429286A^{P}, GSK-269962^{P}, KD025‡, LX7101‡,
OXA-06^{P}, RKI-1447^{P}, SB-772077B^{P}, SR-3677^{P}, Y-39983‡, Y-27632^{P}
Estrogen receptor antagonists: Tamoxifen†, Acolbifene‡, Afimoxifene‡, Clomifene†,
Danazol†, Endoxifen‡, Fulvestrant†, G-15^{P}, GDC-0927‡, PHTPP^{P}, Raloxifene†,
Toremifene†, Y-134^{P}, ZK-164015^{P}
AR: Androstenedione, BMS-641988, Enzalutamide†, Flutamide†, Nilutamide†
SNH: SANT-1^{P}, SANT-2^{P}
STAT1: Sinensetin
EPAS1: Cephaeline, YC-1^{P}, TC-S-7009^{P}, PT-2385‡
GATA3: Cyclosporin-a†, Simvastatin†, Tacrolimus†
TEAD2: Oleoylethanolamide^{P}
IKZF3: Lenalidomide†
ATF2: Ephedrine-(racemic)†
ITGB2: BMS-587101‡, Simvastatin†
TLR3: CU-CPT-4a^{P}
Anti-IL6: Imiquimod†, PF-04236921‡, Siltuximab†, Sirukumab‡, Sarilumab†, Tocilizumab†,
Vobarilizumab‡
Drugs Predicted As Upstream Regulators (Inhibited): 15-deoxy-delta-12,14-PGJ 2,
Hydrocortisone†, SB203580, JAK inhibitor I, UM101, Z-DEVD-FMK, Triptolide‡,
Etretinate\hat{ }
Antiviral: Paxlovid, Molnupiravir, REGEN-COV, Bamlanivimab and etesevimab,
Sotrovimab, AZD7442, Remdesivir (Veklury)
Legend:
†FDA-approved;
‡Drug in development/clinical trials;
^{P}Preclinical;
\hat{ }Withdrawn from market

TABLE 8B
(Unique IPA-predicted drugs by mechanism of action in blood, lung, or airway)

PBMC (blood)
Abl kinase inhibitor, ACE inhibitor, activator of soluble guanylyl cyclase, ALK tyrosine
kinase receptor mutant inhibitor, Anti-CD38, Anti-IFNAR1, Anti-IFNG, Anti-IL6, Anti-IL6
receptor, Anti-TNF, apoptosis stimulant, bacterial DNA gyrase inhibitor, Benzodiazepine
receptor agonist, CCR expression inhibitor, cell cycle inhibitor, COX inhibitor, CSFR
antagonist, cytochrome C release inhibitor, dopamine precursor, ErbB2 tyrosine kinase
inhibitor, FGFR inhibitor, glucokinase inhibitor, GnRH agonist, hypercalcaemic agent,
hypoxia inducible factor inhibitor, IFNA inhibitor, inhibitor of methylation of endogenous
isoprenylated proteins, insulin growth factor receptor inhibitor, insulin receptor ligand,
interferon inducer, interleukin inhibitor, kinase inhibitor, KIT inhibitor, Leucine rich repeat
kinase inhibitor, MCL1 inhibitor, membrane integrity inhibitor, NADH-ubiquinone
oxidoreductase (Complex I) inhibitor, NFκB pathway inhibitor, PDGFR inhibitor, phorbol
ester-induced ornithine decarboxylase (ODC) activity suppressor, PPAR receptor antagonist,
Proteasome inhibitor, RAF inhibitor, receptor tyrosine protein kinase inhibitor, RET tyrosine
kinase inhibitor, SIRT activator, Src inhibitor, Src tyrosine kinase inhibitor, steroid 5alpha-
reductase inhibitor, survivin inhibitor, T cell inhibitor, TGF beta receptor inhibitor,
thromboxane synthase inhibitor, thymidylate synthase inhibitor, TLR agonist, TNKS
inhibitor, tyrosine kinase inhibitor, VEGFR inhibitor, vitamin D receptor agonist, XIAP
inhibitor
Lung
Abl kinase inhibitor, acetylcholinesterase inhibitor, adiponectin receptor agonist, adrenergic
receptor agonist, aldehyde dehydrogenase inhibitor, aldose reductase inhibitor, algicide, ALK
expression enhancer, Anti-CD2, Anti-CD40LG, Anti-ICOSLG, Anti-IFNAR1, Anti-IFNB1,
Anti-IFNG, Anti-IFNW1, Anti-IL1, Anti-IL17, Anti-IL17 receptor, antimalarial agent, Anti-
TNF, Anti-TNFSF12, Anti-TNFSF13, Anti-TNFSF13B, AP inhibitor, apoptosis stimulant,
Bcr-Abl kinase inhibitor, CCN expression inhibitor, chitinase inhibitor, cholesterol inhibitor,
coagulation factor inhibitor, corticosteroid agonist, COX inhibitor, cytochrome P450 inhibitor,
cytokine production inhibitor, diacylglycerol O acyltransferase inhibitor, differentiation
inducer, disease modifying antirheumatic drug, DNA inhibitor, DNA methyltransferase
inhibitor, endoglin expression enhancer, ErbB2 tyrosine kinase inhibitor, Estrogen receptor
agonist, free radical scavenger, FtsZ inhibitor, glucocorticoid receptor modulator, glucose 6
phosphatase inhibitor, glucosidase inhibitor, GnRH agonist, growth factor receptor inhibitor,
histone lysine methyltransferase inhibitor, histone N-acetyltransferase inhibitor, HIV integrase
inhibitor, HIV protease inhibitor, IFNA inhibitor, IL12/23 inhibitor, immunosuppressant,
lipocortin synthesis stimulant, lipoxygenase inhibitor, MAPK inhibitor, melanin inhibitor,
metallic radical formation stimulant, mitotic inhibitor, monoamine oxidase inhibitor, NADPH
oxidase inhibitor, NFκB pathway activator, NFκB pathway inhibitor, nitric oxide synthase
inhibitor, phospholipase inhibitor, polar auxin transport inhibitor, potassium channel blocker,
prostanoid receptor antagonist, Proteasome inhibitor, proto-oncogene tyrosine protein kinase
inhibitor, quorum sensing signaling modulator, SARS coronavirus 3C-like protease inhibitor,
selective estrogen receptor modulator, SIRT activator, sodium channel blocker, STAT
inhibitor, steroidal receptor agonist, tau aggregation inhibitor, thioredoxin reductase inhibitor,
tissue transglutaminase inhibitor, TP53 activator, transglutaminase inhibitor, tropomyosin
receptor kinase inhibitor, tyrosine kinase inhibitor, WNT pathway inhibitor, xanthine oxidase
inhibitor
BAL (airway)
Abl kinase inhibitor, activator of soluble guanylyl cyclase, adrenergic receptor agonist, ALK
expression enhancer, Anti-B4GALNT1, Anti-CD19, Anti-CD3, Anti-FCER1A, Anti-IL13,
Anti-IL17, Anti-IL17 receptor, Anti-IL2R, Anti-IL3R, Anti-IL6, Anti-IL6 receptor, Anti-
MS4A2, Anti-TNF, apoptosis stimulant, AR antagonist, ATP citrase lyase inhibitor, Bcr-Abl
kinase inhibitor, binding of RNA helicase A to the transcription factor EWS-FLI1 inhibitor,
calcium channel activator, calcium sensitizer, cell cycle inhibitor, cereblon inhibitor,
cholesterol inhibitor, coagulation factor inhibitor, colony stimulating factor, corticosteroid
agonist, COX inhibitor, cyclin inhibitor, cyclophilin inhibitor, cytochrome P450 inhibitor,
disease modifying antirheumatic drug, DNA directed DNA polymerase inhibitor, endoglin
expression enhancer, ErbB2 tyrosine kinase inhibitor, Estrogen receptor agonist, estrogen
receptor modulator, EWS-FLI1 inhibitor, FK506-binding protein inhibitor, FXR agonist,
glucose-dependent insulinotropic receptor agonist, histamine release inhibitor, hypoxia
inducible factor inhibitor, IL12/23 inhibitor, immunostimulant, immunosuppressant, insulin
expression inhibitor, integrin antagonist, interferon inducer, interleukin inhibitor, kinase
inhibitor, KIT inhibitor, LCK inhibitor, Leucine rich repeat kinase inhibitor, LIM kinase
inhibitor, lipase inhibitor, lipocortin synthesis stimulant, luteinizing hormone releasing
hormone antagonist, macrolide calcineurin inhibitor, MAPK inhibitor, membrane integrity
inhibitor, metallic radical formation stimulant, mitotic inhibitor, multi targeted kinase
inhibitor, opioid receptor ligand, PKA inhibitor, potassium channel blocker, PPAR receptor
agonist, PPAR receptor antagonist, protease inhibitor, pyruvate dehydrogenase kinase
inhibitor, RAF inhibitor, RET tyrosine kinase inhibitor, retinoid receptor agonist, RNA
directed RNA polymerase inhibitor, ROCK inhibitor, rotamase inhibitor, selective estrogen
receptor modulator, SIRT inhibitor, soluble epoxide hydrolase inhibitor, Src inhibitor, STAT
inhibitor, steroid derivative with antigonadotropic and anti-estrogenic activities, stress
activated protein kinase inhibitor, T cell inhibitor, testosterone receptor agonist, TLR agonist,
TLR inhibitor, TNF modulator, tumor apoptosis inducer, tyrosine kinase inhibitor, VEGFR
inhibitor
Legend:
† FDA-approved;
‡ Drug in development/clinical trials;
P Preclinical;
\hat{ } Withdrawn from market

TABLE 9
Module: B Cell signature genes
AICDA, BANK1, BLK, BLNK, CD19, CD22, CD79A, CD79B, CLEC17A, CR2, DAPP1, DTX1,
FCRL1, FCRL2, FCRL3, FCRL4, FCRL5, HLA-DOB, IGHD, IGHM, LY6D, MS4A1, PAX5,
POU2AF1, SH2B2, TNFRSF13B, TNFRSF13C, VPREB1, VPREB3, ZBTB32, ZNF318
Module: Endothelial Cell signature genes
DLC1, ECSCR, EMCN, FLT1, KDR, LDB2, LRRC32, MEIS2, PLAT, PTPRB, SELE, TM4SF1,
TM4SF18, VWF
Module: Erythrocyte signature genes
BSG, GFI1B, GYPA, GYPB, GYPE, ICAM4, KEL, NFE2, RHD, SLC4A1, TRIM10, TSPO2
Module: Fibroblast signature genes
43894, ADAM33, ADAMTS6, AGTR1, ALPK2, ANGPTL2, ANKRD45, ANO2, ANPEP,
ARMC9, ASPN, BDKRB2, BDNF, BMPER, C11orf87, C1R, C1S, CATSPER3, CCDC102B,
CCDC80, CCDC81, CEMIP, CHAC1, CHRM2, CLMP, CNN1, COL14A1, COL3A1, COL5A1,
COLEC10, CPXM2, CPZ, CRABP2, CXCL12, DCN, DDR2, DKK1, DMRTA1, EGFL6,
ELOVL2, EMILIN1, FAM180A, FBLN7, FBN1, FGF5, FMN2, FOXF2, FST, FSTL1, GFRA1,
GLIS1, GLT8D2, GPR176, GREM1, GREM2, GRIK2, GUCY1A2, HSD17B2, HSPA2, HSPB3,
IL19, IQCD, KCNMB2, KIRREL3, KRT34, KRTAP1-5, KRTAP3-1, L1CAM, LAYN, LMOD1,
LOXL4, LUM, LY6K, MFAP4, MFAP5, MGARP, MGP, MKX, MMP2, MXRA5, MXRA8,
MYPN, NEXN, NFASC, NID2, NTF3, OLFML3, P3H3, P4HA3, PAMR1, PAX3, PCDHGA2,
PCDHGA3, PCDHGA7, PDE8B, PDGFRA, PDGFRL, PDZRN3, PLA2R1, PLEKHA4, PLPP4,
PLPPR4, PRKG1, PSG5, PTPRQ, PTRF, SEMA3A, SEMA5A, SEMA6D, SHOX, SLC16A2,
SMIM2, SPARCL1, SPHKAP, SSTR1, STC1, STXBP6, SUSD5, SVEP1, TBX15, THBS2,
TIMP2, TIMP3, TMEM119, TMEM130, TMEM47, TRHDE, TRPC4, UACA, UBL4B, VAT1L,
VEGFC, WNT5A, WNT5B, ZFPM2
Module: GC B Cell signature genes
FCRLA, GCSAM, KLHL6, LRMP, NUGGC, RGS13
Module: Granulocyte signature genes
CLC, HSH2D, MS4A2, PGLYRP1, PRG2, SYNE1
Module: Keratinocyte signature genes
ABLIM, AKR1C1, ALDH8, ALOX12B, ANXA8, AQP3, ATDC, BPAG1, CA12, CCND2,
CD24, CDH3, CDKN1A, CDSN, COL17A1, CST6, CSTA, DD96, DSC1, DSG1, DSP, EGFR,
EVPL, FLG, G0S2, GJA1, GLUL, GNA15, HBP17, IFI27, IFITM1, IGFBP7, ITGA3, ITGB4,
IVL, JUNB, JUP, KLK11, KLK7, KLK8, KRT1, KRT14, KRT15, KRT16, KRT2A, KRT5,
KRT6A, LAMA3, LGALS7, LOR, NOTCH3, PPL, PRDX2, PRSS11, PRSS2, PRSS4, S100A2,
SERPINB2, SERPINB3, SERPINB4, SERPINB5, SERPINB7, SERPINE1, SFN, SPINK5,
SPRR1B, TACSTD2, TFAP2A, TGM1, TP63, TUBA1, XP5
Module: Langerhans Cell signature genes
CD1B, CD1C, CD1E, CD207
Module: LDG signature genes
AZU1, CAMP, CEACAM3, CEACAM4, CEACAM6, CEACAM8, CTSG, DEFA4, ELANE,
MPO, OLFM4, RNASE3
Module: Melanocyte signature genes
ASIP, CITED1, DCT, GPNMB, GPR143, MITF, MLANA, MLPH, OCA2, PMEL, SLC24A5,
SLC45A2, TYR, TYRP1
Module: Monocyte signature genes
ADGRE1, C1QA, C1QB, C1QC, C2, CD14, CD300C, CD300E, CD5L, CD68, CLEC5A,
CSF1R, CYBB, FOLR2, LILRA1, MARCO, MERTK, MS4A7, MSR1, SPIC
Module: Monocyte/Myeloid Cell signature genes
ADGRE2, ADGRE3, AIF1, APOC1, BPI, BST1, C4A, C4B, C4BPA, C4BPB, C5, C6, C8A, C9,
CD163, CD1D, CD209, CD300LF, CD33, CFD, CFP, CHIT1, CLEC12A, CLEC12B, CLEC1A,
CLEC4A, CLEC4D, CLEC4E, CLEC4G, CLEC6A, CLEC7A, CRISP3, CSF2RA, CSF2RB,
CST3, CTSS, F12, FCER1A, FCER1G, FCGR1A, FCGR1B, FCGR2A, FCGR2C, FLT3, GRN,
IGSF6, ITGAX, LGALS12, LGALS4, LGALS9, LILRA2, LILRA5, LILRA6, LILRB2, LY86,
LYVE1, LYZ, MEFV, MMP8, MNDA, MPEG1, MS4A4A, MS4A6A, NLRP12, NLRP3, NOD2,
OLR1, OSCAR, OSM, PILRA, PRAM1, RETN, S100A12, S100A8, S100A9, SCARB1,
SECTM1, SEMA4A, SERPING1, SGK1, SIGLEC1, SIGLEC10, SIGLEC14, SIGLEC5,
SLC11A1, SLITRK4, SMPDL3B, SPI1, TEK, THBD, TLR2, TLR8, TNFSF13B, TREM1,
TREML4, TYROBP, VENTX, VSIG4, VSTM1
Module: Neutrophil signature genes
ARG1, BMX, CD177, CSF3R, DEFA1, DEFA1B, DEFA3, DEFB103A, DEFB103B,
DEFB106B, DEFB136, DEFB4A, FPR2, OR1J2, PRTN3, SLC2A3, SLPI
Module: NK Cell signature genes
KIR2DL4, KLRC3, KLRF1, NCAM1, NCR1, SH2D1B, TNFSF11, TXK
Module: pDC signature genes
CLEC4C, IRF7, LILRA4, NRP1, PACSIN1, PLA2G5, PLAC8, PTCRA, SERPINF1, SLC15A4,
TCF4
Module: Plasma Cell signature genes
CD38, CRELD2, ELL2, FKBP11, IGKV4-1, IGLV2-14, ITM2C, JCHAIN, MANF, MZB1,
PDIA4, PRDX4, SDF2L1, SPATS2, TNFRSF17, UAP1
Module: Platelet signature genes
GP1BA, GP5, GP6, GP9, PF4, PF4V1, PLEK, PPBP, SLC35D3
Module: Skin-specific DC signature genes
CLEC10A, CLEC9A, GPR31, MRC1, XCR1
Module: T- Cell signature genes
BCL11B, CAMK4, CD28, CD3D, CD3G, CD5, CD6, GPR171, ITK, RGCC, TESPA1, THEMIS,
TRAT1
Module: Th17 signature genes
CCR6, CXCR3, IL17A, IL17F, IL22, IL26, KLRB1, RORA, RORC
Module: AA Metabolism signature genes
AASS, AGXT, AGXT2, ALDH4A1, ALDH7A1, ARG1, GAD1, GLUD1, GOT1, GPT2,
GRHPR, IVD, MCCC2, OAT, OTC, OXCT1, PC, PSAT1, SDS, IL1RN, SOCS3, TNFAIP3
Module: Apoptosis signature genes
AIFM1, BAD, BAK1, BAX, BID, CASP10, CASP3, CASP6, CASP7, CASP8, CASP9, ENDOG,
FAS, HTRA2, TNFRSF1A
Module: Cell Cycle signature genes
ASPM, AURKA, AURKB, BRCA1, CCNB1, CCNB2, CCNE1, CDC20, CENPM, CEP55, E2F3,
GINS2, MCM10, MCM2, MKI67, NCAPG, NDC80, PTTG1, TYMS
Module: Complement Proteins signature genes
C1QA, C1QB, C1QC, C1R, C1S, C2, C3, C4A, C4B, C5, C6, C7, C8A, C8B, C8G, C9
Module: FAAO signature genes
HAAO, HACL1, PEX13, PHYH, SLC27A2
Module: FABO signature genes
ABCD1, ABCD2, ABCD3, ACAA2, ACACB, ACAD11, ACADL, ACADM, ACADS,
ACADVL, ACAT1, ACAT2, ACOX1, ACOX2, ACOX3, ACOXL, ACSBG2, ACSL5,
ADIPOQ, AKT2, AUH, BDH2, CPT1A, CPT2, CROT, DECR1, ECHDC1, ECHDC2, ECHS1,
ECI1, ECI2, EHHADH, ETFA, ETFB, ETFDH, FABP1, GCDH, HADH,
HADHA, HADHB, HIBCH, HSD17B4, IRS1, IRS2, IVD, LEP, PEX2, PEX5, PEX7, SESN2,
SLC25A17, SLC27A2, TWIST1
Module: Glycolysis signature genes
ALDOA, ALDOB, ALDOC, ENO1, ENO2, ENO3, GAPDH, GCK, GPI, HK1, HK2, HK3,
HKDC1, LDHA, LDHAL6A, LDHAL6B, LDHB, LDHC, PFKFB1, PFKFB2, PFKFB3,
PFKFB4, PFKL, PFKM, PFKP, PGAM1, PGAM2, PGK1, PGK2, PKM, SLC2A1, SLC2A3,
SLC2A4, TPI1
Module: IFN signature genes
EIF2AK2, GBP1, GBP2, GBP4, HERC5, HERC6, IFI27, IFI30, IFI35, IFI44, IFI44L, IFI6,
IFIT1, IFIT2, IFIT3, IFIT5, IFITM1, IFITM2, IFITM3, ISG15, ISG20, MX1, MX2, OAS1,
OAS2, OAS3, OASL, RSAD2, SAMD9, SAMD9L, SP100, SP110
Module: IL1 Cytokines signature genes
IL18, IL1B
Module: IL12 Complex signature genes
IL12A, IL12B, IL12RB1, IL12RB2
Module: IL12 signature genes
ACLY, AKAP10, APOL3, BACH2, BRCA2, CALD1, CASK, CASP1, CCR5, CDKN3,
CXCL10, CXCL9, CYBB, DEFA1, ETAA1, FASLG, FBXL2, FCER2, FCGR1A, GBP1, GBP2,
GLS, GNPDA1, GSTM5, GZMB, HHEX, HP, HSPA6, IFNG, IL16, IL18BP, IL18R1, IL1A,
INPP5D, INSIG1, IRF1, KLF2, KRT8, LIMK1, LINC00597, LY75, MMP25, NIN, NLRP1,
PCDH9, SELL, SERPIND1, SLAMF1, SOCS1, STAT1, TAP2, TBX21, TFF1, TNFAIP2,
TNFAIP3, TNFSF10, TXK
Module: IL17 Complex signature genes
IL17A, IL17F, IL17RA, IL17RC, TRAF3, TRAF3IP2
Module: IL21 Complex signature genes
IL21, IL21R, IL2RG
Module: IL23 Complex signature genes
IL12B, IL12RB1, IL23A, IL23R
Module: Immunoproteasome signature genes
PSMB10, PSMB8, PSMB9
Module: Inflammasome signature genes
AIM2, CASP1, CASP5, CTSB, GSDMB, GSDMD, NAIP, NEK7, NLRC4, NLRP1, NLRP3,
NOD2, P2RX7, PANX1, PYCARD, RIPK1
Module: OXPHOS signature genes
ATP5A1, ATP5B, ATP5D, ATP5E, ATP5F1, ATP5G1, ATP5G2, ATP5G3, ATP5H, ATP5I,
ATP5J, ATP5J2, ATP5L, ATP5O, ATP5S, BCS1L, CEP89, COA1,
COA3, COA4, COA5, COA6, COA7, COX10, COX10-AS1, COX11, COX14, COX15, COX16,
COX17, COX18, COX19, COX20, COX411, COX412, COX5A,
COX5B, COX6A1, COX6A2, COX6B1, COX6B2, COX6C, COX7A1, COX7A2, COX7A2L,
COX7B, COX7B2, COX7C, COX8A, COX8C, CYC1, CYCS, DNAJC15,
MT-ATP6, MT-ATP8, MT-CO1, MT-CO2, MT-CO3, MT-CYB, MT-ND1, MT-ND2, MT-ND3,
MT-ND4, MT-ND4L, MT-ND5, MT-ND6, NDUFA1, NDUFA10,
NDUFA11, NDUFA12, NDUFA13, NDUFA2, NDUFA3, NDUFA4, NDUFA4L2, NDUFA5,
NDUFA6, NDUFA7, NDUFA8, NDUFA9, NDUFAB1, NDUFAF1,
NDUFAF2, NDUFAF3, NDUFAF4, NDUFAF5, NDUFAF6, NDUFAF7, NDUFAF8, NDUFB1,
NDUFB10, NDUFB11, NDUFB2, NDUFB2-AS1, NDUFB3, NDUFB4,
NDUFB5, NDUFB6, NDUFB7, NDUFB8, NDUFB9, NDUFC1, NDUFC2, NDUFS1, NDUFS2,
NDUFS3, NDUFS4, NDUFS5, NDUFS6, NDUFS7, NDUFS8,
NDUFV1, NDUFV2, NDUFV3, NUBPL, OXA1L, RFESD, SCO1, SCO2, SLC25A4, SURF1,
TACO1, TIMMDC1, TMEM126B, TRAP1, TTC19, UQCC1, UQCC2,
UQCC3, UQCR10, UQCR11, UQCRB, UQCRC1, UQCRC2, UQCRFS1, UQCRH, UQCRHL,
UQCRQ
Module: Pentose Phosphate signature genes
G6PD, H6PD, PGD, PRPS1, PRPS1L1, PRPS2, RBKS, RGN, RPE, RPIA, TALDO1, TKT,
TKTL1, TKTL2
Module: Peroxisome signature genes
ABCD3, ACAA1, ACOX1, ACOX2, ACOX3, CAT, DDO, DECR2, EHHADH, HAO2,
HMGCL, HSDL2, ISOC1, KXD1, PAOX, PEX1, PEX10, PEX11A, PEX12,
PEX16, PEX19, PEX26, PEX3, PEX5, PEX6, PEX7, PHYH, PIPOX, PMVK, PXMP2, SCP2,
SLC25A17
Module: Proteasome signature genes
ADRM1, NGLY1, PAAF1, POMP, PSMA1, PSMA2, PSMA3, PSMA3-AS1, PSMA4, PSMA5,
PSMA6, PSMA7, PSMA8, PSMB1, PSMB11, PSMB2, PSMB3, PSMB4,
PSMB5, PSMB6, PSMB7, PSMC1, PSMC2, PSMC3, PSMC4, PSMC5, PSMC6, PSMD1,
PSMD10, PSMD11, PSMD12, PSMD13, PSMD14, PSMD2, PSMD3,
PSMD4, PSMD5, PSMD5-AS1, PSMD6, PSMD6-AS2, PSMD7, PSMD8, PSMD9, PSME1,
PSME2, PSME3, PSME4, PSMF1, PSMG1, PSMG2, PSMG3, PSMG3-
AS1, PSMG4, RAD23B, SHFM1, UBLCP1, UBQLN1, UCHL5, ZFAND2A
Module: ROS Production signature genes
GPX1, GPX3
Module: T Cell IL12 Signature genes
CCL5, CXCR1, GZMH, IFNG, IL12RB2, IL1RL1, LIFR, TNFSF10, TNFSF13B
Module: T Cell IL23 Signature genes
CCL17, CCL20, CCL22, CCL7, CCR1, CSF2, CXCL2, CXCR5, IL17A, IL17F, IL17RC, IL1R1,
IL23R, IL6, ITGA3, PDGFB, TNF
Module: TCA cycle signature genes
ACO2, CS, DLAT, DLD, DLST, FH, GLUD1, IDH1, IDH2, IDH3A, IDH3B, IDH3G, MDH2,
MPC1, MPC2, OGDH, OGDHL, PDHA1, PDHA2, PDHB, PDHX, PDK1,
PDK2, PDK3, PDK4, PDP1, PDP2, PDPR, SDHA, SDHAF1, SDHAF2, SDHAF3, SDHAF4,
SDHB, SDHC, SDHD, SUCLA2, SUCLG1, SUCLG2, SUGCT
Module: TNF signature genes
ACLY, ACSL1, ADGRE2, AK3, AKAP10, AMPD3, APOL3, ARID3A, ARSE, ASAP1,
B4GALT5, BCL2A1, BHLHE41, BHMT, BIRC3, BRCA1, CALD1, CASP1,
CASP10, CCL15, CCL20, CCL23, CCL3L1, CD37, CD38, CD83, CDKN3, CKB, CR2,
CTNND2, CXCL1, CXCL2, CXCL3, CXCL8, CYP27B1, DAB2, EBI3, EGR1,
EGR2, EPB41, EREG, ETAA1, F3, FABP1, FBXL2, FCER2, FCGR2A, FLJ11129, FLNA,
G0S2, GBP1, GCH1, GJB2, GLS, GMIP, GP1BA, GRK3, HCAR3,
HHEX, HOMER2, HP, ICAM1, IDO1, IFI44, IKBKG, IL16, IL18, IL1A, IL1B, IL1RN, IL6,
INHBA, INSIG1, ITGA6, KITLG, KLF1, KMO, LGALS3BP, MAP3K4,
MARCKS, MGLL, MMP19, MN1, MRPS15, MSC, MTF1, MX1, NAMPT, NELL2, NFKB1,
NFKB2, NFKBIA, NFKBIZ, NKX3-2, NR3C1, OAS3, PATJ, PDE4DIP,
PDPN, PIAS4, PLAUR, PTGES, PTGS2, RELB, RPGR, RPS9, SDC4, SERPIND1, SFRP1,
SH3BP5, SLAMF1, SLC30A4, SOD2, SPI1, SSPN, STAT4, TAF15,
TAP2, TBX3, TFF1, TNF, TNFAIP2, TNFAIP3, TNFRSF11A, TRAF1, TSC22D1, TYROBP,
UBE2C, VEGFA, WT1
Module: Unfolded Protein signature genes
B4GALT3, CALR, CALU, CANX, CDS2, CHST12, CHST2, DERL1, DERL2, DNAJC3,
EDEM2, EDEM3, EMC9, ERAP1, ERGIC2, ERO1L, EXT1, GALNT2,
GOLT1B, HERPUD1, HYOU1, IER3IP1, IMPAD1, KDELC1, KDELR2, LMAN2, LPGAT1,
MAN1A1, MANEA, MANF, NUCB2, PDIA4, PDIA6, PIGK, PPIB, SEC24D,
SEC61G, SPCS3, SSR1, SSR3, TRAM1, TRAM2, UGGT1, XBP1
Module: TGFB Fibroblast signature genes
ABTB2, ACOX1, ACTA2, ACTC, ACTN1, ACTN3, ADAM12, ADAM19, ADAMTS4,
ADCY7, AK3, ALS2CR4, AMIGO2, ANGPTL4, AQP1, ARK5, ARL4A, ARNTL,
ASE-1, ASNS, ATOH8, ATP10A, ATP1B1, AVP, AXUD1, B4GALT1, BAG3, BFAR,
BHLHB2, BLOC1S2, BM039, BMP6, BMPR2, C10orf22, C10orf30, C14orf138,
C14orf31, C16orf30, C18orf1, C20orf139, C20orf39, C21orf93, C5orf13, C6orf145, C6orf85,
C9orf19, C9orf3, C9orf62, CALM2, CARD4, CBFB, CCDC8, CCL2,
CDH2, CDKN2B, CEBPA, CH25H, CHIC2, CHST11, CHST5, CHSY1, CLC, CMKOR1, CNN3,
COL4A1, COL4A2, COL5A1, COL5A2, COMP, CREB3L2, CRLF1,
CRY1, CSRP1, CSRP2, CTGF, CTPS, CXCL12, CXXC5, CYR61, DACT1, DDIT4, DLC1,
DLX2, DNAJB4, DNAJB5, DNAJB9, DOK5L, DSP, DTR, DUSP1,
DYRK2, E2F7, EIF4EBP1, ELN, ENC1, ENPP1, EPHB3, ERN1, EYA2, FBXO32, FGF18,
FGF2, FGFR3, FGFRL1, FHL2, FLJ10350, FLJ10357, FLJ10378,
FLJ14054, FLJ20364, FLJ20366, FLJ20701, FLJ22938, FLJ39370, FLJ45248, FN5, FOXP1,
FSTL3, FUS, FZD8, GABRE, GADD45B, GARS, GAS7, GATA6,
GDF15, GDF6, GEM, GLS, GNPNAT1, GOPC, GPAM, GPR68, GPT2, GSTT2, HCMOGT-1,
HES1, HIF1A, HILS1, HNRPAB, HNRPK, HOMER1, HOXB2,
HOXC8, HSPA5, HSPB7, HSXIAPAF1, ID1, ID3, ID4, IER3, IER5L, IGF1, IL11, IL21R, IL4R,
IL6, ITR, IVNS1ABP, JUNB, K-ALPHA-1, KCNE4, KCNG1, KCNK1,
KCNN4, KCNS3, KCTD11, KIAA0033, KIAA0280, KIAA1102, KIAA1644, KIAA1754,
KLF10, KLF13, KLF2, LDHA, LHFPL2, LIF, LIM, LIMK1, LIMK2, LIMS3,
LMCD1, LMO4, LOC222171, LOC283824, LOC284454, LOC339047, LOC440502, LOC51333,
LRIG1, LRRC8, LTBP2, MAP3K2, MBD4, MGC14376, MGC15476,
MGC16121, MGC29875, MGC4504, MGC45871, MGC8685, MGLL, MICAL2, MICAL-L1,
MIR100HG, MONDOA, MRC2, MSX1, MTCH1, MTHFD2, NEDD4,
NEDD9, NET1, NFATC1, NFYC, NGEF, NID67, NKD2, NLF1, NNMT, NP, NPAS1, NPTX1,
NRBF2, NRG1, NUP98, ODC1, P4HA2, P4HA3, PACSIN2, PAWR,
PDGFA, PDLIM4, PFKP, PGK1, PGM2L1, PGM3, PHF17, PHLDA2, PHLDB1, PICALM,
PIM1, PITX2, PKM2, PLAU, PLAUR, PLEKHA1, PLK3, PLOD2, PNMA1,
PODXL, POFUT2, PPP1R13L, PPP1R14C, PPP1R3B, PRICKLE2, PRKAB2, PRO1855, PRPS1,
PRPS1L1, PRRX2, PSAT1, PTDSR, PTPNS1, RAI14, RAI17,
RASL11B, RGS3, RKHD3, RNF126, RPL21, RPL5, RTTN, RUNX1, RUNX2, RUSC2,
S100A16, SAMD11, SARS, SCD, SCHIP1, SDFR1, SERP1, SERPINE1,
SERTAD1, SERTAD4, SGCG, SGK, SH3MD1, SIAT4A, SKIL, SLC10A3, SLC16A3,
SLC19A2, SLC1A5, SLC20A1, SLC26A1, SLC2A1, SLC38A5, SLC39A14,
SLC4A2, SLC7A11, SLC7A5, SMAD7, SMARCB1, SNAI1, SNF1LK, SNX24, SOX4, SOX9,
SPARC, SPHK1, SRF, STC2, STCH, STK38L, SYNJ2, SYVN1, TBX3,
TD-60, TES, TGFB1, TGFBR1, TGM2, TIMP3, TIPARP, TMEPAI, TMPO, TNC, TNFRSF12A,
TNFRSF19L, TPM1, TRIB1, TRIB2, TRIB3, TSK, TUBA3, TUBA6,
TUBB2, TUBB3, TUBB4, TUBB6, TUFT1, UAP1, UCK2, UGDH, ULK1, UNC5B, UPP1,
USP35, VEGF, VLDLR, VMP1, WNT5B, XBP1, ZNF281, ZNF336, ZNF469, ZNF537
Module: Anti-inflammation signature genes
IL1RN, SOCS3, TNFAIP3

TABLE 10
For each module Genes are Listed by: Gene Symbol | Gene Entrez ID ||)

Module: Anergic/Activated T cells
Feature co-efficient = −0.03; Absolute feature co-efficient = 0.03
CD160 | 11126 || CD244 | 51744 || CTLA4 | 1493 || HAVCR2 | 84868 || ICOS | 29851 ||
KLRG1 | 10219 || LAG3 | 3902 || PDCD1 | 5133 ||
Module: Anti inflammation
Feature co-efficient = 0.75; Absolute feature co-efficient = 0.75
IL1RN | 3557 || SOCS3 | 9021 || TNFAIP3 | 7128 ||
Module: B cells
Feature co-efficient = −0.06; Absolute feature co-efficient = 0.06
BANK1 | 55024 || BLK | 640 || BLNK | 29760 || CD19 | 930 || CD22 | 933 || CD79A | 973 ||
CD79B | 974 || DAPP1 | 27071 || FCRL1 | 115350 || FCRL2 | 79368 || FCRL3 | 115352 ||
FCRLA | 84824 || GON4L | 54856 || GPR183 | 1880 || IGHD | 3495 || IGHM | 3507 ||
KLHL6 | 89857 || MS4A1 | 931 || PAX5 | 5079 || PLCL2 | 23228 || SH3BP5 | 9467 ||
VPREB1 | 7441 || ZNF318 | 24149 ||
Module: Cell Cycle
Feature co-efficient = 0.78; Absolute feature co-efficient = 0.78
ASPM | 259266 || AURKA | 6790 || AURKB | 9212 || BRCA1 | 672 || CCNB1 | 891 ||
CCNB2 | 9133 || CCNE1 | 898 || CDC20 | 991 || CENPM | 79019 || CEP55 | 55165 || E2F3 |
1871 || GINS2 | 51659 || MCM10 | 55388 || MCM2 | 4171 || MKI67 | 4288 || NCAPG |
64151 || NDC80 | 10403 || PTTG1 | 9232 || TYMS | 7298 ||
Module: Dendritic
Feature co-efficient = −0.49; Absolute feature co-efficient = 0.49
CLEC10A | 10462 || CLEC12A | 160364 || CLEC9A | 283420 || CSF1R | 1436 || IGIP |
492311 || LILRA4 | 23547 || LY75 | 4065 || XCR1 | 2829 ||
Module: gd T cells
Feature co-efficient = −0.26; Absolute feature co-efficient = 0.26
TRG-AS1 | 100506776 || BLK | 640 || TARP | 445347 || TRDC | 28526 || CD3E | 916 ||
CD3G | 917 ||
Module: Granulocyte
Feature co-efficient = 0.48; Absolute feature co-efficient = 0.48
CD177 | 57126 || CLC | 1178 || CTSS | 1520 || CXCR2 | 3579 || DEFA1 | 1667 || FUT7 |
2529 || LTB4R | 1241 || MMP25 | 64386 || OSM | 5008 || RETN | 56729 ||
Module: IFN
Feature co-efficient = 1.04; Absolute feature co-efficient = 1.04
EIF2AK2 | 5610 || GBP1 | 2633 || GBP2 | 2634 || GBP4 | 115361 || HERC5 | 51191 ||
HERC6 | 55008 || IFI27 | 3429 || IFI30 | 10437 || IFI35 | 3430 || IFI44 | 10561 || IFI44L |
10964 || IFI6 | 2537 || IFIT1 | 3434 || IFIT2 | 3433 || IFIT3 | 3437 || IFIT5 | 24138 || IFITM1
| 8519 || IFITM2 | 10581 || IFITM3 | 10410 || ISG15 | 9636 || ISG20 | 3669 || MX1 | 4599 ||
MX2 | 4600 || OAS1 | 4938 || OAS2 | 4939 || OAS3 | 4940 || OASL | 8638 || RSAD2 |
91543 || SAMD9 | 54809 || SAMD9L | 219285 || SP100 | 6672 || SP110 | 3431 ||
Module: IG CHAINS
Feature co-efficient = 0.32; Absolute feature co-efficient = 0.32
IGHA1 | 3493 || IGHA2 | 3494 || IGHD2-15 | 28503 || IGHD2-2 | 28505 || IGHD2-21 |
28502 || IGHD3-10 | 28499 || IGHD3-16 | 28498 || IGHD3-3 | 28501 || IGHD3-9 | 28500 ||
IGHG1 | 3500 || IGHG2 | 3501 || IGHG3 | 3502 || IGHG4 | 3503 || IGHJ1 | 28483 || IGHJ2 |
28481 || IGHJ3 | 28479 || IGHJ4 | 28477 || IGHJ5 | 28476 || IGHJ6 | 28475 || IGHV1-18 |
28468 || IGHV1-2 | 28474 || IGHV1-24 | 28467 || IGHV1-3 | 28473 || IGHV1-45 | 28466 ||
IGHV1-46 | 28465 || IGHV1-58 | 28464 || IGHV1-69-2 | 28458 || IGHV2-26 | 28455 ||
IGHV2-5 | 28457 || IGHV2-70 | 28454 || IGHV3-13 | 28449 || IGHV3-15 | 28448 ||
IGHV3-20 | 28445 || IGHV3-21 | 28444 || IGHV3-23 | 28442 || IGHV3-33 | 28434 ||
IGHV3-43 | 28426 || IGHV3-48 | 28424 || IGHV3-49 | 28423 || IGHV3-53 | 28420 ||
IGHV3-62 | 28416 || IGHV3-64 | 28414 || IGHV3-7 | 28452 || IGHV3-72 | 28410 ||
IGHV3-73 | 28409 || IGHV3-74 | 28408 || IGHV4-28 | 28400 || IGHV4-30-2 | 28398 ||
IGHV4-34 | 28395 || IGHV4-39 | 28394 || IGHV4-59 | 28392 || IGHV4-61 | 28391 ||
IGHV5-51 | 28388 || IGHV6-1 | 28385 || IGHV7-81 | 28378 || IGKC | 3514 || IGKJ1 |
28950 || IGKJ2 | 28949 || IGKJ3 | 28948 || IGKJ4 | 28947 || IGKJ5 | 28946 || IGKV1-16 |
28938 || IGKV1-17 | 28937 || IGKV1-27 | 28935 || IGKV1-5 | 28299 || IGKV1-6 | 28943 ||
IGKV1-9 | 28941 || IGKVID-16 | 28901 || IGKVID-17 | 28900 || IGKVID-43 | 28891 ||
IGKV1D-8 | 28904 || IGKV2-24 | 28923 || IGKV2D-26 | 28884 || IGKV2D-29 | 28882 ||
IGKV2D-30 | 28881 || IGKV3-20 | 28912 || IGKV3D-20 | 28874 || IGKV3D-7 | 28877 ||
IGKV4-1 | 28908 || IGKV5-2 | 28907 || IGLC2 | 3538 || IGLC7 || 28834 | IGLJ6 | 28828 ||
IGLV10-54 | 28772 || IGLV1-36 | 28826 || IGLV1-40 | 28825 || IGLV1-47 | 28822 ||
IGLV2-11 | 28816 || IGLV2-18 | 28814 || IGLV2-23 | 28813 || IGLV2-33 | 28811 || IGLV2-
8 | 28817 || IGLV3-1 | 28809 || IGLV3-10 | 28803 || IGLV3-12 | 28802 || IGLV3-16 | 28799
|| IGLV3-19 | 28797 || IGLV3-21 | 28796 || IGLV3-25 | 28793 || IGLV3-27 | 28791 ||
IGLV3-32 | 28787 || IGLV4-3 | 28786 || IGLV4-60 | 28785 || IGLV4-69 | 28784 || IGLV5-
37 | 28783 || IGLV5-45 | 28781 || IGLV6-57 | 28778 || IGLV7-43 | 28776 || IGLV8-61 |
28774 || IGLV9-49 | 28773 || IGLVI-70 | 28763 ||
Module: IL1 Pathway
Feature co-efficient = 0.17; Absolute feature co-efficient = 0.17
CHUK | 1147 || IKBKB | 3551 || NFKB1 | 4790 || MAP2K1 | 5604 || MAP2K4 | 6416 ||
IKBKG | 8517 || IL1A | 3552 || IL1R1 | 3554 || IRAK1 | 3654 || MAP3K3 | 4215 || MYD88
| 4615 || MAP2K6 | 5608 || MAP3K7 | 6885 || TRAF6 | 7189 || TAB1 | 10454 || TAB2 |
23118 || IRAK4 | 51135 || TOLLIP | 54472 || MAP3K8 | 1326 || IL1RAP | 3556 || IRAK2 |
3656 || MAPK8 | 5599 || RELA | 5970 || TAB3 | 257397 || IL1B | 3553 || UBE2N | 7334 ||
SQSTM1 | 8878 || IRAK3 | 11213 ||
Module: IL23 Complex
Feature co-efficient = −0.29; Absolute feature co-efficient = 0.29
IL12B | 3593 || IL12RB1 | 3594 || IL23A | 51561 || IL23R | 149233 ||
Module: Immunoproteosome
Feature co-efficient = 0.61; Absolute feature co-efficient = 0.61
PSMB10 | 5699 || PSMB8 | 5696 || PSMB9 | 5698 ||
Module: Inflammasome
Feature co-efficient = 0.57; Absolute feature co-efficient = 0.57
AIM2 | 9447 || CASP1 | 834 || CASP5 | 838 || CTSB | 1508 || GSDMB | 55876 || GSDMD |
79792 || NAIP | 4671 || NEK7 | 140609 || NLRC4 | 58484 || NLRP1 | 22861 || NLRP3 |
114548 || NOD2 | 64127 || P2RX7 | 5027 || PANX1 | 24145 || PYCARD | 29108 || RIPK1 |
8737
Module: Inflammatory Cytokines
Feature co-efficient = 0.69; Absolute feature co-efficient = 0.69
IL1A | 3552 || CXCL10 | 3627 || CXCL11 | 6373 || CXCL9 | 4283 || TNFSF13B | 10673 ||
CCL8 | 6355 || TNF | 7124 || IL1B | 3553 || IL18 | 3606 ||
Module: Inhibitory Macs
Feature co-efficient = 0.60; Absolute feature co-efficient = 0.60
AOAH | 313 || BACH1 | 571 || CD200R1 | 131450 || CD300A | 11314 || CD163 | 9332 ||
CLEC7A | 64581 || SCARB1 | 949 || MS4A4A | 51338 || IL10 | 3586 ||
Module: LDG
Feature co-efficient = 0.65; Absolute feature co-efficient = 0.65
AZU1 | 566 || CAMP | 820 || CEACAM6 | 4680 || CEACAM8 | 1088 || CTSG | 1511 ||
DEFA4 | 1669 || ELANE | 1991 || LCN2 | 3934 || LTF | 4057 || MPO | 4353 || OLFM4 |
10562 || RNASE3 | 6037 ||
Module: MHC II
Feature co-efficient = −0.28; Absolute feature co-efficient = 0.28
HLA-DMA | 3108 || HLA-DMB | 3109 || HLA-DPA1 | 3113 || HLA-DPB1 | 3115 || HLA-
DPB2 | 3116 || HLA-DQA1 | 3117 || HLA-DQA2 | 3118 || HLA-DQB1 | 3119 || HLA-
DQB2 | 3120 || HLA-DRA | 3122 || HLA-DRB1 | 3123 || HLA-DRB3 | 3125 || HLA-DRB4
| 3126 || HLA-DRB5 | 3127 || HLA-DRB6 | 3128 ||
Module: Monocyte
Feature co-efficient = 0.87; Absolute feature co-efficient = 0.87
ACE | 1636 || ADAM8 | 101 || ADAMDEC1 | 27299 || ADGRE1 | 2015 || ADGRE2 |
30817 || APOBEC3B | 9582 || APOBEC3G | 60489 || APOBR | 55911 || ART4 | 420 ||
C1QA | 712 || C1QC | 714 || C2 | 717 || C4A | 720 || C4B | 721 || C4BPA | 722 || C4BPB |
725 || C5 | 727 || C6 | 729 || C8A | 731 || C9 | 735 || CCL17 | 6361 || CCL18 | 6362 || CCL22
| 6367 || CCL28 | 56477 || CCL7 | 6354 || CCL8 | 6355 || CD14 | 929 || CD163 | 9332 ||
CD209 | 30835 || CD300C | 10871 || CD300E | 342510 || CD5L | 922 || CD80 | 941 || CFB |
629 || CFD | 1675 || CFP | 5199 || CHI3L1 | 1116 || CHIT1 | 1118 || CLEC12A | 160364 ||
CLEC12B | 387837 || CLEC4D | 338339 || CLEC4E | 26253 || CLEC5A | 23601 ||
CLEC7A | 64581 || CLEC9A | 283420 || CSF1R | 1436 || CXCL1 | 2919 || CXCL10 | 3627
|| CXCL11 | 6373 || CXCL13 | 10563 || CXCL8 | 3576 || CXCL9 | 4283 || CYBB | 1536 ||
F12 | 2161 | FCGR3B | 2215 | FFAR2 | 2867 | HMMR | 3161 | HVCN1 | 84329 || IGSF6 |
10261 || IL10RA | 3587 || IL12A | 3592 || IL12B | 3593 || IL1RAP | 3556 || IL20 | 50604 |
IL23A | 51561 || IL27 | 246778 || IL31RA | 133396 || LAMP3 | 27074 || LGALS12 | 85329
|| LGALS4 | 3960 || LGALS9 | 3965 || LGALS9B | 284194 || LGALS9C | 654346 ||
LILRA2 | 11027 || LILRA5 | 353514 || LILRA6 | 79168 || LILRB5 | 10990 || LMNB1 |
4001 || LY86 | 9450 || LYZ | 4069 || MARCO | 8685 || MEGF10 | 84466 || MERTK | 10461
|| MFGE8 | 4240 || MPEG1 | 219972 || MRC1 | 4360 || MS4A4A | 51338 || MSR1 | 4481 ||
NTSR1 | 4923 || OCM | 654231 || OLR1 | 4973 || OSCAR | 126014 || OTOF | 9381 ||
PDCD1LG2 | 80380 || PILRA | 29992 || PLA2G2D | 26279 || PLA2G5 | 5322 || PYHIN1 |
149628 || S100A8 | 6279 || S100A9 | 6280 || S1PR5 | 53637 || SCARB1 | 949 || SCARF2 |
91179 || SECTM1 | 6398 || SEMA4A | 64218 || SERPINB9 | 5272 || SERPING1 | 710 ||
SIGLEC1 | 6614 || SIGLEC14 | 100049587 || SIGLEC5 | 8778 || SIGLEC7 | 27036 ||
SLC11A1 | 6556 || SLITRK4 | 139065 || SMPDL3B | 27293 || SPIC | 121599 || STAB2 |
55576 || STAP2 | 55620 || TEK | 7010 || TGM2 | 7052 || TIMD4 | 91937 || TLR2 | 7097 ||
TLR8 | 51311 || TNF | 7124 || TNFAIP8L2 | 79626 || TNFRSF1B | 7133 || TNIP3 | 79931 ||
TULP1 | 7287 || UBD | 10537 || VENTX | 27287 || VSTM1 | 284415 ||
Module: Neutrophil
Feature co-efficient = 0.72; Absolute feature co-efficient = 0.72
ABHD3 | 171586 || ADAM8 | 101 || CD177 | 57126 || CD83 | 9308 || CLEC6A | 93978 ||
LY6E | 4061 || S100A6 | 6277 ||
Module: NK
Feature co-efficient = −0.46; Absolute feature co-efficient = 0.46
KLRF1 | 51348 || NCAM1 | 4684 || NCR1 | 9437 || NCR3 | 259197 || SH2D1B | 117157 ||
Module: Oxidative Phosphorylation
Feature co-efficient = −0.01; Absolute feature co-efficient = 0.01
ATP5A1 | 498 || ATP5B | 506 || ATP5D | 513 || ATP5E | 514 || ATP5F1 | 515 || ATP5G1 |
516 || ATP5G2 | 517 || ATP5G3 | 518 || ATP5H | 10476 || ATP5I | 521 || ATP5J | 522 ||
ATP5J2 | 9551 || ATP5L | 10632 || ATP5O | 539 || ATP5S | 27109 || BCS1L | 617 || CEP89
| 84902 || COA1 | 55744 || COA3 | 28958 || COA4 | 51287 || COA5 | 493753 || COA6 |
388753 || COA7 | 65260 || COX10 | 1352 || COX10-AS1 | 100874058 || COX11 | 1353 ||
COX14 | 84987 || COX15 | 1355 || COX16 | 51241 || COX17 | 10063 || COX18 | 285521 ||
COX19 | 90639 || COX20 | 116228 || COX4I1 | 1327 || COX4I2 | 84701 || COX5A | 9377 ||
COX5B | 1329 || COX6A1 | 1337 || COX6A2 | 1339 || COX6B1 | 1340 || COX6B2 |
125965 || COX6C | 1345 || COX7A1 | 1346 || COX7A2 | 1347 || COX7A2L | 9167 ||
COX7B | 1349 || COX7B2 | 170712 || COX7C | 1350 || COX8A | 1351 || COX8C | 341947
|| CYC1 | 1537 || CYCS | 54205 || DNAJC15 | 29103 || MT-ATP6 | 4508 || MT-ATP8 |
4509 || MT-CO1 | 4512 || MT-CO2 | 4513 || MT-CO3 | 4514 || MT-CYB | 4519 || MT-ND1
| 4535 || MT-ND2 | 4536 || MT-ND3 | 4537 || MT-ND4 | 4538 || MT-ND4L | 4539 || MT-
ND5 | 4540 || MT-ND6 | 4541 || NDUFA1 | 4694 || NDUFA10 | 4705 || NDUFA11 |
126328 || NDUFA12 | 55967 || NDUFA13 | 51079 || NDUFA2 | 4695 || NDUFA3 | 4696 ||
NDUFA4 | 4697 || NDUFA4L2 | 56901 || NDUFA5 | 4698 || NDUFA6 | 4700 || NDUFA7 |
4701 || NDUFA8 | 4702 || NDUFA9 | 4704 || NDUFAB1 | 4706 || NDUFAF1 | 51103 ||
NDUFAF2 | 91942 || NDUFAF3 | 25915 || NDUFAF4 | 29078 || NDUFAF5 | 79133 ||
NDUFAF6 | 137682 || NDUFAF7 | 55471 || NDUFAF8 | 284184 || NDUFB1 | 4707 ||
NDUFB10 | 4716 || NDUFB11 | 54539 || NDUFB2 | 4708 || NDUFB2-AS1 | 100134713 ||
NDUFB3 | 4709 || NDUFB4 | 4710 || NDUFB5 | 4711 || NDUFB6 | 4712 || NDUFB7 | 4713
|| NDUFB8 | 4714 || NDUFB9 | 4715 || NDUFC1 | 4717 || NDUFC2 | 4718 || NDUFS1 |
4719 || NDUFS2 | 4720 || NDUFS3 | 4722 || NDUFS4 | 4724 || NDUFS5 | 4725 || NDUFS6
| 4726 || NDUFS7 | 374291 || NDUFS8 | 4728 || NDUFV1 | 4723 || NDUFV2 | 4729 ||
NDUFV3 | 4731 || NUBPL | 80224 || OXA1L | 5018 || RFESD | 317671 || SCO1 | 6341 ||
SCO2 | 9997 || SLC25A4 | 291 || SURF1 | 6834 || TACO1 | 51204 || TIMMDC1 | 51300 ||
TMEM126B | 55863 || TRAP1 | 10131 || TTC19 | 54902 || UQCC1 | 55245 || UQCC2 |
84300 || UQCC3 | 790955 || UQCR10 | 29796 || UQCR11 | 10975 || UQCRB | 7381 ||
UQCRC1 | 7384 || UQCRC2 | 7385 || UQCRFS1 | 7386 || UQCRH | 7388 || UQCRHL |
440567 || UQCRQ | 27089 ||
Module: pDC
Feature co-efficient = −0.47; Absolute feature co-efficient = 0.47
CLEC4C | 170482 || NRP1 | 8829 || IL3RA | 3563 ||
Module: Plasma Cells
Feature co-efficient = 0.54; Absolute feature co-efficient = 0.54
C19orf10 | 56005 || IGH | 3492 || IGHD | 3495 || IGHG1 | 3500 || IGHMBP2 | 3508 ||
IGHV2-5 | 28457 || IGHV3-20 | 28445 || IGHV3-23 | 28442 || IGHV4-28 | 28400 ||
IGHV4-31 | 28396 || IGHV4-34 | 28395 || IGK | 50802 || IGKC | 3514 || IGL | 3535 ||
IGLJ3 | 28831 || IGLL1 | 3543 || IGLV@ | 3546 || IGLV1-40 | 28825 || IGLV1-44 | 28823 ||
IGLV2-14 | 28815 || IGLV2-5 | 28818 || IGLV3-1 | 28809 || IGLV3-19 | 28797 || IGLV3-25
| 28793 || IGLV4-3 | 28786 || IGLV4-60 | 28785 || IGLV5-45 | 28781 || IGLV6-57 | 28778 ||
IGLVI-70 | 28763 || MZB1 | 51237 || PRDM1 | 639 || SDC1 | 6382 || THEMIS2 | 9473 ||
TNFRSF17 | 608 ||
Module: SNOR LOW Up
Feature co-efficient = 0.36; Absolute feature co-efficient = 0.36
CEACAM1 | 634 || FCGR1A | 2209 || LGALS1 | 3956 || SNORD24 | 26820 || SNORD44 |
26806 || SNORD47 | 26802 || SNORD80 | 26774 ||
Module: T Cells
Feature co-efficient = −0.20; Absolute feature co-efficient = 0.20
CCR3 | 1232 || CD226 | 10666 || CD247 | 919 || CD28 | 940 || CD3D | 915 || CD3E | 916 ||
CD3G | 917 || CD4 | 920 || CD5 | 921 || CD8A | 925 || CD8B | 926 || ETS1 | 2113 || GATA3
| 2625 || GRAP2 | 9402 || LEF1 | 51176 || SH2D1A | 4068 || TRAC | 28755 || TRBC1 |
28639 || TRDC | 28526 ||
Module: T reg
Feature co-efficient = −0.02; Absolute feature co-efficient = 0.02
FOXP3 | 50943 || IKZF2 | 22807 ||
Module: TCRA
Feature co-efficient = −0.44; Absolute feature co-efficient = 0.44
TRAV10 | 28676 || TRAV1-1 | 28693 || TRAV1-2 | 28692 || TRAV12-1 | 28674 ||
TRAV12-2 | 28673 || TRAV12-3 | 28672 || TRAV13-1 | 28671 || TRAV13-2 | 28670 ||
TRAV14DV4 | 28669 || TRAV16 | 28667 || TRAV17 | 28666 || TRAV18 | 28665 ||
TRAV19 | 28664 || TRAV2 | 28691 || TRAV20 | 28663 || TRAV21 | 28662 || TRAV22 |
28661 || TRAV23DV6 | 28660 || TRAV24 | 28659 || TRAV25 | 28658 || TRAV26-1 |
28657 || TRAV26-2 | 28656 || TRAV27 | 28655 || TRAV29DV5 | 28653 || TRAV3 | 28690
|| TRAV30 | 28652 || TRAV34 | 28648 || TRAV35 | 28647 || TRAV36DV7 | 28646 ||
TRAV38-1 | 28644 || TRAV38-2DV8 | 28643 || TRAV39 | 28642 || TRAV4 | 28689 ||
TRAV40 | 28641 || TRAV41 | 28640 || TRAV5 | 28688 || TRAV7 | 28686 || TRAV8-1 |
28685 || TRAV8-2 | 28684 || TRAV8-3 | 28683 || TRAV8-4 | 28682 || TRAV8-6 | 28680 ||
TRAV8-7 | 28679 || TRAV9-1 | 28678 || TRAV9-2 | 28677 ||
Module: TCRAJ
Feature co-efficient = −0.50; Absolute feature co-efficient = 0.50
TRAJ10 | 28745 || TRAJ11 | 28744 || TRAJ12 | 28743 || TRAJ13 | 28742 || TRAJ14 | 28741
|| TRAJ15 | 28740 || TRAJ16 | 28739 || TRAJ17 | 28738 || TRAJ18 | 28737 || TRAJ19 |
28736 || TRAJ20 | 28735 || TRAJ21 | 28734 || TRAJ22 | 28733 || TRAJ23 | 28732 ||
TRAJ24 | 28731 || TRAJ25 | 28730 || TRAJ26 | 28729 || TRAJ27 | 28728 || TRAJ28 | 28727
|| TRAJ29 | 28726 || TRAJ3 | 28752 || TRAJ30 | 28725 || TRAJ31 | 28724 || TRAJ32 |
28723 || TRAJ33 | 28722 || TRAJ34 | 28721 || TRAJ35 | 28720 || TRAJ36 | 28719 ||
TRAJ37 | 28718 || TRAJ38 | 28717 || TRAJ39 | 28716 || TRAJ4 | 28751 || TRAJ40 | 28715
|| TRAJ41 | 28714 || TRAJ42 | 28713 || TRAJ43 | 28712 || TRAJ44 | 28711 || TRAJ45 |
28710 || TRAJ46 | 28709 || TRAJ47 | 28708 || TRAJ48 | 28707 || TRAJ49 | 28706 || TRAJ5
| 28750 || TRAJ50 | 28705 || TRAJ52 | 28703 || TRAJ53 | 28702 || TRAJ54 | 28701 ||
TRAJ56 | 28699 || TRAJ57 | 28698 || TRAJ58 | 28697 || TRAJ59 | 28696 || TRAJ6 | 28749
|| TRAJ61 | 28694 || TRAJ7 | 28748 || TRAJ8 | 28747 || TRAJ9 | 28746 ||
Module: TCRB
Feature co-efficient = −0.50; Absolute feature co-efficient = 0.50
TRBC2 | 28638 || TRBJ2-1 | 28629 || TRBJ2-2 | 28628 || TRBJ2-2P | 28627 || TRBJ2-3 |
28626 || TRBJ2-4 | 28625 || TRBJ2-5 | 28624 || TRBJ2-6 | 28623 || TRBJ2-7 | 28622 ||
TRBV1 | 28621 || TRBV10-1 | 28585 || TRBV10-2 | 28584 || TRBV11-1 | 28582 ||
TRBV11-2 | 28581 || TRBV19 | 28568 || TRBV2 | 28620 || TRBV20-1 | 28567 || TRBV21-
1 | 28566 || TRBV23-1 | 28564 || TRBV24-1 | 28563 || TRBV25-1 | 28562 || TRBV27 |
28560 || TRBV28 | 28559 || TRBV3-1 | 28619 || TRBV4-1 | 28617 || TRBV4-2 | 28616 ||
TRBV5-1 | 28614 || TRBV5-3 | 28612 || TRBV5-4 | 28611 || TRBV5-5 | 28610 || TRBV5-6
| 28609 || TRBV5-7 | 28608 || TRBV6-1 | 28606 || TRBV6-4 | 28603 || TRBV6-5 | 28602 ||
TRBV6-6 | 28601 || TRBV6-7 | 28600 || TRBV6-8 | 28599 || TRBV7-1 | 28597 || TRBV7-3
| 28595 || TRBV7-4 | 28594 || TRBV7-5 | 28593 || TRBV7-6 | 28592 || TRBV7-7 | 28591 ||
TRBV9 | 28586 ||
Module: TCRD
Feature co-efficient = −0.41; Absolute feature co-efficient = 0.41
TRDC | 28526 || TRDJ1 | 28522 || TRDJ2 | 28521 || TRDJ3 | 28520 || TRDJ4 | 28519 ||
TRDV1 | 28518 || TRDV2 | 28517 || TRDV3 | 28516 ||
Module: TNF_Waddel Up
Feature co-efficient = 0.67; Absolute feature co-efficient = 0.67
ACLY | 47 || ACSL1 | 2180 || ADGRE2 | 30817 || AK3 | 50808 || AKAP10 | 11216 ||
AMPD3 | 272 || APOL3 | 80833 || ARID3A | 1820 || ARSE | 415 || ASAP1 | 50807 ||
B4GALT5 | 9334 || BCL2A1 | 597 || BHLHE41 | 79365 || BHMT | 635 || BIRC3 | 330 ||
BRCA1 | 672 || CALD1 | 800 || CASP1 | 834 || CASP10 | 843 || CCL15 | 6359 || CCL20 |
6364 || CCL23 | 6368 || CCL3L1 | 6349 || CD37 | 951 || CD38 | 952 || CD83 | 9308 ||
CDKN3 | 1033 || CKB | 1152 || CR2 | 1380 || CTNND2 | 1501 || CXCL1 | 2919 || CXCL2 |
2920 || CXCL3 | 2921 || CXCL8 | 3576 || CYP27B1 | 1594 || DAB2 | 1601 || EBI3 | 10148 ||
EGR1 | 1958 || EGR2 | 1959 || EPB41 | 2035 || EREG | 2069 || ETAA1 | 54465 || F3 | 2152 ||
FABP1 | 2168 || FBXL2 | 25827 || FCER2 | 2208 || FCGR2A | 2212 || FLJ11129 | 54674 ||
FLNA | 2316 || G0S2 | 50486 | GBP1 | 2633 || GCH1 | 2643 || GJB2 | 2706 || GLS | 2744 ||
GMIP | 51291 || GP1BA | 2811 || GRK3 | 157 || HCAR3 | 8843 || HHEX | 3087 || HOMER2
| 9455 || HP | 3240 || ICAM1 | 3383 || IDO1 | 3620 || IFI44 | 10561 || IKBKG | 8517 || IL16 |
3603 || IL18 | 3606 || IL1A | 3552 || IL1B | 3553 || IL1RN | 3557 || IL6 | 3569 || INHBA |
3624 || INSIG1 | 3638 || ITGA6 | 3655 || KITLG | 4254 || KLF1 | 10661 || KMO | 8564 ||
LGALS3BP | 3959 || MAP3K4 | 4216 || MARCKS | 4082 || MGLL | 11343 || MMP19 |
4327 || MN1 | 4330 || MRPS15 | 64960 || MSC | 9242 || MTF1 | 4520 || MX1 | 4599 ||
NAMPT | 10135 || NELL2 | 4753 || NFKB1 | 4790 || NFKB2 | 4791 || NFKBIA | 4792 ||
NFKBIZ | 64332 || NKX3-2 | 579 || NR3C1 | 2908 || OAS3 | 4940 || PATJ | 10207 ||
PDE4DIP | 9659 || PDPN | 10630 || PIAS4 | 51588 || PLAUR | 5329 || PTGES | 9536 ||
PTGS2 | 5743 || RELB | 5971 || RPGR | 6103 || RPS9 | 6203 || SDC4 | 6385 || SERPIND1 |
3053 || SFRP1 | 6422 || SH3BP5 | 9467 || SLAMF1 | 6504 || SLC30A4 | 7782 || SOD2 |
6648 || SPI1 | 6688 || SSPN | 8082 || STAT4 | 6775 || TAF15 | 8148 || TAP2 | 6891 || TBX3 |
6926 || TFF1 | 7031 || TNF | 7124 || TNFAIP2 | 7127 || TNFAIP3 | 7128 || TNFRSF11A |
8792 || TRAF1 | 7185 || TSC22D1 | 8848 || TYROBP | 7305 || UBE2C | 11065 || VEGFA |
7422 || WT1 | 7490 ||
Module: Unfolded Protein
Feature co-efficient = 0.14; Absolute feature co-efficient = 0.14
B4GALT3 | 8703 || CALR | 811 || CALU | 813 || CANX | 821 || CDS2 | 8760 || CHST12 |
55501 || CHST2 | 9435 || DERL1 | 79139 || DERL2 | 51009 || DNAJC3 | 5611 || EDEM2 |
55741 || EDEM3 | 80267 || EMC9 | 51016 || ERAP1 | 51752 || ERGIC2 | 51290 || ERO1L |
30001 || EXT1 | 2131 || GALNT2 | 2590 || GOLT1B | 51026 || HERPUD1 | 9709 || HYOU1
| 10525 || IER3IP1 | 51124 || IMPAD1 | 54928 || KDELC1 | 79070 || KDELR2 | 11014 ||
LMAN2 | 10960 || LPGAT1 | 9926 || MAN1A1 | 4121 || MANEA | 79694 || MANF | 7873 ||
NUCB2 | 4925 || PDIA4 | 9601 || PDIA6 | 10130 || PIGK | 10026 || PPIB | 5479 || SEC24D |
9871 || SEC61G | 23480 || SPCS3 | 60559 || SSR1 | 6745 || SSR3 | 6747 || TRAM1 | 23471 ||
TRAM2 | 9697 || UGGT1 | 56886 || XBP1 | 7494 ||

TABLE 11A
Top 20 genes for classification of Covid vs healthy patients.

CCNB1, CCNB2, CD177, CDC20, CEP55, CLC, COX6C, COX7B,
COX7C, GINS2, MCM10, MKI67, NDUFAF5, NDUFAF7, NDUFB3,
OSM, PTTG1, SLC2A3, TMEM126B, UQCRB

TABLE 11B
Top 20 genes for classification of non-
critical Covid vs healthy patients.

CARD16, CCNB1, CCNB2, CCNE1, CDC20, CEP55, CFD, CFL1,
CLEC4D, COX7B, COX7C, DNAJC15, DUSP4, E2F3, FAS, H2BC21,
NDUFA5, NDUFAF7, TMEM126B, UQCRB

TABLE 11C
Top 20 genes for classification of critical
Covid vs non critical Covid patients.

CARD16, CAST, CCR3, CD177, CD28, CREM, DERL1, EIF2AK2,
EOMES, ERAP1, FKBP5, H2BC21, HIF1A, MCEMP1, MTPN,
NT5C3A, OXCT1, SGK1, SP100, SSR1

TABLE 11D
Top 20 genes for classification of Covid
ICU vs non-Covid ICU patients.

AMPD3, ASAP1, CAMP, CDKN3, CS, EGR1, FBXL2, FCER2, G6PD,
GBP4, GMIP, GP1BA, GYPA, H6PD, IFITM1, IFITM3, MCM10,
PTTG1, SDC1, SDHC

### Big Data Analysis Tools and Drug/Target Scoring Algorithms

The present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof. Systems and methods of the present disclosure may use one or more of the following: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool (e.g., P-Scope).

A method to assess a condition (e.g., COVID-19) of a subject may comprise using one or more data analysis tools and/or algorithms. The method may comprise receiving a dataset of a biological sample of a subject. Next, the method may comprise selecting one or more data analysis tools and/or algorithms. For example, the data analysis tools and/or algorithms may comprise a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), or a combination thereof. Next, the method may comprise processing the dataset using selected data analysis tools and/or algorithms to generate a data signature of the biological sample of the subject. Next, the method may comprise assessing the condition of the subject based on the data signature.

The BIG-C(Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of: Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOM5 datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyze tissues with suspected immune infiltrations that should also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, SIP1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that could be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

### BIG-C™ Big Data Analysis Tool

BIG-C® is a fast and efficient cloud-based tool to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® can be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25 (10), 1150-1170, which is incorporated herein by reference in its entirety). Using a knowledge base of over 5000 patients with systemic lupus erythematosus (SLE), over 16432 genes may be each placed into one of 53 BIG-C® functional categories, and statistical analysis may be performed to identify enriched categories. BIG-C® categories may be cross-examined with the GO and KEGG terms to obtain additional information and insights.

A sample BIG-C® workflow may comprise the following steps. First, SLE genomic datasets are derived from whole blood, peripheral blood mononuclear cells, affected tissues, and purified immune cells. Second, datasets are analyzed using DE analysis or Weighted Gene Coexpression Network Analysis (WGCNA). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-C® is leveraged to separate the individual annotated genes into one of 53 functional categories shown in Table 4 (e.g., as described by Labonte et al. 2018, “Identification of alterations in macrophage activation associated with disease activity in systemic lupus erythematosus,” PloS one, 13 (12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

TABLE 4
BIG-C Categories

| Immune | General | | | | | | | Pat. |
|---|---|---|---|---|---|---|---|---|
| Cell | Cell | Immune | Intracellular | MHC | MHC | Secreted | Secreted | Recog. |
| Surface | Surface | Signaling | Signaling | Class I | Class II | Immune | ECM | Receptors |
| Interferon | PRO- | Anti- | PRO | Anti | Unfold | Proteasome | Autophagy | Ubiquitylation |
| Gene | Cell | Cell | Apoptosis | Apoptosis | Prot. | | | |
| Sig | Cycle | Cycle | | | Stress | | | |
| General | Transcript. | Nuc. | Chromatin | DNA | mRNA | mRNA | MicroRNA | Cytoskeleton |
| Transcript. | Factors | Horm. | Remodel | Repair | Translation | Splicing | Processing | |
| | | Receptors | | | | | | |
| Integrin | RAS | WNT | Lysosome | Endocytosis | Endosome | Endoplas. | Oxidative | TCA |
| Pathway | Superfamily | Signaling | | | & | Retic. | Phosphor. | Cycle |
| | | | | | Vesicles | | | |
| Mito. | Mito | FA | Transporters | Cytoplasm | Peroxisomes | ROS | Nuclear | Active |
| DNA | | Biosynth | | Biochem | | Protection | & Nucleolus | RNA |
| to RNA | | | | | | | | |
| Micro | Melanosome | Unknown | Pseudogenes | Transposon | Golgi | Glycolysis | Palmitoylation | |
| RNA | | | | Control | | | | |

### I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ can be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories shown in Table 5, ultimately allowing for cellular activity analysis across multiple samples and disease states. When combined with BIG-C® categories, the cellular activity can be correlated to specific functions within a given cell type.

TABLE 5
I-Scope ™ Cell Sub-Categories

| | Plasma | | | | | |
|---|---|---|---|---|---|---|
| Monos/Macs | Cells | T-Cells | B-Cells | Dendritic | T&B Cells | CD8 T |
| Myeloid | Tact | LDG | Hematopoietic | Neutrophil | Ag | Granulocytes |
| Cells | | | | | Presentation | |
| Platelets | pDC | T, B, Mono | Langerhans | Bact | Mono and B | Erythrocytes |
| Mast Cell | T reg | Gd T | T anergic | FDC | CD4T | T/NK/NKT |
| | | | | | | Cells |

A sample I-Scope™ workflow may comprise the following steps. First, candidate genes are identified from datasets (associated with a disease state or condition) potentially associated with immune cell expression. Second, using HPA, GTEx, and FANTOM5 datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. An I-Scope™ signature analysis for a given sample may lead to the I-Scope™ signature analysis across multiple samples and disease states.

### T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, IL, which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ can be used downstream of the BIG-C® (Biologically Informed Gene-Clustering) tool to understand which tissue cell types are present. In conjunction with I-Scope™ (which provides information related to immune cells), T-Scope™ can be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories (as shown in Table 6), ultimately allowing for cellular activity analysis across multiple samples and disease states. When combined with BIG-C® categories, the cellular activity can be correlated to specific functions within a given tissue cell type.

TABLE 6
T-Scope^{TM} 45 Categories of Tissue Cells

Adipose Tissue; Adrenal Gland; Breast; Cartilage; Cerebral Cortex;
Cervix, Uterine; Chondrocyte; Colon; Dendritic; Duodenum;
Endometrium; Endothelial; Epididymis; Erythrocyte; Esophagus;
Fallopian Tube; Fibroblast; Gallbladder; Heart Muscle; Keratinocyte;
Keratinocyte Skin; Kidney; Kidney Distal Tubules; Kidney Loop;
Kidney Proximal Tubules; Kidney Tubule Duct; Kidney Tubule;
Langherhans; Liver; Lung; Melanocyte; Podocyte; Prostate; Rectum;
Salivary Gland; Seminal Vesicle; Skeletal Muscle; Skin; Small
Intestine; Smooth Muscle; Stomach; Synoviocyte; Testis; Thyroid
Gland; Urinary Bladder

A sample T-Scope™ workflow may comprise the following steps. First, candidate genes are identified from differential expression datasets (associated with a disease state or condition) potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Results may be obtained using T-Scope™ in combination with I-Scope™ for identification of cells post-DE-analysis.

### CellScan Big Data Analysis Tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+ autoimmune patients. The cloud-based genomic platform may leverage results from RNAseq and microarray experiments in conjunction with clinical information, such as medication and lab tests, to provide previously undiscovered insights.

CellScan™ may go beyond typical ‘omics analysis by performing one or more of the following: functionally categorizing genes and their products (e.g., using BIG-C®); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics; and prioritizing FDA-approved drugs and drugs-in-development for treatment in patients or pre-clinical models.

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of: lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of: NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. Table 7 shows an example list of reference databases for the content in CellScan™, with both human and mouse species-specific identifiers supported.

TABLE 7
Reference Databases for Content in CellScan ™

| Affymetrix | Entrez Gene | HPA | scRNAseq |
|---|---|---|---|
| Agilent | FANTOM5 | Illumina | STITCH |
| BrainArray | GenBank | Interactome | Mouse Genome |
| | | | Database (MGD) |
| CAS Registry | Gene Symbol - | KEGG | UCSC (hg18) |
| Number | human | | |
| | (Hugo/HGNC) | | |
| Clinicaltrials.gov | Gene Symbol - | LINCS/CLUE | UCSC (hg19) |
| | mouse (Entrez | | |
| | Gene) | | |
| CodeLink | GNF Tissue | Mosby's Drug | Unigene |
| | Expression | Consult | |
| | Body Atlas | | |
| DrugBank | GO terms | NCBI PubMed | Uniprot/Swiss- |
| | | | Prot |
| | | | Accession |
| Drugs@FDA | Goodman & | NCI-60 | |
| | Gilman's | Cell Line | |
| | Pharmacological | Expression | |
| | Basis of | Atlas | |
| | Therapeutics | | |
| Ensembl | GTEx | Refseq | |

### MS (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPAR (Ingenuity Pathway Analysis) tool that does not contain many immunologically relevant pathways. Similar to IPAR, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between −100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between-1 and +1 indicating levels of down-regulation and up-regulation respectively.

A sample MS-Scoring™ 2 workflow may comprise the following steps. First, a signaling pathway of interest is selected from the MS-Scoring™ 2 menu. Second, a raw gene expression data is inputted into the MS-Scoring™ 2 tool. Third, enrichment of signaling pathway(s) is assessed on a patient by patient basis. Fourth, the data can then be used to drive insight for the target signaling pathways in individual patient samples.

### Gene Set Variation Analysis (GSVA)

Gene Set Variation Analysis (GSVA) algorithms may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety) and as described by [R Core Team (2020). R: A language and environment for statistical computing. R Foundation for Statistical Computing, Vienna, Austria. www.R-project.org/], which is incorporated herein by reference in its entirety. The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

A GSVA-based data analysis tool may be developed for use in analyzing specific sets of gene pathways. The GSVA-based data analysis tool (e.g., P-Scope) may use a GSVA statistical test-based tool using different sets of genes to analyze certain pathways. Such sets of genes may include, for example, human genes, mouse genes, or a combination thereof.

### Computer Systems

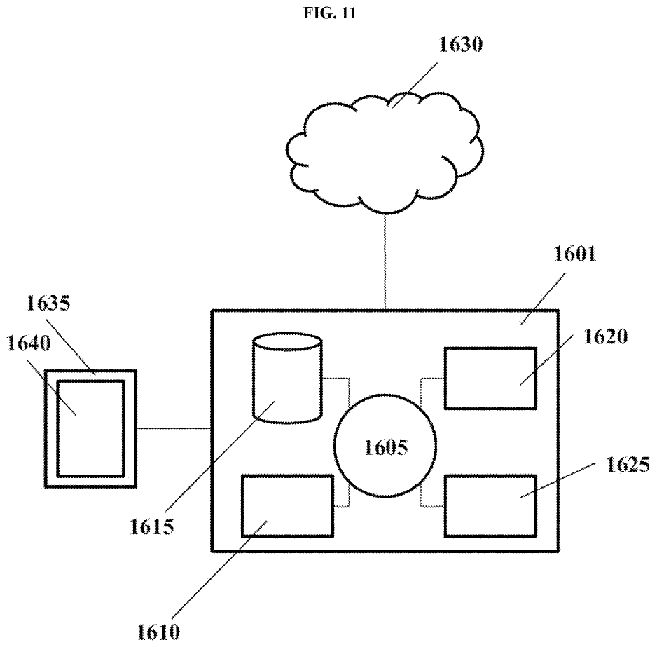

The present disclosure provides computer systems that are programmed to implement methods of the disclosure. FIG. 11 shows a computer system 1601 that is programmed or otherwise configured to, for example, perform methods of the disclosure.

The computer system 1601 can regulate various aspects of analysis, calculation, and generation of the present disclosure, such as, for example, performing methods of the disclosure. The computer system 1601 can be an electronic device of a user or a computer system that is remotely located with respect to the electronic device. The electronic device can be a mobile electronic device.

The computer system 1601 includes a central processing unit (CPU, also “processor” and “computer processor” herein) 1605, which can be a single core or multi core processor, or a plurality of processors for parallel processing. The computer system 1601 also includes memory or memory location 1610 (e.g., random-access memory, read-only memory, flash memory), electronic storage unit 1615 (e.g., hard disk), communication interface 1620 (e.g., network adapter) for communicating with one or more other systems, and peripheral devices 1625, such as cache, other memory, data storage and/or electronic display adapters. The memory 1610, storage unit 1615, interface 1620 and peripheral devices 1625 are in communication with the CPU 1605 through a communication bus (solid lines), such as a motherboard. The storage unit 1615 can be a data storage unit (or data repository) for storing data. The computer system 1601 can be operatively coupled to a computer network (“network”) 1630 with the aid of the communication interface 1620. The network 1630 can be the Internet, an internet and/or extranet, or an intranet and/or extranet that is in communication with the Internet.

The network 1630 in some cases is a telecommunication and/or data network. The network 1630 can include one or more computer servers, which can enable distributed computing, such as cloud computing. For example, one or more computer servers may enable cloud computing over the network 1630 (“the cloud”) to perform various aspects of analysis, calculation, and generation of the present disclosure, such as, for example, performing methods of the disclosure. Such cloud computing may be provided by cloud computing platforms such as, for example, Amazon Web Services (AWS), Microsoft Azure, Google Cloud Platform, and IBM cloud. The network 1630, in some cases with the aid of the computer system 1601, can implement a peer-to-peer network, which may enable devices coupled to the computer system 1601 to behave as a client or a server.

The CPU 1605 may comprise one or more computer processors and/or one or more graphics processing units (GPUs). The CPU 1605 can execute a sequence of machine-readable instructions, which can be embodied in a program or software. The instructions may be stored in a memory location, such as the memory 1610. The instructions can be directed to the CPU 1605, which can subsequently program or otherwise configure the CPU 1605 to implement methods of the present disclosure. Examples of operations performed by the CPU 1605 can include fetch, decode, execute, and writeback.

The CPU 1605 can be part of a circuit, such as an integrated circuit. One or more other components of the system 1601 can be included in the circuit. In some cases, the circuit is an application specific integrated circuit (ASIC).

The storage unit 1615 can store files, such as drivers, libraries and saved programs. The storage unit 1615 can store user data, e.g., user preferences and user programs. The computer system 1601 in some cases can include one or more additional data storage units that are external to the computer system 1601, such as located on a remote server that is in communication with the computer system 1601 through an intranet or the Internet.

The computer system 1601 can communicate with one or more remote computer systems through the network 1630. For instance, the computer system 1601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1601 via the network 1630.

Methods as described herein can be implemented by way of machine (e.g., computer processor) executable code stored on an electronic storage location of the computer system 1601, such as, for example, on the memory 1610 or electronic storage unit 1615. The machine executable or machine-readable code can be provided in the form of software. During use, the code can be executed by the processor 1605. In some cases, the code can be retrieved from the storage unit 1615 and stored on the memory 1610 for ready access by the processor 1605. In some situations, the electronic storage unit 1615 can be precluded, and machine-executable instructions are stored on memory 1610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1601, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 1601 can include or be in communication with an electronic display 1635 that comprises a user interface (UI) 1640 for providing, for example, a visual display. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1605. The algorithm can, for example, perform methods of the disclosure.

### Exemplary Embodiments

Embodiments include:
1. A method for obtaining a gene set capable of classifying a patient between a first state and a second state, the method comprising:
   a) training a first machine learning model based on a first data set comprising or derived from gene expression measurements of genes within a plurality of gene modules, to classify a plurality of reference patients between the first state and the second state, wherein the gene expression measurements are obtained from a plurality of reference biological samples derived or obtained from the plurality of reference patients;
   b) selecting N gene modules from the plurality of gene modules based on feature importance values and/or absolute SHAP values of input features of the first machine learning model for classifying the plurality of reference patients between the first state and the second state, wherein N is an integer;
   c) optionally removing redundant genes from genes within the N gene modules to obtain a first gene set;
   d) training a second machine learning model based on a second data set comprising gene expression measurements of genes within the first gene set to classify the plurality of reference patients between the first state and the second state;
   e) selecting M genes from the genes within the first gene set based on feature importance values and/or absolute SHAP values of input features of the second machine learning model for classifying the plurality of reference patients between the first state and the second state, wherein M is an integer wherein the selected M genes form the gene set capable of classifying the patient between the first state and the second state, and wherein a first portion of the plurality of reference patients belong to the first state and a second portion of the plurality of reference patients belong to the second state.
2. The method of embodiment 1, wherein the plurality of gene modules forms the input features of the first machine learning model.
3. The method of embodiment 2, wherein for each gene module of the plurality of gene modules, an enrichment value of the gene module in a reference biological sample of the plurality of reference biological samples forms an input feature value of the gene module for the reference biological sample, for the training of the first machine learning model.
4. The method of embodiment 3, wherein the enrichment value is a GSVA score.
5. The method of any one of embodiments 1 to 4, wherein the N gene modules are the gene modules having the top N feature importance values and/or absolute SHAP values among the input features of the first machine learning model for classifying the plurality of reference patients between the first state and the second state.
6. The method of any one of embodiments 1 to 5, wherein Nis 5 to 15.
7. The method of any one of embodiments 1 to 6, wherein genes in the first gene set forms the input features of the second machine learning model.
8. The method of embodiment 7, wherein for each gene in the first gene set, an expression value of the gene in a reference biological sample of the plurality of reference biological samples, forms an input feature value of the gene for the reference biological sample, for the training of the second machine learning model.
9. The method of embodiment 8, wherein the expression value is a log2 expression value.
10. The method of any one of embodiments 1 to 9, wherein the M genes are the genes having the top M feature importance values and/or absolute SHAP values among the input features of the second machine learning model for classifying the plurality of reference patients between the first state and the second state.
11. The method of any one of embodiments 1 to 10, wherein M is 15 to 30.
12. The method of any one of embodiments 1 to 11, wherein the redundant genes have correlation coefficients greater than a threshold value, wherein the correlation coefficients are determined based on the expression values of the genes in the N gene modules, in the plurality of reference biological samples.
13. The method of embodiment 12, wherein the threshold value is 0.8.
14. The method of any one of embodiments 1 to 13, wherein the plurality of biological samples comprise blood samples, isolated peripheral blood mononuclear cells (PBMCs), tissue biopsy samples, nasal fluids, saliva, urine, stool, or any derivative thereof.
15. The method of any one of embodiments 1 to 13, wherein the first state is having COVID-19 disease, the second state is not having COVID-19 disease, and the plurality of gene modules are the gene modules listed in Table 1.
16. The method of any one of embodiments 1 to 13, wherein the first state is having critical COVID-19 disease, the second state is having non-critical COVID-19 disease, and the plurality of gene modules are the gene modules listed in Table 1.
17. The method of any one of embodiments 1 to 13, wherein the first state is having lupus, the second state is not having lupus, and the plurality of gene modules are the gene modules listed in Table 9.
18. The method of any one of embodiments 1 to 13, wherein the first state is having psoriasis, the second state is not having psoriasis, and the plurality of gene modules are the gene modules listed in Table 9.
19. The method of any one of embodiments 1 to 13, wherein the first state is having atopic dermatitis, the second state is not having atopic dermatitis, and the plurality of gene modules are the gene modules listed in Table 9.
20. The method of any one of embodiments 1 to 13, wherein the first state is having systemic sclerosis, the second state is not having systemic sclerosis, and the plurality of gene modules are the gene modules listed in Table 9.
21. The method of any one of embodiments 1 to 13, wherein the first state is having lupus, the second state is having psoriasis, and the plurality of gene modules are the gene modules listed in Table 9.
22. The method of any one of embodiments 1 to 13, wherein the first state is having lupus, the second state is having atopic dermatitis, and the plurality of gene modules are the gene modules listed in Table 9.
23. The method of any one of embodiments 1 to 13, wherein the first state is having lupus, the second state is having systemic sclerosis, and the plurality of gene modules are the gene modules listed in Table 9.
24. The method of any one of embodiments 1 to 13, wherein the first state is responsiveness to a lupus drug selected from a neutrophil function inhibitor, a TNF inhibitor, an IL1 inhibitor, a Plasma cell inhibitor, a NK cell inhibitor, or a B cell inhibitor, the second state is non-responsiveness to a lupus drug selected from neutrophil function inhibitor, a TNF inhibitor, an IL1 inhibitor, a Plasma cell inhibitor, a NK cell inhibitor, or a B cell inhibitor, and the plurality of gene modules are the gene modules listed in Table 10.
25. The method of embodiment 24, wherein the Plasma cell inhibitor comprises Mycophenolate, Bortezomib, Carfilzomib, Ixazomib, Daratumumab, Isatuximab, Elotuzumab, or any combinations thereof; the IL1 inhibitor comprises Anakinra, Canakinumab, or any combinations thereof; the TNF inhibitor comprises Adalimumab, Certolizumab pegol, Etanercept, Golimumab, Infliximab, or any combination thereof; the Neutrophil function inhibitor comprise Dasatinib, Apremilast, Roflumilast, or any combinations thereof; the NK cell inhibitor comprises Azathioprine; and the B cell inhibitor comprises Belimumab, Rituximab, Obinutuzumab, Ocrelizumab, Ofatumumab, Inebilizumab, or any combination thereof.
26. A method for classifying a patient between a first state and a second state, the method comprising:
   analyzing a patient data set comprising gene expression measurements of the genes of a gene set obtained by the method of any one of embodiments 1 to 25, from a biological sample obtained or derived from the patient.
27. The method of embodiment 26, wherein analyzing the patient data set comprises providing the patient data set as an input to a machine learning model trained to generate an inference of whether the patient data set is indicative of the patient classification between the first state and the second state.
28. The method of embodiment 27, further comprising:
   receiving, as an output of the machine-learning model, the inference; and
   electronically outputting a report classifying the patient between the first state and the second state.
29. The method of any one of embodiments 26 to 28, wherein the biological sample comprises a blood sample, isolated peripheral blood mononuclear cells (PBMCs), tissue biopsy sample, nasal fluid, saliva, urine, stool, or any derivative thereof.
30. The method of any one of embodiments 26 to 29, further comprising recommending, selecting and/or administering a treatment to the patient based on the classification of the patient.

### EXAMPLES

The following illustrative examples are representative of embodiments of the software applications, systems, and methods described herein and are not meant to be limiting in any way.

### Example 1: Classification of COVID-19 Patients into Clinically Relevant Subsets by Machine Learning Using Transcriptomic Features

The persistent impact of the COVID-19 pandemic and heterogeneity in disease manifestations point to a need for innovative approaches to identify drivers of immune pathology and predict whether infected patients will present with mild/moderate or severe disease. We have developed a novel iterative machine learning pipeline that utilizes gene enrichment profiles from blood transcriptome data to stratify COVID-19 patients based on disease severity and differentiate severe COVID cases from other patients with acute hypoxic respiratory failure. The pattern of gene module enrichment in COVID-19 patients overall reflected broad cellular expansion and metabolic dysfunction whereas increased neutrophils, activated B cells, T cell lymphopenia, and proinflammatory cytokine production were specific to severe COVID patients. Using this pipeline, we also identified small blood gene signatures indicative of COVID-19 diagnosis and severity that could be used as biomarker panels in the clinical setting.

Since its emergence in December 2019, infection by severe acute respiratory syndrome coronavirus 2 (SARS-COV-2), the causative agent of coronavirus disease 2019 (COVID-19), has led to the deaths of over 6.5 million individuals worldwide [1-3]. COVID-19 is characterized by a wide range of disease presentations from mild with flu-like symptoms to severe hypoxic respiratory failure (AHRF) requiring hospitalization and admittance to the intensive care unit (ICU) [4]. In addition, symptoms may persist for months after patients present with acute illness [5]. A number of pre-existing clinical risk factors (age, gender, obesity, respiratory conditions, diabetes, immunodeficiency) [6,7] and post-infection, multi-organ disease complications, including cardiac injury, thrombosis, renal disease, and liver injury [8-10] have been associated with severe COVID. Despite intense study, however, the major drivers of severe and potentially fatal COVID-19 have not been fully elucidated.

Considerable resources have been applied to study the immune response to SARS-COV-2 infection as a means to understand COVID-19 pathogenesis in greater detail [11,12]. Among these efforts, our group and others have employed a bioinformatics-based approach using multi-omic, and in particular, transcriptomic data to construct immune profiles of COVID-19 patients at various stages of disease onset and severity [13-22]. These studies have identified numerous abnormalities in the strength and nature of the adaptive and innate immune responses of severe COVID patients, including neutrophilia, T cell lymphopenia, plasmablast expansion, antibody or autoantibody production, and IFN levels. With this accumulated knowledge base, the focus has evolved to attempt to apply the information to identify subsets of individuals with COVID-19 with prognostic relevance.

Recent advances in computer science and the use of artificial intelligence (AI) and machine learning (ML) for clinical applications offer a promising approach to identify biomarkers and predict the risk of SARS-COV-2 infected individuals to develop severe and possibly fatal disease [23,24]. Several recent studies have employed ML or deep learning approaches to patient demographic data [25], lung CT scan images [26-28], and transcriptomic data [29-33] to stratify COVID-19 patients with varying degrees of success. However, more work is required to improve model performance and reproducibility before biomarkers of severe COVID-19 identified by ML can be translated into a clinical setting. This is dependent on: (1) developing a standardized approach to dealing with high dimensional data; (2) selecting the best ML algorithm for each application; and (3) selecting the most informative features as input for ML applications. To that end, we have developed an iterative ML pipeline based on curated gene signatures previously used to characterize immune profiles of COVID-19 patients from whole blood gene expression data [13,14]. As a result, we have identified immunological signatures and individual genes with strong predictive capacity for classifying COVID-19 patients most at risk of severe disease that could be employed as a diagnostic and prognostic tool in the clinic.

### Results

A gene expression-based iterative machine learning approach to classification of COVID-19 patients. We employed three publicly available whole blood transcriptome datasets (GSE161731, GSE172114, and PRJNA777938,) that had previously been analyzed for immune profiling of COVID-19 patients based on gene signature enrichment by gene set variation analysis (GSVA) [14]. The combined datasets consisted of individuals with COVID-19, individuals with non-COVID-induced AHRF, individuals admitted to the ICU without AHRF, and healthy controls. COVID-19 patients were further categorized as “non-critical” if they exhibited symptoms, but were not hospitalized or were admitted to a non-critical care ward and as “critical” if they exhibited more severe symptoms requiring hospitalization and admittance to the ICU.

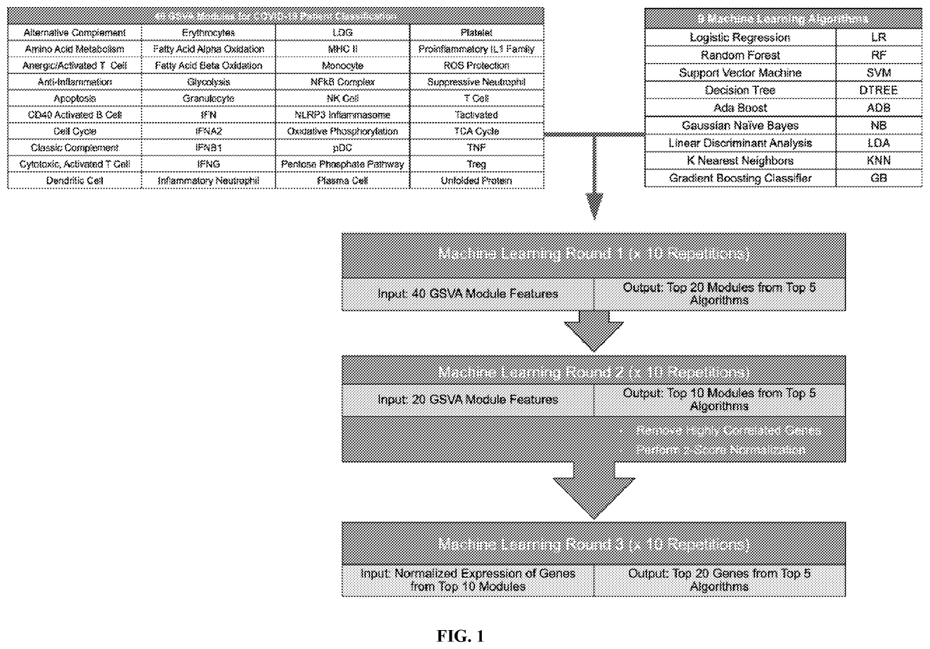

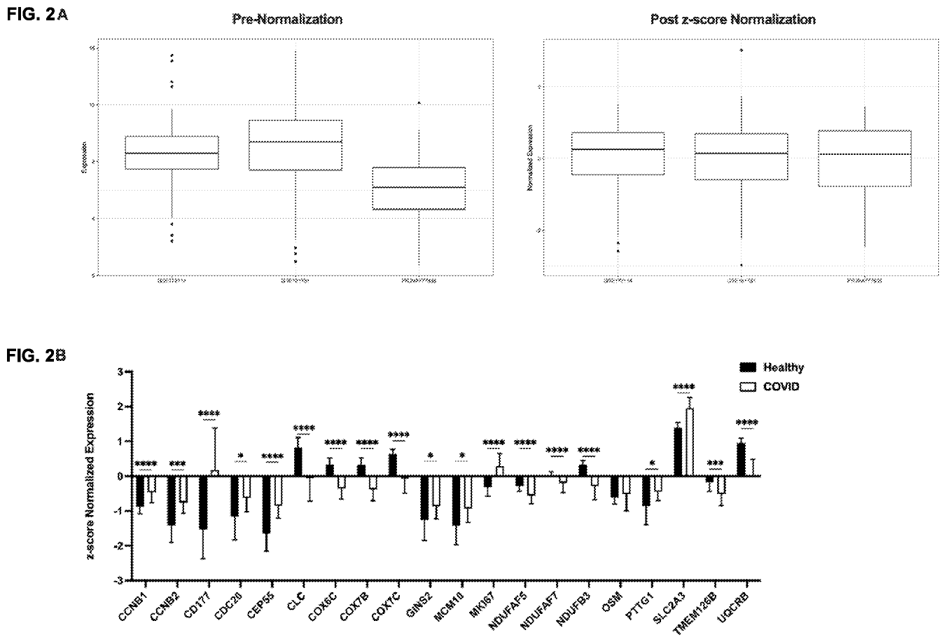

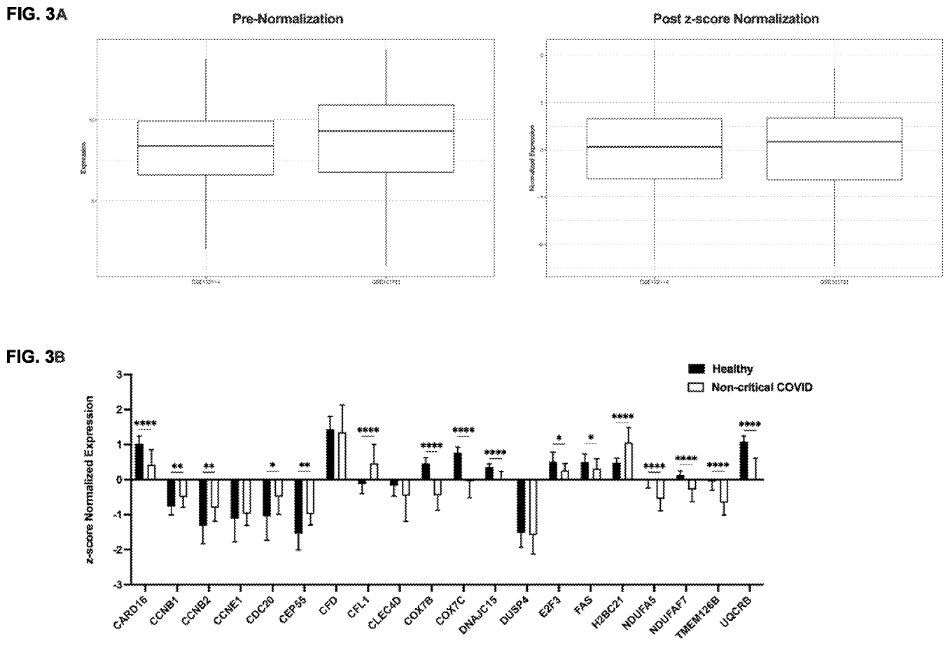

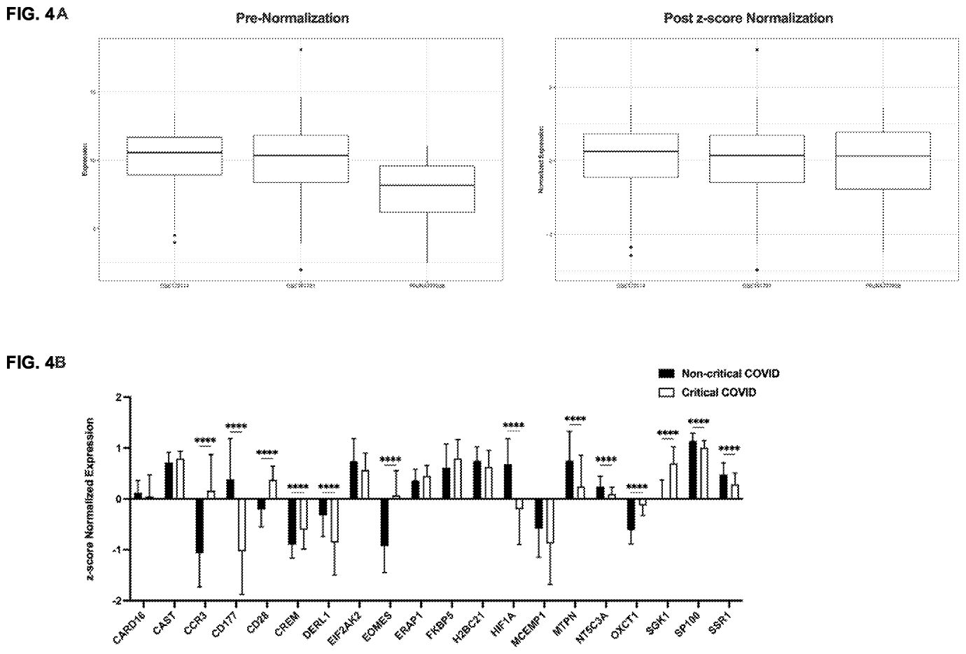

To determine the top immune signatures and genes differentiating subsets of COVID-19 patients from healthy individuals and other ICU patients, combined GSVA enrichment scores for 40 curated immune cell and pathway gene signatures [13,14] were used as features to train 9 ML algorithms (FIG. 1, Table 1). Each ML algorithm was used for 4 binary classifications: COVID patients versus healthy individuals, non-critical COVID patients versus healthy individuals, critical versus non-critical COVID patients, and COVID ICU patients versus non-COVID ICU patients. Then, the top 5 performing ML algorithms were employed in an iterative approach to identify the GSVA modules contributing most to each classification (FIG. 1). After each iteration, feature importance was calculated for each ML algorithm and the top 50% of features were used for the next round of ML. Then, the log2 expression values of genes composing the final 10 gene modules were used as features for ML algorithms to identify the top 20 individual genes that could effectively classify COVID-19 patients in each comparison. Expression values used as input for the final ML iteration were normalized to account for batch effects between separate RNA-seq datasets (FIGS. 2A, 3A, 4A, and 5A). To ensure reproducibility of the results, each ML iteration was repeated 10 times and the most represented module or gene features were used moving forward. Overall, the ML algorithms successfully performed each binary classification and the top performing algorithms for each comparison achieved accuracies of >0.7 for all iterations of ML. In addition, accuracies improved between the first and last ML iteration as the gene signature features were refined and distilled into individual gene features and final accuracies reached >0.9 for the top 5 algorithms in each classification (Tables 2A-D). Notably, the final gene lists derived from this iterative ML approach reflected distinct immune cell and inflammatory pathways providing the greatest contribution to each binary classification of COVID-19 patients (Tables 3A-D).

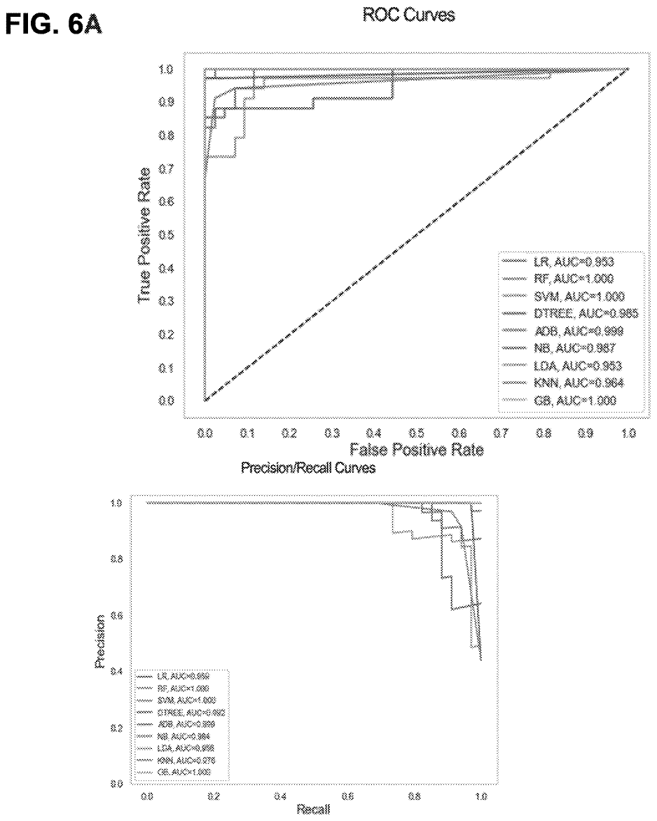

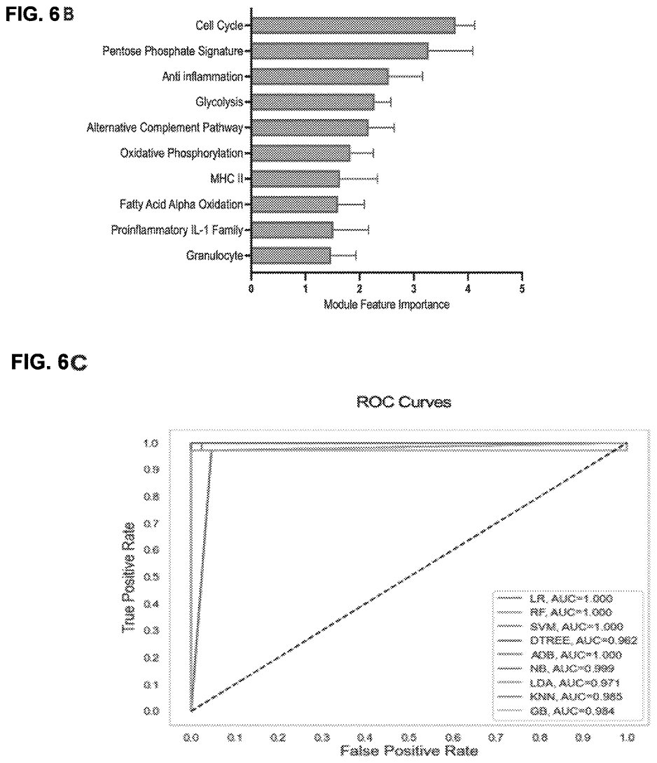

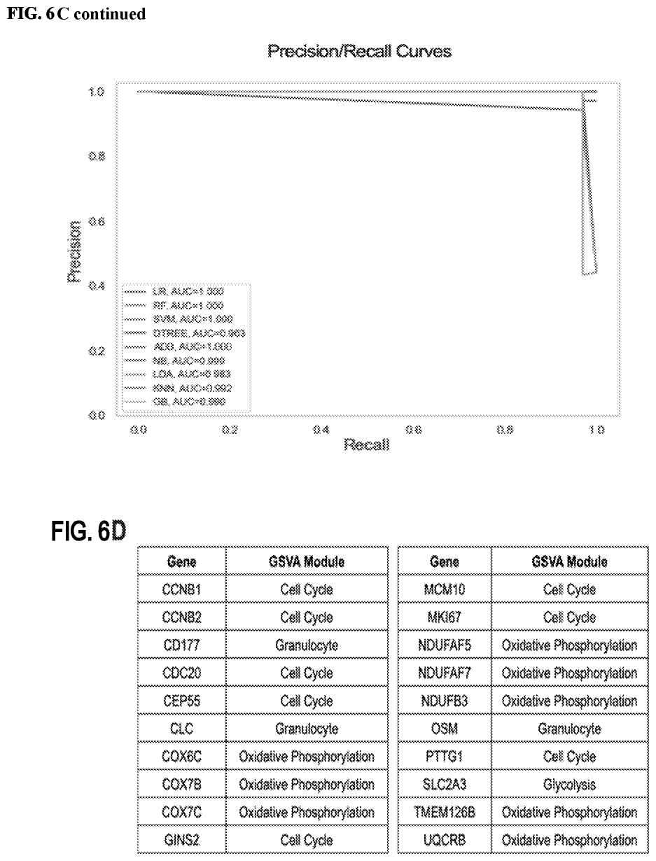

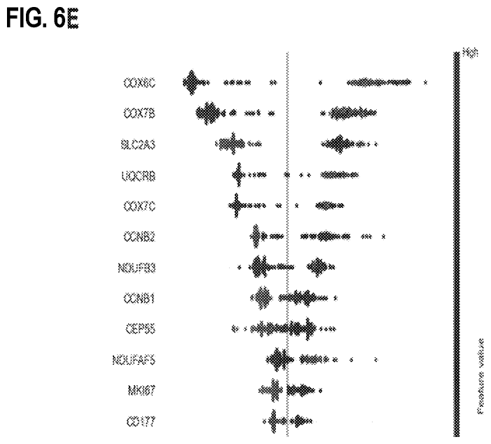

Machine learning models reveal genes critical for classification of COVID-19 patients from healthy individuals. The iterative ML pipeline was first applied to determine the top modules and individual genes for classification of COVID-19 patients from healthy individuals. For the initial iteration, support vector machine (SVM) was consistently the top performing algorithm with average areas under the receiver operating characteristic and precision/recall curves (AU-ROC and AU-PR) of 1.0 (FIG. 6A), as well as an average overall accuracy of 1.0 indicative of perfect model performance across 10 repetitions (Table 2A). The 10 modules with the highest feature importance were divided between pathways related to cell proliferation/metabolism (Cell Cycle, Pentose Phosphate Signature, Glycolysis, Oxidative Phosphorylation (OxPhos), Fatty Acid Alpha Oxidation (FAAO)) and inflammatory modules (Anti inflammation, Alternative Complement Pathway, Granulocyte, MHC II, Proinflammatory IL-1 Family) (FIG. 6B, Table 3A). In the final ML iteration, the perfect classification metrics observed in the first iteration were maintained, but linear regression (LR) and random forest (RF) were the top performing algorithms (FIG. 6C, Table 2A). This result highlights the importance of comparing multiple algorithms when inputting different sets of features to the ML pipeline. The final 20 genes with greatest performance in classifying COVID-19 patients from healthy individuals were dominated by genes from modules of the Cell Cycle (CCNB1, CCNB2, CDC20, CEP55, GINS2, MCM10, MKI67, PTTG1) and OxPhos (COX6C, COX7B, COX7C, NDUFAF5, NDUFAF7, NDUFB3, TMEM126B, UQCRB), but also included genes in the Glycolysis (SLC2A3), and Granulocyte (CD177, CLC, OSM) modules (FIG. 6C, FIG. 2B, Table 3A). To determine the exact relationship between expression of these 20 genes and COVID-19 classification, we used SHapley Additive Explanations (SHAP) values, which provide information on the magnitude and directionality of the contribution of each gene to classification of an individual as a COVID-19 patient or a healthy control (FIG. 6E). SHAP values are displayed in a summary plot (FIG. 6E) in which each dot represents the expression of each gene in an individual patient and higher gene expression values in conjunction with increased SHAP values are indicative of a positive association with COVID-19 classification and lower gene expression values in conjunction with increased SHAP values indicate a negative correlation with COVID-19 classification. Overall, we found that increased expression of cell cycle and glycolysis genes and decreased expression of OxPhos genes were associated with COVID-19 classification (FIG. 6E). Genes in the granulocyte module were split with increased expression of CD177 and decreased expression of CLC and OSM contributing to COVID classification (FIG. 6E).

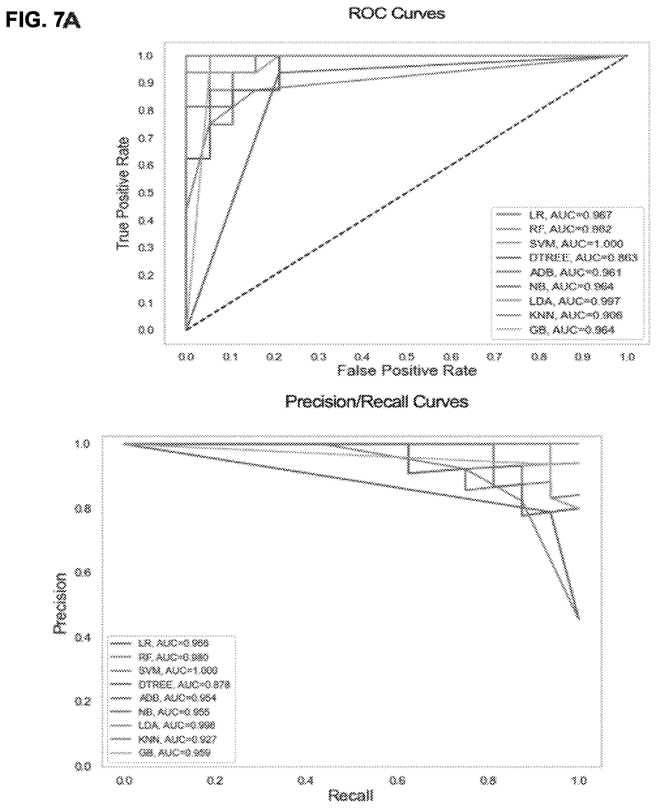

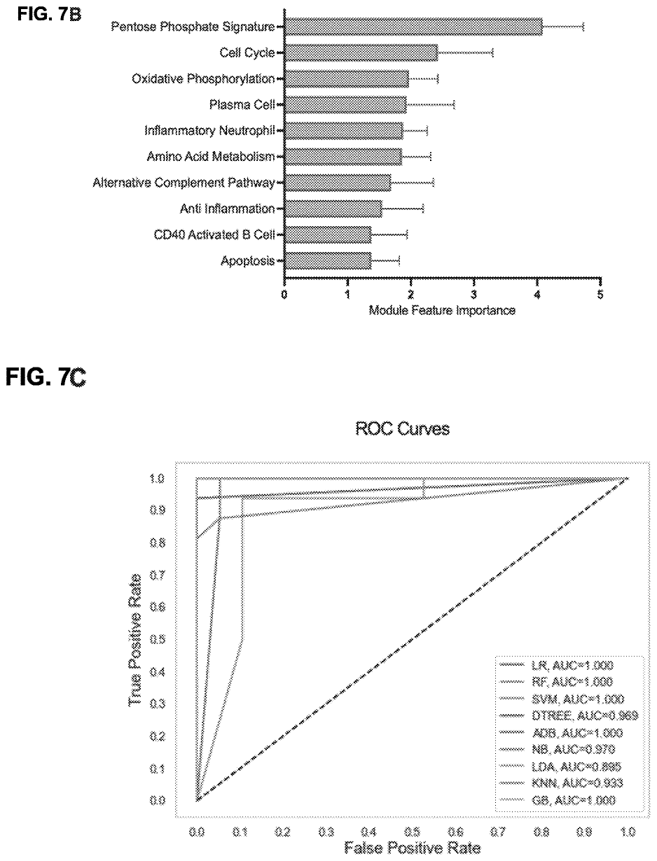

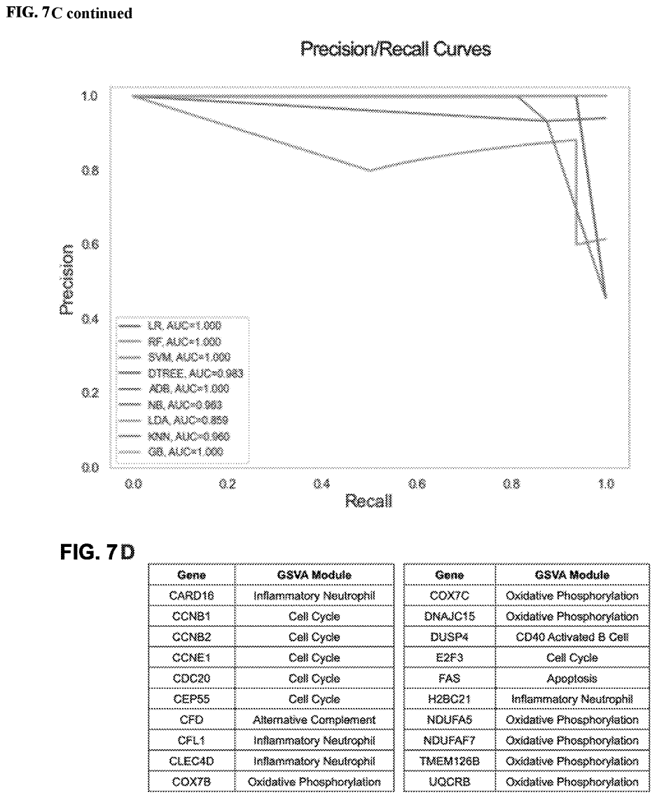

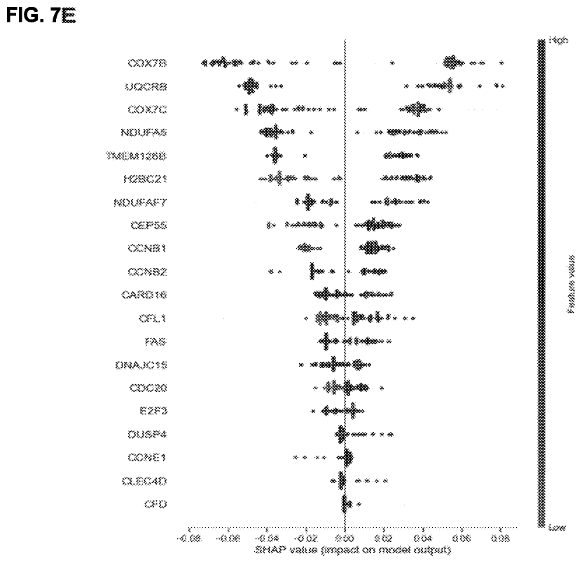

The COVID-19 patient cohort consisted of individuals with mild, non-critical disease as well as individuals with severe disease requiring ICU admission. Therefore, we sought to determine whether an iterative ML approach could also effectively identify subtle differences between COVID-19 patients with mild disease and healthy individuals (FIGS. 7A-E). Interestingly, the non-critical COVID vs healthy classifier performed similarly to classification including COVID-19 patients with severe disease. In the first ML iteration, SVM was the top performing algorithm over 10 repetitions with an average AU-ROC of 0.994, AU-PR of 0.992, and accuracy of 0.976 (FIG. 7A, Table 2B). The top 10 gene modules for the non-critical COVID-19 vs healthy classifier shared common proliferation/metabolism pathways with results when including all COVID-19 patients but, interestingly, had more representation of immune cell gene modules including Plasma Cells (PCs), CD40 Activated B Cells, and Inflammatory Neutrophils (FIG. 7B, Table 3B). Algorithm performance improved by the final ML iteration and LR and RF algorithms performed the best with perfect classification metrics in 9 out of 10 repetitions (FIG. 7C, Table 2B). The additional immune modules selected as top features identifying non-critical COVID-19 patients from healthy controls were reflected in the final list of genes as in addition to Cell Cycle (CCNB1, CCNB2, CCNE1, CDC20, CEP55, E2F3) and OxPhos (COX7B, COX7C, DNAJC15, NDUFA5, NDUFAF7, TMEM126B, UQCRB), genes in the Inflammatory Neutrophil (CARD16, CFL1, CLEC4D, HBC21), CD40 Activated B Cell (DUSP4), Alternative Complement Pathway (CFD), and Apoptosis (FAS) modules were also included (FIG. 7D, FIG. 3B, Table 3B). SHAP values for the top 20 genes indicated that increased expression of genes in the Cell Cycle, Alternative Complement Pathway, CD40 Activated B Cell, and Inflammatory Neutrophil modules and decreased expression of genes in the OxPhos and Apoptosis modules were associated with classification of non-critical COVID-19 patients as compared to healthy individuals (FIG. 7E). Thus, an iterative ML approach effectively differentiated COVID-19 patients with varying levels of disease severity from healthy individuals based solely on expression of gene modules and individual genes in each patient.

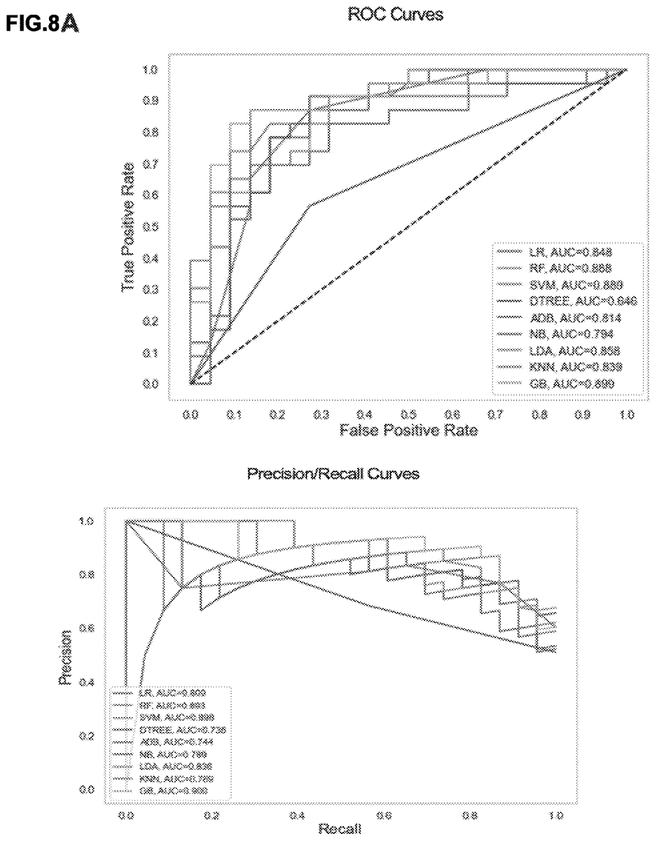

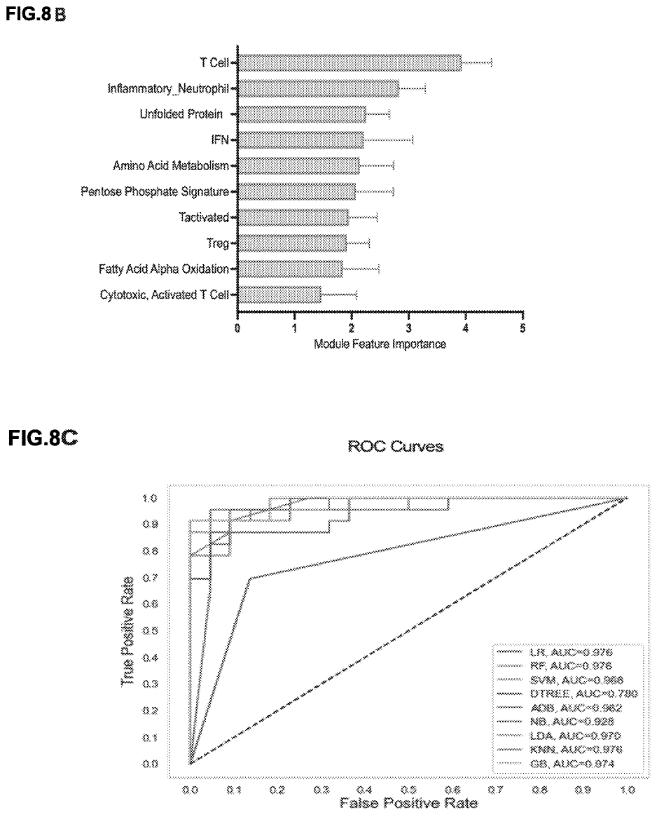

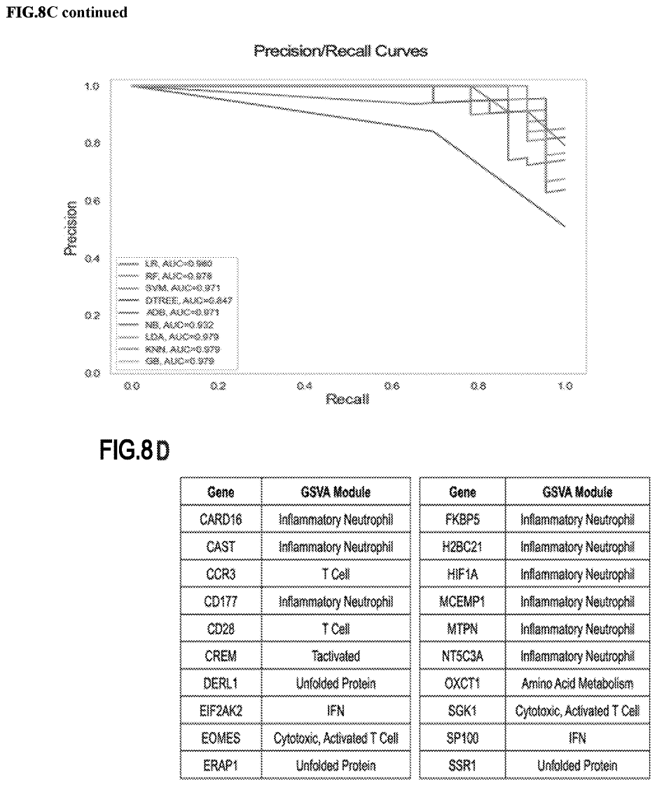

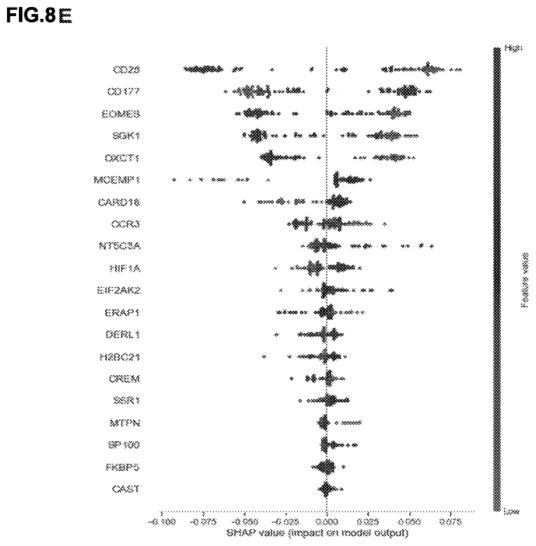

Iterative machine learning effectively predicts disease severity of COVID-19 patients based on gene expression. To extend the clinical applications of gene expression-based classification of COVID-19 patients, we employed the iterative ML pipeline to classification of COVID-19 patients with critical, severe disease requiring hospitalization from patients with a mild, non-critical form of the disease (FIGS. 8A-E). For the critical vs non-critical COVID-19 classifier, the Gradient Boosting (GB) algorithm performed best in the first iteration with an average AUC-ROC and AUC-PR of 0.82 and an average accuracy of 0.83 across 10 repetitions (FIG. 8A, Table 2C). The top 10 gene modules distinguishing critical from non-critical COVID-19 largely consisted of inflammatory cell/pathway modules and, in particular, T cells (T Cell, Inflammatory Neutrophil, IFN, Tactivated, Treg, Cytotoxic, Activated T Cell) as well as metabolic pathways (Amino Acid (AA) Metabolism, Pentose Phosphate Signature, FAAO) and the stress response (Unfolded Protein) (FIG. 8B, Table 3C). As with the COVID-19 vs healthy classifier, performance across all algorithms improved by the final iteration and GB remained the top performer with an average AUC-ROC of 0.98, AUC-PR of 0.98, and overall accuracy of 0.93 (FIG. 8C, Table 2C). As a result, the majority of the top 20 individual genes identifying critical COVID-19 patients with more severe disease were derived from the Inflammatory Neutrophil module (CARD16, CAST, CD177, FKBP5, H2BC21, HIFIA, MCEMP1, NT5C3A) as well as genes from the IFN (EIF2AK2, SP100), Unfolded Protein (DERL1, ERAPI, SSR1), AA Metabolism (OXCT1), and T cell-related modules (CCR2, CD28, CREM, EOMES, SGK1) (FIG. 8D, FIG. 4B, Table 3C). SHAP analysis of the directionality of relationships between relative expression of the top 20 genes and classification of critical COVID-19 revealed that, as a whole, critical classification was associated with increased expression of inflammatory neutrophil and IFN genes and decreased expression of T cell, metabolism, and stress response genes (FIG. 8E). These results emphasize the differences in the nature of the inflammation present in critical COVID-19 patients as compared to those with mild disease.

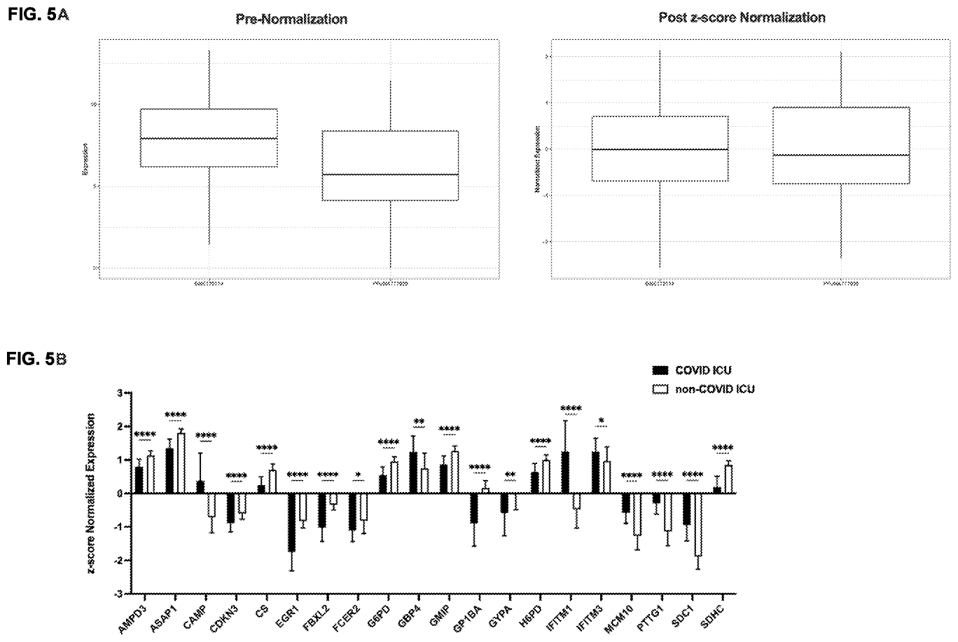

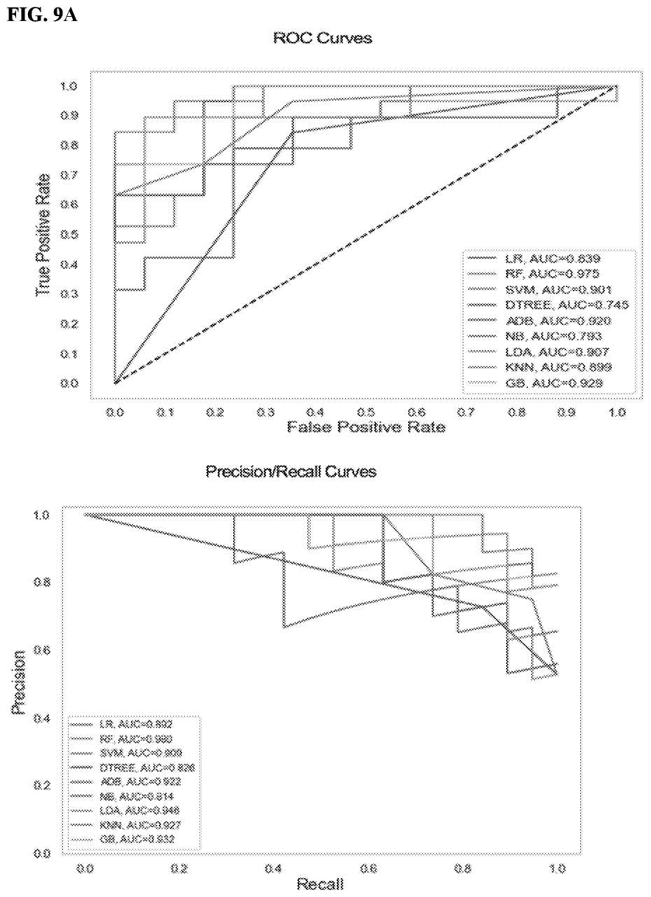

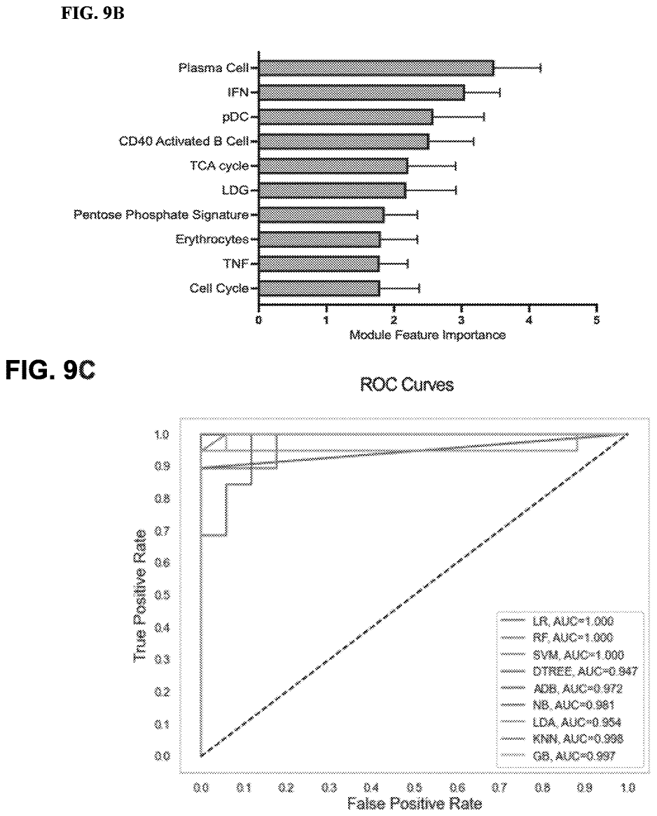

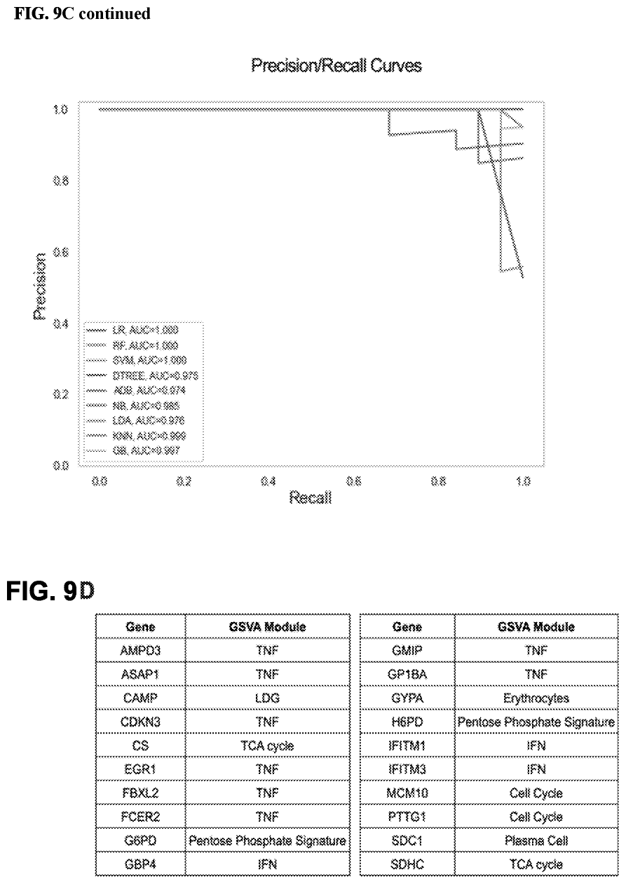

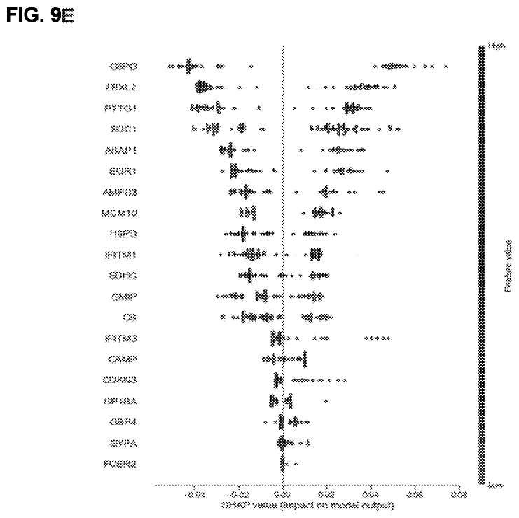

Gene expression-based machine learning approach identifies genes distinguishing COVID-19 ICU patients from other patients admitted to the ICU. Our previous analysis of patients admitted to the ICU with or without COVID-19-induced AHRF found few differences in gene-expression based immune profiles, predominantly centered on the nature and magnitude of the PC antibody response [14]. To explore these differences in greater depth, we utilized the iterative ML pipeline for classification of COVID-19 vs non-COVID-19 ICU patients (FIGS. 9A-E). The first iteration of this classifier had the weakest initial algorithm performance and SVM performed best in 5 out of 10 repetitions with an average AUC-ROC of 0.77, AUC-PR of 0.81, and accuracy of 0.76 (FIG. 9A, Table 2D). Of the top 10 gene modules for the COVID-19 vs non-COVID-19 ICU classifier, the PC module had the greatest overall feature importance as expected from our previous work (FIG. 9B, Table 3D). The remaining modules in the top 10 included those related to the inflammatory response (IFN, pDC, CD40 Activated B Cell, LDG, Erythrocytes, TNF) as well as cell proliferation and metabolism (Cell Cycle, TCA Cycle, Pentose Phosphate Signature). Algorithm performance improved dramatically by the final ML iteration in the pipeline and linear regression (LR) was the best overall algorithm with average AUC-ROC of 0.91, AUC-PR of 0.99, and accuracy of 0.97 (FIG. 9C, Table 2D). The top 20 genes predominantly originated from the TNF module (AMPD3, ASAP1, CDKN3, EGRI, FBXL2, FCER2, GMIP, GPIBA), but also included genes from the PC (SDC1), IFN (GBP4, IFITM1, IFITM3), Cell Cycle (MCM10, PTTG1), TCA cycle (CS, SDHC), Pentose Phosphate Signature (G6PD, H6PD), LDG (CAMP), and Erythrocyte (GYPA) modules (FIG. 9D, FIG. 5B, Table 3D). Interestingly, the SHAP summary plot indicated that decreased expression of most of these genes including those from the TNF and metabolism-related modules and increased expression of genes from the PC, IFN, and LDG modules was associated with COVID-19 classification from all patients admitted to the ICU (FIG. 9E). Altogether, these results demonstrate that gene expression data can effectively classify subsets of COVID-19 patients and provide insight into inflammatory cells and pathways with the greatest contribution to disease severity.

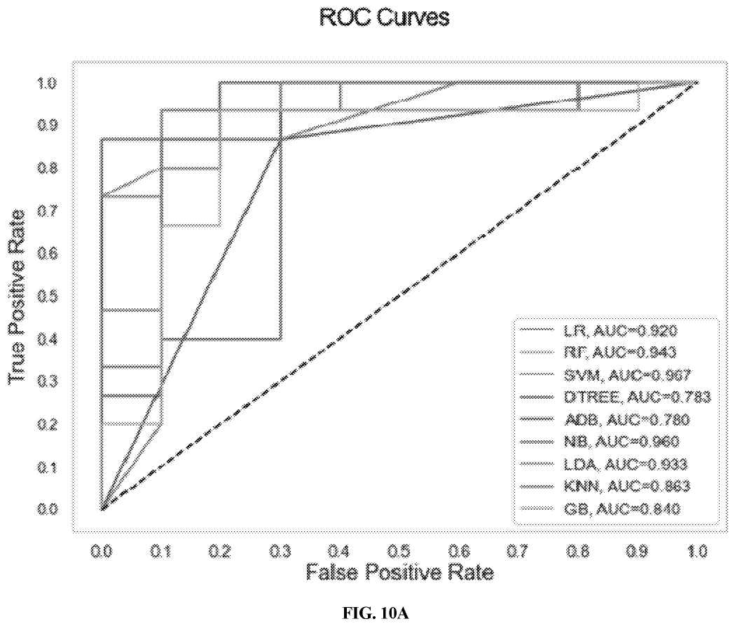

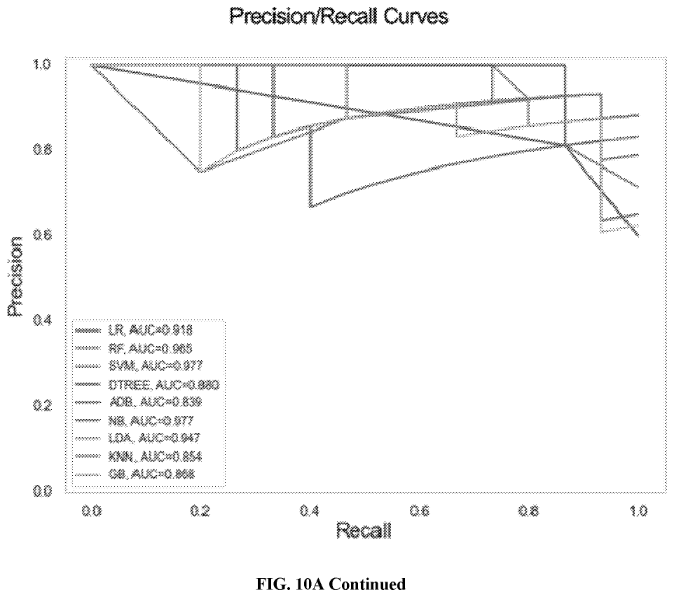

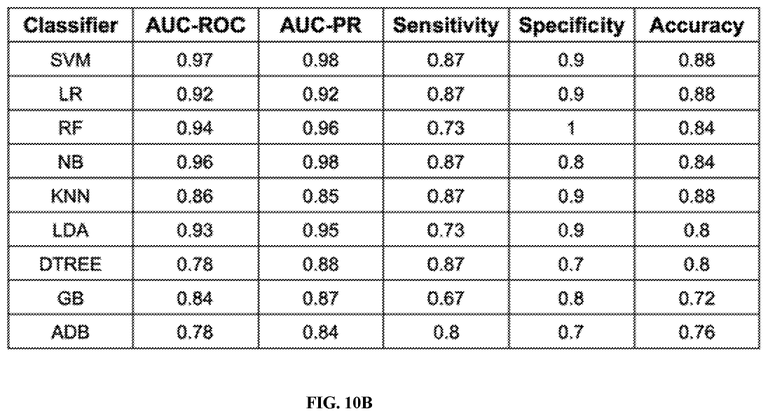

Validation of the iterative ML pipeline in an independent COVID-19 patient dataset. Next, we determined whether the small gene signature output from our iterative ML pipeline could distinguish COVID-19 patients with mild/moderate and severe disease in an independent dataset (GSE157103) [21]. Blood RNA-seq data from 100 hospitalized COVID-19 patients (50 admitted to the ICU and 50 non-ICU) was used to validate the 20 gene signature from the critical vs. non-critical COVID-19 classifier (FIG. 8D, Table 3D). Scaled log2 expression values for the 20 genes were used as input for 9 ML algorithms and combined ROC and PR curves were generated (FIG. 10A). SVM was the top performing algorithm with excellent AUC-ROC and AUC-PRs of 0.97. Classification performance metrics were high for all algorithms with sensitivities ranging from 0.67-0.87, specificities from 0.7-1, and accuracies from 0.72-0.88 (FIG. 10B). Therefore, gene signatures derived from our iterative ML pipeline can be successfully applied to classification of COVID-19 severity in an independent patient cohort and achieve a comparable level of model performance.

### Discussion

The heterogeneity of COVID-19 and wide range of individuals affected by acute and/or persistent disease manifestations necessitates an accurate and reproducible approach to identify drivers of pathology and disease trajectory over time. Once established, this information could be leveraged to predict whether an individual that tests positive for COVID-19 will develop a mild/moderate case warranting standard of care treatment or a severe case requiring more careful monitoring and deployment of targeted therapeutics to prevent multi-organ damage and death. We have developed an iterative ML approach to identify key molecular signatures stratifying COVID-19 patients based on disease severity and differentiate them from healthy individuals and those with other respiratory or non-respiratory conditions. This pipeline extends and improves upon previous efforts at ML-based stratification of COVID-19 patients, resulting in four separate gene biomarker panels distinguishing COVID-19 patients from healthy individuals, critical from non-critical COVID-19 patients and critical COVID-19 patients from those admitted to the ICU for non-COVID-19 AHRF. Furthermore, each classifier achieved excellent ML performance metrics across 9 different ML algorithms and over 10 repetitions of each binary classification and effectively translated to an independent validation cohort of COVID-19 patients.

In developing a novel ML pipeline that would yield the most reliable and reproducible results, we considered a number of critical factors including (1) the type of input data to use, (2) the ML algorithm(s) that would be most appropriate, and (3) the method to select the most informative, non-redundant features. First, we used gene expression data as input for the ML pipeline as it produces thousands of data points and, thus, provides a comprehensive overview of COVID-19 disease profiles that is easily obtainable from a single blood draw [34-36]. In addition, our previous studies demonstrated the utility of bulk transcriptome data to characterize COVID-19 immunopathology across different tissues and levels of disease activity [13,14], results that have been validated by other multi-omics studies [15-22]. Second, to account for differences and potential biases in ML algorithms used for patient stratification, we utilized 9 different supervised classifiers spanning a wide range of approaches to model construction. These included if/then (DTREE), regression (LR), ensemble (RF, ADB, GBM), Bayesian (NB), dimensionality reduction (LDA), and instance-based algorithms (SVM, KNN) [37-39]. In addition, multiple repetitions of each step in the ML pipeline further increased confidence in the reliability and reproducibility of the results. Finally, in working with high-throughput gene expression data, we faced the issue of feature selection and how to reduce transcriptome-wide datasets into a manageable number of gene features that would optimize ML algorithm performance [40,41]. We accounted for this by using curated gene modules representative of immune cells and pathways previously associated with COVID-19 pathogenesis. Then, using an iterative approach in which the most informative features were selected moving forward allowed us to reduce the number of genes used for the final iteration and select the top 20 overall genes able to distinguish COVID-19 patients in each classifier.

This iterative ML pipeline was used for classification of COVID-19 patients from healthy individuals, critical from non-critical COVID-19 patients, and COVID-19 from non-COVID-19 patients admitted to the ICU with AHRF. Overall, the pathologic cell types and processes represented in the resulting modules and genes reflected previous characterizations of COVID-19 patients, providing validation for these results. Comparing differences in the output of each classifier provided additional insights into the distinguishing features of severe manifestations of COVID-19 as compared to other conditions. Classification of all COVID-19 patients from healthy individuals was dependent on increased expression of genes involved in the cell cycle and decreased expression of genes involved in mitochondrial function through OxPhos. Dysregulated expression of cell cycle genes in COVID-19 patients has been reported in several studies in conjunction with outgrowth of peripheral blood leukocyte populations and, thus may serve as a marker of generalized inflammation in infected individuals [42-44]. The decreased expression of OxPhos genes is indicative of mitochondrial dysfunction and oxidative stress, both of which are a general feature of the response to viral infection, and have also been implicated specifically in COVID-19 patients [45-47]. Interestingly, isolating non-critical COVID-19 patients in comparison to healthy individuals yielded a final gene list that, in addition to cell cycle and OxPhos genes, incorporated genes linked with pro-inflammatory cell populations, including inflammatory neutrophils and activated B cells. Our group and others have previously associated neutrophil-driven inflammation and rapid outgrowth of antibody secreting cells (ASCs) with early SARS-COV-2 infection [14,48], suggesting that these signatures are early signs of mild/moderate disease and that continued expansion of these populations could result in critical illness.

The final 20 gene list from the critical vs non-critical COVID-19 classifier indicated that increased expression of inflammatory neutrophil, IFN, and stress response pathway genes in conjunction with decreased T cell genes were hallmarks of severe COVID-19. Most of the 20 gene signature was composed of inflammatory neutrophil genes, which were originally identified in the blood of severe COVID-19 patients [49,50]. Of these, CARD16 and H2BC21 were shared in the signature differentiating non-critical COVID-19 patients from healthy individuals. However, their increased expression in critical patients emphasizes the greater prevalence of pathologic neutrophils in promoting severe disease complications. An elevated IFN response has also been associated with severe COVID-19 in numerous studies in line with a dysregulated systemic inflammatory response and increased expression of stress response genes as tissue damage occurs [16,51,52]. As pro-inflammatory response signatures were increased in severe COVID-19 patients, this was accompanied by a decrease in immunoregulatory population genes and, in particular, T cells indicative of T cell lymphopenia, which has been frequently described in severe COVID-19 [15,53,54]. Notably, the specific inclusion of cytotoxic T cell-specific genes (EOMES and SGK1) in the 20 gene signature differentiating critical patients further supports a role for inability to clear viral infection in COVID-19 severity.

We also used our iterative ML pipeline to differentiate ICU patients with COVID-19 from those admitted with non-COVID-19 conditions. The most prevalent module for genes resulting from this classifier were indicative of a high TNF response in COVID-19 ICU patients and high expression of TNF family members has been found in damaged tissue of COVID-19 patients indicative of severe disease [55]. This result suggests that while numerous inflammatory cytokines have been implicated in COVID-19 pathogenesis, a TNF response is unique to COVID-19 associated AHRF compared with other conditions requiring admittance to a critical care ward and provides evidence that TNF inhibitors may be a viable option for treatment specifically of COVID-19 [56]. In addition, inclusion of the PC gene SDC1 in the final gene list distinguishing COVID-19 from non-COVID-19 ICU patients is in agreement with our previous work in which PC enrichment was the major difference in these cohorts [14].

Overall, our novel iterative ML pipeline for COVID-19 patient stratification offers a robust method of identifying critical inflammatory processes driving progression to severe disease. In addition, the excellent performance of the resulting algorithms instills confidence that they can be applied to predict disease trajectories of newly diagnosed individuals as demonstrated by the effectiveness in distinguishing severe COVID-19 patients, admitted to the ICU from non-ICU COVID-19 patients. We propose that this approach be used for development of rapidly deployable blood-based PCR tests employing a small number of highly informative genes to provide clinical support for COVID-19 patient risk assessment in the clinic.

### Materials and Methods

Study Datasets. Three publicly available whole blood bulk RNA-seq datasets previously analyzed for characterization of COVID-19 patients with varying degrees of severity were used as input for machine learning classification algorithms and an additional dataset was analyzed for validation. Raw SRA files were downloaded from Gene Expression Omnibus (GEO) accessions: GSE161731 [19], GSE172114 [22], PRJNA777938 [14], and GSE157103 [21], converted into FASTQ files, and processed into log2 gene expression values as previously described [14].

Gene Set Variation Analysis (GSVA). The R/Bioconductor package GSVA (v1.36.3) was used as a non-parametric, unsupervised method for determining enrichment of pre-defined gene modules in individual gene expression samples as previously described [14]. GSVA enrichment scores for 40 gene modules (Table 1) previously used for characterization of immune profiles in blood from COVID-19 patients [13,14] were calculated for each patient and used as input for the iterative machine learning pipeline.

Iterative Machine Learning (ML) Pipeline. The iterative ML pipeline is outlined in FIG. 1. Briefly, GSVA enrichment scores for 40 gene modules (Table 1) from publicly available whole blood bulk RNA-seq datasets were used as input for 9 ML algorithms in each of 4 binary classifications. The pipeline consisted of 3 ML iterations each of which was repeated 10 times. After the first 2 ML iterations, the top 50% of features from the top 5 performing algorithms were used as input for the next iteration. Then, the top 10 gene modules were filtered to remove redundant genes with correlation coefficients >0.8 and log2 gene expression values from the remaining genes were normalized across datasets. Normalized z-scores of gene expression values were used as input for the final ML iteration to select the top 20 gene features for each classifier. Detailed methods for each step in the pipeline are described below.

ML Classification Algorithms. Binary classifications were carried out in Python (v3.8.2) using scikit-learn (v0.24.1) [58]. Nine ML algorithms were implemented to distinguish COVID-19 patients from healthy individuals, critical from non-critical COVID-19 patients, and COVID-19 patients admitted to the ICU from non-COVID ICU patients (Logistic Regression (LR), Random Forest (RF), Support Vector Machine (SVM), Decision Tree (DTREE), Ada Boost (ADB), Gaussian Naïve Bayes (NB), Linear Discriminant Analysis (LDA), k Nearest Neighbors (KNN), and Gradient Boosting Classifier (GB)). Synthetic Minority Oversampling Technique (SMOTE) was used to account for class imbalances between patient groups used for each classification by expanding the number of samples in the minority class to ensure equal numbers between classes used for ML. For each classification, datasets were split into 70% training and 30% validation and SMOTE was applied to the training data. Classification models for each ML algorithm were then built on the training set using default parameters from the scikit-learn library and evaluated on the validation set. Average algorithm performance was calculated based on the following metrics: sensitivity, specificity, accuracy, and areas under the Receiver Operating Characteristic (ROC) and Precision-Recall (PR) curves. ROC and PR curves were plotted using the Python library matplotlib (v3.3.4) [60]. Binary classification of ICU and non-ICU COVID patients from an independent dataset were used as validation for results of the iterative ML pipeline. For this classification, SMOTE was not applied. To optimize model performance on the validation dataset, a 75% training, 25% testing split was used and the class_weight=‘balanced’ parameter was added to the SVM algorithm.

Feature Importance Calculation. Feature importance was calculated for the top 5 performing algorithms in each ML iteration. For each input feature a higher feature importance score indicates how relevant that feature is towards the output of each classifier. The method of calculating feature importance was determined based the appropriate metric for each algorithm and implemented through the scikit-learn Python library. Gini importance, or average decrease in impurity, was used for RF, DTREE, ADB, GB, LR, and LDA while permutation importance was used for SVM, KNN, and NB.

Gene feature correlation analysis. Feature redundancy was calculated for genes in the top 10 modules of each classifier using Pearson correlations between each feature and every other feature. When two genes shared a correlation coefficient >0.8, the gene with the highest degree of correlation with all other genes in the module was removed. Pearson correlations were computed using the cor( ) function in R and plotted using the corplot library.

z-score Gene Expression Normalization. Log2 transformed gene expression values were normalized before the final ML iteration to account for batch effects across separate datasets. To do this, z-scores were calculated for each gene across samples in each dataset using the scale( ) function in R and then normalized datasets were combined into a dataframe used as input for the 9 ML classification algorithms. The pre- and post-normalization expression values for each dataset combination are summarized as boxplots (FIG. 2A, 3A, 4A, 5A).

SHAP Value Calculation. The precise contribution (magnitude and directionality) of the top 20 gene features output from each classifier was determined using Shapley Additive Explanations (SHAP) [61]. SHAP values were calculated based on the RF algorithm for the final iteration of each classifier. SHAP summary plots were visualized in Python using the shape library (v0.39.0).

Statistical Analysis. Normalized gene expression values of the top 20 genes were compared between the two classes of each binary classification using multiple unpaired t-tests with Welch's correction in GraphPad Prism (v9.1.0; www.graphpad.com). A two-tailed p-value<0.05 was considered statistically significant.

TABLE 1
For each module Genes are Listed by: Gene Symbol | Gene Entrez ID;)

Module: Alternative Complement
C3 | 718; C5 | 727; C6 | 729; C7 | 730; C8A | 731; C9 | 735; CFB | 629; CFD | 1675; CFH |
3075; CFHR5 | 81494; CFP | 5199; CR1 | 1378; GZMM | 3004; MIR520B | 574473;
MIR520E | 574461; VSIG4 | 11326;
Module: Amino Acid Metabolism
AASS | 10157; AGXT | 189; AGXT2 | 64902; ALDH4A1 | 8659; ALDH7A1 | 501; ARG1 |
383; GAD1 | 2571; GLUD1 | 2746; GOT1 | 2805; GPT2 | 84706; GRHPR | 9380; IVD | 3712;
MCCC2 | 64087; OAT | 4942; OTC | 5009; OXCT1 | 5019; PC | 5091; PSAT1 | 29968; SDS |
10993;
Module: Anergic_Activated T cells
CD160 | 11126; CD244 | 51744; CTLA4 | 1493; HAVCR2 | 84868; ICOS | 29851; KLRG1 |
10219; LAG3 | 3902; PDCD1 | 5133;
Module: Anti inflammation
IL1RN | 3557; SOCS3 | 9021; TNFAIP3 | 7128;
Module: Apoptosis
AIFM1 | 9131; APAF1 | 317; BAD | 572; BAK1 | 578; BAX | 581; BCL2L11 | 10018; BID |
637; CASP10 | 843; CASP2 | 835; CASP3 | 836; CASP6 | 839; CASP7 | 840; CASP8 | 841;
CASP9 | 842; DIABLO | 56616; ENDOG | 2021; FAS | 355; FASLG | 356; HTRA2 | 27429;
TNFRSF1A | 7132; TP53 | 7157;
Module: CD40 Activated B Cell
BUB1B | 701; CCL22 | 6367; CD58 | 965; DBI | 1622; DUSP4 | 1846; FABP5 | 2171; FDPS |
2224; FEN1 | 2237; GAPDH | 2597; GARS | 2617; GRHPR | 9380; H2AFX | 3014; H2AFZ |
3015; HMGB2 | 3148; HMMR | 3161; HPRT1 | 3251; IMPDH2 | 3615; LDHA | 3939; LDHB |
3945; LMNB1 | 4001; LPXN | 9404; MCM2 | 4171; MCM3 | 4172; MCM6 | 4175; MSH6 |
2956; MTHFD2 | 10797; MYBL2 | 4605; NDC80 | 10403; NME1 | 4830; PAICS | 10606;
PCNA | 5111; PKM | 5315; PRDX3 | 10935; RFTN1 | 23180; RGS10 | 6001; SLAMF1 |
6504; TK1 | 7083; TOP2A | 7153; TPX2 | 22974; TRAF1 | 7185; TUBA1B | 10376; TXN |
7295; TYMS | 7298; UBE2S | 27338; VDAC1 | 7416; WEE1 | 7465; WSB2 | 55884; ZWINT |
11130;
Module: Cell Cycle
ASPM | 259266; AURKA | 6790; AURKB | 9212; BRCA1 | 672; CCNB1 | 891; CCNB2 |
9133; CCNE1 | 898; CDC20 | 991; CENPM | 79019; CEP55 | 55165; E2F3 | 1871; GINS2 |
51659; MCM10 | 55388; MCM2 | 4171; MKI67 | 4288; NCAPG | 64151; NDC80 | 10403;
PTTG1 | 9232; TYMS | 7298;
Module: Classical Complement
C1QA | 712; C1QC | 714; C1R | 715; C1S | 716; C2 | 717; C3 | 718; C4A | 720; C4B | 721;
C4B_2 | 100293534; C5 | 727; C6 | 729; C7 | 730; C8A | 731; C9 | 735; CIQB | 713;
Module: Cytotoxic_Activated T Cell
CD69 | 969; EOMES | 8320; GZMB | 3002; GZMH | 2999; IFNG | 3458; IL2RB | 3560; PRF1 |
5551; SGK1 | 6446; TAGAP | 117289; TBX21 | 30009; TFRC | 7037; ZNF683 | 257101;
Module: Dendritic Cell
CLEC10A | 10462; CLEC12A | 160364; CLEC9A | 283420; CSF1R | 1436; IGIP | 492311;
LILRA4 | 23547; LY75 | 4065; XCR1 | 2829;
Module: Erythrocytes
EPO | 2056; GFI1B | 8328; GYPA | 2993; GYPB | 2994; GYPE | 2996; ICAM4 | 3386; NFE2 |
4778; SLC4A1 | 6521; TRIM10 | 10107; TSPO2 | 222642; ZNF367 | 195828;
Module: Fatty Acid Alpha Oxidation
HAAO | 23498; HACL1 | 26061; PEX13 | 5194; PHYH | 5264; SLC27A2 | 11001;
Module: Fatty Acid Beta Oxidation
ABCD1 | 215; ABCD2 | 225; ABCD3 | 5825; ACAA2 | 10449; ACACB | 32; ACAD11 |
84129; ACADL | 33; ACADM | 34; ACADS | 35; ACADVL | 37; ACAT1 | 38; ACAT2 | 39;
ACOX1 | 51; ACOX2 | 8309; ACOX3 | 8310; ACOXL | 55289; ACSBG2 | 81616; ACSL5 |
51703; ADIPOQ | 9370; AKT2 | 208; AUH | 549; BDH2 | 56898; CPT1A | 1374; CPT2 |
1376; CROT | 54677; DECR1 | 1666; ECHDC1 | 55862; ECHDC2 | 55268; ECHS1 | 1892;
ECI1 | 1632; ECI2 | 10455; EHHADH | 1962; ETFA | 2108; ETFB | 2109; ETFDH | 2110;
FABP1 | 2168; GCDH | 2639; HADH | 3033; HADHA | 3030; HADHB | 3032; HIBCH
26275; HSD17B4 | 3295; IRS1 | 3667; IRS2 | 8660; IVD | 3712; LEP | 3952; PEX2 | 5828;
PEX5 | 5830; PEX7 | 5191; SESN2 | 83667; SLC25A17 | 10478; SLC27A2 | 11001; TWIST1 |
7291;
Module: Glycolysis
G6PC2 | 57818; HKDC1 | 80201; LDHAL6A | 160287; LDHAL6B | 92483; PFKFB2 | 5208;
PFKFB3 | 5209; PFKFB4 | 5210; PKM | 5315; SLC2A1 | 6513; SLC2A3 | 6515; SLC2A4 |
6517; SLC2A5 | 6518;
Module: Granulocyte
CD177 | 57126; CLC | 1178; CTSS | 1520; CXCR2 | 3579; DEFA1 | 1667; FUT7 | 2529;
LTB4R | 1241; MMP25 | 64386; OSM | 5008; RETN | 56729;
Module: IFN
EIF2AK2 | 5610; GBP1 | 2633; GBP2 | 2634; GBP4 | 115361; HERC5 | 51191; HERC6 |
55008; IFI27 | 3429; IFI30 | 10437; IFI35 | 3430; IFI44 | 10561; IFI44L | 10964; IFI6 | 2537;
IFIT1 | 3434; IFIT2 | 3433; IFIT3 | 3437; IFIT5 | 24138; IFITM1 | 8519; IFITM2 | 10581;
IFITM3 | 10410; ISG15 | 9636; ISG20 | 3669; MX1 | 4599; MX2 | 4600; OAS1 | 4938; OAS2 |
4939; OAS3 | 4940; OASL | 8638; RSAD2 | 91543; SAMD9 | 54809; SAMD9L | 219285;
SP100 | 6672; SP110 | 3431;
Module: IFNA2 Signature
ACSL1 | 2180; ADAR | 103; AGT | 183; AIM2 | 9447; AKAP2 | 11217; APOBEC3B | 9582;
APOBEC3G | 60489; APOL3 | 80833; ATF3 | 467; ATF5 | 22809; BAG1 | 573; BARD1 |
580; BCL7B | 9275; BLVRA | 644; BRCA1 | 672; BRCA2 | 675; BST2 | 684; BUB1 | 699;
C2 | 717; CACNA1A | 773; CAD | 790; CAMK2A | 815; CASP1 | 834; CASP10 | 843;
CASP5 | 838; CBR1 | 873; CBWD1 | 55871; CCL13 | 6357; CCL7 | 6354; CCL8 | 6355;
CCNA1 | 8900; CCND2 | 894; CD2AP | 23607; CD38 | 952; CD4 | 920; CD69 | 969;
CDC42EP1 | 11135; CDK4 | 1019; CDKN1A | 1026; CFB | 629; CH25H | 9023; CHKA |
1119; CNTN6 | 27255; COL3A1 | 1281; CTSL | 1514; CXCL10 | 3627; CXCL11 | 6373;
CXCL9 | 4283; CXCR2 | 3579; CYP2J2 | 1573; DAB2 | 1601; DEFB1 | 1672; DLL1 | 28514;
DSC2 | 1824; DUSP5 | 1847; DUSP7 | 1849; DYNLT1 | 6993; DYSF | 8291; ECE1 | 1889;
EDN1 | 1906; EIF2AK2 | 5610; EIF2B1 | 1967; EIF4ENIF1 | 56478; ENPP2 | 5168; EPB41 |
2035; ETV4 | 2118; F8 | 2157; FAF1 | 11124; FAS | 355; FGF1 | 2246; FLNA | 2316; FOXO1 |
2308; FTL | 2512; FUT4 | 2526; GADD45B | 4616; GBAP1 | 2630; GBP1 | 2633; GBP2 |
2634; GCH1 | 2643; GCNT1 | 2650; GLB1 | 2720; GLS | 2744; GMPR | 2766; GPR161 |
23432; GUK1 | 2987; HBG2 | 3048; HCAR3 | 8843; HIST2H2AA3 | 8337; HLA-DOA |
3111; HLA-DRB5 | 3127; HS6ST1 | 9394; HSP90AA1 | 3320; IDO1 | 3620; IFI16 | 3428;
IFI27 | 3429; IFI35 | 3430; IFI44 | 10561; IFI44L | 10964; IFI6 | 2537; IFIT1 | 3434; IFIT5 |
24138; IFITM1 | 8519; IFITM2 | 10581; IFITM3 | 10410; IFNG | 3458; IFRD1 | 3475; IGL |
3535; IKBKG | 8517; IL15 | 3600; IL15RA | 3601; IL1RN | 3557; IL6 | 3569; INPPL1 | 3636;
IRF2 | 3660; IRF7 | 3665; ISG15 | 9636; ISG20 | 3669; ITIH2 | 3698; JAK2 | 3717; JUP |
3728; KCNA3 | 3738; KDELR2 | 11014; KIF20B | 9585; KLF6 | 1316; KPNB1 | 3837; KRT8|
3856; LAG3 | 3902; LAMP3 | 27074; LAP3 | 51056; LEPR | 3953; LGALS2 | 3957;
LGALS3BP | 3959; LGALS9 | 3965; LGMN | 5641; LMNB1 | 4001; LMO2 | 4005; LY6E |
4061; MAP2K5 | 5607; MCL1 | 4170; MED1 | 5469; MGLL | 11343; MMP16 | 4325; MNDA |
4332; MRPS15 | 64960; MSR1 | 4481; MX1 | 4599; MX2 | 4600; MYD88 | 4615; NAMPT |
10135; NFE2L3 | 9603; NKTR | 4820; NMI | 9111; NR3C1 | 2908; NUB1 | 51667; NUPRI |
26471; OAS1 | 4938; OAS2 | 4939; OAS3 | 4940; OSBPL1A | 114876; PATJ | 10207;
PDGFB | 5155; PDGFRL | 5157; PGGT1B | 5229; PKD2 | 5311; PLSCR1 | 5359; PMAIP1 |
5366; PML | 5371; PRKRA | 8575; PSMB9 | 5698; PTCH1 | 5727; RBCK1 | 10616; RET |
5979; RGS1 | 5996; RGS6 | 9628; RPS9 | 6203; RTP4 | 64108; SAT1 | 6303; SCARB2 | 950;
SERPING1 | 710; SIT1 | 27240; SLAMF1 | 6504; SOCS1 | 8651; SP100 | 6672; SP110 | 3431;
SP140 | 11262; SPIB | 6689; ST3GAL5 | 8869; STAP1 | 26228; STAT1 | 6772; STAT2 |
6773; STX11 | 8676; SUPT3H | 8464; SYN2 | 6854; TAF5L | 27097; TAP1 | 6890; TAP2 |
6891; TARBP1 | 6894; TCN2 | 6948; TFDP2 | 7029; TGM1 | 7051; TLR3 | 7098; TLR7 |
51284; TNFRSF11A | 8792; TNFSF10 | 8743; TNFSF6 | 356; TNK2 | 10188; TOR1B |
27348; TRA2B | 6434; TRD | 6964; TRIM21 | 6737; TRIM22 | 10346; TRIM26 | 7726;
TRIM34 | 53840; TRIM38 | 10475; UBA7 | 7318; UBE2L6 | 9246; UBE2S | 27338; UBE3A |
7337; UNC93B1 | 81622; USP18 | 11274; VAMP5 | 10791; WARS | 7453; WT1 | 7490;
XAF1 | 54739;
Module: IFNB1 Signature
ACLY | 47; ACSL1 | 2180; ADAM19 | 8728; ADAP2 | 55803; ADAR | 103; ADGRE2 |
30817; ADM | 133; AFF3 | 3899; AGT | 183; AIM2 | 9447; AKAP10 | 11216; AKAP2 |
11217; ALOX12 | 239; ALOX5 | 240; ANXA4 | 307; APOBEC3B | 9582; APOBEC3G |
60489; APOL3 | 80833; ATF3 | 467; ATF5 | 22809; ATM | 472; ATP13A1 | 57130; B4GAT1 |
11041; BAG1 | 573; BAK1 | 578; BARD1 | 580; BCL11A | 53335; BCL7B | 9275; BGN |
633; BLNK | 29760; BLVRA | 644; BLZF1 | 8548; BRCA1 | 672; BRCA2 | 675; BST2 | 684;
BUB1 | 699; C3AR1 | 719; CACNA1A | 773; CAD | 790; CALD1 | 800; CAMK2A | 815;
CAPN2 | 824; CASP1 | 834; CASP10 | 843; CASP5 | 838; CBR1 | 873; CBWD1 | 55871;
CCL13 | 6357; CCL3L1 | 6349; CCL4 | 6351; CCL7 | 6354; CCL8 | 6355; CCNA1 | 8900;
CCND2 | 894; CCR1 | 1230; CCR5 | 1234; CCRL2 | 9034; CD163 | 9332; CD164 | 8763;
CD2AP | 23607; CD38 | 952; CD4 | 920; CD59 | 966; CD69 | 969; CD72 | 971; CD86 | 942;
CDK17 | 5128; CDKN1A | 1026; CENPA | 1058; CENPE | 1062; CFB | 629; CFLAR | 8837;
CH25H | 9023; CHI3L2 | 1117; CHKA | 1119; CISH | 1154; CKB | 1152; CMAHP | 8418;
CNTN6 | 27255; CNTRL | 11064; COL3A1 | 1281; COX17 | 10063; CSF2RB | 1439; CTSL |
1514; CXCL10 | 3627; CXCL11 | 6373; CXCL2 | 2920; CXCL9 | 4283; CXCR2 | 3579;
CYBB | 1536; CYP19A1 | 1588; CYP2J2 | 1573; DAB2 | 1601; DEFA1 | 1667; DEFB1 |
1672; DHFR | 1719; DLL1 | 28514; DMXL1 | 1657; DNMT1 | 1786; DRAP1 | 10589; DSC2 |
1824; DUSP5 | 1847; DUSP7 | 1849; DYNLT1 | 6993; DYSF | 8291; E2F1 | 1869; ECE1 |
1889; EDN1 | 1906; EGR1 | 1958; EIF2AK2 | 5610; EIF2B1 | 1967; EIF4ENIF1 | 56478;
ELF1 | 1997; ELF4 | 2000; ENPP2 | 5168; EPB41 | 2035; ETV4 | 2118; ETV6 | 2120; F8 |
2157; FAF1 | 11124; FAS | 355; FBXW2 | 26190; FCGR1A | 2209; FCMR | 9214; FGF1 |
2246; FLNA | 2316; FMR1 | 2332; FOXO1 | 2308; FPR2 | 2358; FTL | 2512; FUT4 | 2526;
GADD45B | 4616; GBAP1 | 2630; GBP1 | 2633; GBP2 | 2634; GCH1 | 2643; GCNT1 | 2650;
GLS | 2744; GMPR | 2766; GPI | 2821; GPR161 | 23432; GUK1 | 2987; HBG2 | 3048;
HCAR3 | 8843; HHEX | 3087; HIST2H2AA3 | 8337; HK2 | 3099; HLA-DOA | 3111;
HS6ST1 | 9394; HSP90AA1 | 3320; HSPA1A | 3303; HSPA1L | 3305; IDO1 | 3620; IFI16 |
3428; IFI27 | 3429; IFI35 | 3430; IFI44 | 10561; IFI6 | 2537; IFIT1 | 3434; IFIT5 | 24138;
IFITM1 | 8519; IFITM2 | 10581; IFITM3 | 10410; IFNG | 3458; IFRD1 | 3475; IGL | 3535;
IKBKE | 9641; IKBKG | 8517; IL15 | 3600; IL15RA | 3601; IL18BP | 10068; IL18R1 | 8809;
IL1RN | 3557; IL6 | 3569; IL7 | 3574; INPP5D | 3635; INPPL1 | 3636; IRF1 | 3659; IRF2 |
3660; IRF4 | 3662; IRF7 | 3665; IRF9 | 10379; ISG15 | 9636; ISG20 | 3669; ITGAL | 3683;
ITGAX | 3687; JAK2 | 3717; JCHAIN | 3512; JUP | 3728; KCNA3 | 3738; KCNMB1 | 3779;
KDELR2 | 11014; KIF20B | 9585; KLF2 | 10365; KLF6 | 1316; KLRB1 | 3820; KPNB1 |
3837; KRT8 | 3856; LAG3 | 3902; LAMP3 | 27074; LANCL1 | 10314; LAP3 | 51056; LBR |
3930; LEPR | 3953; LGALS2 | 3957; LGALS3BP | 3959; LGALS9 | 3965; LGMN | 5641;
LILRA1 | 11024; LINC00597 | 81698; LMNB1 | 4001; LMO2 | 4005; LTA | 4049; LTB4R |
1241; LY6E | 4061; LYN | 4067; MAP2K5 | 5607; MAP3K8 | 1326; MARCKS | 4082;
MBNL | 4154; MCL1 | 4170; MED1 | 5469; MEF2A | 4205; MFHAS1 | 9258; MGLL |
11343; MNDA | 4332; MRPS15 | 64960; MS4A7 | 58475; MSR1 | 4481; MX1 | 4599; MX2 |
4600; MYD88 | 4615; NAMPT | 10135; NAPSA | 9476; NBN | 4683; NCF1 | 653361;
NCOA2 | 10499; NEBL | 10529; NEK4 | 6787; NFE2L3 | 9603; NKTR | 4820; NMI | 9111;
NOTCH1 | 4851; NR3C1 | 2908; NR4A3 | 8013; NUB1 | 51667; NUPR1 | 26471; OAS1 |
4938; OAS2 | 4939; OAS3 | 4940; PATJ | 10207; PAX5 | 5079; PAX8 | 7849; PDE4B | 5142;
PDGFB | 5155; PDGFRL | 5157; PFKFB3 | 5209; PFKP | 5214; PIM2 | 11040; PKD2 | 5311;
PLEK | 5341; PLSCR1 | 5359; PMAIP1 | 5366; PML | 5371; PMS2 | 5395; PPP2R2A | 5520;
PRKAG1 | 5571; PRKRA | 8575; PRKX | 5613; PSMB8 | 5696; PSMB9 | 5698; PTCH1 |
5727; PTGER2 | 5732; RALB | 5899; RASGRP1 | 10125; RBBP6 | 5930; RBCK1 | 10616;
RERE | 473; RGS1 | 5996; RGS6 | 9628; RIN1 | 9610; RIPK1 | 8737; RIPK3 | 11035;
RIPOR2 | 9750; RNF114 | 55905; RPS6KA5 | 9252; RPS9 | 6203; RRBP1 | 6238; RTP4 |
64108; SAT1 | 6303; SCARB2 | 950; SDS | 10993; SELL | 6402; SERPIND1 | 3053;
SERPING1 | 710; SFTPB | 6439; SIDT2 | 51092; SIT1 | 27240; SLAMF1 | 6504; SMO |
6608; SNX2 | 6643; SOCS1 | 8651; SOS1 | 6654; SP100 | 6672; SP110 | 3431; SP140 | 11262;
SPIB | 6689; SPTA1 | 6708; SPTLC2 | 9517; SRRM2 | 23524; SSB | 6741; ST3GAL5 | 8869;
STAP1 | 26228; STAT1 | 6772; STAT2 | 6773; STOML2 | 30968; STX11 | 8676; SUPT3H |
8464; TANK | 10010; TAP1 | 6890; TAP2 | 6891; TAPBP | 6892; TARBP1 | 6894; TBX21 |
30009; TCN2 | 6948; TFDP2 | 7029; TFF1 | 7031; TGM1 | 7051; THY1 | 7070; TLR1 | 7096;
TLR3 | 7098; TLR7 | 51284; TNFAIP2 | 7127; TNFRSF11A | 8792; TNFSF10 | 8743;
TNFSF6 | 356; TNK2 | 10188; TOR1B | 27348; TRA2B | 6434; TRD | 6964; TRG | 6965;
TRIM21 | 6737; TRIM22 | 10346; TRIM26 | 7726; TRIM34 | 53840; TRIM38 | 10475;
TSPAN15 | 23555; TXK | 7294; UBA7 | 7318; UBE2L6 | 9246; UBE2S | 27338; UBE3A |
7337; UBQLN2 | 29978; UNC93B1 | 81622; USP15 | 9958; USP18 | 11274; USP25 | 29761;
USPL1 | 10208; UVRAG | 7405; VAMP5 | 10791; WARS | 7453; WIPF1 | 7456; WT1 | 7490;
XAF1 | 54739; ZNF107 | 51427;
Module: IFNG Signature
ACLY | 47; ACSL1 | 2180; AFF2 | 2334; AIM2 | 9447; AKAP10 | 11216; APOL3 | 80833;
ATF3 | 467; ATM | 472; C1QB | 713; C4A | 720; CALD1 | 800; CASP1 | 834; CASP10 | 843;
CCL8 | 6355; CCND2 | 894; CCR5 | 1234; CD38 | 952; CDKN1A | 1026; CFB | 629; CKB |
1152; CLEC10A | 10462; CPT1B | 1375; CSF2RB | 1439; CTNND2 | 1501; CXCL10 | 3627;
CXCL11 | 6373; CXCL9 | 4283; CYBB | 1536; EDN1 | 1906; EPB41 | 2035; ETAA1 | 54465;
ETV4 | 2118; F8 | 2157; FAS | 355; FBLN1 | 2192; FBXL2 | 25827; FCGR1A | 2209; FLII |
2314; GADD45B | 4616; GBP1 | 2633; GBP2 | 2634; GCH1 | 2643; GCNT1 | 2650; GLS |
2744; GSTM5 | 2949; HBG2 | 3048; HHEX | 3087; HP | 3240; ICAM1 | 3383; IDO1 | 3620;
IFI27 | 3429; IFI44 | 10561; IL15 | 3600; IL15RA | 3601; IL18BP | 10068; IL1A | 3552; IL7 |
3574; IRF1 | 3659; IRF8 | 3394; JAK2 | 3717; JCHAIN | 3512; KLF2 | 10365; LAP3 | 51056;
LIMK2 | 3985; LMNB1 | 4001; MMP25 | 64386; MRPS15 | 64960; MSR1 | 4481; NET1 |
10276; NIN | 51199; NKTR | 4820; NLRP1 | 22861; NR3C1 | 2908; OAS1 | 4938; OAS3
4940; P2RY13 | 53829; PCDH9 | 5101; PLA2G4C | 8605; PLEK | 5341; POLR2B | 5431;
PSMB9 | 5698; PTCH1 | 5727; RALB | 5899; RGS1 | 5996; SERPIND1 | 3053; SERPING1 |
710; SFTPB | 6439; SLAMF1 | 6504; SLC1A5 | 6510; SOCS1 | 8651; SP100 | 6672; SPRY4 |
81848; SRRM2 | 23524; STAT1 | 6772; STAT2 | 6773; STX11 | 8676; TAP1 | 6890; TAP2 |
6891; TBX21 | 30009; TENM1 | 10178; TFF1 | 7031; TNFAIP2 | 7127; TNFSF10 | 8743;
UBD | 10537; UBE2C | 11065; UBE2L6 | 9246; UBE3A | 7337; VAMP5 | 10791; VSNL1 |
7447; WARS | 7453; XRN1 | 54464;
Module: Inflammatory Neutrophil
AC245128 | NA; ACSL1 | 2180; ADAR | 103; ADD3 | 120; ADM | 133; ALOX5AP | 241;
ALPL | 249; ANXA1 | 301; ANXA3 | 306; APOBEC3A | 200315; APOL2 | 23780; APOL6 |
80830; B4GALT5 | 9334; BAZ1A | 11177; BRI3 | 25798; BST1 | 683; C1orf162 | 128346;
C3AR1 | 719; C4orf3 | 401152; CAPZA1 | 829; CARD16 | 114769; CASP1 | 834; CASP4 |
837; CAST | 831; CCR1 | 1230; CD177 | 57126; CD37 | 951; CD44 | 960; CD53 | 963; CD55 |
1604; CD63 | 967; CD82 | 3732; CDKN2D | 1032; CEACAM1 | 634; CFL1 | 1072; CKAP4 |
10970; CLEC4D | 338339; CLEC4E | 26253; CR1 | 1378; CST7 | 8530; CYSTM1 | 84418;
DDX58 | 23586; DDX60 | 55601; DDX60L | 91351; DYSF | 8291; EIF2AK2 | 5610; EMB |
133418; EPSTI1 | 94240; FCER1G | 2207; FCGR1A | 2209; FFAR2 | 2867; FGR | 2268;
FKBP1A | 2280; FKBP5 | 2289; FLOT1 | 10211; FYB1 | 2533; GAPDH | 2597; GBP1 | 2633;
GBP2 | 2634; GBP4 | 115361; GBP5 | 115362; GCA | 25801; GIMAP4 | 55303; GLRX |
2745; GNG5 | 2787; GRINA | 2907; GRN | 2896; GSTK1 | 373156; GYG1 | 2992; H2AC6
8334; H2BC21 | 8349; H2BC5 | 3017; HERC5 | 51191; HIF1A | 3091; HLA-F | 3134;
HMGB2 | 3148; IFI16 | 3428; IFI44 | 10561; IFI44L | 10964; IFI6 | 2537; IFIH1 | 64135;
IFIT1 | 3434; IFIT2 | 3433; IFIT3 | 3437; IFITM1 | 8519; IFITM3 | 10410; IL1RN | 3557;
IL2RG | 3561; IRF1 | 3659; IRF7 | 3665; ISG15 | 9636; ISG20 | 3669; ITGAM | 3684; JUN |
3725; KCNJ15 | 3772; KLF4 | 9314; LAP3 | 51056; LGALS9 | 3965; LILRA5 | 353514;
LILRA6 | 79168; LILRB3 | 11025; LIMK2 | 3985; LMNB1 | 4001; LRG1 | 116844; LY6E |
4061; LY96 | 23643; MAPK14 | 1432; MAX | 4149; MCEMP1 | 199675; METTL9 | 51108;
MMP9 | 4318; MOB1A | 55233; MSRB1 | 51734; MT2A | 4502; MTPN | 136319; MX1 |
4599; MX2 | 4600; MYL12A | 10627; NBN | 4683; NFE2 | 4778; NFIL3 | 4783; NFKBIA |
4792; NMI | 9111; NT5C3A | 51251; NTNG2 | 84628; NUB1 | 51667; NUCB1 | 4924; OAS1 |
4938; OAS2 | 4939; OAS3 | 4940; OASL | 8638; PARP14 | 54625; PARP9 | 83666; PGD |
5226; PHF11 | 51131; PIK3AP1 | 118788; PIM1 | 5292; PLAC8 | 51316; PLBD1 | 79887;
PLEK | 5341; PLP2 | 5355; PLSCR1 | 5359; PML | 5371; PROK2 | 60675; PRR13 | 54458;
PSMB9 | 5698; PSTPIP2 | 9050; PTEN | 5728; RAC2 | 5880; RBCK1 | 10616; RBMS1 |
5937; RGS19 | 10287; RHOG | 391; RNF10 | 9921; RNF213 | 57674; RPL28 | 6158; RSAD2 |
91543; S100A12 | 6283; S100A6 | 6277; SAMD9 | 54809; SAMD9L | 219285; SAMHD1
25939; SAMSNI | 64092; SAT1 | 6303; SECTMI | 6398; SELL | 6402; SERPINA1 | 5265;
SERPINB1 | 1992; SERPING1 | 710; SH3GLB1 | 51100; SHISA5 | 51246; SLFN5 | 162394;
SNX3 | 8724; SP100 | 6672; SP110 | 3431; SPI1 | 6688; SPTLC2 | 9517; STATI | 6772;
STAT2 | 6773; STXBP2 | 6813; TAP1 | 6890; TMEM123 | 114908; TMSB10 | 9168;
TNFAIP6 | 7130; TNFSF10 | 8743; TNFSF13B | 10673; TPM3 | 7170; TRIM22 | 10346;
TRIM38 | 10475; TXN | 7295; UBE2J1 | 51465; UBE2L6 | 9246; VIM | 7431; VNN2 | 8875;
WSB1 | 26118; XAF1 | 54739; XRN1 | 54464; ZBP1 | 81030; ZCCHC2 | 54877; ZDHHC19 |
131540; ZYX | 7791;
Module: LDG
AZU1 | 566; CAMP | 820; CEACAM6 | 4680; CEACAM8 | 1088; CTSG | 1511; DEFA4 |
1669; ELANE | 1991; LCN2 | 3934; LTF | 4057; MPO | 4353; OLFM4 | 10562; RNASE3 |
6037;
Module: MHCII
HLA-DMA | 3108; HLA-DMB | 3109; HLA-DPA1 | 3113; HLA-DPB1 | 3115; HLA-DPB2 |
3116; HLA-DQA1 | 3117; HLA-DQA2 | 3118; HLA-DQB1 | 3119; HLA-DQB2 | 3120;
HLA-DRA | 3122; HLA-DRB1 | 3123; HLA-DRB3 | 3125; HLA-DRB4 | 3126; HLA-DRB5 |
3127; HLA-DRB6 | 3128;
Module: Monocyte
BST1 | 683; C1QA | 712; C1QB | 713; C1QC | 714; C1R | 715; C1RL | 51279; CCL2 | 6347;
CCL8 | 6355; CD14 | 929; CD163 | 9332; CD300C | 10871; CD33 | 945; CD68 | 968;
CLEC12A | 160364; CLEC4D | 338339; CLEC4E | 26253; CSF2 | 1437; CXCL1 | 2919;
CXCL2 | 2920; CXCR2 | 3579; FCGR1A | 2209; FCGR1B | 2210; FCGR3B | 2215; FUT4 |
2526; GRN | 2896; IK | 3550; IL18RAP | 8807; IL1B | 3553; IL1RN | 3557; LILRA5 |
353514; LILRA6 | 79168; LILRB2 | 10288; LILRB3 | 11025; MNDA | 4332; MRC1 | 4360;
OSCAR | 126014; S100A8 | 6279; SEMA4A | 64218; SIGLEC1 | 6614; THBD | 7056;
Module: NFkB Complex
IKBKB | 3551; IKBKG | 8517; NFKB1 | 4790; NFKB2 | 4791; REL | 5966; RELA | 5970;
RELB | 5971;
Module: NK Cell
KLRF1 | 51348; KLRF1 | 51348; NCAM1 | 4684; NCAM1 | 4684; NCR1 | 9437; NCR1 |
9437; NCR3 | 259197; NCR3 | 259197; SH2D1B | 117157; SH2D1B | 117157;
Module: NLRP3 Inflammasome
APP | 351; ATAT1 | 79969; CARD8 | 22900; CASP1 | 834; CASP4 | 837; CD36 | 948; CPTP |
80772; DHX33 | 56919; EIF2AK2 | 5610; GBP5 | 115362; GSDMD | 79792; HSP90AB1 |
3326; MEFV | 4210; NFKB1 | 4790; NFKB2 | 4791; NLRC3 | 197358; NLRP3 | 114548;
P2RX7 | 5027; PANX1 | 24145; PSTPIP1 | 9051; PYCARD | 29108; PYDC2 | 152138; RELA |
5970; SIRT2 | 22933; SUGT1 | 10910; TLR4 | 7099; TLR6 | 10333; TXN | 7295; TXNIP |
10628; USP50 | 373509;
Module: Oxidative Phosphorylation
ATP5A1 | 498; ATP5B | 506; ATP5D | 513; ATP5E | 514; ATP5F1 | 515; ATP5G1 | 516;
ATP5G2 | 517; ATP5G3 | 518; ATP5H | 10476; ATP51 | 521; ATP5J | 522; ATP5J2 | 9551;
ATP5L | 10632; ATP50 | 539; ATP5S | 27109; BCSIL | 617; CEP89 | 84902; COA1 | 55744;
COA3 | 28958; COA4 | 51287; COA5 | 493753; COA6 | 388753; COA7 | 65260; COX10 |
1352; COX10-AS1 | 100874058; COX11 | 1353; COX14 | 84987; COX15 | 1355; COX16 |
51241; COX17 | 10063; COX18 | 285521; COX19 | 90639; COX20 | 116228; COX411 | 1327;
COX412 | 84701; COX5A | 9377; COX5B | 1329; COX6A1 | 1337; COX6A2 | 1339;
COX6B1 | 1340; COX6B2 | 125965; COX6C | 1345; COX7A1 | 1346; COX7A2 | 1347;
COX7A2L | 9167; COX7B | 1349; COX7B2 | 170712; COX7C | 1350; COX8A | 1351;
COX8C | 341947; CYC1 | 1537; CYCS | 54205; DNAJC15 | 29103; MT-ATP6 | 4508; MT-
ATP8 | 4509; MT-CO1 | 4512; MT-CO2 | 4513; MT-CO3 | 4514; MT-CYB | 4519; MT-ND1
4535; MT-ND2 | 4536; MT-ND3 | 4537; MT-ND4 | 4538; MT-ND4L | 4539; MT-ND5 |
4540; MT-ND6 | 4541; NDUFA1 | 4694; NDUFA10 | 4705; NDUFA11 | 126328; NDUFA12 |
55967; NDUFA13 | 51079; NDUFA2 | 4695; NDUFA3 | 4696; NDUFA4 | 4697;
NDUFA4L2 | 56901; NDUFA5 | 4698; NDUFA6 | 4700; NDUFA7 | 4701; NDUFA8 | 4702;
NDUFA9 | 4704; NDUFAB1 | 4706; NDUFAF1 | 51103; NDUFAF2 | 91942; NDUFAF3 |
25915; NDUFAF4 | 29078; NDUFAF5 | 79133; NDUFAF6 | 137682; NDUFAF7 | 55471;
NDUFAF8 | 284184; NDUFB1 | 4707; NDUFB10 | 4716; NDUFB11 | 54539; NDUFB2|
4708; NDUFB2-AS1 | 100134713; NDUFB3 | 4709; NDUFB4 | 4710; NDUFB5 | 4711;
NDUFB6 | 4712; NDUFB7 | 4713; NDUFB8 | 4714; NDUFB9 | 4715; NDUFC1 | 4717;
NDUFC2 | 4718; NDUFS1 | 4719; NDUFS2 | 4720; NDUFS3 | 4722; NDUFS4 | 4724;
NDUFS5 | 4725; NDUFS6 | 4726; NDUFS7 | 374291; NDUFS8 | 4728; NDUFV1 | 4723;
NDUFV2 | 4729; NDUFV3 | 4731; NUBPL | 80224; OXA1L | 5018; RFESD | 317671; SCO1 |
6341; SCO2 | 9997; SLC25A4 | 291; SURF1 | 6834; TACO1 | 51204; TIMMDC1 | 51300;
TMEM126B | 55863; TRAP1 | 10131; TTC19 | 54902; UQCC1 | 55245; UQCC2 | 84300;
UQCC3 | 790955; UQCR10 | 29796; UQCR11 | 10975; UQCRB | 7381; UQCRC1 | 7384;
UQCRC2 | 7385; UQCRFS1 | 7386; UQCRH | 7388; UQCRHL | 440567; UQCRQ | 27089;
Module: pDC
CLEC4C | 170482; NRP1 | 8829;
Module: Pentose Phosphate Signature
G6PD | 2539; H6PD | 9563; PGD | 5226; PRPS1 | 5631; PRPS1L1 | 221823; PRPS2 | 5634;
RBKS | 64080; RGN | 9104; RPE | 6120; RPIA | 22934; TALDO1 | 6888; TKT | 7086;
TKTL1 | 8277; TKTL2 | 84076;
Module: Plasma Cell
C19orf10 | 56005; IGH | 3492; IGHD | 3495; IGHG1 | 3500; IGHMBP2 | 3508; IGHV2-5 |
28457; IGHV3-20 | 28445; IGHV3-23 | 28442; IGHV4-28 | 28400; IGHV4-31 | 28396;
IGHV4-34 | 28395; IGK | 50802; IGKC | 3514; IGL | 3535; IGLJ3 | 28831; IGLL1 | 3543;
IGLV@ | 3546; IGLV1-40 | 28825; IGLV1-44 | 28823; IGLV2-14 | 28815; IGLV2-5 | 28818;
IGLV3-1 | 28809; IGLV3-19 | 28797; IGLV3-25 | 28793; IGLV4-3 | 28786; IGLV4-60 |
28785; IGLV5-45 | 28781; IGLV6-57 | 28778; IGLVI-70 | 28763; MZB1 | 51237; PRDM1 |
639; SDC1 | 6382; THEMIS2 | 9473; TNFRSF17 | 608;
Module: Platelets
GP1BA | 2811; GP5 | 2814; GP6 | 51206; GP9 | 2815; LY6G6D | 58530; MMRN1 | 22915;
PEAR1 | 375033; PF4 | 5196; PF4V1 | 5197; PPBP | 5473; SLC35D3 | 340146;
Module: Proinflammatory IL-1 Family
IL18 | 3606; IL18R1 | 8809; IL18RAP | 8807; IL1A | 3552; IL1B | 3553; IL1R1 | 3554;
IL1RAP | 3556; IL33 | 90865; IL36A | 27179; IL36B | 27177; IL36G | 56300; ATOX1 | 475;
Module: ROS Protection
CCS | 9973; CYBA | 1535; CYBB | 1536; GPX1 | 2876; GPX2 | 2877; GPX3 | 2878; GPX5 |
2880; GPX6 | 257202; GPX7 | 2882; GPX8 | 493869; GSR | 2936; GSTP1 | 2950; NCF1 |
653361; NCF2 | 4688; NCF4 | 4689; NOX4 | 50507; NOX5 | 79400; NUDT2 | 318; P4HB
5034; PRDX1 | 5052; PRDX2 | 7001; PRDX3 | 10935; PRDX4 | 10549; PRDX5 | 25824;
PRDX6 | 9588; SOD1 | 6647; SOD2 | 6648; SOD3 | 6649; TXN | 7295; TXN2 | 25828;
TXNRD1 | 7296; TXNRD2 | 10587;
Module: Suppressive Neutrophil
APOL6 | 80830; CARD16 | 114769; CCR1 | 1230; CLEC2B | 9976; DDX60L | 91351;
EPSTI1 | 94240; FCER1G | 2207; FFAR2 | 2867; GBP1 | 2633; GBP2 | 2634; GBP5 | 115362;
IFI16 | 3428; IFI44 | 10561; IFI44L | 10964; IFI6 | 2537; IFIH1 | 64135; IFIT1 | 3434; IFIT2
3433; IFIT3 | 3437; IFITM3 | 10410; IL1RN | 3557; IRF7 | 3665; ISG15 | 9636; LIMK2 |
3985; LY6E | 4061; LY96 | 23643; MT2A | 4502; MX1 | 4599; NBN | 4683; NFKBIA | 4792;
OAS1 | 4938; OAS2 | 4939; PLSCR1 | 5359; RNF213 | 57674; RSAD2 | 91543; SAMD9 |
54809; SAMD9L | 219285; SELL | 6402; SERPING1 | 710; SHISA5 | 51246; SP110 | 3431;
TAP1 | 6890; TNFSF10 | 8743; TNFSF13B | 10673; TRIM22 | 10346; XAF1 | 54739;
Module: T Cell
CCR3 | 1232; CD226 | 10666; CD247 | 919; CD28 | 940; CD3D | 915; CD3E | 916; CD3G |
917; CD4 | 920; CD5 | 921; CD8A | 925; CD8B | 926; ETS1 | 2113; GATA3 | 2625; GRAP2 |
9402; LEF1 | 51176; SH2D1A | 4068; TRAC | 28755; TRBC1 | 28639; TRDC | 28526;
Module: Tactivated
CD40LG | 959; CREM | 1390; FASLG | 356; IL17A | 3605; IL17F | 112744; IL23R | 149233;
JAKMIP1 | 152789; KCNA3 | 3738; KCNN4 | 3783; P2RX5 | 5026; PRKCQ | 5588; RELT |
84957; RNF125 | 54941; SATB1 | 6304; TAGAP | 117289; TNFRSF4 | 7293; TNFRSF9 |
3604;
Module: TCA cycle
ACO2 | 50; CS | 1431; DLAT | 1737; DLD | 1738; DLST | 1743; FH | 2271; GLUD1 | 2746;
IDH1 | 3417; IDH2 | 3418; IDH3A | 3419; IDH3B | 3420; IDH3G | 3421; MDH2 | 4191;
MPC1 | 51660; MPC2 | 25874; OGDH | 4967; OGDHL | 55753; PDHA1 | 5160; PDHA2 |
5161; PDHB | 5162; PDHX | 8050; PDK1 | 5163; PDK2 | 5164; PDK3 | 5165; PDK4 | 5166;
PDP1 | 54704; PDP2 | 57546; PDPR | 55066; SDHA | 6389; SDHAF1 | 644096; SDHAF2 |
54949; SDHAF3 | 57001; SDHAF4 | 135154; SDHB | 6390; SDHC | 6391; SDHD | 6392;
SUCLA2 | 8803; SUCLG1 | 8802; SUCLG2 | 8801; SUGCT | 79783;
Module: TNF
ACLY | 47; ACSL1 | 2180; ADGRE2 | 30817; AK3 | 50808; AKAP10 | 11216; AMPD3 |
272; APOL3 | 80833; ARID3A | 1820; ARSE | 415; ASAP1 | 50807; B4GALT5 | 9334;
BCL2A1 | 597; BHLHE41 | 79365; BHMT | 635; BIRC3 | 330; BRCA1 | 672; CALD1 | 800;
CASP1 | 834; CASP10 | 843; CCL15 | 6359; CCL20 | 6364; CCL23 | 6368; CCL3L1 | 6349;
CD37 | 951; CD38 | 952; CD83 | 9308; CDKN3 | 1033; CKB | 1152; CR2 | 1380; CTNND2 |
1501; CXCL1 | 2919; CXCL2 | 2920; CXCL3 | 2921; CXCL8 | 3576; CYP27B1 | 1594;
DAB2 | 1601; EBI3 | 10148; EGR1 | 1958; EGR2 | 1959; EPB41 | 2035; EREG | 2069;
ETAA1 | 54465; F3 | 2152; FABP1 | 2168; FBXL2 | 25827; FCER2 | 2208; FCGR2A | 2212;
FLJ11129 | 54674; FLNA | 2316; GOS2 | 50486; GBP1 | 2633; GCH1 | 2643; GJB2 | 2706;
GLS | 2744; GMIP | 51291; GP1BA | 2811; GRK3 | 157; HCAR3 | 8843; HHEX | 3087;
HOMER2 | 9455; HP | 3240; ICAM1 | 3383; IDO1 | 3620; IFI44 | 10561; IKBKG | 8517;
IL16 | 3603; IL18 | 3606; IL1A | 3552; IL1B | 3553; IL1RN | 3557; IL6 | 3569; INHBA |
3624; INSIG1 | 3638; ITGA6 | 3655; KITLG | 4254; KLF1 | 10661; KMO | 8564;
LGALS3BP | 3959; MAP3K4 | 4216; MARCKS | 4082; MGLL | 11343; MMP19 | 4327;
MN1 | 4330; MRPS15 | 64960; MSC | 9242; MTF1 | 4520; MX1 | 4599; NAMPT | 10135;
NELL2 | 4753; NFKB1 | 4790; NFKB2 | 4791; NFKBIA | 4792; NFKBIZ | 64332; NKX3-2 |
579; NR3C1 | 2908; OAS3 | 4940; PATJ | 10207; PDE4DIP | 9659; PDPN | 10630; PIAS4 |
51588; PLAUR | 5329; PTGES | 9536; PTGS2 | 5743; RELB | 5971; RPGR | 6103; RPS9 |
6203; SDC4 | 6385; SERPIND1 | 3053; SFRP1 | 6422; SH3BP5 | 9467; SLAMF1 | 6504;
SLC30A4 | 7782; SOD2 | 6648; SPI1 | 6688; SSPN | 8082; STAT4 | 6775; TAF15 | 8148;
TAP2 | 6891; TBX3 | 6926; TFF1 | 7031; TNF | 7124; TNFAIP2 | 7127; TNFAIP3 | 7128;
TNFRSF11A | 8792; TRAF1 | 7185; TSC22D1 | 8848; TYROBP | 7305; UBE2C | 11065;
VEGFA | 7422; WT1 | 7490;
Module: Treg
FOXP3 | 50943; IKZF2 | 22807;
Module: Unfolded Protein
B4GALT3 | 8703; CALR | 811; CALU | 813; CANX | 821; CDS2 | 8760; CHST12 | 55501;
CHST2 | 9435; DERL1 | 79139; DERL2 | 51009; DNAJC3 | 5611; EDEM2 | 55741; EDEM3 |
80267; EMC9 | 51016; ERAP1 | 51752; ERGIC2 | 51290; ERO1L | 30001; EXT1 | 2131;
GALNT2 | 2590; GOLT1B | 51026; HERPUD1 | 9709; HYOU1 | 10525; IER3IP1 | 51124;
IMPAD1 | 54928; KDELC1 | 79070; KDELR2 | 11014; LMAN2 | 10960; LPGAT1 | 9926;
MAN1A1 | 4121; MANEA | 79694; MANF | 7873; NUCB2 | 4925; PDIA4 | 9601; PDIA6 |
10130; PIGK | 10026; PPIB | 5479; SEC24D | 9871; SEC61G | 23480; SPCS3 | 60559; SSR1 |
6745; SSR3 | 6747; TRAM1 | 23471; TRAM2 | 9697; UGGT1 | 56886; XBP1 | 7494;

Tables 2A-D: Machine learning algorithm performance. For each ML round values for 10 repetitions are shown.

TABLE 2A
Machine learning algorithm performance for
all COVID vs healthy, classification.

| | | | | | |
|---|---|---|---|---|---|
| | | Precision/ | | | |
| | ROC Curve | Recall | | | |
| Model | AUC | AUC | Sensitivity | Specificity | Accuracy |

Machine Learning Round 1

| | | | | | |
|---|---|---|---|---|---|
| SVM | 1 | 1 | 1 | 1 | 1 |
| RF | 1 | 1 | 0.97 | 1 | 0.99 |
| GB | 1 | 1 | 0.97 | 1 | 0.99 |
| DTREE | 0.985 | 0.992 | 0.97 | 1 | 0.99 |
| ADB | 0.999 | 0.999 | 0.94 | 1 | 0.97 |
| NB | 0.987 | 0.984 | 1 | 0.86 | 0.92 |
| LR | 0.953 | 0.959 | 0.88 | 0.95 | 0.92 |
| LDA | 0.953 | 0.956 | 0.79 | 0.93 | 0.87 |
| KNN | 0.964 | 0.976 | 0.68 | 1 | 0.86 |
| SVM | 1 | 1 | 1 | 1 | 1 |
| RF | 0.999316005 | 0.999147306 | 0.97 | 1 | 0.99 |
| ADB | 0.997948016 | 0.997363956 | 0.97 | 0.98 | 0.97 |
| GB | 0.999316005 | 0.999147306 | 0.94 | 1 | 0.97 |
| NB | 0.987004104 | 0.985557366 | 0.97 | 0.88 | 0.92 |
| LDA | 0.972640219 | 0.978775597 | 0.82 | 1 | 0.92 |
| LR | 0.95622435 | 0.956357977 | 0.82 | 0.98 | 0.91 |
| DTREE | 0.914842681 | 0.942271454 | 0.85 | 0.98 | 0.92 |
| KNN | 0.980848153 | 0.985894458 | 0.68 | 1 | 0.86 |
| SVM | 1 | 1 | 1 | 1 | 1 |
| RF | 0.998974008 | 0.998750481 | 0.97 | 1 | 0.99 |
| GB | 0.996580027 | 0.996179646 | 0.97 | 1 | 0.99 |
| ADB | 0.999316005 | 0.999147306 | 0.94 | 1 | 0.97 |
| NB | 0.991792066 | 0.989398297 | 0.97 | 0.91 | 0.94 |
| LDA | 0.976060192 | 0.98106236 | 0.82 | 1 | 0.92 |
| LR | 0.965800274 | 0.96949542 | 0.85 | 0.98 | 0.92 |
| KNN | 0.985294118 | 0.991787624 | 0.68 | 1 | 0.86 |
| DTREE | 0.871409029 | 0.883817163 | 0.88 | 0.86 | 0.87 |
| SVM | 1 | 1 | 1 | 1 | 1 |
| GB | 0.997264022 | 0.996862187 | 0.97 | 1 | 0.99 |
| RF | 0.998290014 | 0.997910144 | 0.97 | 0.95 | 0.96 |
| NB | 0.99247606 | 0.991609408 | 1 | 0.86 | 0.92 |
| ADB | 0.989740082 | 0.987763276 | 0.97 | 0.88 | 0.92 |
| DTREE | 0.93878249 | 0.93773357 | 0.97 | 0.91 | 0.94 |
| LR | 0.945280438 | 0.951476859 | 0.85 | 0.95 | 0.91 |
| LDA | 0.954172367 | 0.959708605 | 0.76 | 0.95 | 0.87 |
| KNN | 0.98255814 | 0.98842628 | 0.62 | 1 | 0.83 |
| SVM | 1 | 1 | 1 | 1 | 1 |
| RF | 1 | 1 | 0.97 | 1 | 0.99 |
| GB | 0.999316005 | 0.999147306 | 0.97 | 1 | 0.99 |
| ADB | 0.997264022 | 0.996430013 | 0.94 | 0.98 | 0.96 |
| DTREE | 0.962038304 | 0.963216196 | 0.97 | 0.95 | 0.96 |
| NB | 0.990424077 | 0.9885144 | 1 | 0.86 | 0.92 |
| LR | 0.952804378 | 0.956143715 | 0.85 | 0.93 | 0.9 |
| KNN | 0.978454172 | 0.983838499 | 0.74 | 1 | 0.88 |
| LDA | 0.965800274 | 0.966928384 | 0.76 | 0.98 | 0.88 |
| SVM | 1 | 1 | 1 | 1 | 1 |
| ADB | 1 | 1 | 1 | 0.98 | 0.99 |
| RF | 0.999316005 | 0.999183007 | 0.97 | 1 | 0.99 |
| GB | 0.995212038 | 0.99491571 | 0.97 | 0.93 | 0.95 |
| NB | 0.990424077 | 0.988393545 | 0.97 | 0.86 | 0.91 |
| KNN | 0.97879617 | 0.983801237 | 0.76 | 1 | 0.9 |
| LDA | 0.957592339 | 0.961506277 | 0.79 | 0.95 | 0.88 |
| LR | 0.949384405 | 0.949922933 | 0.82 | 0.86 | 0.84 |
| DTREE | 0.845075239 | 0.861715898 | 0.85 | 0.84 | 0.84 |
| SVM | 1 | 1 | 1 | 1 | 1 |
| RF | 0.997264022 | 0.996862187 | 0.97 | 1 | 0.99 |
| GB | 0.997264022 | 0.996862187 | 0.97 | 1 | 0.99 |
| ADB | 0.995896033 | 0.994660799 | 0.97 | 0.98 | 0.97 |
| NB | 0.99247606 | 0.990468169 | 0.97 | 0.91 | 0.94 |
| DTREE | 0.955882353 | 0.975362872 | 0.91 | 1 | 0.96 |
| LDA | 0.980848153 | 0.985363542 | 0.85 | 1 | 0.94 |
| LR | 0.97127223 | 0.971012984 | 0.82 | 1 | 0.92 |
| KNN | 0.985294118 | 0.991787624 | 0.65 | 1 | 0.84 |
| SVM | 1 | 1 | 1 | 1 | 1 |
| RF | 1 | 1 | 0.97 | 1 | 0.99 |
| ADB | 0.999316005 | 0.999147306 | 0.97 | 0.98 | 0.97 |
| GB | 0.998632011 | 0.99834267 | 0.94 | 1 | 0.97 |
| LDA | 0.97127223 | 0.979492252 | 0.82 | 1 | 0.92 |
| DTREE | 0.929548564 | 0.951021464 | 0.88 | 0.98 | 0.94 |
| NB | 0.976060192 | 0.971446883 | 0.97 | 0.84 | 0.9 |
| LR | 0.950752394 | 0.955304242 | 0.85 | 0.95 | 0.91 |
| KNN | 0.983926129 | 0.99005752 | 0.71 | 1 | 0.87 |
| SVM | 1 | 1 | 1 | 0.98 | 0.99 |
| RF | 0.997264022 | 0.996862187 | 0.97 | 0.98 | 0.97 |
| GB | 0.997264022 | 0.996862187 | 0.97 | 0.98 | 0.97 |
| ADB | 0.995212038 | 0.993435829 | 0.94 | 0.98 | 0.96 |
| NB | 0.993844049 | 0.992814382 | 0.97 | 0.93 | 0.95 |
| LDA | 0.972640219 | 0.981219819 | 0.79 | 1 | 0.91 |
| DTREE | 0.924076607 | 0.928019693 | 0.94 | 0.91 | 0.92 |
| LR | 0.954172367 | 0.958733845 | 0.85 | 0.93 | 0.9 |
| KNN | 0.985294118 | 0.991787624 | 0.62 | 1 | 0.83 |
| SVM | 1 | 1 | 1 | 1 | 1 |
| RF | 0.999658003 | 0.999579832 | 0.97 | 1 | 0.99 |
| NB | 0.997948016 | 0.997582141 | 0.97 | 1 | 0.99 |
| GB | 0.996580027 | 0.996179646 | 0.97 | 1 | 0.99 |
| DTREE | 0.985294118 | 0.991787624 | 0.97 | 1 | 0.99 |
| ADB | 0.999316005 | 0.999147306 | 0.94 | 1 | 0.97 |
| LR | 0.967852257 | 0.96939651 | 0.85 | 1 | 0.94 |
| LDA | 0.976744186 | 0.983924668 | 0.79 | 1 | 0.91 |
| KNN | 0.969220246 | 0.981792717 | 0.62 | 1 | 0.83 |

Machine Learning Round 2

| | | | | | |
|---|---|---|---|---|---|
| SVM | 0.997 | 0.997 | 0.94 | 0.98 | 0.96 |
| RF | 0.996 | 0.995 | 0.94 | 0.93 | 0.94 |
| ADB | 0.979 | 0.97 | 0.94 | 0.93 | 0.94 |
| GB | 0.991 | 0.989 | 0.91 | 0.93 | 0.92 |
| KNN | 0.967 | 0.979 | 0.79 | 1 | 0.91 |
| LDA | 0.949 | 0.957 | 0.85 | 0.95 | 0.91 |
| NB | 0.975 | 0.971 | 0.97 | 0.79 | 0.87 |
| DTREE | 0.906 | 0.922 | 0.88 | 0.93 | 0.91 |
| LR | 0.941 | 0.945 | 0.85 | 0.84 | 0.84 |
| SVM | 1 | 1 | 1 | 1 | 1 |
| RF | 0.997264022 | 0.996862187 | 0.97 | 0.98 | 0.97 |
| ADB | 0.994528044 | 0.992902974 | 1 | 0.93 | 0.96 |
| GB | 0.995212038 | 0.994396296 | 0.91 | 1 | 0.96 |
| NB | 0.993160055 | 0.993237781 | 0.97 | 0.91 | 0.94 |
| LDA | 0.934336525 | 0.952271824 | 0.82 | 1 | 0.92 |
| DTREE | 0.924076607 | 0.928019693 | 0.94 | 0.91 | 0.92 |
| KNN | 0.98119015 | 0.986745607 | 0.74 | 1 | 0.88 |
| LR | 0.91997264 | 0.939803033 | 0.79 | 1 | 0.91 |
| SVM | 1 | 1 | 0.97 | 1 | 0.99 |
| GB | 0.997948016 | 0.997582141 | 0.97 | 0.98 | 0.97 |
| RF | 1 | 1 | 0.94 | 1 | 0.97 |
| NB | 0.99247606 | 0.991718859 | 0.94 | 0.98 | 0.96 |
| ADB | 0.995896033 | 0.994880965 | 0.94 | 0.95 | 0.95 |
| DTREE | 0.958960328 | 0.968423733 | 0.94 | 0.98 | 0.96 |
| LR | 0.94254446 | 0.950846412 | 0.82 | 1 | 0.92 |
| KNN | 0.955882353 | 0.975362872 | 0.79 | 1 | 0.91 |
| LDA | 0.959644323 | 0.9698986 | 0.79 | 1 | 0.91 |
| RF | 1 | 1 | 1 | 1 | 1 |
| SVM | 1 | 1 | 1 | 1 | 1 |
| ADB | 0.997948016 | 0.997363956 | 1 | 0.98 | 0.99 |
| GB | 0.997948016 | 0.997582141 | 0.97 | 1 | 0.99 |
| LDA | 0.967168263 | 0.969767323 | 0.88 | 1 | 0.95 |
| NB | 0.989056088 | 0.987988697 | 0.97 | 0.86 | 0.91 |
| KNN | 0.968536252 | 0.980901451 | 0.71 | 1 | 0.87 |
| LR | 0.93502052 | 0.941356668 | 0.85 | 0.84 | 0.84 |
| DTREE | 0.827291382 | 0.851604278 | 0.79 | 0.86 | 0.83 |
| SVM | 1 | 1 | 1 | 1 | 1 |
| RF | 1 | 1 | 0.97 | 1 | 0.99 |
| ADB | 0.991792066 | 0.989273539 | 0.94 | 0.95 | 0.95 |
| GB | 0.997264022 | 0.996862187 | 0.85 | 1 | 0.94 |
| NB | 0.983584131 | 0.979570491 | 0.97 | 0.88 | 0.92 |
| LDA | 0.968536252 | 0.970612796 | 0.82 | 1 | 0.92 |
| KNN | 0.954172367 | 0.973065078 | 0.79 | 1 | 0.91 |
| DTREE | 0.911764706 | 0.950725745 | 0.82 | 1 | 0.92 |
| LR | 0.959644323 | 0.958847044 | 0.85 | 0.93 | 0.9 |
| SVM | 1 | 1 | 0.97 | 1 | 0.99 |
| RF | 0.999316005 | 0.999147306 | 0.97 | 1 | 0.99 |
| ADB | 0.995896033 | 0.994971561 | 0.94 | 0.95 | 0.95 |
| GB | 0.99247606 | 0.990504665 | 0.88 | 0.98 | 0.94 |
| NB | 0.987004104 | 0.98434637 | 0.94 | 0.91 | 0.92 |
| LR | 0.959644323 | 0.966863792 | 0.82 | 1 | 0.92 |
| LDA | 0.943228454 | 0.962372763 | 0.82 | 1 | 0.92 |
| KNN | 0.970588235 | 0.983575248 | 0.76 | 1 | 0.9 |
| DTREE | 0.888508892 | 0.917392412 | 0.82 | 0.95 | 0.9 |
| SVM | 1 | 1 | 1 | 1 | 1 |
| RF | 0.99623803 | 0.995272532 | 0.97 | 0.95 | 0.96 |
| GB | 0.995896033 | 0.994798149 | 0.94 | 0.95 | 0.95 |
| NB | 0.989056088 | 0.986007618 | 0.97 | 0.91 | 0.94 |
| DTREE | 0.953488372 | 0.947368421 | 1 | 0.91 | 0.95 |
| ADB | 0.993844049 | 0.99304568 | 0.97 | 0.88 | 0.92 |
| LDA | 0.939808482 | 0.958362758 | 0.82 | 1 | 0.92 |
| LR | 0.934336525 | 0.947963629 | 0.85 | 0.91 | 0.88 |
| KNN | 0.933652531 | 0.956529582 | 0.71 | 1 | 0.87 |
| RF | 1 | 1 | 0.97 | 1 | 0.99 |
| DTREE | 0.985294118 | 0.991787624 | 0.97 | 1 | 0.99 |
| SVM | 1 | 1 | 0.94 | 1 | 0.97 |
| GB | 0.998632011 | 0.99834267 | 0.94 | 1 | 0.97 |
| ADB | 0.993160055 | 0.991604122 | 0.91 | 0.98 | 0.95 |
| NB | 0.988372093 | 0.987158239 | 0.94 | 0.93 | 0.94 |
| LDA | 0.974008208 | 0.977313711 | 0.82 | 1 | 0.92 |
| LR | 0.96374829 | 0.964348023 | 0.85 | 0.98 | 0.92 |
| KNN | 0.970588235 | 0.983575248 | 0.76 | 1 | 0.9 |
| SVM | 1 | 1 | 0.97 | 1 | 0.99 |
| RF | 0.997948016 | 0.997582141 | 0.97 | 1 | 0.99 |
| GB | 0.991792066 | 0.990993444 | 0.94 | 1 | 0.97 |
| ADB | 0.998632011 | 0.998269147 | 0.94 | 0.98 | 0.96 |
| LDA | 0.961012312 | 0.961911399 | 0.85 | 0.98 | 0.92 |
| NB | 0.977428181 | 0.972486602 | 0.94 | 0.86 | 0.9 |
| KNN | 0.985294118 | 0.991787624 | 0.76 | 1 | 0.9 |
| LR | 0.926812585 | 0.933028636 | 0.82 | 0.91 | 0.87 |
| DTREE | 0.84746922 | 0.882576855 | 0.76 | 0.93 | 0.86 |
| SVM | 1 | 1 | 1 | 0.98 | 0.99 |
| RF | 0.998632011 | 0.998269147 | 0.97 | 0.98 | 0.97 |
| GB | 0.997264022 | 0.99655932 | 0.94 | 0.98 | 0.96 |
| ADB | 0.993844049 | 0.991985148 | 0.97 | 0.95 | 0.96 |
| NB | 0.991792066 | 0.989253609 | 0.97 | 0.91 | 0.94 |
| DTREE | 0.947332421 | 0.954163484 | 0.94 | 0.95 | 0.95 |
| KNN | 0.994870041 | 0.993665541 | 0.82 | 1 | 0.92 |
| LR | 0.969904241 | 0.969515135 | 0.85 | 0.98 | 0.92 |
| LDA | 0.961012312 | 0.962684415 | 0.82 | 0.95 | 0.9 |

Machine Learning Round 3

| | | | | | |
|---|---|---|---|---|---|
| LR | 1 | 1 | 1 | 1 | 1 |
| RF | 1 | 1 | 1 | 1 | 1 |
| ADB | 1 | 1 | 1 | 1 | 1 |
| NB | 0.999316005 | 0.999147306 | 1 | 0.98 | 0.99 |
| SVM | 1 | 1 | 0.97 | 1 | 0.99 |
| GB | 0.983926129 | 0.990197799 | 0.97 | 1 | 0.99 |
| LDA | 0.970588235 | 0.983467191 | 0.94 | 1 | 0.97 |
| KNN | 0.985294118 | 0.991787624 | 0.91 | 1 | 0.96 |
| DTREE | 0.962038304 | 0.963216196 | 0.97 | 0.95 | 0.96 |
| RF | 1 | 1 | 1 | 1 | 1 |
| ADB | 1 | 1 | 1 | 1 | 1 |
| NB | 0.99931601 | 0.999147306 | 1 | 0.98 | 0.99 |
| LR | 1 | 1 | 0.97 | 1 | 0.99 |
| SVM | 1 | 1 | 0.97 | 1 | 0.99 |
| GB | 0.99863201 | 0.998269147 | 0.97 | 0.98 | 0.97 |
| LDA | 0.97058824 | 0.983467191 | 0.94 | 1 | 0.97 |
| KNN | 0.98529412 | 0.991787624 | 0.91 | 1 | 0.96 |
| DTREE | 0.95896033 | 0.968423733 | 0.94 | 0.98 | 0.96 |
| LR | 1 | 1 | 1 | 1 | 1 |
| RF | 1 | 1 | 1 | 1 | 1 |
| ADB | 0.99863201 | 0.99826915 | 1 | 0.98 | 0.99 |
| SVM | 1 | 1 | 0.97 | 1 | 0.99 |
| GB | 1 | 1 | 0.97 | 1 | 0.99 |
| DTREE | 0.98529412 | 0.99178762 | 0.97 | 1 | 0.99 |
| NB | 0.9880301 | 0.98487395 | 1 | 0.95 | 0.97 |
| LDA | 0.97058824 | 0.98346719 | 0.94 | 1 | 0.97 |
| KNN | 0.98153215 | 0.98719204 | 0.91 | 1 | 0.96 |
| LR | 1 | 1 | 1 | 1 | 1 |
| RF | 1 | 1 | 1 | 1 | 1 |
| ADB | 1 | 1 | 1 | 1 | 1 |
| NB | 0.99931601 | 0.99914731 | 1 | 0.98 | 0.99 |
| GB | 0.99863201 | 0.99826915 | 1 | 0.98 | 0.99 |
| SVM | 1 | 1 | 0.97 | 1 | 0.99 |
| LDA | 0.97058824 | 0.98346719 | 0.94 | 1 | 0.97 |
| KNN | 0.98255814 | 0.98837685 | 0.91 | 1 | 0.96 |
| DTREE | 0.95896033 | 0.96842373 | 0.94 | 0.98 | 0.96 |
| LR | 1 | 1 | 1 | 1 | 1 |
| RF | 1 | 1 | 1 | 1 | 1 |
| ADB | 1 | 1 | 1 | 1 | 1 |
| SVM | 1 | 1 | 0.97 | 1 | 0.99 |
| NB | 0.99794802 | 0.99746451 | 1 | 0.95 | 0.97 |
| GB | 0.99760602 | 0.9970567 | 1 | 0.95 | 0.97 |
| LDA | 0.97058824 | 0.98346719 | 0.94 | 1 | 0.97 |
| KNN | 0.98529412 | 0.99178762 | 0.91 | 1 | 0.96 |
| DTREE | 0.94733242 | 0.95416348 | 0.94 | 0.95 | 0.95 |
| LR | 1 | 1 | 1 | 1 | 1 |
| RF | 1 | 1 | 1 | 1 | 1 |
| ADB | 1 | 1 | 1 | 1 | 1 |
| SVM | 1 | 1 | 0.97 | 1 | 0.99 |
| DTREE | 0.98837209 | 0.98571429 | 1 | 0.98 | 0.99 |
| GB | 0.9880301 | 0.98487395 | 0.97 | 0.98 | 0.97 |
| NB | 0.99931601 | 0.99914731 | 1 | 0.93 | 0.96 |
| KNN | 0.99863201 | 0.99836601 | 0.91 | 1 | 0.96 |
| LDA | 0.97058824 | 0.98346719 | 0.94 | 1 | 0.97 |
| LR | 1 | 1 | 1 | 1 | 1 |
| RF | 1 | 1 | 1 | 1 | 1 |
| ADB | 1 | 1 | 1 | 1 | 1 |
| GB | 0.999316005 | 0.999147306 | 1 | 0.98 | 0.99 |
| SVM | 1 | 1 | 0.97 | 1 | 0.99 |
| NB | 0.999316005 | 0.999147306 | 1 | 0.95 | 0.97 |
| LDA | 0.970588235 | 0.983467191 | 0.94 | 1 | 0.97 |
| KNN | 0.985294118 | 0.991787624 | 0.91 | 1 | 0.96 |
| DTREE | 0.958960328 | 0.968423733 | 0.94 | 0.98 | 0.96 |
| RF | 1 | 1 | 1 | 1 | 1 |
| ADB | 1 | 1 | 1 | 1 | 1 |
| NB | 0.99931601 | 0.99914731 | 1 | 0.98 | 0.99 |
| LR | 1 | 1 | 0.97 | 1 | 0.99 |
| SVM | 1 | 1 | 0.97 | 1 | 0.99 |
| DTREE | 0.98529412 | 0.99178762 | 0.97 | 1 | 0.99 |
| GB | 0.98461012 | 0.99094729 | 0.97 | 1 | 0.99 |
| LDA | 0.97058824 | 0.98346719 | 0.94 | 1 | 0.97 |
| KNN | 0.98426813 | 0.99049005 | 0.91 | 1 | 0.96 |
| RF | 1 | 1 | 1 | 1 | 1 |
| ADB | 1 | 1 | 1 | 1 | 1 |
| LR | 1 | 1 | 0.97 | 1 | 0.99 |
| SVM | 1 | 1 | 0.97 | 1 | 0.99 |
| LDA | 0.97058824 | 0.98346719 | 0.94 | 1 | 0.97 |
| NB | 0.97606019 | 0.97061158 | 0.97 | 0.95 | 0.96 |
| KNN | 0.97811218 | 0.98279778 | 0.88 | 0.95 | 0.92 |
| GB | 0.90424077 | 0.8996379 | 0.97 | 0.81 | 0.88 |
| DTREE | 0.89227086 | 0.89422665 | 0.97 | 0.81 | 0.88 |
| LR | 1 | 1 | 1 | 1 | 1 |
| RF | 1 | 1 | 1 | 1 | 1 |
| ADB | 1 | 1 | 1 | 1 | 1 |
| SVM | 1 | 1 | 0.97 | 1 | 0.99 |
| NB | 1 | 1 | 0.97 | 1 | 0.99 |
| GB | 1 | 1 | 0.94 | 1 | 0.97 |
| DTREE | 0.97058824 | 0.98357525 | 0.94 | 1 | 0.97 |
| LDA | 0.97058824 | 0.98346719 | 0.94 | 1 | 0.97 |
| KNN | 0.98529412 | 0.99178762 | 0.88 | 1 | 0.95 |

TABLE 2B
Machine learning algorithm performance for non-
critical COVID vs healthy classification

| | | | | | |
|---|---|---|---|---|---|
| | | Precision/ | | | |
| | ROC Curve | Recall | | | |
| Model | AUC | AUC | Sensitivity | Specificity | Accuracy |

Machine Learning Round 1

| | | | | | |
|---|---|---|---|---|---|
| SVM | 1 | 1 | 1 | 1 | 1 |
| LDA | 0.997 | 0.996 | 0.88 | 1 | 0.94 |
| RF | 0.982 | 0.98 | 0.94 | 0.95 | 0.94 |
| LR | 0.967 | 0.966 | 0.81 | 1 | 0.91 |
| GB | 0.964 | 0.959 | 1 | 0.79 | 0.89 |
| ADB | 0.961 | 0.954 | 1 | 0.79 | 0.89 |
| NB | 0.964 | 0.955 | 1 | 0.63 | 0.8 |
| DTREE | 0.863 | 0.878 | 0.94 | 0.79 | 0.86 |
| KNN | 0.906 | 0.927 | 0.44 | 1 | 0.74 |
| SVM | 1 | 1 | 1 | 0.95 | 0.97 |
| ADB | 0.993421053 | 0.992172181 | 1 | 0.95 | 0.97 |
| LDA | 0.996710526 | 0.99620864 | 0.94 | 0.95 | 0.94 |
| GB | 0.983552632 | 0.98095311 | 1 | 0.89 | 0.94 |
| RF | 0.978618421 | 0.974937996 | 1 | 0.89 | 0.94 |
| LR | 0.967105263 | 0.962430548 | 0.88 | 0.89 | 0.89 |
| NB | 0.957236842 | 0.948167409 | 1 | 0.74 | 0.86 |
| DTREE | 0.837171053 | 0.858035714 | 0.94 | 0.74 | 0.83 |
| KNN | 0.856907895 | 0.882700893 | 0.44 | 1 | 0.74 |
| SVM | 1 | 1 | 1 | 0.95 | 0.97 |
| LDA | 0.996710526 | 0.99620864 | 0.94 | 1 | 0.97 |
| RF | 0.978618421 | 0.973132025 | 1 | 0.89 | 0.94 |
| ADB | 0.993421053 | 0.992172181 | 1 | 0.84 | 0.91 |
| LR | 0.973684211 | 0.973647661 | 0.88 | 0.95 | 0.91 |
| GB | 0.980263158 | 0.978828044 | 0.94 | 0.84 | 0.89 |
| NB | 0.930921053 | 0.907135012 | 1 | 0.79 | 0.89 |
| DTREE | 0.858552632 | 0.877836134 | 0.88 | 0.84 | 0.86 |
| KNN | 0.911184211 | 0.931215659 | 0.38 | 1 | 0.71 |
| SVM | 1 | 1 | 1 | 1 | 1 |
| LDA | 0.996710526 | 0.99620864 | 0.94 | 1 | 0.97 |
| ADB | 0.986842105 | 0.98449937 | 1 | 0.84 | 0.91 |
| LR | 0.983552632 | 0.98150552 | 0.88 | 0.95 | 0.91 |
| NB | 0.967105263 | 0.95911274 | 1 | 0.84 | 0.91 |
| GB | 0.947368421 | 0.94444444 | 1 | 0.84 | 0.91 |
| DTREE | 0.921052632 | 0.92105263 | 1 | 0.84 | 0.91 |
| RF | 0.973684211 | 0.96853408 | 0.81 | 0.89 | 0.86 |
| KNN | 0.919407895 | 0.94091117 | 0.44 | 1 | 0.74 |
| SVM | 0.980263158 | 0.972767628 | 1 | 0.84 | 0.91 |
| LDA | 0.970394737 | 0.956847164 | 1 | 0.84 | 0.91 |
| RF | 0.949013158 | 0.935823643 | 0.88 | 0.89 | 0.89 |
| LR | 0.9375 | 0.940637001 | 0.88 | 0.89 | 0.89 |
| GB | 0.953947368 | 0.93390606 | 0.69 | 0.95 | 0.83 |
| NB | 0.925986842 | 0.915889262 | 1 | 0.68 | 0.83 |
| ADB | 0.898026316 | 0.879215986 | 0.88 | 0.79 | 0.83 |
| DTREE | 0.786184211 | 0.85275974 | 0.62 | 0.95 | 0.8 |
| KNN | 0.911184211 | 0.931215659 | 0.38 | 1 | 0.71 |
| SVM | 1 | 1 | 1 | 1 | 1 |
| LDA | 1 | 1 | 0.94 | 1 | 0.97 |
| RF | 0.993421053 | 0.99285131 | 1 | 0.89 | 0.94 |
| GB | 0.967105263 | 0.96755803 | 0.94 | 0.95 | 0.94 |
| LR | 0.973684211 | 0.97213254 | 0.81 | 0.95 | 0.89 |
| NB | 0.957236842 | 0.95086199 | 1 | 0.68 | 0.83 |
| ADB | 0.888157895 | 0.83446812 | 0.81 | 0.79 | 0.8 |
| KNN | 0.866776316 | 0.89461996 | 0.38 | 1 | 0.71 |
| DTREE | 0.733552632 | 0.78282967 | 0.62 | 0.84 | 0.74 |
| SVM | 1 | 1 | 1 | 0.95 | 0.97 |
| RF | 0.995065789 | 0.994255515 | 1 | 0.95 | 0.97 |
| LDA | 0.996710526 | 0.99620864 | 0.94 | 0.95 | 0.94 |
| ADB | 0.990131579 | 0.989857456 | 0.94 | 0.95 | 0.94 |
| LR | 0.983552632 | 0.979863384 | 0.81 | 0.95 | 0.89 |
| NB | 0.947368421 | 0.940018129 | 1 | 0.74 | 0.86 |
| GB | 0.939144737 | 0.926552094 | 0.81 | 0.89 | 0.86 |
| KNN | 0.919407895 | 0.941964286 | 0.38 | 1 | 0.71 |
| DTREE | 0.738486842 | 0.781845238 | 0.69 | 0.79 | 0.74 |
| LDA | 1 | 1 | 1 | 0.95 | 0.97 |
| SVM | 0.986842105 | 0.983220716 | 1 | 0.95 | 0.97 |
| RF | 0.963815789 | 0.957422535 | 0.94 | 0.84 | 0.89 |
| GB | 0.917763158 | 0.893494368 | 1 | 0.84 | 0.91 |
| NB | 0.927631579 | 0.913230198 | 1 | 0.79 | 0.89 |
| LR | 0.960526316 | 0.95644298 | 0.81 | 0.84 | 0.83 |
| DTREE | 0.858552632 | 0.877836134 | 0.88 | 0.84 | 0.86 |
| ADB | 0.935855263 | 0.908109772 | 0.75 | 0.89 | 0.83 |
| KNN | 0.909539474 | 0.932860644 | 0.62 | 1 | 0.83 |
| SVM | 1 | 1 | 1 | 1 | 1 |
| RF | 1 | 1 | 0.94 | 1 | 0.97 |
| LDA | 1 | 1 | 0.94 | 1 | 0.97 |
| LR | 0.986842105 | 0.987171053 | 0.88 | 1 | 0.94 |
| ADB | 0.963815789 | 0.958116334 | 0.94 | 0.84 | 0.89 |
| DTREE | 0.879934211 | 0.913392857 | 0.81 | 0.95 | 0.89 |
| NB | 0.963815789 | 0.955972423 | 1 | 0.68 | 0.83 |
| GB | 0.958881579 | 0.954913897 | 0.75 | 0.89 | 0.83 |
| KNN | 0.870065789 | 0.869012605 | 0.44 | 0.95 | 0.71 |
| LDA | 0.996710526 | 0.99620864 | 1 | 0.95 | 0.97 |
| SVM | 0.976973684 | 0.966801719 | 1 | 0.95 | 0.97 |
| RF | 0.980263158 | 0.974826754 | 0.88 | 0.95 | 0.91 |
| LR | 0.950657895 | 0.953726119 | 0.88 | 0.95 | 0.91 |
| NB | 0.947368421 | 0.936260829 | 1 | 0.79 | 0.89 |
| GB | 0.967105263 | 0.961871414 | 0.69 | 0.95 | 0.83 |
| ADB | 0.927631579 | 0.879005877 | 0.75 | 0.89 | 0.83 |
| KNN | 0.912828947 | 0.933619505 | 0.44 | 1 | 0.74 |
| DTREE | 0.728618421 | 0.790340909 | 0.56 | 0.89 | 0.74 |

Machine Learning Round 2

| | | | | | |
|---|---|---|---|---|---|
| SVM | 1 | 1 | 1 | 1 | 1 |
| LDA | 1 | 1 | 1 | 1 | 1 |
| GB | 1 | 1 | 1 | 1 | 1 |
| RF | 1 | 1 | 1 | 0.95 | 0.97 |
| ADB | 0.99 | 0.988 | 1 | 0.89 | 0.94 |
| LR | 0.984 | 0.983 | 0.94 | 0.89 | 0.91 |
| KNN | 0.965 | 0.979 | 0.69 | 1 | 0.86 |
| NB | 0.947 | 0.929 | 0.88 | 0.84 | 0.86 |
| DTREE | 0.822 | 0.861 | 0.75 | 0.89 | 0.83 |
| RF | 0.983552632 | 0.983552632 | 1 | 0.95 | 0.97 |
| SVM | 0.970394737 | 0.970394737 | 1 | 0.95 | 0.97 |
| LDA | 0.996710526 | 0.996710526 | 0.94 | 0.95 | 0.94 |
| ADB | 0.957236842 | 0.957236842 | 0.94 | 0.84 | 0.89 |
| LR | 0.950657895 | 0.950657895 | 0.81 | 0.95 | 0.89 |
| GB | 0.967105263 | 0.967105263 | 0.75 | 0.95 | 0.86 |
| NB | 0.940789474 | 0.940789474 | 0.88 | 0.84 | 0.86 |
| KNN | 0.940789474 | 0.940789474 | 0.69 | 0.95 | 0.83 |
| DTREE | 0.863486842 | 0.863486842 | 0.94 | 0.79 | 0.86 |
| LDA | 1 | 1 | 1 | 1 | 1 |
| SVM | 1 | 1 | 1 | 0.95 | 0.97 |
| LR | 0.94736842 | 0.94736842 | 0.88 | 0.89 | 0.89 |
| KNN | 0.92434211 | 0.92434211 | 0.75 | 1 | 0.89 |
| DTREE | 0.89473684 | 0.89473684 | 1 | 0.79 | 0.89 |
| ADB | 0.9375 | 0.9375 | 0.94 | 0.79 | 0.86 |
| GB | 0.93092105 | 0.93092105 | 0.88 | 0.84 | 0.86 |
| RF | 0.93421053 | 0.93421053 | 0.88 | 0.79 | 0.83 |
| NB | 0.90460526 | 0.90460526 | 0.88 | 0.74 | 0.8 |
| SVM | 1 | 1 | 1 | 1 | 1 |
| LDA | 1 | 1 | 1 | 1 | 1 |
| RF | 0.996710526 | 0.99671053 | 0.94 | 0.95 | 0.94 |
| LR | 0.990131579 | 0.99013158 | 0.88 | 1 | 0.94 |
| DTREE | 0.942434211 | 0.94243421 | 0.94 | 0.95 | 0.94 |
| NB | 0.967105263 | 0.96710526 | 1 | 0.84 | 0.91 |
| GB | 0.958881579 | 0.95888158 | 0.88 | 0.95 | 0.91 |
| ADB | 0.980263158 | 0.98026316 | 0.94 | 0.84 | 0.89 |
| KNN | 0.949013158 | 0.94901316 | 0.69 | 1 | 0.86 |
| SVM | 1 | 1 | 1 | 1 | 1 |
| LDA | 0.99671053 | 0.99671053 | 0.94 | 0.95 | 0.94 |
| RF | 0.97697368 | 0.97697368 | 0.88 | 0.89 | 0.89 |
| GB | 0.96381579 | 0.96381579 | 0.94 | 0.84 | 0.89 |
| LR | 0.96052632 | 0.96052632 | 0.81 | 0.95 | 0.89 |
| ADB | 0.95394737 | 0.95394737 | 0.94 | 0.74 | 0.83 |
| KNN | 0.89638158 | 0.89638158 | 0.62 | 1 | 0.83 |
| NB | 0.90460526 | 0.90460526 | 0.81 | 0.79 | 0.8 |
| DTREE | 0.83223684 | 0.83223684 | 0.88 | 0.79 | 0.83 |
| SVM | 1 | 1 | 1 | 1 | 1 |
| LDA | 1 | 1 | 1 | 1 | 1 |
| DTREE | 0.96875 | 0.96875 | 0.94 | 1 | 0.97 |
| GB | 0.96217105 | 0.96217105 | 0.94 | 1 | 0.97 |
| RF | 0.99671053 | 0.99671053 | 0.94 | 0.95 | 0.94 |
| LR | 0.98355263 | 0.98355263 | 0.81 | 1 | 0.91 |
| KNN | 0.95230263 | 0.95230263 | 0.69 | 1 | 0.86 |
| NB | 0.94078947 | 0.94078947 | 0.88 | 0.79 | 0.83 |
| ADB | 0.94078947 | 0.94078947 | 0.75 | 0.89 | 0.83 |
| LDA | 0.99671053 | 0.99671053 | 1 | 0.95 | 0.97 |
| SVM | 0.97039474 | 0.97039474 | 1 | 0.89 | 0.94 |
| GB | 0.94407895 | 0.94407895 | 0.94 | 0.89 | 0.91 |
| DTREE | 0.91611842 | 0.91611842 | 0.94 | 0.89 | 0.91 |
| ADB | 0.96710526 | 0.96710526 | 1 | 0.74 | 0.86 |
| RF | 0.96052632 | 0.96052632 | 0.94 | 0.74 | 0.83 |
| LR | 0.91447368 | 0.91447368 | 0.88 | 0.84 | 0.86 |
| NB | 0.92763158 | 0.92763158 | 0.94 | 0.63 | 0.77 |
| KNN | 0.92434211 | 0.92434211 | 0.56 | 0.89 | 0.74 |
| LDA | 1 | 1 | 1 | 1 | 1 |
| RF | 0.976973684 | 0.97697368 | 1 | 0.95 | 0.97 |
| DTREE | 0.96875 | 0.96875 | 0.94 | 1 | 0.97 |
| ADB | 1 | 1 | 1 | 0.89 | 0.94 |
| SVM | 0.993421053 | 0.99342105 | 0.94 | 0.95 | 0.94 |
| GB | 0.993421053 | 0.99342105 | 0.94 | 0.95 | 0.94 |
| NB | 0.953947368 | 0.95394737 | 0.94 | 0.89 | 0.91 |
| LR | 0.963815789 | 0.96381579 | 0.75 | 0.95 | 0.86 |
| KNN | 0.912828947 | 0.91282895 | 0.5 | 1 | 0.77 |
| LDA | 0.99671053 | 0.99671053 | 1 | 0.89 | 0.94 |
| KNN | 0.96710526 | 0.96710526 | 0.75 | 1 | 0.89 |
| SVM | 0.98026316 | 0.98026316 | 1 | 0.74 | 0.86 |
| GB | 0.90460526 | 0.90460526 | 0.94 | 0.74 | 0.83 |
| NB | 0.86842105 | 0.86842105 | 1 | 0.68 | 0.83 |
| RF | 0.86019737 | 0.86019737 | 1 | 0.68 | 0.83 |
| ADB | 0.86842105 | 0.86842105 | 1 | 0.63 | 0.8 |
| LR | 0.90460526 | 0.90460526 | 0.88 | 0.68 | 0.77 |
| DTREE | 0.76480263 | 0.76480263 | 0.69 | 0.84 | 0.77 |
| LDA | 0.996710526 | 0.996710526 | 1 | 0.95 | 0.97 |
| SVM | 1 | 1 | 1 | 0.89 | 0.94 |
| RF | 0.988486842 | 0.988486842 | 0.94 | 0.95 | 0.94 |
| ADB | 0.983552632 | 0.983552632 | 0.94 | 0.95 | 0.94 |
| GB | 0.976973684 | 0.976973684 | 0.88 | 0.89 | 0.89 |
| NB | 0.950657895 | 0.950657895 | 0.94 | 0.84 | 0.89 |
| KNN | 0.962171053 | 0.962171053 | 0.75 | 1 | 0.89 |
| DTREE | 0.889802632 | 0.889802632 | 0.94 | 0.84 | 0.89 |
| LR | 0.957236842 | 0.957236842 | 0.81 | 0.84 | 0.83 |

Machine Learning Round 3

| | | | | | |
|---|---|---|---|---|---|
| LR | 1 | 1 | 1 | 1 | 1 |
| RF | 1 | 1 | 1 | 1 | 1 |
| ADB | 1 | 1 | 1 | 0.95 | 0.97 |
| SVM | 1 | 1 | 0.94 | 1 | 0.97 |
| GB | 1 | 1 | 0.94 | 1 | 0.97 |
| NB | 0.970394737 | 0.970394737 | 1 | 0.95 | 0.97 |
| DTREE | 0.96875 | 0.96875 | 0.94 | 1 | 0.97 |
| LDA | 0.894736842 | 0.894736842 | 0.94 | 0.89 | 0.91 |
| KNN | 0.932565789 | 0.932565789 | 0.62 | 1 | 0.83 |
| LR | 1 | 1 | 1 | 1 | 1 |
| ADB | 0.996710526 | 0.99671053 | 1 | 0.95 | 0.97 |
| RF | 1 | 1 | 0.94 | 1 | 0.97 |
| SVM | 1 | 1 | 0.94 | 1 | 0.97 |
| LDA | 1 | 1 | 0.94 | 1 | 0.97 |
| NB | 0.970394737 | 0.97039474 | 1 | 0.95 | 0.97 |
| KNN | 1 | 1 | 0.81 | 1 | 0.91 |
| DTREE | 0.942434211 | 0.94243421 | 0.94 | 0.95 | 0.94 |
| GB | 0.9375 | 0.9375 | 0.94 | 0.95 | 0.94 |
| LR | 1 | 1 | 1 | 1 | 1 |
| RF | 1 | 1 | 0.94 | 1 | 0.97 |
| ADB | 0.996710526 | 0.996710526 | 0.94 | 1 | 0.97 |
| SVM | 0.996710526 | 0.996710526 | 0.94 | 0.95 | 0.94 |
| NB | 0.970394737 | 0.970394737 | 1 | 0.89 | 0.94 |
| KNN | 0.957236842 | 0.957236842 | 0.69 | 1 | 0.86 |
| GB | 0.935855263 | 0.935855263 | 0.94 | 0.79 | 0.86 |
| DTREE | 0.863486842 | 0.863486842 | 0.94 | 0.79 | 0.86 |
| LDA | 0.731907895 | 0.731907895 | 0.69 | 0.84 | 0.77 |
| LR | 1 | 1 | 1 | 0.95 | 0.97 |
| RF | 1 | 1 | 0.94 | 1 | 0.97 |
| SVM | 0.996710526 | 0.996710526 | 0.94 | 0.95 | 0.94 |
| NB | 0.934210526 | 0.934210526 | 1 | 0.84 | 0.91 |
| LDA | 0.986842105 | 0.986842105 | 0.94 | 0.79 | 0.86 |
| KNN | 0.919407895 | 0.919407895 | 0.81 | 1 | 0.91 |
| ADB | 0.935855263 | 0.935855263 | 0.94 | 0.79 | 0.86 |
| DTREE | 0.863486842 | 0.863486842 | 0.94 | 0.79 | 0.86 |
| GB | 0.863486842 | 0.863486842 | 0.94 | 0.79 | 0.86 |
| LR | 1 | 1 | 1 | 1 | 1 |
| RF | 1 | 1 | 0.94 | 1 | 0.97 |
| SVM | 1 | 1 | 0.94 | 1 | 0.97 |
| ADB | 0.99671053 | 0.99671053 | 0.94 | 1 | 0.97 |
| DTREE | 0.97368421 | 0.97368421 | 1 | 0.95 | 0.97 |
| NB | 0.97039474 | 0.97039474 | 1 | 0.95 | 0.97 |
| KNN | 0.99671053 | 0.99671053 | 0.75 | 1 | 0.89 |
| LDA | 0.93421053 | 0.93421053 | 0.94 | 0.89 | 0.91 |
| GB | 0.95888158 | 0.95888158 | 0.94 | 0.74 | 0.83 |
| LR | 1 | 1 | 1 | 1 | 1 |
| RF | 1 | 1 | 0.94 | 1 | 0.97 |
| SVM | 1 | 1 | 0.94 | 1 | 0.97 |
| ADB | 0.99671053 | 0.99671053 | 1 | 0.89 | 0.94 |
| NB | 0.96710526 | 0.96710526 | 1 | 0.89 | 0.94 |
| KNN | 0.99342105 | 0.99342105 | 0.69 | 1 | 0.86 |
| LDA | 0.85197368 | 0.85197368 | 1 | 0.74 | 0.86 |
| DTREE | 0.84210526 | 0.84210526 | 1 | 0.68 | 0.83 |
| GB | 0.84210526 | 0.84210526 | 1 | 0.68 | 0.83 |
| LR | 1 | 1 | 1 | 1 | 1 |
| RF | 1 | 1 | 0.94 | 1 | 0.97 |
| SVM | 1 | 1 | 0.94 | 1 | 0.97 |
| ADB | 1 | 1 | 0.94 | 1 | 0.97 |
| LDA | 0.986842105 | 0.98684211 | 0.94 | 1 | 0.97 |
| NB | 0.970394737 | 0.97039474 | 1 | 0.95 | 0.97 |
| DTREE | 0.942434211 | 0.94243421 | 0.94 | 0.95 | 0.94 |
| GB | 0.940789474 | 0.94078947 | 0.94 | 0.89 | 0.91 |
| KNN | 0.996710526 | 0.99671053 | 0.69 | 1 | 0.86 |
| RF | 1 | 1 | 1 | 1 | 1 |
| ADB | 1 | 1 | 1 | 0.95 | 0.97 |
| SVM | 0.98684211 | 0.98684211 | 1 | 0.89 | 0.94 |
| LR | 0.98355263 | 0.98355263 | 1 | 0.89 | 0.94 |
| NB | 0.94736842 | 0.94736842 | 1 | 0.89 | 0.94 |
| GB | 0.97039474 | 0.97039474 | 1 | 0.84 | 0.91 |
| DTREE | 0.92105263 | 0.92105263 | 1 | 0.84 | 0.91 |
| KNN | 0.92105263 | 0.92105263 | 0.62 | 1 | 0.83 |
| LDA | 0.70065789 | 0.70065789 | 0.62 | 0.84 | 0.74 |
| RF | 1 | 1 | 1 | 1 | 1 |
| ADB | 1 | 1 | 1 | 0.95 | 0.97 |
| LR | 0.99671053 | 0.99671053 | 1 | 0.95 | 0.97 |
| SVM | 0.99342105 | 0.99342105 | 0.94 | 0.95 | 0.94 |
| NB | 0.97039474 | 0.97039474 | 1 | 0.89 | 0.94 |
| DTREE | 0.91611842 | 0.91611842 | 0.94 | 0.89 | 0.91 |
| LDA | 0.92763158 | 0.92763158 | 0.94 | 0.84 | 0.89 |
| GB | 0.93585526 | 0.93585526 | 0.94 | 0.79 | 0.86 |
| KNN | 0.99342105 | 0.99342105 | 0.62 | 1 | 0.83 |
| LR | 1 | 1 | 1 | 1 | 1 |
| RF | 1 | 1 | 0.94 | 1 | 0.97 |
| ADB | 0.98848684 | 0.98848684 | 1 | 0.95 | 0.97 |
| DTREE | 0.97368421 | 0.97368421 | 1 | 0.95 | 0.97 |
| SVM | 0.99671053 | 0.99671053 | 0.94 | 0.95 | 0.94 |
| LDA | 0.99671053 | 0.99671053 | 0.94 | 0.95 | 0.94 |
| GB | 0.99342105 | 0.99342105 | 0.94 | 0.89 | 0.91 |
| KNN | 0.98519737 | 0.98519737 | 0.69 | 1 | 0.86 |
| NB | 0.88157895 | 0.88157895 | 1 | 0.79 | 0.89 |

TABLE 2C
Machine learning algorithm performance for critical
vs non-critical COVID classification.

| | | | | | |
|---|---|---|---|---|---|
| | ROC | Precision/ | | | |
| | Curve | Recall | | | |
| Model | AUC | AUC | Sensitivity | Specificity | Accuracy |

Machine Learning Round 1

| | | | | | |
|---|---|---|---|---|---|
| RF | 0.88833992 | 0.894 | 0.74 | 0.91 | 0.82 |
| GB | 0.89920949 | 0.9 | 0.7 | 0.91 | 0.8 |
| SVM | 0.88932806 | 0.898 | 0.74 | 0.86 | 0.8 |
| LR | 0.84782609 | 0.809 | 0.78 | 0.82 | 0.8 |
| LDA | 0.85770751 | 0.836 | 0.7 | 0.86 | 0.78 |
| ADB | 0.81422925 | 0.744 | 0.78 | 0.82 | 0.8 |
| KNN | 0.83893281 | 0.789 | 0.65 | 0.86 | 0.76 |
| NB | 0.7944664 | 0.789 | 0.7 | 0.73 | 0.71 |
| DTREE | 0.64624506 | 0.736 | 0.57 | 0.73 | 0.64 |
| GB | 0.91897233 | 0.895 | 0.83 | 0.91 | 0.87 |
| ADB | 0.8715415 | 0.864 | 0.83 | 0.82 | 0.82 |
| SVM | 0.86758893 | 0.873 | 0.78 | 0.82 | 0.8 |
| RF | 0.87648221 | 0.858 | 0.7 | 0.86 | 0.78 |
| LR | 0.84782609 | 0.79 | 0.74 | 0.82 | 0.78 |
| DTREE | 0.77667984 | 0.837 | 0.83 | 0.73 | 0.78 |
| NB | 0.79644269 | 0.79 | 0.78 | 0.73 | 0.76 |
| KNN | 0.82114625 | 0.75 | 0.7 | 0.82 | 0.76 |
| LDA | 0.85573123 | 0.822 | 0.57 | 0.82 | 0.69 |
| GB | 0.90316206 | 0.901 | 0.74 | 0.86 | 0.8 |
| SVM | 0.88537549 | 0.896 | 0.74 | 0.86 | 0.8 |
| RF | 0.88142292 | 0.88 | 0.7 | 0.91 | 0.8 |
| LR | 0.85375494 | 0.817 | 0.78 | 0.82 | 0.8 |
| KNN | 0.83300395 | 0.81 | 0.65 | 0.86 | 0.76 |
| LDA | 0.85177866 | 0.833 | 0.65 | 0.82 | 0.73 |
| NB | 0.80237154 | 0.795 | 0.74 | 0.73 | 0.73 |
| ADB | 0.81620553 | 0.84 | 0.52 | 0.86 | 0.69 |
| DTREE | 0.55533597 | 0.676 | 0.57 | 0.55 | 0.56 |
| GB | 0.90513834 | 0.88 | 0.74 | 0.95 | 0.84 |
| SVM | 0.87944664 | 0.893 | 0.78 | 0.86 | 0.82 |
| RF | 0.885375494 | 0.875 | 0.7 | 0.91 | 0.8 |
| LR | 0.845849802 | 0.803 | 0.78 | 0.82 | 0.8 |
| LDA | 0.865612648 | 0.83 | 0.7 | 0.82 | 0.76 |
| KNN | 0.824110672 | 0.805 | 0.7 | 0.82 | 0.76 |
| NB | 0.794466403 | 0.788 | 0.7 | 0.73 | 0.71 |
| ADB | 0.772727273 | 0.813 | 0.39 | 0.91 | 0.64 |
| DTREE | 0.555335968 | 0.676 | 0.57 | 0.55 | 0.56 |
| GB | 0.893280632 | 0.901 | 0.83 | 0.91 | 0.87 |
| SVM | 0.899209486 | 0.901 | 0.87 | 0.86 | 0.87 |
| ADB | 0.863636364 | 0.891 | 0.7 | 0.86 | 0.78 |
| LR | 0.859683794 | 0.812 | 0.78 | 0.82 | 0.8 |
| RF | 0.884387352 | 0.892 | 0.7 | 0.82 | 0.76 |
| LDA | 0.863636364 | 0.832 | 0.7 | 0.86 | 0.78 |
| DTREE | 0.779644269 | 0.847 | 0.7 | 0.86 | 0.78 |
| KNN | 0.85770751 | 0.809 | 0.7 | 0.82 | 0.76 |
| NB | 0.798418972 | 0.792 | 0.74 | 0.73 | 0.73 |
| GB | 0.91501976 | 0.928 | 0.74 | 0.91 | 0.82 |
| SVM | 0.89328063 | 0.897 | 0.83 | 0.82 | 0.82 |
| RF | 0.88339921 | 0.885 | 0.74 | 0.86 | 0.8 |
| ADB | 0.86363636 | 0.825 | 0.87 | 0.73 | 0.8 |
| KNN | 0.83102767 | 0.782 | 0.83 | 0.82 | 0.82 |
| LR | 0.85177866 | 0.791 | 0.78 | 0.82 | 0.8 |
| LDA | 0.86166008 | 0.829 | 0.61 | 0.86 | 0.73 |
| NB | 0.80632411 | 0.797 | 0.78 | 0.73 | 0.76 |
| DTREE | 0.71541502 | 0.812 | 0.52 | 0.91 | 0.71 |
| GB | 0.88142292 | 0.866 | 0.78 | 0.86 | 0.82 |
| SVM | 0.89328063 | 0.9 | 0.74 | 0.86 | 0.8 |
| RF | 0.86462451 | 0.876 | 0.7 | 0.86 | 0.78 |
| LR | 0.84782609 | 0.815 | 0.78 | 0.82 | 0.8 |
| LDA | 0.85770751 | 0.849 | 0.65 | 0.86 | 0.76 |
| KNN | 0.84683794 | 0.818 | 0.7 | 0.82 | 0.76 |
| NB | 0.79644269 | 0.79 | 0.74 | 0.73 | 0.73 |
| ADB | 0.78458498 | 0.791 | 0.78 | 0.64 | 0.71 |
| DTREE | 0.5770751 | 0.696 | 0.61 | 0.55 | 0.58 |
| GB | 0.901185771 | 0.907 | 0.74 | 0.91 | 0.82 |
| SVM | 0.893280632 | 0.899 | 0.74 | 0.86 | 0.8 |
| ADB | 0.873517787 | 0.896 | 0.7 | 0.91 | 0.8 |
| LR | 0.837944664 | 0.784 | 0.78 | 0.82 | 0.8 |
| LDA | 0.847826087 | 0.834 | 0.7 | 0.86 | 0.78 |
| RF | 0.87944664 | 0.886 | 0.61 | 0.86 | 0.73 |
| DTREE | 0.756916996 | 0.826 | 0.7 | 0.82 | 0.76 |
| KNN | 0.811264822 | 0.787 | 0.65 | 0.82 | 0.73 |
| NB | 0.796442688 | 0.79 | 0.7 | 0.73 | 0.71 |
| GB | 0.90909091 | 0.902 | 0.78 | 0.95 | 0.87 |
| SVM | 0.90118577 | 0.904 | 0.78 | 0.86 | 0.82 |
| LR | 0.84782609 | 0.798 | 0.83 | 0.82 | 0.82 |
| RF | 0.89426877 | 0.878 | 0.65 | 0.91 | 0.78 |
| ADB | 0.89130435 | 0.891 | 0.57 | 0.95 | 0.76 |
| LDA | 0.85968379 | 0.839 | 0.61 | 0.86 | 0.73 |
| KNN | 0.83201581 | 0.781 | 0.65 | 0.82 | 0.73 |
| NB | 0.80039526 | 0.791 | 0.7 | 0.73 | 0.71 |
| DTREE | 0.57608696 | 0.703 | 0.65 | 0.5 | 0.58 |
| GB | 0.891304348 | 0.916 | 0.78 | 0.86 | 0.82 |
| RF | 0.876482213 | 0.878 | 0.74 | 0.91 | 0.82 |
| SVM | 0.891304348 | 0.892 | 0.74 | 0.86 | 0.8 |
| LR | 0.861660079 | 0.815 | 0.74 | 0.82 | 0.78 |
| KNN | 0.826086957 | 0.793 | 0.74 | 0.82 | 0.78 |
| NB | 0.798418972 | 0.792 | 0.78 | 0.73 | 0.76 |
| LDA | 0.85770751 | 0.825 | 0.61 | 0.82 | 0.71 |
| ADB | 0.778656126 | 0.787 | 0.65 | 0.82 | 0.73 |
| DTREE | 0.735177866 | 0.81 | 0.65 | 0.82 | 0.73 |

Machine Learning Round 2

| | | | | | |
|---|---|---|---|---|---|
| RF | 0.85079051 | 0.859438734 | 0.78 | 0.86 | 0.82 |
| LDA | 0.90909091 | 0.912235245 | 0.7 | 0.86 | 0.78 |
| SVM | 0.86363636 | 0.882592585 | 0.74 | 0.86 | 0.8 |
| LR | 0.85770751 | 0.847981639 | 0.74 | 0.86 | 0.8 |
| GB | 0.85573123 | 0.830705282 | 0.74 | 0.86 | 0.8 |
| ADB | 0.82608696 | 0.81149257 | 0.78 | 0.77 | 0.78 |
| NB | 0.83003953 | 0.805166704 | 0.7 | 0.82 | 0.76 |
| KNN | 0.78656126 | 0.757551185 | 0.7 | 0.77 | 0.73 |
| DTREE | 0.62351779 | 0.718719807 | 0.57 | 0.68 | 0.62 |
| RF | 0.885375494 | 0.90129449 | 0.7 | 0.91 | 0.8 |
| GB | 0.853754941 | 0.87230409 | 0.7 | 0.91 | 0.8 |
| SVM | 0.885375494 | 0.90946163 | 0.7 | 0.86 | 0.78 |
| LDA | 0.865612648 | 0.84289571 | 0.74 | 0.86 | 0.8 |
| KNN | 0.87944664 | 0.8820139 | 0.65 | 0.91 | 0.78 |
| ADB | 0.835968379 | 0.86370288 | 0.74 | 0.86 | 0.8 |
| LR | 0.843873518 | 0.81074036 | 0.7 | 0.86 | 0.78 |
| NB | 0.81027668 | 0.81250134 | 0.7 | 0.86 | 0.78 |
| DTREE | 0.599802372 | 0.70869565 | 0.61 | 0.59 | 0.6 |
| GB | 0.89130435 | 0.89970731 | 0.7 | 0.91 | 0.8 |
| RF | 0.83003953 | 0.85300818 | 0.57 | 0.91 | 0.73 |
| SVM | 0.81422925 | 0.85142204 | 0.57 | 0.86 | 0.71 |
| NB | 0.78063241 | 0.81137922 | 0.7 | 0.77 | 0.73 |
| DTREE | 0.73320158 | 0.8057971 | 0.74 | 0.73 | 0.73 |
| KNN | 0.74703557 | 0.8145332 | 0.61 | 0.82 | 0.71 |
| ADB | 0.77667984 | 0.80446597 | 0.7 | 0.68 | 0.69 |
| LR | 0.7944664 | 0.78453196 | 0.61 | 0.73 | 0.67 |
| LDA | 0.78853755 | 0.76353874 | 0.52 | 0.73 | 0.62 |
| GB | 0.90316206 | 0.90386371 | 0.74 | 0.91 | 0.82 |
| RF | 0.90217391 | 0.90482568 | 0.74 | 0.91 | 0.82 |
| SVM | 0.87747036 | 0.85791917 | 0.83 | 0.82 | 0.82 |
| KNN | 0.82509881 | 0.82248432 | 0.78 | 0.86 | 0.82 |
| ADB | 0.82213439 | 0.78499928 | 0.78 | 0.82 | 0.8 |
| LDA | 0.8458498 | 0.79161668 | 0.74 | 0.82 | 0.78 |
| LR | 0.84387352 | 0.79824887 | 0.7 | 0.77 | 0.73 |
| NB | 0.7944664 | 0.77257237 | 0.7 | 0.77 | 0.73 |
| DTREE | 0.68972332 | 0.7721187 | 0.65 | 0.73 | 0.69 |
| RF | 0.862648221 | 0.85081565 | 0.83 | 0.86 | 0.84 |
| GB | 0.881422925 | 0.87220909 | 0.7 | 0.91 | 0.8 |
| LR | 0.849802372 | 0.80085247 | 0.83 | 0.82 | 0.82 |
| DTREE | 0.800395257 | 0.85595081 | 0.78 | 0.82 | 0.8 |
| SVM | 0.883399209 | 0.88934047 | 0.7 | 0.82 | 0.76 |
| NB | 0.812252964 | 0.80331685 | 0.78 | 0.82 | 0.8 |
| LDA | 0.855731225 | 0.82383041 | 0.65 | 0.86 | 0.76 |
| ADB | 0.754940711 | 0.8032888 | 0.7 | 0.82 | 0.76 |
| KNN | 0.838932806 | 0.85327758 | 0.61 | 0.82 | 0.71 |
| RF | 0.89229249 | 0.90237323 | 0.65 | 0.91 | 0.78 |
| GB | 0.8715415 | 0.83778986 | 0.7 | 0.86 | 0.78 |
| LDA | 0.86166008 | 0.88427077 | 0.61 | 0.91 | 0.76 |
| SVM | 0.87747036 | 0.87996781 | 0.65 | 0.82 | 0.73 |
| ADB | 0.80434783 | 0.83217761 | 0.7 | 0.82 | 0.76 |
| KNN | 0.81521739 | 0.76405936 | 0.65 | 0.86 | 0.76 |
| NB | 0.82213439 | 0.82948771 | 0.61 | 0.82 | 0.71 |
| LR | 0.82608696 | 0.82792061 | 0.57 | 0.82 | 0.69 |
| DTREE | 0.64426877 | 0.7410628 | 0.65 | 0.64 | 0.64 |
| SVM | 0.875494071 | 0.89121479 | 0.78 | 0.86 | 0.82 |
| KNN | 0.873517787 | 0.86777558 | 0.78 | 0.82 | 0.8 |
| RF | 0.858695652 | 0.87273856 | 0.74 | 0.86 | 0.8 |
| LDA | 0.885375494 | 0.88035725 | 0.65 | 0.86 | 0.76 |
| NB | 0.802371542 | 0.78962774 | 0.74 | 0.82 | 0.78 |
| ADB | 0.837944664 | 0.81056776 | 0.7 | 0.77 | 0.73 |
| LR | 0.830039526 | 0.80815027 | 0.65 | 0.82 | 0.73 |
| GB | 0.816205534 | 0.74538266 | 0.65 | 0.86 | 0.76 |
| DTREE | 0.555335968 | 0.6763285 | 0.57 | 0.55 | 0.56 |
| RF | 0.91897233 | 0.89452984 | 0.78 | 0.86 | 0.82 |
| GB | 0.88735178 | 0.84303561 | 0.78 | 0.82 | 0.8 |
| SVM | 0.87351779 | 0.8909124 | 0.74 | 0.77 | 0.76 |
| KNN | 0.85079051 | 0.85979721 | 0.7 | 0.82 | 0.76 |
| LR | 0.84189723 | 0.79197886 | 0.74 | 0.82 | 0.78 |
| NB | 0.81422925 | 0.79965212 | 0.74 | 0.82 | 0.78 |
| LDA | 0.8458498 | 0.84037804 | 0.61 | 0.77 | 0.69 |
| ADB | 0.75296443 | 0.75656973 | 0.83 | 0.64 | 0.73 |
| DTREE | 0.68675889 | 0.78019324 | 0.78 | 0.59 | 0.69 |
| RF | 0.88241107 | 0.89925268 | 0.65 | 0.91 | 0.78 |
| DTREE | 0.80237154 | 0.87004831 | 0.7 | 0.91 | 0.8 |
| GB | 0.88339921 | 0.88927003 | 0.61 | 0.91 | 0.76 |
| SVM | 0.86363636 | 0.88542275 | 0.7 | 0.82 | 0.76 |
| LR | 0.84980237 | 0.82411397 | 0.74 | 0.82 | 0.78 |
| NB | 0.8201581 | 0.81930552 | 0.7 | 0.86 | 0.78 |
| KNN | 0.79249012 | 0.84542819 | 0.7 | 0.82 | 0.76 |
| LDA | 0.84980237 | 0.83266016 | 0.61 | 0.86 | 0.73 |
| ADB | 0.84782609 | 0.84413253 | 0.57 | 0.82 | 0.69 |
| RF | 0.880434783 | 0.89438822 | 0.65 | 0.95 | 0.8 |
| SVM | 0.867588933 | 0.87925772 | 0.74 | 0.86 | 0.8 |
| KNN | 0.874505929 | 0.89029628 | 0.7 | 0.82 | 0.76 |
| LDA | 0.85770751 | 0.8307274 | 0.74 | 0.82 | 0.78 |
| GB | 0.85770751 | 0.871808 | 0.61 | 0.91 | 0.76 |
| ADB | 0.804347826 | 0.80109072 | 0.74 | 0.82 | 0.78 |
| LR | 0.847826087 | 0.82062935 | 0.65 | 0.86 | 0.76 |
| NB | 0.794466403 | 0.79176685 | 0.65 | 0.73 | 0.69 |
| DTREE | 0.690711462 | 0.77276888 | 0.61 | 0.77 | 0.69 |

Machine Learning Round 3

| | | | | | |
|---|---|---|---|---|---|
| GB | 0.9743083 | 0.97945045 | 0.87 | 1 | 0.93 |
| LDA | 0.970355731 | 0.97889763 | 0.91 | 0.95 | 0.93 |
| SVM | 0.968379447 | 0.97129576 | 0.91 | 0.91 | 0.91 |
| ADB | 0.962450593 | 0.97092689 | 0.87 | 0.95 | 0.91 |
| LR | 0.976284585 | 0.98001924 | 0.87 | 0.91 | 0.89 |
| KNN | 0.976284585 | 0.97940457 | 0.87 | 0.91 | 0.89 |
| RF | 0.976284585 | 0.97756591 | 0.87 | 0.91 | 0.89 |
| NB | 0.927865613 | 0.93159375 | 0.87 | 0.86 | 0.87 |
| DTREE | 0.779644269 | 0.8466565 | 0.7 | 0.86 | 0.78 |
| GB | 0.98320158 | 0.98575119 | 0.87 | 1 | 0.93 |
| LDA | 0.96640316 | 0.9745459 | 0.87 | 1 | 0.93 |
| ADB | 0.98418972 | 0.98541136 | 0.87 | 0.95 | 0.91 |
| RF | 0.97727273 | 0.97827592 | 0.91 | 0.91 | 0.91 |
| SVM | 0.96640316 | 0.97202326 | 0.91 | 0.91 | 0.91 |
| LR | 0.96442688 | 0.97364736 | 0.87 | 0.91 | 0.89 |
| KNN | 0.95849802 | 0.97161088 | 0.87 | 0.91 | 0.89 |
| NB | 0.92391304 | 0.92882217 | 0.87 | 0.77 | 0.82 |
| DTREE | 0.8013834 | 0.86123188 | 0.74 | 0.86 | 0.8 |
| GB | 0.98418972 | 0.98608548 | 0.87 | 1 | 0.93 |
| LDA | 0.97035573 | 0.97954786 | 0.87 | 1 | 0.93 |
| RF | 0.98023715 | 0.98102836 | 0.91 | 0.91 | 0.91 |
| ADB | 0.97233202 | 0.96637593 | 0.87 | 0.95 | 0.91 |
| SVM | 0.96640316 | 0.9702084 | 0.91 | 0.91 | 0.91 |
| LR | 0.96640316 | 0.97503099 | 0.87 | 0.91 | 0.89 |
| KNN | 0.95849802 | 0.97131938 | 0.87 | 0.91 | 0.89 |
| NB | 0.92786561 | 0.93158487 | 0.87 | 0.82 | 0.84 |
| DTREE | 0.8013834 | 0.86123188 | 0.74 | 0.86 | 0.8 |
| LDA | 0.96837945 | 0.97752452 | 0.87 | 1 | 0.93 |
| ADB | 0.98023715 | 0.98246667 | 0.87 | 0.95 | 0.91 |
| GB | 0.97826087 | 0.98140286 | 0.87 | 0.95 | 0.91 |
| SVM | 0.97035573 | 0.97313906 | 0.91 | 0.91 | 0.91 |
| RF | 0.97628458 | 0.97723662 | 0.87 | 0.91 | 0.89 |
| LR | 0.96640316 | 0.97503099 | 0.87 | 0.91 | 0.89 |
| KNN | 0.96047431 | 0.97297345 | 0.87 | 0.91 | 0.89 |
| NB | 0.92391304 | 0.92882217 | 0.87 | 0.86 | 0.87 |
| DTREE | 0.8013834 | 0.86123188 | 0.74 | 0.86 | 0.8 |
| GB | 0.98814229 | 0.98915765 | 0.87 | 1 | 0.93 |
| ADB | 0.97233202 | 0.97328943 | 0.91 | 0.95 | 0.93 |
| LDA | 0.96640316 | 0.97426705 | 0.83 | 1 | 0.91 |
| SVM | 0.97233202 | 0.97562557 | 0.91 | 0.91 | 0.91 |
| RF | 0.97826087 | 0.97895584 | 0.87 | 0.91 | 0.89 |
| LR | 0.96640316 | 0.97503099 | 0.87 | 0.91 | 0.89 |
| KNN | 0.96047431 | 0.97297345 | 0.87 | 0.91 | 0.89 |
| NB | 0.92391304 | 0.92881329 | 0.87 | 0.86 | 0.87 |
| DTREE | 0.8013834 | 0.86123188 | 0.74 | 0.86 | 0.8 |
| GB | 0.98221344 | 0.98458883 | 0.87 | 1 | 0.93 |
| ADB | 0.9743083 | 0.98201842 | 0.91 | 0.95 | 0.93 |
| RF | 0.97826087 | 0.97901395 | 0.91 | 0.91 | 0.91 |
| SVM | 0.97233202 | 0.97562557 | 0.91 | 0.91 | 0.91 |
| LDA | 0.96245059 | 0.97187278 | 0.83 | 1 | 0.91 |
| LR | 0.96442688 | 0.97364736 | 0.87 | 0.91 | 0.89 |
| KNN | 0.96047431 | 0.97297345 | 0.87 | 0.91 | 0.89 |
| NB | 0.92588933 | 0.92983158 | 0.87 | 0.77 | 0.82 |
| DTREE | 0.8013834 | 0.86123188 | 0.74 | 0.86 | 0.8 |
| GB | 0.98221344 | 0.98526951 | 0.91 | 1 | 0.96 |
| LDA | 0.97233202 | 0.97936814 | 0.91 | 1 | 0.96 |
| RF | 0.97924901 | 0.97966809 | 0.96 | 0.91 | 0.93 |
| ADB | 0.98221344 | 0.98173461 | 0.87 | 0.95 | 0.91 |
| SVM | 0.97233202 | 0.97562557 | 0.91 | 0.91 | 0.91 |
| LR | 0.97826087 | 0.98140286 | 0.87 | 0.91 | 0.89 |
| KNN | 0.97826087 | 0.98063609 | 0.87 | 0.91 | 0.89 |
| NB | 0.92588933 | 0.93057545 | 0.87 | 0.86 | 0.87 |
| DTREE | 0.8013834 | 0.86123188 | 0.74 | 0.86 | 0.8 |
| GB | 0.98814229 | 0.98973295 | 0.91 | 1 | 0.96 |
| LDA | 0.96640316 | 0.97666758 | 0.91 | 1 | 0.96 |
| RF | 0.97233202 | 0.97273693 | 0.91 | 0.91 | 0.91 |
| SVM | 0.97035573 | 0.97378226 | 0.91 | 0.91 | 0.91 |
| ADB | 0.95454545 | 0.9311301 | 0.87 | 0.95 | 0.91 |
| LR | 0.96640316 | 0.97503099 | 0.87 | 0.91 | 0.89 |
| KNN | 0.96047431 | 0.97297345 | 0.87 | 0.91 | 0.89 |
| NB | 0.92391304 | 0.92942925 | 0.87 | 0.86 | 0.87 |
| DTREE | 0.8013834 | 0.86123188 | 0.74 | 0.86 | 0.8 |
| GB | 0.97826087 | 0.98140286 | 0.87 | 1 | 0.93 |
| ADB | 0.98023715 | 0.98299642 | 0.91 | 0.95 | 0.93 |
| LDA | 0.96640316 | 0.97602787 | 0.83 | 1 | 0.91 |
| SVM | 0.97035573 | 0.97313906 | 0.91 | 0.91 | 0.91 |
| LR | 0.96640316 | 0.97503099 | 0.87 | 0.91 | 0.89 |
| KNN | 0.96047431 | 0.97297345 | 0.87 | 0.91 | 0.89 |
| RF | 0.97628458 | 0.97757532 | 0.83 | 0.91 | 0.87 |
| NB | 0.92588933 | 0.92984046 | 0.87 | 0.82 | 0.84 |
| DTREE | 0.8013834 | 0.86123188 | 0.74 | 0.86 | 0.8 |
| GB | 0.98418972 | 0.98636696 | 0.87 | 1 | 0.93 |
| ADB | 0.97826087 | 0.97851794 | 0.91 | 0.95 | 0.93 |
| LDA | 0.96837945 | 0.97751401 | 0.83 | 1 | 0.91 |
| SVM | 0.97035573 | 0.97378226 | 0.91 | 0.91 | 0.91 |
| RF | 0.97727273 | 0.97831111 | 0.87 | 0.91 | 0.89 |
| LR | 0.96442688 | 0.97364736 | 0.87 | 0.91 | 0.89 |
| KNN | 0.96047431 | 0.97297345 | 0.87 | 0.91 | 0.89 |
| NB | 0.92588933 | 0.93057545 | 0.87 | 0.82 | 0.84 |
| DTREE | 0.8013834 | 0.86123188 | 0.74 | 0.86 | 0.8 |

TABLE 2D
COVID vs non-COVID ICU

| | | | | | |
|---|---|---|---|---|---|
| | ROC | Precision/ | | | |
| | Curve | Recall | | | |
| Model | AUC | AUC | Sensitivity | Specificity | Accuracy |

Machine Learning Round 1

| | | | | | |
|---|---|---|---|---|---|
| RF | 0.92105263 | 0.89411255 | 0.89 | 0.88 | 0.89 |
| GB | 0.93188854 | 0.94776336 | 0.84 | 0.88 | 0.86 |
| DTREE | 0.8622291 | 0.90716374 | 0.84 | 0.88 | 0.86 |
| SVM | 0.90402477 | 0.89748379 | 0.79 | 0.82 | 0.81 |
| ADB | 0.91021672 | 0.85319067 | 0.89 | 0.71 | 0.81 |
| LDA | 0.81733746 | 0.867791 | 0.74 | 0.88 | 0.81 |
| LR | 0.80804954 | 0.83985847 | 0.79 | 0.82 | 0.81 |
| KNN | 0.8498452 | 0.88726551 | 0.53 | 0.94 | 0.72 |
| NB | 0.86068111 | 0.88253356 | 0.79 | 0.65 | 0.72 |
| ADB | 0.947368421 | 0.934164881 | 1 | 0.88 | 0.94 |
| RF | 0.950464396 | 0.946670592 | 0.95 | 0.88 | 0.92 |
| SVM | 0.959752322 | 0.960381938 | 0.89 | 0.88 | 0.89 |
| GB | 0.93498452 | 0.910219908 | 0.84 | 0.88 | 0.86 |
| LR | 0.876160991 | 0.923705573 | 0.74 | 0.94 | 0.83 |
| LDA | 0.891640867 | 0.932018363 | 0.68 | 0.94 | 0.81 |
| NB | 0.904024768 | 0.90680627 | 0.95 | 0.65 | 0.81 |
| KNN | 0.922600619 | 0.925105231 | 0.63 | 0.94 | 0.78 |
| DTREE | 0.783281734 | 0.85877193 | 0.68 | 0.88 | 0.78 |
| SVM | 0.9752322 | 0.97622458 | 0.89 | 0.94 | 0.92 |
| RF | 0.96130031 | 0.95844156 | 0.89 | 0.88 | 0.89 |
| ADB | 0.95356037 | 0.96251343 | 0.95 | 0.76 | 0.86 |
| LR | 0.88544892 | 0.93119633 | 0.79 | 0.94 | 0.86 |
| LDA | 0.85758514 | 0.9050636 | 0.74 | 0.94 | 0.83 |
| KNN | 0.88544892 | 0.91913374 | 0.68 | 0.94 | 0.81 |
| DTREE | 0.8003096 | 0.86150984 | 0.89 | 0.71 | 0.81 |
| GB | 0.86068111 | 0.8501041 | 0.84 | 0.71 | 0.78 |
| NB | 0.91640867 | 0.93580518 | 0.95 | 0.47 | 0.72 |
| RF | 0.9752322 | 0.97952754 | 0.95 | 0.76 | 0.86 |
| LDA | 0.90712074 | 0.94604424 | 0.74 | 1 | 0.86 |
| GB | 0.92879257 | 0.93196139 | 0.89 | 0.82 | 0.86 |
| ADB | 0.91950464 | 0.92181438 | 1 | 0.71 | 0.86 |
| SVM | 0.90092879 | 0.90860983 | 0.89 | 0.76 | 0.83 |
| KNN | 0.8993808 | 0.92681459 | 0.63 | 1 | 0.81 |
| LR | 0.83900929 | 0.89222961 | 0.79 | 0.65 | 0.72 |
| DTREE | 0.74458204 | 0.82635566 | 0.84 | 0.65 | 0.75 |
| NB | 0.79256966 | 0.81442307 | 0.95 | 0.47 | 0.72 |
| SVM | 0.959752322 | 0.96820825 | 0.89 | 0.88 | 0.89 |
| GB | 0.931888545 | 0.93510228 | 0.89 | 0.88 | 0.89 |
| LR | 0.904024768 | 0.9399445 | 0.84 | 0.88 | 0.86 |
| RF | 0.948916409 | 0.9537073 | 0.95 | 0.71 | 0.83 |
| DTREE | 0.862229102 | 0.90716374 | 0.84 | 0.88 | 0.86 |
| LDA | 0.876160991 | 0.92741566 | 0.74 | 0.94 | 0.83 |
| ADB | 0.904024768 | 0.92511774 | 0.95 | 0.65 | 0.81 |
| KNN | 0.894736842 | 0.93457602 | 0.58 | 1 | 0.78 |
| NB | 0.86996904 | 0.88958948 | 0.95 | 0.53 | 0.75 |
| RF | 0.99380805 | 0.99486216 | 0.79 | 1 | 0.89 |
| GB | 0.9628483 | 0.95549315 | 0.89 | 0.94 | 0.92 |
| SVM | 0.9876161 | 0.98946049 | 0.79 | 1 | 0.89 |
| DTREE | 0.92105263 | 0.9627193 | 0.84 | 1 | 0.92 |
| LR | 0.86377709 | 0.92196469 | 0.79 | 1 | 0.89 |
| LDA | 0.90092879 | 0.9504939 | 0.74 | 1 | 0.86 |
| ADB | 0.92260062 | 0.90980703 | 0.84 | 0.88 | 0.86 |
| NB | 0.89473684 | 0.91661918 | 0.79 | 0.71 | 0.75 |
| KNN | 0.86842105 | 0.90320498 | 0.47 | 1 | 0.72 |
| SVM | 0.9628483 | 0.95549315 | 0.79 | 0.94 | 0.86 |
| RF | 0.95665635 | 0.95579661 | 0.84 | 0.88 | 0.86 |
| LDA | 0.90402477 | 0.94768148 | 0.74 | 1 | 0.86 |
| GB | 0.9504644 | 0.96045145 | 0.79 | 0.82 | 0.81 |
| LR | 0.86996904 | 0.90602501 | 0.74 | 0.88 | 0.81 |
| ADB | 0.88854489 | 0.89142856 | 0.74 | 0.82 | 0.78 |
| DTREE | 0.78637771 | 0.87455016 | 0.63 | 0.94 | 0.78 |
| KNN | 0.89318885 | 0.91359649 | 0.47 | 0.94 | 0.69 |
| NB | 0.85758514 | 0.87679336 | 0.89 | 0.53 | 0.72 |
| SVM | 0.91331269 | 0.92067913 | 0.89 | 0.82 | 0.86 |
| LDA | 0.87925697 | 0.92362156 | 0.74 | 0.94 | 0.83 |
| RF | 0.8993808 | 0.89957355 | 0.95 | 0.71 | 0.83 |
| ADB | 0.86996904 | 0.9071888 | 0.89 | 0.71 | 0.81 |
| LR | 0.83900929 | 0.89747071 | 0.84 | 0.76 | 0.81 |
| KNN | 0.86687307 | 0.9061516 | 0.63 | 0.94 | 0.78 |
| NB | 0.86068111 | 0.90075389 | 0.89 | 0.59 | 0.75 |
| DTREE | 0.77399381 | 0.84367168 | 0.84 | 0.71 | 0.78 |
| GB | 0.78328173 | 0.74396594 | 0.84 | 0.65 | 0.75 |
| SVM | 0.96594427 | 0.97540358 | 0.89 | 1 | 0.94 |
| RF | 0.97832817 | 0.98425823 | 0.89 | 0.94 | 0.92 |
| GB | 0.95665635 | 0.9693939 | 0.89 | 0.94 | 0.92 |
| ADB | 0.98142415 | 0.98594969 | 0.79 | 1 | 0.89 |
| DTREE | 0.88854489 | 0.92251462 | 0.89 | 0.88 | 0.89 |
| LDA | 0.89473684 | 0.9466381 | 0.74 | 1 | 0.86 |
| LR | 0.87925697 | 0.93133146 | 0.79 | 0.88 | 0.83 |
| KNN | 0.86996904 | 0.91354919 | 0.63 | 1 | 0.81 |
| NB | 0.84829721 | 0.8862209 | 0.84 | 0.47 | 0.67 |
| DTREE | 0.91176471 | 0.93181818 | 1 | 0.82 | 0.92 |
| RF | 0.9380805 | 0.93303411 | 0.89 | 0.82 | 0.86 |
| SVM | 0.9504644 | 0.95545242 | 0.79 | 0.88 | 0.83 |
| GB | 0.90712074 | 0.87598591 | 0.89 | 0.82 | 0.86 |
| ADB | 0.91640867 | 0.91992399 | 0.95 | 0.71 | 0.83 |
| LDA | 0.86996904 | 0.92470932 | 0.74 | 0.94 | 0.83 |
| KNN | 0.82352941 | 0.88177285 | 0.58 | 0.94 | 0.75 |
| LR | 0.83591331 | 0.89023732 | 0.74 | 0.71 | 0.72 |
| NB | 0.79876161 | 0.83815267 | 0.89 | 0.41 | 0.67 |

Machine Learning Round 2

| | | | | | |
|---|---|---|---|---|---|
| RF | 0.95510836 | 0.96613602 | 0.95 | 0.71 | 0.83 |
| SVM | 0.94736842 | 0.95854819 | 0.89 | 0.76 | 0.83 |
| LDA | 0.87306502 | 0.90871242 | 0.79 | 0.94 | 0.86 |
| GB | 0.9380805 | 0.94806137 | 0.95 | 0.65 | 0.81 |
| ADB | 0.90092879 | 0.91835658 | 0.95 | 0.65 | 0.81 |
| LR | 0.86377709 | 0.92303602 | 0.84 | 0.71 | 0.78 |
| KNN | 0.83900929 | 0.90068922 | 0.58 | 1 | 0.78 |
| DTREE | 0.71826625 | 0.80743525 | 0.79 | 0.65 | 0.72 |
| NB | 0.82972136 | 0.87688498 | 0.95 | 0.29 | 0.64 |
| RF | 0.93498452 | 0.94012754 | 0.84 | 0.82 | 0.83 |
| ADB | 0.86377709 | 0.8627008 | 0.84 | 0.88 | 0.86 |
| SVM | 0.91640867 | 0.93637378 | 0.79 | 0.82 | 0.81 |
| NB | 0.91021672 | 0.93973848 | 0.89 | 0.71 | 0.81 |
| GB | 0.88544892 | 0.8847855 | 0.89 | 0.76 | 0.83 |
| DTREE | 0.82972136 | 0.8799081 | 0.89 | 0.76 | 0.83 |
| KNN | 0.86996904 | 0.90927978 | 0.58 | 0.94 | 0.75 |
| LR | 0.82662539 | 0.89260325 | 0.79 | 0.71 | 0.75 |
| LDA | 0.76470588 | 0.85606102 | 0.63 | 0.88 | 0.75 |
| RF | 0.92414861 | 0.92873775 | 0.89 | 0.82 | 0.86 |
| ADB | 0.92260062 | 0.89632845 | 0.89 | 0.82 | 0.86 |
| GB | 0.87616099 | 0.8796076 | 0.89 | 0.82 | 0.86 |
| LDA | 0.87925697 | 0.92771665 | 0.74 | 0.94 | 0.83 |
| NB | 0.89473684 | 0.91295386 | 0.95 | 0.71 | 0.83 |
| DTREE | 0.83281734 | 0.88377193 | 0.84 | 0.82 | 0.83 |
| SVM | 0.93188854 | 0.94022453 | 0.84 | 0.71 | 0.78 |
| LR | 0.87925697 | 0.91445165 | 0.79 | 0.76 | 0.78 |
| KNN | 0.87306502 | 0.89778257 | 0.63 | 0.82 | 0.72 |
| SVM | 0.94117647 | 0.94884096 | 0.84 | 0.82 | 0.83 |
| LDA | 0.87306502 | 0.9283434 | 0.74 | 0.94 | 0.83 |
| ADB | 0.86687307 | 0.8812401 | 0.89 | 0.76 | 0.83 |
| RF | 0.91021672 | 0.92554849 | 0.84 | 0.71 | 0.78 |
| GB | 0.90402477 | 0.91992599 | 0.89 | 0.65 | 0.78 |
| KNN | 0.8869969 | 0.91203506 | 0.63 | 0.88 | 0.75 |
| DTREE | 0.77089783 | 0.84471142 | 0.89 | 0.65 | 0.78 |
| LR | 0.83281734 | 0.89071016 | 0.74 | 0.71 | 0.72 |
| NB | 0.82662539 | 0.84903768 | 0.79 | 0.59 | 0.69 |
| RF | 0.96439628 | 0.9627786 | 0.95 | 0.94 | 0.94 |
| SVM | 0.96904025 | 0.97170941 | 0.84 | 0.94 | 0.89 |
| GB | 0.94736842 | 0.94963614 | 0.89 | 0.88 | 0.89 |
| ADB | 0.92879257 | 0.91466265 | 0.89 | 0.82 | 0.86 |
| NB | 0.91950464 | 0.94159928 | 0.89 | 0.71 | 0.81 |
| KNN | 0.8993808 | 0.92934718 | 0.63 | 0.94 | 0.78 |
| LDA | 0.82043344 | 0.900412 | 0.68 | 0.94 | 0.81 |
| LR | 0.87616099 | 0.92627779 | 0.84 | 0.65 | 0.75 |
| DTREE | 0.69504644 | 0.78654971 | 0.68 | 0.71 | 0.69 |
| SVM | 0.94427245 | 0.95972366 | 0.84 | 0.94 | 0.89 |
| DTREE | 0.88854489 | 0.92251462 | 0.89 | 0.88 | 0.89 |
| ADB | 0.87616099 | 0.89625868 | 0.84 | 0.88 | 0.86 |
| RF | 0.93343653 | 0.9292991 | 0.89 | 0.76 | 0.83 |
| GB | 0.88235294 | 0.86913012 | 0.89 | 0.82 | 0.86 |
| KNN | 0.87616099 | 0.91979016 | 0.53 | 1 | 0.75 |
| LDA | 0.80804954 | 0.88268148 | 0.53 | 0.94 | 0.72 |
| LR | 0.81424149 | 0.87231799 | 0.63 | 0.82 | 0.72 |
| NB | 0.86068111 | 0.9075927 | 0.79 | 0.59 | 0.69 |
| ADB | 0.96904025 | 0.96348572 | 1 | 0.88 | 0.94 |
| SVM | 0.95665635 | 0.95973278 | 0.84 | 0.94 | 0.89 |
| RF | 0.94427245 | 0.9188571 | 0.89 | 0.88 | 0.89 |
| GB | 0.95975232 | 0.95580999 | 0.79 | 0.94 | 0.86 |
| NB | 0.90092879 | 0.91843944 | 0.89 | 0.71 | 0.81 |
| LDA | 0.85448916 | 0.9097028 | 0.68 | 0.94 | 0.81 |
| DTREE | 0.80650155 | 0.86695906 | 0.79 | 0.82 | 0.81 |
| LR | 0.84520124 | 0.88720604 | 0.79 | 0.76 | 0.78 |
| KNN | 0.85448916 | 0.88678242 | 0.53 | 0.94 | 0.72 |
| RF | 0.97678019 | 0.98256172 | 0.95 | 0.88 | 0.92 |
| SVM | 0.95975232 | 0.96810328 | 0.79 | 0.94 | 0.86 |
| LR | 0.86377709 | 0.92253044 | 0.79 | 0.94 | 0.86 |
| GB | 0.96904025 | 0.9787039 | 0.95 | 0.65 | 0.81 |
| ADB | 0.89473684 | 0.90331184 | 0.95 | 0.65 | 0.81 |
| DTREE | 0.82662539 | 0.87887745 | 0.95 | 0.71 | 0.83 |
| NB | 0.88854489 | 0.91312519 | 0.84 | 0.71 | 0.78 |
| LDA | 0.78947368 | 0.87480434 | 0.63 | 1 | 0.81 |
| KNN | 0.87151703 | 0.92141813 | 0.47 | 1 | 0.72 |
| SVM | 1 | 1 | 0.84 | 1 | 0.92 |
| GB | 0.99380805 | 0.99445214 | 0.79 | 1 | 0.89 |
| RF | 0.98452012 | 0.98645052 | 0.84 | 0.94 | 0.89 |
| ADB | 0.87306502 | 0.87114927 | 0.89 | 0.88 | 0.89 |
| LDA | 0.85448916 | 0.91671432 | 0.68 | 1 | 0.83 |
| DTREE | 0.83591331 | 0.89146887 | 0.79 | 0.88 | 0.83 |
| LR | 0.85448916 | 0.91106992 | 0.84 | 0.76 | 0.81 |
| KNN | 0.97213622 | 0.98308271 | 0.26 | 1 | 0.61 |
| NB | 0.83281734 | 0.85434833 | 0.79 | 0.59 | 0.69 |
| GB | 0.96904025 | 0.97598952 | 0.89 | 0.94 | 0.92 |
| RF | 0.96594427 | 0.97443545 | 0.95 | 0.88 | 0.92 |
| SVM | 0.9628483 | 0.96998959 | 0.95 | 0.88 | 0.92 |
| ADB | 0.90092879 | 0.89738741 | 0.95 | 0.88 | 0.92 |
| LDA | 0.86996904 | 0.90612138 | 0.79 | 0.82 | 0.81 |
| KNN | 0.90712074 | 0.93274854 | 0.68 | 0.88 | 0.78 |
| DTREE | 0.8126935 | 0.88972431 | 0.68 | 0.94 | 0.81 |
| NB | 0.85448916 | 0.8844958 | 0.95 | 0.59 | 0.78 |
| LR | 0.87306502 | 0.91793593 | 0.79 | 0.65 | 0.72 |

Machine Learning Round 3

| | | | | | |
|---|---|---|---|---|---|
| RF | 1 | 1 | 0.95 | 1 | 0.97 |
| LR | 1 | 1 | 0.95 | 1 | 0.97 |
| KNN | 0.99845201 | 0.998684211 | 0.95 | 1 | 0.97 |
| GB | 0.99690402 | 0.997299169 | 0.95 | 1 | 0.97 |
| NB | 0.98142415 | 0.984945318 | 0.89 | 1 | 0.94 |
| LDA | 0.95356037 | 0.97642837 | 0.89 | 1 | 0.94 |
| DTREE | 0.94736842 | 0.975146199 | 0.89 | 1 | 0.94 |
| SVM | 1 | 1 | 0.79 | 1 | 0.89 |
| ADB | 0.97213622 | 0.973540501 | 0.79 | 0.94 | 0.86 |
| ADB | 1 | 1 | 1 | 1 | 1 |
| LR | 1 | 1 | 0.95 | 1 | 0.97 |
| RF | 0.99690402 | 0.99729917 | 0.95 | 1 | 0.97 |
| KNN | 0.99535604 | 0.99641148 | 0.95 | 1 | 0.97 |
| NB | 0.98452012 | 0.98779162 | 0.89 | 1 | 0.94 |
| LDA | 0.95356037 | 0.97642837 | 0.89 | 1 | 0.94 |
| SVM | 1 | 1 | 0.79 | 1 | 0.89 |
| GB | 0.92879257 | 0.95787392 | 0.79 | 0.88 | 0.83 |
| DTREE | 0.83591331 | 0.89146887 | 0.79 | 0.88 | 0.83 |
| LR | 1 | 1 | 0.95 | 1 | 0.97 |
| RF | 0.99690402 | 0.99729917 | 0.95 | 1 | 0.97 |
| ADB | 0.99690402 | 0.99729917 | 0.95 | 1 | 0.97 |
| KNN | 0.99845201 | 0.99868421 | 0.84 | 1 | 0.92 |
| NB | 0.98452012 | 0.98725042 | 0.89 | 0.94 | 0.92 |
| SVM | 0.99690402 | 0.99729917 | 0.79 | 1 | 0.89 |
| GB | 0.93498452 | 0.96339258 | 0.89 | 0.94 | 0.92 |
| DTREE | 0.86532508 | 0.9190424 | 0.79 | 0.94 | 0.86 |
| LDA | 0.8374613 | 0.81025665 | 0.89 | 0.82 | 0.86 |
| LR | 1 | 1 | 0.95 | 1 | 0.97 |
| ADB | 1 | 1 | 0.95 | 1 | 0.97 |
| RF | 0.99690402 | 0.99729917 | 0.95 | 1 | 0.97 |
| GB | 0.99380805 | 0.99486216 | 0.95 | 1 | 0.97 |
| NB | 0.9876161 | 0.98980505 | 0.89 | 1 | 0.94 |
| KNN | 0.99690402 | 0.99749373 | 0.84 | 1 | 0.92 |
| SVM | 0.99690402 | 0.99729917 | 0.79 | 1 | 0.89 |
| LDA | 0.92569659 | 0.95036038 | 0.95 | 0.88 | 0.92 |
| DTREE | 0.91795666 | 0.94736842 | 0.89 | 0.94 | 0.92 |
| LR | 1 | 1 | 0.95 | 1 | 0.97 |
| RF | 1 | 1 | 0.95 | 1 | 0.97 |
| KNN | 0.99845201 | 0.99868421 | 0.95 | 1 | 0.97 |
| GB | 0.99380805 | 0.99486216 | 0.95 | 1 | 0.97 |
| NB | 0.99071207 | 0.99201513 | 0.89 | 1 | 0.94 |
| LDA | 0.95665635 | 0.97732257 | 0.89 | 1 | 0.94 |
| DTREE | 0.94427245 | 0.96125731 | 0.95 | 0.94 | 0.94 |
| ADB | 0.99071207 | 0.99201513 | 0.89 | 0.94 | 0.92 |
| SVM | 0.99690402 | 0.99729917 | 0.79 | 1 | 0.89 |
| LR | 1 | 1 | 0.95 | 1 | 0.97 |
| RF | 1 | 1 | 0.95 | 1 | 0.97 |
| ADB | 1 | 1 | 0.89 | 1 | 0.94 |
| GB | 1 | 1 | 0.89 | 1 | 0.94 |
| NB | 0.98452012 | 0.98725042 | 0.89 | 1 | 0.94 |
| LDA | 0.95665635 | 0.97732257 | 0.89 | 1 | 0.94 |
| KNN | 0.99845201 | 0.99868421 | 0.84 | 1 | 0.92 |
| SVM | 0.99690402 | 0.99729917 | 0.79 | 1 | 0.89 |
| DTREE | 0.86532508 | 0.9190424 | 0.79 | 0.94 | 0.86 |
| LR | 1 | 1 | 0.95 | 1 | 0.97 |
| ADB | 1 | 1 | 0.95 | 1 | 0.97 |
| KNN | 0.99690402 | 0.99749373 | 0.95 | 1 | 0.97 |
| RF | 0.99690402 | 0.99729917 | 0.95 | 1 | 0.97 |
| GB | 0.99380805 | 0.99486216 | 0.95 | 0.94 | 0.94 |
| NB | 0.97832817 | 0.98293189 | 0.89 | 1 | 0.94 |
| DTREE | 0.94427245 | 0.96125731 | 0.95 | 0.94 | 0.94 |
| SVM | 1 | 1 | 0.79 | 1 | 0.89 |
| LDA | 0.85758514 | 0.87492127 | 0.74 | 0.76 | 0.75 |
| ADB | 1 | 1 | 0.95 | 1 | 0.97 |
| RF | 0.99071207 | 0.99265208 | 0.95 | 1 | 0.97 |
| DTREE | 0.97368421 | 0.9875731 | 0.95 | 1 | 0.97 |
| GB | 0.97368421 | 0.9875731 | 0.95 | 1 | 0.97 |
| LR | 0.99690402 | 0.99729917 | 0.95 | 0.94 | 0.94 |
| KNN | 0.99690402 | 0.99749373 | 0.84 | 1 | 0.92 |
| SVM | 0.99071207 | 0.99265208 | 0.79 | 1 | 0.89 |
| NB | 0.97832817 | 0.98222701 | 0.89 | 0.88 | 0.89 |
| LDA | 0.89783282 | 0.89333152 | 0.89 | 0.82 | 0.86 |
| LR | 1 | 1 | 0.95 | 1 | 0.97 |
| KNN | 0.99690402 | 0.99749373 | 0.95 | 1 | 0.97 |
| RF | 0.99690402 | 0.99729917 | 0.95 | 1 | 0.97 |
| ADB | 1 | 1 | 0.89 | 1 | 0.94 |
| NB | 0.98142415 | 0.98494532 | 0.89 | 1 | 0.94 |
| GB | 0.96749226 | 0.98209449 | 0.95 | 0.94 | 0.94 |
| DTREE | 0.94427245 | 0.96125731 | 0.95 | 0.94 | 0.94 |
| SVM | 1 | 1 | 0.79 | 1 | 0.89 |
| LDA | 0.88854489 | 0.89660661 | 0.84 | 0.88 | 0.86 |
| ADB | 1 | 1 | 1 | 1 | 1 |
| LR | 1 | 1 | 0.95 | 1 | 0.97 |
| RF | 0.99535604 | 0.99610869 | 0.95 | 1 | 0.97 |
| NB | 0.9876161 | 0.98946049 | 0.89 | 1 | 0.94 |
| KNN | 0.99845201 | 0.99868421 | 0.84 | 1 | 0.92 |
| LDA | 0.9504644 | 0.97558602 | 0.89 | 1 | 0.94 |
| SVM | 0.99690402 | 0.99729917 | 0.79 | 1 | 0.89 |
| GB | 0.9380805 | 0.96600877 | 0.79 | 0.94 | 0.86 |
| DTREE | 0.86532508 | 0.9190424 | 0.79 | 0.94 | 0.86 |

Tables 3A-3D: Top features from the ML classifications. For each ML round, the top features for 10 repetitions are shown. To determine the top features, for each ML round and each repetition, feature importance values of the features of the top 5 performing machine learning algorithms are summed to obtain overall feature importance values, and based on the overall feature importance values the top features are determined. For example, top 20 features have top (e.g., highest) 20 overall feature importance values. For each

### Machine Learning Algorithm Performance.

TABLE 3A
Top features from ML classification of all COVID vs healthy.

Machine Learning Round 1: Top 20 features from each repeatations.
1
Alternative Complement Pathway, Amino Acid Metabolism, Anergic / Activated T cells, Anti
inflammation, Apoptosis, CD40 Activated B Cell, Cell Cycle, Classical Complement
Pathway, Dendritic Cell, Glycolysis, Granulocyte, IFN, Inflammatory_Neutrophil, MHC II,
Oxidative Phosphorylation, Pentose Phosphate Signature, Proinflammatory IL-1 Family,
Tactivated, TCA cycle, Treg
2
Alternative Complement Pathway, Amino Acid Metabolism, Anergic / Activated T cells, Anti
inflammation, Apoptosis, CD40 Activated B Cell, Cell Cycle, Classical Complement
Pathway, Dendritic Cell, Erythrocytes, Glycolysis, Granulocyte, IFN,
Inflammatory_Neutrophil, MHC II, Oxidative Phosphorylation, pDC, Pentose Phosphate
Signature, Plasma Cell, T Cell
3
Alternative Complement Pathway, Amino Acid Metabolism, Anergic / Activated T cells, Anti
inflammation, Apoptosis, CD40 Activated B Cell, Cell Cycle, Cytotoxic, Activated T Cell,
Dendritic Cell, Fatty Acid Alpha Oxidation, Glycolysis, Inflammatory_Neutrophil, MHC II,
Oxidative Phosphorylation, Pentose Phosphate Signature, Plasma Cell, Platelets, T Cell,
Tactivated, TNF
4
Alternative Complement Pathway, Anergic / Activated T cells, Anti inflammation, CD40
Activated B Cell, Cell Cycle, Erythrocytes, Fatty Acid Alpha Oxidation, Glycolysis,
Granulocyte, IFN, MHC II, NFkB Complex, NK Cell, pDC, Pentose Phosphate Signature,
Plasma Cell, Proinflammatory IL-1 Family, ROS Protection, TCA cycle, Unfolded Protein
5
Alternative Complement Pathway, Amino Acid Metabolism, Anergic / Activated T cells, Anti
inflammation, Apoptosis, CD40 Activated B Cell, Cell Cycle, Classical Complement
Pathway, Dendritic Cell, Glycolysis, Granulocyte, IFN, Inflammatory_Neutrophil, MHC II,
Oxidative Phosphorylation, pDC, Pentose Phosphate Signature, Proinflammatory IL-1
Family, TCA cycle, Unfolded Protein
6
Alternative Complement Pathway, Anti inflammation, Apoptosis, CD40 Activated B Cell,
Cell Cycle, Classical Complement Pathway, Cytotoxic, Activated T Cell, Dendritic Cell, Fatty
Acid Alpha Oxidation, Granulocyte, MHC II, NFkB Complex, NLRP3 Inflammasome,
Oxidative Phosphorylation, Pentose Phosphate Signature, Plasma Cell, Platelets, T Cell,
Tactivated, TCA cycle
7
Amino Acid Metabolism, Anergic / Activated T cells, Anti inflammation, CD40 Activated B
Cell, Cell Cycle, Erythrocytes, Fatty Acid Alpha Oxidation, Fatty Acid Beta Oxidation,
Glycolysis, Granulocyte, Inflammatory_Neutrophil, LDG, MHC II, Oxidative
Phosphorylation, Pentose Phosphate Signature, Plasma Cell, Platelets, T Cell, TCA cycle,
TNF
8
Alternative Complement Pathway, Amino Acid Metabolism, Anergic / Activated T cells, Anti
inflammation, Apoptosis, CD40 Activated B Cell, Cell Cycle, Classical Complement
Pathway, Cytotoxic, Activated T Cell, Fatty Acid Alpha Oxidation, Glycolysis, Granulocyte,
Oxidative Phosphorylation, Pentose Phosphate Signature, Proinflammatory IL-1 Family, T
Cell, Tactivated, TCA cycle, TNF, Treg
9
Alternative Complement Pathway, Amino Acid Metabolism, Anergic / Activated T cells, Anti
inflammation, CD40 Activated B Cell, Cell Cycle, Cytotoxic, Activated T Cell, Dendritic
Cell, Fatty Acid Alpha Oxidation, Fatty Acid Beta Oxidation, Glycolysis, Granulocyte, IFN,
Inflammatory_Neutrophil, MHC II, Pentose Phosphate Signature, Plasma Cell,
Proinflammatory IL-1 Family, T Cell, TCA cycle
10
Alternative Complement Pathway, Amino Acid Metabolism, Anergic / Activated T cells, Anti
inflammation, CD40 Activated B Cell, Cell Cycle, Classical Complement Pathway,
Cytotoxic, Activated T Cell, Erythrocytes, Fatty Acid Alpha Oxidation, Glycolysis,
Granulocyte, IFN, Inflammatory_Neutrophil, Oxidative Phosphorylation, Pentose Phosphate
Signature, Proinflammatory IL-1 Family, ROS Protection, Tactivated, TCA cycle
Machine Learning Round 2: Top 10 features from each repeatations.
1
Amino Acid Metabolism, Apoptosis, Cell Cycle, Classical Complement Pathway, Glycolysis,
Granulocyte, Oxidative Phosphorylation, Pentose Phosphate Signature, Proinflammatory IL-1
Family, TCA cycle
2
Alternative Complement Pathway, Anti inflammation, Cell Cycle, Erythrocytes, Glycolysis,
Inflammatory_Neutrophil, Oxidative Phosphorylation, pDC, Pentose Phosphate Signature, T
Cell
3
Alternative Complement Pathway, Amino Acid Metabolism, Anti inflammation, Apoptosis,
Cell Cycle, Glycolysis, MHC II, Oxidative Phosphorylation, Pentose Phosphate Signature,
Platelets
4
Alternative Complement Pathway, Anti inflammation, CD40 Activated B Cell, Cell Cycle,
Fatty Acid Alpha Oxidation, Glycolysis, Granulocyte, Pentose Phosphate Signature,
Proinflammatory IL-1 Family, TCA cycle
5
Alternative Complement Pathway, Amino Acid Metabolism, Anergic / Activated T cells, Anti
inflammation, CD40 Activated B Cell, Cell Cycle, Glycolysis, Granulocyte, Pentose
Phosphate Signature, Proinflammatory IL-1 Family
6
Alternative Complement Pathway, Anti inflammation, Apoptosis, Cell Cycle, Fatty Acid
Alpha Oxidation, MHC II, NLRP3 Inflammasome, Oxidative Phosphorylation, Pentose
Phosphate Signature, Tactivated
7
Anti inflammation, Cell Cycle, Fatty Acid Alpha Oxidation, Glycolysis,
Inflammatory_Neutrophil, MHC II, Oxidative Phosphorylation, Pentose Phosphate Signature,
Plasma Cell, Platelets
8
Alternative Complement Pathway, Anti inflammation, Apoptosis, Cell Cycle, Classical
Complement Pathway, Glycolysis, Oxidative Phosphorylation, Pentose Phosphate Signature,
Proinflammatory IL-1 Family, TCA cycle
9
Alternative Complement Pathway, Anti inflammation, Cell Cycle, Glycolysis, Granulocyte,
MHC II, Pentose Phosphate Signature, Plasma Cell, T Cell, TCA cycle
10
Alternative Complement Pathway, Anergic / Activated T cells, Anti inflammation, Cell Cycle,
Erythrocytes, Fatty Acid Alpha Oxidation, Glycolysis, Granulocyte, IFN, Pentose Phosphate
Signature
Machine Learning Round 3: Top 20 features (genes) from each repeatations.
1
ASPM, C3, CCNB2, CD177, CEP55, CLC, COX6C, COX7B, COX7C, CXCR2, DNAJC15,
HAAO, HLA-DPB1, IL1R1, NDUFAF7, NDUFB3, OSM, SLC2A3, TMEM126B, UQCRB
2
CCNB1, CCNB2, CDC20, CEP55, CLC, COA1, COX11, COX6C, COX7B, CTSS, HLA-
DMA, MKI67, NDUFA2, NDUFAF7, NDUFB3, OSM, PTTG1, SLC2A3, TMEM126B,
UQCRB
3
ASPM, CCNB2, CD177, CDC20, CLC, COX6C, COX7B, COX7C, GINS2, MCM10,
MCM2, MKI67, NDUFAF5, NDUFAF7, NDUFB3, OSM, SLC2A3, TMEM126B, TRAP1,
UQCRB
4
CCNB2, CD177, CDC20, CEP55, CLC, COX6C, COX7B, COX7C, IL1B, MCM10, MKI67,
NDUFA2, NDUFAF5, NDUFAF7, NDUFB3, NDUFS1, OSM, PTTG1, SLC2A3, UQCRB
5
C3, CCNB1, CCNB2, CD177, CDC20, CEP55, COX6C, COX7B, COX7C, GINS2, HLA-
DMA, HLA-DPB1, HLA-DRB1, IL1B, MKI67, NDUFAF7, NDUFB3, SLC2A3,
TMEM126B, UQCRB
6
CCNB2, CD177, CDC20, CEP55, CLC, COX6C, COX7B, COX7C, IL18RAP, IL1R1,
MCM10, MKI67, NDUFA2, NDUFAF7, NDUFB3, OSM, PFKFB2, SLC2A3, TMEM126B,
UQCRB
7
CCNB2, CD177, CDC20, CEP55, CLC, COX6C, COX7B, COX7C, E2F3, GINS2, HAAO,
HLA-DMA, HLA-DRB1, MKI67, NDUFAF5, NDUFAF7, NDUFB3, SLC2A1, SLC2A3,
UQCRB
8
C3, CCNB2, CD177, CEP55, CLC, COX20, COX6C, COX7B, COX7C, CR1, CXCR2,
HLA-DPB1, HLA-DQA1, HLA-DRB1, MCM10, MKI67, NDUFB3, SLC2A3, TMEM126B,
UQCRB
9
CCNB2, CDC20, CEP55, CLC, COX6C, COX7B, COX7C, E2F3, GINS2, HAAO, MKI67,
NDUFAF5, NDUFAF7, NDUFB3, OSM, PTTG1, SLC2A3, TMEM126B, TTC19, UQCRB
10
AURKA, CCNB1, CCNB2, CD177, CDC20, CEP55, CLC, COX6C, COX7B, COX7C, CR1,
GINS2, MKI67, NDUFAF7, NDUFB3, PFKFB2, PTTG1, SLC2A1, SLC2A3, UQCRB

TABLE 3B
Top features from ML classification of non-critical COVID vs healthy.

Machine Learning Round 1: Top 20 features from each repeatations.
1
Alternative Complement Pathway, Amino Acid Metabolism, Anergic / Activated T cells, Anti
inflammation, Apoptosis, CD40 Activated B Cell, Cell Cycle, Classical Complement Pathway
Cytotoxic, Activated T Cell, Fatty Acid Beta Oxidation, IFN, Monocyte, Oxidative
Phosphorylation, Pentose Phosphate Signature, Plasma Cell, Platelets, T Cell, Tactivated,
TCA cycle, TNF
2
Alternative Complement Pathway, Amino Acid Metabolism, Anergic / Activated T cells, Anti
inflammation, Apoptosis, CD40 Activated B Cell, Cell Cycle, Classical Complement
Pathway, Dendritic Cell, Fatty Acid Beta Oxidation, Granulocyte, IFN,
Inflammatory_Neutrophil, MHC II, Oxidative Phosphorylation, Pentose Phosphate Signature,
Plasma Cell, Tactivated, TCA cycle, TNF
3
Alternative Complement Pathway, Amino Acid Metabolism, Anti inflammation, Apoptosis,
CD40 Activated B Cell, Cell Cycle, Cytotoxic, Activated T Cell, Erythrocytes, Fatty Acid
Beta Oxidation, LDG, MHC II, Monocyte, Oxidative Phosphorylation, Pentose Phosphate
Signature, Plasma Cell, Platelets, Proinflammatory IL-1 Family, Tactivated, TNF, Unfolded
Protein
4
Alternative Complement Pathway, Amino Acid Metabolism, Anti inflammation, Apoptosis,
CD40 Activated B Cell, Cell Cycle, Classical Complement Pathway, Cytotoxic, Activated T
Cell, Dendritic Cell, Erythrocytes, Fatty Acid Beta Oxidation, IFN, Inflammatory_Neutrophil,
LDG, Oxidative Phosphorylation, Pentose Phosphate Signature, Plasma Cell, Tactivated,
TNF, Unfolded Protein
5
Alternative Complement Pathway, Amino Acid Metabolism, Apoptosis, CD40 Activated B
Cell, Cell Cycle, Classical Complement Pathway, Cytotoxic, Activated T Cell, Erythrocytes,
Fatty Acid Beta Oxidation, Glycolysis, Granulocyte, Inflammatory_Neutrophil, LDG, MHC
II, Oxidative Phosphorylation, Pentose Phosphate Signature, Plasma Cell, Platelets,
Proinflammatory IL-1 Family, ROS Protection
6
Alternative Complement Pathway, Amino Acid Metabolism, Anergic / Activated T cells, Anti
inflammation, Apoptosis, CD40 Activated B Cell, Cell Cycle, Classical Complement
Pathway, Fatty Acid Beta Oxidation, Granulocyte, IFN, LDG, MHC II, Oxidative
Phosphorylation, pDC, Pentose Phosphate Signature, Plasma Cell, Platelets, Tactivated,
Unfolded Protein
7
Alternative Complement Pathway, Amino Acid Metabolism, Anti inflammation, Apoptosis,
CD40 Activated B Cell, Cell Cycle, Classical Complement Pathway, Cytotoxic, Activated T
Cell, Fatty Acid Beta Oxidation, LDG, MHC II, Monocyte, NLRP3 Inflammasome, Oxidative
Phosphorylation, pDC, Pentose Phosphate Signature, Plasma Cell, Proinflammatory IL-1
Family, TCA cycle, Unfolded Protein
8
Alternative Complement Pathway, Amino Acid Metabolism, Anti inflammation, Apoptosis,
Cell Cycle, Classical Complement Pathway, Cytotoxic, Activated T Cell, Dendritic Cell,
Erythrocytes, Fatty Acid Beta Oxidation, Glycolysis, Granulocyte, IFN,
Inflammatory_Neutrophil, LDG, Monocyte, Oxidative Phosphorylation, pDC, Pentose
Phosphate Signature, TNF
9
Alternative Complement Pathway, Amino Acid Metabolism, Anergic / Activated T cells, Anti
inflammation, Apoptosis, CD40 Activated B Cell, Cell Cycle, Classical Complement
Pathway, Fatty Acid Beta Oxidation, Glycolysis, IFN, MHC II, Oxidative Phosphorylation,
Pentose Phosphate Signature, Plasma Cell, Proinflammatory IL-1 Family, T Cell, Tactivated,
TNF, Unfolded Protein
10
Alternative Complement Pathway, Amino Acid Metabolism, Anergic / Activated T cells,
Apoptosis, CD40 Activated B Cell, Cell Cycle, Cytotoxic, Activated T Cell, Fatty Acid Alpha
Oxidation, Fatty Acid Beta Oxidation, IFN, Inflammatory_Neutrophil, NFkB Complex,
Oxidative Phosphorylation, Pentose Phosphate Signature, Plasma Cell, Proinflammatory IL-1
Family, ROS Protection, T Cell, TCA cycle, Unfolded Protein
Machine Learning Round 2: Top 10 features from each repeatations.
1
Alternative Complement Pathway, Amino Acid Metabolism, Anti inflammation, CD40
Activated B Cell, Cell Cycle, Oxidative Phosphorylation, Pentose Phosphate Signature,
Plasma Cell, T Cell, TCA cycle
2
Alternative Complement Pathway, Amino Acid Metabolism, Anti inflammation, Apoptosis,
Cell Cycle, Oxidative Phosphorylation, Pentose Phosphate Signature, Plasma Cell, TCA
cycle, TNF
3
Amino Acid Metabolism, Anti inflammation, Apoptosis, Cell Cycle, Monocyte, Oxidative
Phosphorylation, Pentose Phosphate Signature, Platelets, Proinflammatory IL-1 Family, TNF
4
Amino Acid Metabolism, CD40 Activated B Cell, Cell Cycle, Classical Complement
Pathway, IFN, Inflammatory_Neutrophil, Oxidative Phosphorylation, Pentose Phosphate
Signature, Plasma Cell, Unfolded Protein
5
Alternative Complement Pathway, Amino Acid Metabolism, Apoptosis, Cell Cycle,
Cytotoxic, Activated T Cell, Inflammatory_Neutrophil, Oxidative Phosphorylation, Pentose
Phosphate Signature, Plasma Cell, Platelets
6
Alternative Complement Pathway, Amino Acid Metabolism, CD40 Activated B Cell, Cell
Cycle, Classical Complement Pathway, LDG, Oxidative Phosphorylation, Pentose Phosphate
Signature, Plasma Cell, Unfolded Protein
7
Amino Acid Metabolism, Apoptosis, Cytotoxic, Activated T Cell, Monocyte, Oxidative
Phosphorylation, pDC, Pentose Phosphate Signature, Plasma Cell, Proinflammatory IL-1
Family, Unfolded Protein
8
Alternative Complement Pathway, Amino Acid Metabolism, Anti inflammation, Apoptosis,
Cell Cycle, IFN, Inflammatory_Neutrophil, Monocyte, Oxidative Phosphorylation, Pentose
Phosphate Signature
9
TNF, Alternative Complement Pathway, Apoptosis, CD40 Activated B Cell, Cell Cycle,
Classical Complement Pathway, IFN, Pentose Phosphate Signature, T Cell, Unfolded Protein
10
Alternative Complement Pathway, Anergic / Activated T cells, CD40 Activated B Cell, Cell
Cycle, Inflammatory_Neutrophil, Oxidative Phosphorylation, Pentose Phosphate Signature,
Plasma Cell, Proinflammatory IL-1 Family, T Cell
Machine Learning Round 3: Top 20 features (genes) from each repeatations
1
CARD16, CCNB1, CCNB2, CDC20, CEP55, CFL1, COX7B, COX7C, E2F3, H2AC6,
H2BC21, H2BC5, HMGB2, LAP3, NDUFA5, NDUFAF7, PGD, SDC1, TMEM126B,
UQCRB
2
CARD16, CASP7, CCNB1, CCNB2, CDC20, CEP55, CFL1, COX7B, COX7C, DNAJC15,
DUSP4, E2F3, H2BC21, H2BC5, NDUFA5, NDUFAF5, NDUFAF7, TMEM126B, UQCRB,
WSB2
3
CASP3, CCNB1, CCNE1, CD177, CDC20, CEP55, CFD, CFL1, CLEC4D, COX7B,
COX7C, CR1, DNAJC15, DUSP4, E2F3, FAS, H2BC21, PTTG1, TMEM126B, UQCRB
4
C3, CCNB1, CCNB2, CCNE1, CD177, CD58, CDC20, CEP55, CFD, CLEC4D, COX7B,
COX7C, CYCS, DNAJC15, DUSP4, E2F3, H2BC21, TMEM126B, TPX2, UQCRB
5
CARD16, CCNB1, CCNB2, CD177, CDC20, CEP55, CFD, COX7B, COX7C, DNAJC15,
DUSP4, H2BC5, LAP3, MSH6, NDUFA5, NDUFAF7, NT5C3A, TKTL1, TMEM126B,
UQCRB
6
ANXA3, APAF1, CCNB1, CCNE1, CEP55, CLEC4D, COX7B, COX7C, CR1, DNAJC15,
DUSP4, FAS, H2BC21, HIF1A, NDUFA5, NDUFAF5, NDUFAF7, NT5C3A, TMEM126B,
UQCRB
7
BRCA1, CARD16, CASP7, CCNB1, CCNE1, CEP55, CFL1, CLEC4D, COX7B, COX7C,
DNAJC15, DUSP4, E2F3, H2BC21, H2BC5, NDUFAF5, NDUFAF7, NT5C3A,
TMEM126B, UQCRB
8
CARD16, CCNB2, CD58, CDC20, CEP55, CFD, CLEC4D, COX18, COX20, COX7B,
COX7C, DNAJC15, DUSP4, E2F3, FAS, NDUFA5, NDUFAF7, TMEM126B, TPX2,
UQCRB,
9
CASP3, CCNB2, CCNE1, CD58, CDC20, CEP55, CFD, CFL1, CLEC4D, COX7B, COX7C,
DNAJC15, DUSP4, FAS, NDUFA5, NDUFAF5, NDUFAF7, TMEM126B, TPX2, UQCRB
10
CARD16, CCNB2, CEP55, CFD, CFL1, CLEC4D, COX7B, COX7C, DNAJC15, DUSP4,
E2F3, FAS, GINS2, H2BC21, H2BC5, HIF1A, NDUFA5, NDUFAF7, TMEM126B, UQCRB

TABLE 3C
Top features from ML classification of critical vs non-critical COVID.

Machine Learning Round 1: Top 20 features from each repeatations.
1
Amino Acid Metabolism, CD40 Activated B Cell, Cell Cycle, Dendritic Cell, Fatty Acid
Alpha Oxidation, Granulocyte, IFN, Inflammatory_Neutrophil, LDG, MHC II, NK Cell,
NLRP3 Inflammasome, pDC, Pentose Phosphate Signature, Plasma Cell, ROS Protection, T
Cell, Tactivated, Treg, Unfolded Protein
2
Alternative Complement Pathway, Amino Acid Metabolism, Apoptosis, CD40 Activated B
Cell, Cytotoxic, Activated T Cell, Dendritic Cell, Fatty Acid Alpha Oxidation, Fatty Acid
Beta Oxidation, IFN, Inflammatory_Neutrophil, LDG, MHC II, NFkB Complex, Oxidative
Phosphorylation, Pentose Phosphate Signature, T Cell, Tactivated, TNF, Treg, Unfolded
Protein
3
Amino Acid Metabolism, Anergic / Activated T cells, Apoptosis, Cytotoxic, Activated T Cell,
Dendritic Cell, Erythrocytes, Fatty Acid Alpha Oxidation, Fatty Acid Beta Oxidation,
Glycolysis, Granulocyte, Inflammatory_Neutrophil, LDG, MHC II, NLRP3 Inflammasome,
Proinflammatory IL-1 Family, T Cell, Tactivated, TNF, Treg, Unfolded Protein
4
Amino Acid Metabolism, Anergic / Activated T cells, Apoptosis, Cell Cycle, Classical
Complement Pathway, Cytotoxic, Activated T Cell, Dendritic Cell, Erythrocytes, Fatty Acid
Alpha Oxidation, Fatty Acid Beta Oxidation, Glycolysis, IFN, Inflammatory_Neutrophil,
LDG, Monocyte, Pentose Phosphate Signature, Plasma Cell, T Cell, Tactivated, Unfolded
Protein
5
Alternative Complement Pathway, Amino Acid Metabolism, Anergic / Activated T cells,
Apoptosis, Cytotoxic, Activated T Cell, Dendritic Cell, Fatty Acid Beta Oxidation, IFN,
Inflammatory_Neutrophil, LDG, MHC II, Monocyte, NFkB Complex, pDC, Pentose
Phosphate Signature, ROS Protection, T Cell, Tactivated, TNF, Unfolded Protein
6
Amino Acid Metabolism, Apoptosis, Classical Complement Pathway, Cytotoxic, Activated T
Cell, Dendritic Cell, Erythrocytes, Fatty Acid Alpha Oxidation, Fatty Acid Beta Oxidation,
Glycolysis, IFN, Inflammatory_Neutrophil, NFkB Complex, NK Cell, pDC, Pentose
Phosphate Signature, Plasma Cell, T Cell, Tactivated, Treg, Unfolded Protein
7
Alternative Complement Pathway, Amino Acid Metabolism, Anergic / Activated T cells,
CD40 Activated B Cell, Cell Cycle, Cytotoxic, Activated T Cell, Fatty Acid Alpha Oxidation,
IFN, Inflammatory_Neutrophil, LDG, MHC II, Monocyte, NFkB Complex, Pentose
Phosphate Signature, Plasma Cell, ROS Protection, T Cell, Tactivated, Treg, Unfolded Protein
8
Alternative Complement Pathway, Amino Acid Metabolism, Apoptosis, Classical
Complement Pathway, Cytotoxic, Activated T Cell, Fatty Acid Alpha Oxidation, Granulocyte,
IFN, Inflammatory_Neutrophil, LDG, MHC II, Oxidative Phosphorylation, Pentose
Phosphate Signature, Plasma Cell, Platelets, Proinflammatory IL-1 Family, ROS Protection, T
Cell, Treg, Unfolded Protein
9
Alternative Complement Pathway, Amino Acid Metabolism, Anergic / Activated T cells,
Cytotoxic, Activated T Cell, Dendritic Cell, Fatty Acid Alpha Oxidation, Fatty Acid Beta
Oxidation, IFN, Inflammatory_Neutrophil, LDG, MHC II, Monocyte, Oxidative
Phosphorylation, pDC, Pentose Phosphate Signature, Plasma Cell, T Cell, Tactivated, Treg,
Unfolded Protein
10
Amino Acid Metabolism, Anergic / Activated T cells, Classical Complement Pathway,
Cytotoxic, Activated T Cell, Dendritic Cell, Fatty Acid Alpha Oxidation, Fatty Acid Beta
Oxidation, Glycolysis, IFN, Inflammatory_Neutrophil, LDG, MHC II, Monocyte, NLRP3
Inflammasome, Pentose Phosphate Signature, Plasma Cell, T Cell, Tactivated, TCA cycle,
Unfolded Protein
Machine Learning Round 2: Top 10 features from each repeatations
1
Amino Acid Metabolism, CD40 Activated B Cell, Fatty Acid Alpha Oxidation, Granulocyte,
Inflammatory Neutrophil, Pentose Phosphate Signature, T Cell, Tactivated, Treg, Unfolded
Protein
2
Amino Acid Metabolism, Cytotoxic, Activated T Cell, Fatty Acid Alpha Oxidation, Fatty
Acid Beta Oxidation, Inflammatory_Neutrophil, MHC II, T Cell, TNF, Treg, Unfolded
Protein
3
Anergic / Activated T cells, Apoptosis, Cytotoxic, Activated T Cell, Dendritic Cell,
Granulocyte, Inflammatory_Neutrophil, MHC II, T Cell, Tactivated, Unfolded Protein
4
IFN, Amino Acid Metabolism, Apoptosis, Cytotoxic, Activated T Cell, Fatty Acid Beta
Oxidation, Inflammatory_Neutrophil, LDG, T Cell, Tactivated, Unfolded Protein
5
Amino Acid Metabolism, Fatty Acid Beta Oxidation, IFN, Inflammatory_Neutrophil, LDG,
Pentose Phosphate Signature, ROS Protection, T Cell, Tactivated, Unfolded Protein
6
Amino Acid Metabolism, Apoptosis, IFN, Inflammatory_Neutrophil, NFkB Complex,
Pentose Phosphate Signature, T Cell, Tactivated, Treg, Unfolded Protein
7
Cytotoxic, Activated T Cell, IFN, Inflammatory_Neutrophil, Monocyte, Pentose Phosphate
Signature, Plasma Cell, T Cell, Tactivated, Treg, Unfolded Protein
8
Amino Acid Metabolism, Fatty Acid Alpha Oxidation, Granulocyte, IFN,
Inflammatory_Neutrophil, Oxidative Phosphorylation, Pentose Phosphate Signature, T Cell,
Treg, Unfolded Protein
9
Dendritic Cell, IFN, Inflammatory_Neutrophil, LDG, MHC II, Pentose Phosphate Signature,
T Cell, Tactivated, Treg, Unfolded Protein
10
Amino Acid Metabolism, Classical Complement Pathway, Cytotoxic, Activated T Cell, Fatty
Acid Alpha Oxidation, Inflammatory_Neutrophil, NLRP3 Inflammasome, Pentose Phosphate
Signature, T Cell, Tactivated, Unfolded Protein
Machine Learning Round 3: Top 20 features (genes) from each repeatations
1
C3AR1, CD28, CREM, EIF2AK2, ERAP1, FKBP5, GSTK1, H2BC21, H2BC5, HAAO,
HIF1A, IFI44L, IL23R, MAN1A1, MCEMP1, PIGK, SGK1, SP100, SSR1, TKTL1
2
ADD3, ARG1, CD177, CD28, CREM, EIF2AK2, EOMES, ERAP1, ERGIC2, FKBP5,
H2BC5, HIF1A, ISG20, LAP3, MCEMP1, OAT, SAMD9, SGK1, SP100, SSR1
3
CANX, CARD16, CAST, CD177, CD28, CREM, DERL1, EIF2AK2, EOMES, ERAP1,
HIF1A, IKZF2, MCEMP1, MTPN, NT5C3A, OAT, OXCT1, SGK1, SP100, SSR1
4
ADD3, CAST, CCR3, CD177, CD28, CREM, DERL1, EIF2AK2, EOMES, ERAP1,
ERGIC2, FKBP5, H2BC5, MCEMP1, NT5C3A, OAT, OXCT1, SGK1, SP100, SSR1
5
CD177, CD28, CREM, EIF2AK2, EOMES, ERAP1, FKBP5, GBP5, GOLT1B, GSTK1,
H2BC21, HIF1A, MCEMP1, NT5C3A, OXCT1, PGD, PIGK, SGK1, SP100, SSR1
6
ARG1, CAST, CCR3, CD28, CD69, CREM, DERL1, EIF2AK2, EOMES, ERAP1, FKBP5,
HAAO, HIF1A, IFI44L, MCEMP1, OXCT1, PGD, SGK1, SLFN5, SP100
7
ADD3, ANXA3, CARD16, CAST, CD177, CD28, CLEC4D, CREM, EOMES, ERGIC2,
FKBP5, MCEMP1, MTPN, NT5C3A, OXCT1, PRPS2, RPE, SGK1, SP100, SSR1
8
ANXA3, CARD16, CAST, CCR3, CD28, CREM, DERL1, EIF2AK2, EOMES, FKBP5,
H2BC21, HIF1A, IFI44L, IKZF2, MAN1A1, MCEMP1, PRPS2, SGK1, SP100, SSR1
9
CCR3, CD177, CD28, DERL1, EIF2AK2, EOMES, ERAP1, FKBP5, GOLT1B, GSTK1,
H2BC21, HIF1A, MAN1A1, MCEMP1, MTPN, SGK1, SLFN5, SP100, SSR1, TKTL1
10
ANXA3, CARD16, CAST, CD177, CD28, EIF2AK2, EOMES, ERAP1, FKBP5, H2BC21,
HAAO, HIF1A, LAP3, MAN1A1, MCEMP1, MTPN, NT5C3A, PGD, SGK1, SP100

TABLE 3D
Top features from ML classification of COVID vs non-COVID ICU.

Machine Learning Round 1: Top 20 features from each repeatations.
1
Anti inflammation, Cell Cycle, Cytotoxic, Activated T Cell, Dendritic Cell, Erythrocytes,
Glycolysis, Granulocyte, IFN, Inflammatory_Neutrophil, LDG, MHC II, NFkB Complex, NK
Cell, NLRP3 Inflammasome, pDC, Pentose Phosphate Signature, Plasma Cell,
Proinflammatory IL-1 Family, ROS Protection, Treg
2
IFN, Anergic / Activated T cells, Apoptosis, CD40 Activated B Cell, Classical Complement
Pathway, Cytotoxic, Activated T Cell, Erythrocytes, Glycolysis, Granulocyte,
Inflammatory_Neutrophil, LDG, NK Cell, pDC, Pentose Phosphate Signature, Plasma Cell,
ROS Protection, T Cell, Tactivated, TCA cycle, Unfolded Protein
3
Amino Acid Metabolism, Anergic / Activated T cells, Apoptosis, CD40 Activated B Cell,
Cell Cycle, Cytotoxic, Activated T Cell, Erythrocytes, Fatty Acid Beta Oxidation,
Granulocyte, IFN, Inflammatory_Neutrophil, LDG, Monocyte, NK Cell, pDC, Pentose
Phosphate Signature, Plasma Cell, T Cell, TCA cycle, TNF
4
IFN, Amino Acid Metabolism, Anergic / Activated T cells, Anti inflammation, CD40
Activated B Cell, Cell Cycle, Cytotoxic, Activated T Cell, Dendritic Cell, Erythrocytes,
Granulocyte, Inflammatory_Neutrophil, LDG, Monocyte, pDC, Pentose Phosphate Signature,
Plasma Cell, ROS Protection, T Cell, TCA cycle, TNF
5
Amino Acid Metabolism, Anergic / Activated T cells, Apoptosis, CD40 Activated B Cell,
Erythrocytes, IFN, Inflammatory_Neutrophil, LDG, MHC II, NFkB Complex, NK Cell,
Oxidative Phosphorylation, pDC, Pentose Phosphate Signature, Plasma Cell, Proinflammatory
IL-1 Family, T Cell, Tactivated, TCA cycle, Unfolded Protein
6
Anergic / Activated T cells, Anti inflammation, Apoptosis, CD40 Activated B Cell, Cell
Cycle, Classical Complement Pathway, Cytotoxic, Activated T Cell, Erythrocytes,
Granulocyte, IFN, MHC II, NK Cell, pDC, Pentose Phosphate Signature, Plasma Cell,
Proinflammatory IL-1 Family, ROS Protection, T Cell, Tactivated, TCA cycle
7
Amino Acid Metabolism, Anergic / Activated T cells, CD40 Activated B Cell, Cell Cycle,
Erythrocytes, Granulocyte, IFN, Inflammatory_Neutrophil, LDG, MHC II, Monocyte, pDC,
Pentose Phosphate Signature, Plasma Cell, Proinflammatory IL-1 Family, ROS Protection, T
Cell, TCA cycle, TNF, Treg
8
Amino Acid Metabolism, Anergic / Activated T cells, Anti inflammation, Apoptosis, CD40
Activated B Cell, Cell Cycle, Cytotoxic, Activated T Cell, Erythrocytes, IFN,
Inflammatory_Neutrophil, LDG, MHC II, Monocyte, pDC, Pentose Phosphate Signature,
Plasma Cell, Platelets, ROS Protection, Tactivated, TNF
9
Amino Acid Metabolism, CD40 Activated B Cell, Cell Cycle, Erythrocytes, Fatty Acid Alpha
Oxidation, Granulocyte, IFN, Inflammatory_Neutrophil, LDG, MHC II, Monocyte, NFKB
Complex, NLRP3 Inflammasome, pDC, Plasma Cell, Proinflammatory IL-1 Family,
Tactivated, TCA cycle, TNF, Treg
10
Amino Acid Metabolism, Anergic / Activated T cells, Anti inflammation, Apoptosis, CD40
Activated B Cell, Cell Cycle, Classical Complement Pathway, Dendritic Cell, Erythrocytes,
IFN, Inflammatory_Neutrophil, LDG, NFkB Complex, NK Cell, pDC, Pentose Phosphate
Signature, Plasma Cell, T Cell, TNF, Treg
Machine Learning Round 2: Top 10 features from each repeatations.
1
Cell Cycle, Erythrocytes, IFN, Inflammatory_Neutrophil, NFkB Complex, NK Cell, NLRP3
Inflammasome, Pentose Phosphate Signature, Plasma Cell, ROS Protection
2
CD40 Activated B Cell, Erythrocytes, IFN, LDG, NK Cell, pDC, Plasma Cell, ROS
Protection, TCA cycle, Unfolded Protein
3
CD40 Activated B Cell, Cell Cycle, Erythrocytes, IFN, Inflammatory_Neutrophil, LDG, NK
Cell, Plasma Cell, TCA cycle, TNF
4
CD40 Activated B Cell, Cell Cycle, Erythrocytes, IFN, Inflammatory_Neutrophil, LDG, pDC,
Pentose Phosphate Signature, Plasma Cell, TNF
5
IFN, Apoptosis, CD40 Activated B Cell, Erythrocytes, LDG, NK Cell, pDC, Plasma Cell,
TCA cycle, Unfolded Protein
6
Apoptosis, CD40 Activated B Cell, Cell Cycle, Erythrocytes, IFN, NK Cell, pDC, Pentose
Phosphate Signature, Plasma Cell, TCA cycle
7
CD40 Activated B Cell, Erythrocytes, IFN, Inflammatory_Neutrophil, LDG, pDC, Plasma
Cell, TCA cycle, TNF, Treg
8
Anergic / Activated T cells, Apoptosis, CD40 Activated B Cell, IFN, LDG, MHC II, pDC,
Pentose Phosphate Signature, Plasma Cell, ROS Protection
9
CD40 Activated B Cell, Cell Cycle, Erythrocytes, IFN, Monocyte, pDC, Plasma Cell, TCA
cycle, TNF, Treg
10
IFN, Apoptosis, CD40 Activated B Cell, Cell Cycle, LDG, pDC, Pentose Phosphate
Signature, Plasma Cell, TNF, Treg
Machine Learning Round 3: Top 20 features (genes) from each repeatations.
1
ASAP1, CAMP, CLEC4C, CS, CXCL1, DLAT, EGR1, FBXL2, G6PD, GBP4, GFI1B,
GMIP, GP1BA, GYPA, IFITM1, MKI67, PRPS2, SDC1, SDHAF3, WEE1
2
AMPD3, ASAP1, CAMP, CDKN3, E2F3, EGR1, EREG, FBXL2, FCER2, G6PD, GBP4,
GFI1B, GMIP, GP1BA, GYPA, H6PD, IFITM1, IFITM3, PTTG1, SDC1
3
ADGRE2, CCNB2, CD38, CXCL1, CXCL8, EGR1, FBXL2, FCER2, G6PD, GMIP,
MCM10, MCM2, MGLL, MKI67, MPO, NFE2, OGDH, OGDHL, PTTG1, SDC1
4
ASAP1, CAMP, CDKN3, CS, EGR1, FBXL2, G6PD, GBP4, GMIP, GP1BA, GYPA, H6PD,
IFITM1, IL18, INHBA, MCM10, PTTG1, SDC1, SDHAF3, SDHC
5
ASAP1, AURKA, CCNB1, CDC20, CS, CXCL8, DLAT, EGR1, EREG, FBXL2, FCER2,
G6PD, GBP4, GFI1B, GYPA, H6PD, IFITM1, PTTG1, SDC1, TNFAIP3
6
AMPD3, CAMP, CD38, EGR1, FBXL2, FCER2, G6PD, GBP4, GYPA, H6PD, IGHMBP2,
MCM10, MPO, MYBL2, OGDH, PDK2, PTGS2, PTTG1, SDC1, SDHC
7
AMPD3, ASAP1, CAMP, CCNB2, CD38, CDKN3, EGR1, FBXL2, G6PD, GBP4, GMIP,
GP1BA, GYPA, H6PD, IFITM1, IFITM3, PTTG1, SDC1, SDHC, TNFRSF17
8
AMPD3, ASAP1, CAMP, CD38, CDKN3, EGR1, FBXL2, G6PD, GBP4, GYPA, IFITM1,
MCM10, MCM2, MKI67, MPO, PTTG1, SDC1, SDHAF3, SDHC, VEGFA
9
AMPD3, ASAP1, CCNB2, CDKN3, CS, CXCL1, DLAT, DUSP4, EGR1, FBXL2, G6PD,
GP1BA, HOMER2, IFITM3, INHBA, MYBL2, PDK4, PTTG1, SDC1, SDHC
10
AMPD3, ASAP1, CAMP, CASP10, CDKN3, CEACAM6, EGR1, FBXL2, G6PD, GBP4,
GMIP, HOMER2, IFITM1, IFITM3, IL18, PTTG1, SDC1, SDHA, SDHAF3, SDHC

### REFERENCES

1. Guan, W. J.; Ni, Z. Y.; Hu, Y.; Liang, W. H.; Ou, C. Q.; He, J. X.; Liu, L.; Shan, H.; Lei, C. L.; Hui, D. S. C.; et al. Clinical Characteristics of Coronavirus Disease 2019 in China. N. Engl. J. Med. 2020, 382, 1708-1720, doi: 10.1056/NEJMoa2002032.
2. Wang, D.; Hu, B.; Hu, C.; Zhu, F.; Liu, X.; Zhang, J.; Wang, B.; Xiang, H.; Cheng, Z.; Xiong, Y.; et al. Clinical Characteristics of 138 Hospitalized Patients with 2019 Novel Coronavirus-Infected Pneumonia in Wuhan, China. JAMA-J. Am. Med. Assoc. 2020, 323, 1061-1069, doi: 10.1001/jama.2020.1585.
3. WHO WHO Coronavirus WHO Coronavirus Available online: https://covid19.who.int/.
4. Wiersinga, W. J.; Rhodes, A.; Cheng, A. C.; Peacock, S. J.; Prescott, H. C. Pathophysiology, Transmission, Diagnosis, and Treatment of Coronavirus Disease 2019 (COVID-19): A Review. JAMA 2020, 324, 782-793, doi: 10.1001/jama.2020.12839.
5. Nalbandian, A.; Sehgal, K.; Gupta, A.; Madhavan, M. V; McGroder, C.; Stevens, J. S.; Cook, J. R.; Nordvig, A. S.; Shalev, D.; Sehrawat, T. S.; et al. Post-Acute COVID-19 Syndrome. Nat. Med. 2021, 27, 601-615, doi: 10.1038/s41591-021-01283-z.
6. Williamson, E. J.; Walker, A. J.; Bhaskaran, K.; Bacon, S.; Bates, C.; Morton, C. E.; Curtis, H. J.; Mehrkar, A.; Evans, D.; Inglesby, P.; et al. Factors Associated with COVID-19-Related Death Using OpenSAFELY. Nature 2020, 584, 430-436, doi: 10.1038/s41586-020-2521-4.
7. Takahashi, T.; Ellingson, M. K.; Wong, P.; Israelow, B.; Lucas, C.; Klein, J.; Silva, J.; Mao, T.; Oh, J. E.; Tokuyama, M.; et al. Sex Differences in Immune Responses That Underlie COVID-19 Disease Outcomes. Nature 2020, 588, 315-320, doi: 10.1038/s41586-020-2700-3.
8. Mokhtari, T.; Hassani, F.; Ghaffari, N.; Ebrahimi, B.; Yarahmadi, A.; Hassanzadeh, G. COVID-19 and Multiorgan Failure: A Narrative Review on Potential Mechanisms. J. Mol. Histol. 2020, 51, 613-628, doi: 10.1007/s10735-020-09915-3.
9. Michalski, J. E.; Kurche, J. S.; Schwartz, D. A. From ARDS to Pulmonary Fibrosis: The next Phase of the COVID-19 Pandemic? Transl. Res. 2022, 241, 13-24, doi: 10.1016/j.trsl.2021.09.001.
10. Chen, Y. T.; Shao, S. C.; Hsu, C. K.; Wu, I. W.; Hung, M. J.; Chen, Y. C. Incidence of Acute Kidney Injury in COVID-19 Infection: A Systematic Review and Meta-Analysis. Crit. Care 2020, 24, 346, doi: 10.1186/s13054-020-03009-y.
11. Merad, M.; Blish, C. A.; Sallusto, F.; Iwasaki, A. The Immunology and Immunopathology of COVID-19. Science 2022, 375, 1122-1127, doi: 10.1126/science.abm8108.
12. Ilieva, M.; Tschaikowski, M.; Vandin, A.; Uchida, S. The Current Status of Gene Expression Profilings in COVID-19 Patients. Clin. Transl. Discov. 2022, 2, e104, doi: 10.1002/ctd2.104.
13. Daamen, A. R.; Bachali, P.; Owen, K. A.; Kingsmore, K. M.; Hubbard, E. L.; Labonte, A. C.; Robl, R.; Shrotri, S.; Grammer, A. C.; Lipsky, P. E. Comprehensive Transcriptomic Analysis of COVID-19 Blood, Lung, and Airway. Sci. Rep. 2021, 11, 7052, doi: 10.1038/s41598-021-86002-x.
14. Daamen, A. R.; Bachali, P.; Bonham, C. A.; Somerville, L.; Sturek, J. M.; Grammer, A. C.; Kadl, A.; Lipsky, P. E. COVID-19 Patients Exhibit Unique Transcriptional Signatures Indicative of Disease Severity. Front. Immunol. 2022, 13, 989556, doi: 10.3389/fimmu.2022.989556.
15. Mathew, D.; Giles, J. R.; Baxter, A. E.; Oldridge, D. A.; Greenplate, A. R.; Wu, J. E.; Alanio, C.; Kuri-Cervantes, L.; Pampena, M. B.; D'Andrea, K.; et al. Deep Immune Profiling of COVID-19 Patients Reveals Distinct Immunotypes with Therapeutic Implications. Science (80-.). 2020, 369, doi: 10.1126/SCIENCE. ABC8511.
16. Lucas, C.; Wong, P.; Klein, J.; Castro, T. B. R.; Silva, J.; Sundaram, M.; Ellingson, M. K.; Mao, T.; Oh, J. E.; Israelow, B.; et al. Longitudinal Analyses Reveal Immunological Misfiring in Severe COVID-19. Nature 2020, 584, 463-469, doi: 10.1038/s41586-020-2588-y.
17. Wilk, A. J.; Rustagi, A.; Zhao, N. Q.; Roque, J.; Martínez-Colón, G. J.; McKechnie, J. L.; Ivison, G. T.; Ranganath, T.; Vergara, R.; Hollis, T.; et al. A Single-Cell Atlas of the Peripheral Immune Response in Patients with Severe COVID-19. Nat. Med. 2020, 26, 1070-1076, doi: 10.1038/s41591-020-0944-y.
18. Wilk, A. J.; Lee, M. J.; Wei, B.; Parks, B.; Pi, R.; Martinez-Colón, G. J.; Ranganath, T.; Zhao, N. Q.; Taylor, S.; Becker, W.; et al. Multi-Omic Profiling Reveals Widespread Dysregulation of Innate Immunity and Hematopoiesis in COVID-19. J. Exp. Med. 2021, 218, doi: 10.1084/jem.20210582.
19. McClain, M. T.; Constantine, F. J.; Henao, R.; Liu, Y.; Tsalik, E. L.; Burke, T. W.; Steinbrink, J. M.; Petzold, E.; Nicholson, B. P.; Rolfe, R.; et al. Dysregulated Transcriptional Responses to SARS-COV-2 in the Periphery. Nat. Commun. 2021, 12, 1-8, doi: 10.1038/s41467-021-21289-y.
20. Stephenson, E.; Reynolds, G.; Botting, R. A.; Calero-Nieto, F. J.; Morgan, M. D.; Tuong, Z. K.; Bach, K.; Sungnak, W.; Worlock, K. B.; Yoshida, M.; et al. Single-Cell Multi-Omics Analysis of the Immune Response in COVID-19. Nat. Med. 2021, doi: 10.1038/s41591-021-01329-2.
21. Overmyer, K. A.; Shishkova, E.; Miller, I. J.; Balnis, J.; Bernstein, M. N.; Peters-Clarke, T. M.; Meyer, J. G.; Quan, Q.; Muehlbauer, L. K.; Trujillo, E. A.; et al. Large-Scale Multi-Omic Analysis of COVID-19 Severity. Cell Syst. 2021, 12, 23-40.e7, doi: 10.1016/j.cels.2020.10.003.
22. Carapito, R.; Li, R.; Helms, J.; Carapito, C.; Gujja, S.; Rolli, V.; Guimaraes, R.; Malagon-Lopez, J.; Spinnhirny, P.; Lederle, A.; et al. Identification of Driver Genes for Critical Forms of COVID-19 in a Deeply Phenotyped Young Patient Cohort. Sci. Transl. Med. 2022, 14, 1-21, doi: 10.1126/scitranslmed. abj7521.
23. Shah, P.; Kendall, F.; Khozin, S.; Goosen, R.; Hu, J.; Laramie, J.; Ringel, M.; Schork, N. Artificial Intelligence and Machine Learning in Clinical Development: A Translational Perspective. npj Digit. Med. 2019, 2, 69, doi: 10.1038/s41746-019-0148-3.
24. Meraihi, Y.; Gabis, A. B.; Mirjalili, S.; Ramdane-Cherif, A.; Alsaadi, F. E. Machine Learning-Based Research for COVID-19 Detection, Diagnosis, and Prediction: A Survey. SN Comput. Sci. 2022, 3, 286, doi: 10.1007/s42979-022-01184-z.
25. Quiroz-Juarez, M. A.; Torres-Gomez, A.; Hoyo-Ulloa, I.; Leon-Montiel, R. de J.; U'Ren, A. B. Identification of High-Risk COVID-19 Patients Using Machine Learning. PLoS One 2021, 16, 1-21, doi: 10.1371/journal.pone. 0257234.
26. Guhan, B.; Almutairi, L.; Sowmiya, S.; Snekhalatha, U.; Rajalakshmi, T.; Aslam, S. M. Automated System for Classification of COVID-19 Infection from Lung CT Images Based on Machine Learning and Deep Learning Techniques. Sci. Rep. 2022, 12, 17417, doi: 10.1038/s41598-022-20804-5.
27. Nguyen, D.; Kay, F.; Tan, J.; Yan, Y.; Ng, Y. S.; Iyengar, P.; Peshock, R.; Jiang, S. Deep Learning-Based COVID-19 Pneumonia Classification Using Chest CT Images: Model Generalizability. Front. Artif. Intell. 2021, 4, doi: 10.3389/frai.2021.694875.
28. Zargari Khuzani, A.; Heidari, M.; Shariati, S. A. COVID-Classifier: An Automated Machine Learning Model to Assist in the Diagnosis of COVID-19 Infection in Chest X-Ray Images. Sci. Rep. 2021, 11, 9887, doi: 10.1038/s41598-021-88807-2.
29. Penrice-Randal, R.; Dong, X.; Shapanis, A. G.; Gardner, A.; Harding, N.; Legebeke, J.; Lord, J.; Vallejo, A. F.; Poole, S.; Brendish, N.J.; et al. Blood Gene Expression Predicts Intensive Care Unit Admission in Hospitalised Patients with COVID-19. Front. Immunol. 2022, 13, 988685, doi: 10.3389/fimmu.2022.988685.
30. Zarei Ghobadi, M.; Emamzadeh, R.; Teymoori-Rad, M.; Afsaneh, E. Exploration of Blood-Derived Coding and Non-Coding RNA Diagnostic Immunological Panels for COVID-19 through a Co-Expressed-Based Machine Learning Procedure. Front. Immunol. 2022, 13, 1001070, doi: 10.3389/fimmu.2022.1001070.
31. Song, X.; Zhu, J.; Tan, X.; Yu, W.; Wang, Q.; Shen, D.; Chen, W. XGBoost-Based Feature Learning Method for Mining COVID-19 Novel Diagnostic Markers. Front. public Heal. 2022, 10, 926069, doi: 10.3389/fpubh.2022.926069.
32. Li, H.; Huang, F.; Liao, H.; Li, Z.; Feng, K.; Huang, T.; Cai, Y. D. Identification of COVID-19-Specific Immune Markers Using a Machine Learning Method. Front. Mol. Biosci. 2022, 9, 952626, doi: 10.3389/fmolb.2022.952626.
33. Li, X.; Zhou, X.; Ding, S.; Chen, L.; Feng, K.; Li, H.; Huang, T.; Cai, Y. D. Identification of Transcriptome Biomarkers for Severe COVID-19 with Machine Learning Methods. Biomolecules 2022, 12, doi: 10.3390/biom12121735.
34. Lohmann, S.; Herold, A.; Bergauer, T.; Belousov, A.; Betzl, G.; Demario, M.; Dietrich, M.; Luistro, L.; Poignée-Heger, M.; Schostack, K.; et al. Gene Expression Analysis in Biomarker Research and Early Drug Development Using Function Tested Reverse Transcription Quantitative Real-Time PCR Assays. Methods 2013, 59, 10-19, doi: https://doi.org/10.1016/j.ymeth.2012.07.003.
35. El-Deiry, W. S.; Goldberg, R. M.; Lenz, H. J.; Shields, A. F.; Gibney, G. T.; Tan, A. R.; Brown, J.; Eisenberg, B.; Heath, E. I.; Phuphanich, S.; et al. The Current State of Molecular Testing in the Treatment of Patients with Solid Tumors, 2019. CA. Cancer J. Clin. 2019, 69, 305-343, doi: 10.3322/caac.21560.
36. Marshall, K. W.; Mohr, S.; Khettabi, F. El; Nossova, N.; Chao, S.; Bao, W.; Ma, J.; Li, X. J.; Liew, C. C. A Blood-Based Biomarker Panel for Stratifying Current Risk for Colorectal Cancer. Int. J. cancer 2010, 126, 1177-1186, doi: 10.1002/ijc.24910.
37. Uddin, S.; Khan, A.; Hossain, M. E.; Moni, M. A. Comparing Different Supervised Machine Learning Algorithms for Disease Prediction. BMC Med. Inform. Decis. Mak. 2019, 19, 281, doi: 10.1186/s12911-019-1004-8.
38. Mazlan, A. U.; Sahabudin, N. A.; Remli, M. A.; Ismail, N. S.; Mohamad, M. S.; Nies, H. W.; Abd Warif, N. B. A Review on Recent Progress in Machine Learning and Deep Learning Methods for Cancer Classification on Gene Expression Data. Processes 2021, 9.
39. Kingsmore, K. M.; Puglisi, C. E.; Grammer, A. C.; Lipsky, P. E. An Introduction to Machine Learning and Analysis of Its Use in Rheumatic Diseases. Nat. Rev. Rheumatol. 2021, 17, 710-730, doi: 10.1038/s41584-021-00708-w.
40. Saeys, Y.; Inza, I.; Larranaga, P. A Review of Feature Selection Techniques in Bioinformatics. Bioinformatics 2007, 23, 2507-2517, doi: 10.1093/bioinformatics/btm344.
41. Ang, J. C.; Mirzal, A.; Haron, H.; Hamed, H. N. A. Supervised, Unsupervised, and Semi-Supervised Feature Selection: A Review on Gene Selection. IEEE/ACM Trans. Comput. Biol. Bioinforma. 2016, 13, 971-989, doi: 10.1109/TCBB.2015.2478454.
42. Samy, A.; Maher, M. A.; Abdelsalam, N. A.; Badr, E. SARS-COV-2 Potential Drugs, Drug Targets, and Biomarkers: A Viral-Host Interaction Network-Based Analysis. Sci. Rep. 2022, 12, 11934, doi: 10.1038/s41598-022-15898-w.
43. Cavalcante, L. T. de F.; da Fonseca, G. C.; Amado Leon, L. A.; Salvio, A. L.; Brustolini, O. J.; Gerber, A. L.; Guimarães, A. P. de C.; Marques, C. A. B.; Fernandes, R. A.; Ramos Filho, C. H. F.; et al. Buffy Coat Transcriptomic Analysis Reveals Alterations in Host Cell Protein Synthesis and Cell Cycle in Severe COVID-19 Patients. Int. J. Mol. Sci. 2022, 23, doi: 10.3390/ijms232113588.
44. Prado, C. A. de S.; Fonseca, D. L. M.; Singh, Y.; Filgueiras, I. S.; Baiocchi, G. C.; Plaça, D. R.; Marques, A. H. C.; Dantas-Komatsu, R. C. S.; Usuda, J. N.; Freire, P. P.; et al. Integrative Systems Immunology Uncovers Molecular Networks of the Cell Cycle That Stratify COVID-19 Severity. J. Med. Virol. 2023, doi: 10.1002/jmv.28450.
45. Duan, C.; Ma, R.; Zeng, X.; Chen, B.; Hou, D.; Liu, R.; Li, X.; Liu, L.; Li, T.; Huang, H. SARS-COV-2 Achieves Immune Escape by Destroying Mitochondrial Quality: Comprehensive Analysis of the Cellular Landscapes of Lung and Blood Specimens From Patients With COVID-19. Front. Immunol. 2022, 13, 946731, doi: 10.3389/fimmu.2022.946731.
46. Chernyak, B. V; Popova, E. N.; Prikhodko, A. S.; Grebenchikov, O. A.; Zinovkina, L. A.; Zinovkin, R. A. COVID-19 and Oxidative Stress. Biochemistry. (Mosc). 2020, 85, 1543-1553, doi: 10.1134/S0006297920120068.
47. Guarnieri, J. W.; Dybas, J. M.; Fazelinia, H.; Kim, M. S.; Frere, J.; Zhang, Y.; Albrecht, Y. S.; Murdock, D. G.; Angelin, A.; Singh, L. N.; et al. Targeted Down Regulation Of Core Mitochondrial Genes During SARS-COV-2 Infection. bioRxiv 2022, 2022.02.19.481089, doi: 10.1101/2022.02.19.481089.
48. McKenna, E.; Wubben, R.; Isaza-Correa, J. M.; Melo, A. M.; Mhaonaigh, A. U.; Conlon, N.; O'Donnell, J. S.; Ní Cheallaigh, C.; Hurley, T.; Stevenson, N.J.; et al. Neutrophils in COVID-19: Not Innocent Bystanders. Front. Immunol. 2022, 13, doi: 10.3389/fimmu.2022.864387.
49. Aschenbrenner, A. C.; Mouktaroudi, M.; Kraemer, B.; Antonakos, N.; Oestreich, M.; Gkizeli, K.; Nuesch-Germano, M.; Saridaki, M.; Bonaguro, L.; Reusch, N.; et al. Disease Severity-Specific Neutrophil Signatures in Blood Transcriptomes Stratify COVID-19 Patients. medRxiv 2020, 2020.07.07.20148395, doi: 10.1101/2020.07.07.20148395.
50. Schulte-Schrepping, J.; Reusch, N.; Paclik, D.; Baßler, K.; Schlickeiser, S.; Zhang, B.; Krämer, B.; Krammer, T.; Brumhard, S.; Bonaguro, L.; et al. Severe COVID-19 Is Marked by a Dysregulated Myeloid Cell Compartment. Cell 2020, 182, 1419-1440.e23, doi: 10.1016/j.cell.2020.08.001.
51. Lee, J. S.; Park, S.; Jeong, H. W.; Ahn, J. Y.; Choi, S. J.; Lee, H.; Choi, B.; Nam, S. K.; Sa, M.; Kwon, J. S.; et al. Immunophenotyping of Covid-19 and Influenza Highlights the Role of Type i Interferons in Development of Severe Covid-19. Sci. Immunol. 2020, 5, doi: 10.1126/sciimmunol.abd1554.
52. Dong, Z.; Yan, Q.; Cao, W.; Liu, Z.; Wang, X. Identification of Key Molecules in COVID-19 Patients Significantly Correlated with Clinical Outcomes by Analyzing Transcriptomic Data. Front. Immunol. 2022, 13, 930866, doi: 10.3389/fimmu.2022.930866.
53. Zhou, R.; To, K. K. W.; Wong, Y. C.; Liu, L.; Zhou, B.; Li, X.; Huang, H.; Mo, Y.; Luk, T. Y.; Lau, T. T. K.; et al. Acute SARS-COV-2 Infection Impairs Dendritic Cell and T Cell Responses. Immunity 2020, 53, 864-877.e5, doi: 10.1016/j.immuni.2020.07.026.
54. Chen, Z.; John Wherry, E. T Cell Responses in Patients with COVID-19. Nat. Rev. Immunol. 2020, 20, 529-536, doi: 10.1038/s41577-020-0402-6.
55. Cross, A. R.; de Andrea, C. E.; Villalba-Esparza, M.; Landecho, M. F.; Cerundolo, L.; Weeratunga, P.; Etherington, R. E.; Denney, L.; Ogg, G.; Ho, L. P.; et al. Spatial Transcriptomic Characterization of COVID-19 Pneumonitis Identifies Immune Circuits Related to Tissue Injury. JCI insight 2022, doi: 10.1172/jci.insight. 157837.
56. Izadi, Z.; Brenner, E. J.; Mahil, S. K.; Dand, N.; Yiu, Z. Z. N.; Yates, M.; Ungaro, R. C.; Zhang, X.; Agrawal, M.; Colombel, J. F.; et al. Association Between Tumor Necrosis Factor Inhibitors and the Risk of Hospitalization or Death Among Patients With Immune-Mediated Inflammatory Disease and COVID-19. JAMA Netw. Open 2021, 4, e2129639-e2129639, doi: 10.1001/jamanetworkopen.2021.29639.
57. Hänzelmann, S.; Castelo, R.; Guinney, J. GSVA: Gene Set Variation Analysis for Microarray and RNA-Seq Data. BMC Bioinformatics 2013, 14, 7, doi: 10.1186/1471-2105-14-7.
58. Pedregosa, F.; Varoquaux, G.; Gramfort, A.; Michel, V.; Thirion, B.; Grisel, O.; Blondel, M.; Prettenhofer, P.; Weiss, R.; Dubourg, V.; et al. Scikit-Learn: Machine Learning in {P}ython. J. Mach. Learn. Res. 2011, 12, 2825-2830.
59. Blagus, R.; Lusa, L. SMOTE for High-Dimensional Class-Imbalanced Data. BMC Bioinformatics 2013, 14, 106, doi: 10.1186/1471-2105-14-106.
60. Hunter, J. D. Matplotlib: A 2D Graphics Environment. Comput. Sci. Eng. 2007, 9, 90-95, doi: 10.1109/MCSE.2007.55.
61. Lundberg, S. M.; Lee, S. I. A Unified Approach to Interpreting Model Predictions. In Proceedings of the Advances in Neural Information Processing Systems; Guyon, I., Luxburg, U. Von, Bengio, S., Wallach, H., Fergus, R., Vishwanathan, S., Garnett, R., Eds.; Curran Associates, Inc., 2017; Vol. 30.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

1. A method for obtaining a gene set capable of classifying a patient between a first state and a second state, the method comprising:
a) training a first machine learning model based on a first data set comprising or derived from gene expression measurements of genes within a plurality of gene modules, to classify a plurality of reference patients between the first state and the second state, wherein the gene expression measurements are obtained from a plurality of reference biological samples derived or obtained from the plurality of reference patients;
b) selecting N gene modules from the plurality of gene modules based on feature importance values and/or absolute SHAP values of input features of the first machine learning model for classifying the plurality of reference patients between the first state and the second state, wherein Nis an integer;
c) optionally removing redundant genes from genes within the N gene modules to obtain a first gene set;
d) training a second machine learning model based on a second data set comprising gene expression measurements of genes within the first gene set to classify the plurality of reference patients between the first state and the second state;
e) selecting M genes from the genes within the first gene set based on feature importance values and/or absolute SHAP values of input features of the second machine learning model for classifying the plurality of reference patients between the first state and the second state, wherein M is an integer
wherein the selected M genes form the gene set capable of classifying the patient between the first state and the second state, and wherein a first portion of the plurality of reference patients belong to the first state and a second portion of the plurality of reference patients belong to the second state.

2. The method of claim 1, wherein the plurality of gene modules forms the input features of the first machine learning model.

3. The method of claim 2, wherein for each gene module of the plurality of gene modules, an enrichment value of the gene module in a reference biological sample of the plurality of reference biological samples forms an input feature value of the gene module for the reference biological sample, for the training of the first machine learning model.

4. The method of claim 3, wherein the enrichment value is a GSVA score.

5. The method of claim 1, wherein the N gene modules are the gene modules having the top N feature importance values and/or absolute SHAP values among the input features of the first machine learning model for classifying the plurality of reference patients between the first state and the second state.

6. The method of claim 1, wherein Nis 5 to 15.

7. The method of claim 1, wherein genes in the first gene set forms the input features of the second machine learning model.

8. The method of claim 7, wherein for each gene in the first gene set, an expression value of the gene in a reference biological sample of the plurality of reference biological samples, forms an input feature value of the gene for the reference biological sample, for the training of the second machine learning model.

9. The method of claim 8, wherein the expression value is a log2 expression value.

10. The method of claim 1, wherein the M genes are the genes having the top M feature importance values and/or absolute SHAP values among the input features of the second machine learning model for classifying the plurality of reference patients between the first state and the second state.

11. The method of claim 1, wherein M is 15 to 30.

12. The method of claim 1, wherein the redundant genes have correlation coefficients greater than a threshold value, wherein the correlation coefficients are determined based on the expression values of the genes in the N gene modules, in the plurality of reference biological samples.

13. The method of claim 12, wherein the threshold value is 0.8.

14. The method of claim 1, wherein the plurality of biological samples comprise blood samples, isolated peripheral blood mononuclear cells (PBMCs), tissue biopsy samples, nasal fluids, saliva, urine, stool, or any derivative thereof.

15. The method of claim 1, wherein the first state is having COVID-19 disease, the second state is not having COVID-19 disease, and the plurality of gene modules are the gene modules listed in Table 1.

16. The method of claim 1, wherein the first state is having critical COVID-19 disease, the second state is having non-critical COVID-19 disease, and the plurality of gene modules are the gene modules listed in Table 1.

17. The method of claim 1, wherein the first state is having lupus, the second state is not having lupus, and the plurality of gene modules are the gene modules listed in Table 9.

18. The method of claim 1, wherein the first state is having psoriasis, the second state is not having psoriasis, and the plurality of gene modules are the gene modules listed in Table 9.

19. The method of claim 1, wherein the first state is having atopic dermatitis, the second state is not having atopic dermatitis, and the plurality of gene modules are the gene modules listed in Table 9.

20. The method of claim 1, wherein the first state is having systemic sclerosis, the second state is not having systemic sclerosis, and the plurality of gene modules are the gene modules listed in Table 9.

21.-30. (canceled)
